SEQUENCE LISTING (240 sequences)

SEQ ID NO: 1 - DNA, 2229 bp, Homo sapiens
  source 1..2229: mol_type = unassigned DNA
cttggtgtatatagtaaaggccgggcgccgcacgcagacacacactcgccaggacacaggcgcacacacgctcacgcacaaccagcagcggcggcggcgacagcgactcggtgacagcggcgacggcggctcccaccgcggcgcacccccttcaggcggccggcggcgaggccagaccggagagttccagcacactcgaccaccccgcggcttcctggaggcggcggccgcgattgcgaggccaagtggcatcagagctgagtgatggacgacctgcagtcccagaacctctccatggacatgactgactcccctcctgccttggctaataacagactggagaatggcatggctcagctgatcaccaccgaggcctggaacatcaactccactgacctggtaaagaaggccctggtgaccgtgccagccccatccattctgaacccccctgccgagtctcagagtggcatggctctgaaggtggcggccactgtgttgcagcccctgtgcctcggggagagcccagtggtgatgcccattcacatgcaggtggagggaagctccgcaccagagctcaatccgaatggcaatgccacctacgtcatgaccacccagggccccgtgcaactgcccgtggtgctggagcagcacgtctttcagcacctcaactcccctctggtcctgccgcaggaggccccatgctcctccagtaccatccacaacaacctcttccagggagcggaggaccccgaggcccagccccagctcctggacctgaggatccccagccagccgcaggagcccactttgccatttgaagctgtgctccagaatttgtttccctcccagggcactctcgggcccccaccctgtcagcctcctcctggctatgcccctgtgcccccacagccttttagctcccccttgtcccccctggtcccaccagccaccctcttggtgccgtatcctgtaatcgtccccttgcctgtgccagtccctattcccatccccatcccggtgcctcagagttctgaatccaagttcagctccagtttccccaagccaccatcttccttcggcctgcacccctttaaaggcacccagacccctctggaaaaagatgaactgaagccctttgacatcctccagcctaaggagtacttccagctcagccgccacacggtcattaagatgggaagtgagaacgaggccctggatctctccatgaagtcagtgccctggctcaaggctggtgaagtcagtcccccaatcttccaggaagatgcagccctagacctgtcagtggcagcccaccggaaatccgagcctccccctgagacactgtatgacagtggtgcatcagtggacagctcaggtcacacagtgatggagaaacttcccagtggcatggaaatttcttttgcccctgccacgtcccatgaggccccagccatgatggatagtcacatcagcagcagtgatgctgctaccgagatgctcagccagcccaaccaccccagcggcgaagtcaaggctgaaaataacattgagatggtgggcgagtcccaggcggccaaggtcattgtctctgtcgaagatgctgtgcctaccatattctgtggcaagatcaaaggcctctcaggggtgtccaccaaaaacttctccttcaaaagagaagactccgtgcttcagggctatgacatcaacagccaaggggaagagtccatgggaaatgcagagccccttaggaaacccatcaaaaaccggagcataaagttaaagaaagtgaactcccaggaaatacacatgctcccaatcaaaaaacaacggctggccaccttttttccaagaaagtaaataacggctttttaaaatttgtatgattataatatggggaaaggtgcattggttttataaaaaggcatttaaaacaaattatctttgttaattattttggggagtagttgggaaatggaaaggtgaattggctctagaggccctgtatgctagtatcattttcttttttaatttttgacttttcacaaatgagtaaataagagcaacctatttttcaagcagattgcacattttttgcagctttaatggaatattgggtgaattagaggggtaaaaaaagctattttcattgccacaaagtgctttgatgatgtaatacctaataaagggtaggatgaatatttcacaataaatgtttgtttgcactaaaaa

SEQ ID NO: 2 - DNA, 2198 bp, Mus musculus
  source 1..2198: mol_type = unassigned DNA
atagtaaaggccaggcgctgcacgcagacacacactctcccggacacaggcaaacacgcacacacacacaaccggcagtggcggtagcgacagcggcttggtgacagcagcgacaggggctcccatccgctgtgcacccccttcaggcgcccagcggcgaggccagacccagagagttcccacactagcgtccaccccgtggcttcctggaggcggcggcccggatcccgaggccacgtggcaccggagccaagtgatggatgacctgcagtcccagaatctttccatggacatgactgactcccctcccactttggctaataacagactggagaaccgcatggcccagctgataactaccgaggcctggaacatcaactccactgacctggtaaagaaggccctggtgaccgttccggccccatcaattctgaaccccccagctgagtcccagagtggcatggctctgaaggtagcagccaccgtgctgcagcccctgtgccttggggaaagcccagtggtgatgcccattcacatgcaggtggagggaagctctgcgccagagctcaaccccaatggcaacgctacctacgttatgacgacacaaggccccgtgcagctaccagtggtgttggaacagcatgttttccagcacctcaactcccctctagtcctgccacaggaggccccgtgctcctccaatgctatccacaacaacctcttccagggggctgaggactctgaggcccagcctcagctcctggacctgaggatcccaagtcagccacaggagcccgcattgccttttgaagctgtgctccagaatttattccccacccaaggctctctgggccctccaccctgccagcctcctcctggctatgctccagtgcctccccagccctttaactcccccttgtcccccctagtcccgccagctaccctcttggtgccctatcctgtgattgtccccttgccagttccagtgcccatccccatccctgtcccagtgcctcagagtactgaatccaagttcagccccactttccccaagccgccatcttccttcggcttgcactcctttaaaggcacccagaccactctggaaaaggacgaactgaagcccttagacatcctccagtcaaaggagtatttccagctcagccgccacacagtcatcaagatggggagtgagaatgaggccctggatctgtccatgaagtcagtgccctggctcaaggctggggaagctagccccccagtctttcaagaggatgcagccctagatctgtctttggcagcccaccgaaaagctgaggctcctccagagacattgtataacaacagtgggtcagcagacatccagggtcacaccatactggagaaactttccagtggcatggaaatgccctttgcccctgccaagtgttctgaagcctcagccatgatggagagccacagcagcaacagcaacggcactgagatggtcagtcagcccagccatcctggcagtgagctgaaggctgaaaataacattgagatcttaagtgagtctcaggcggccaaggtcattgtgtcagttgaagacgccgtgcctgccatcttctgtggcaagattaaaggcctctcaggggtatccaccaaaaacttctccttcaaaagagaagactctgtgcttcagggttatgatatcaacagccaaggagaagagtccctgggaaacgccgagccccttaggaaacccatcaaaaaccggagcataaagttaaagaaagtgaactcccaggaaatacacatgcttccaattaaaaaacaacggctggccacctttttcccaagaaagtaaataagagctttaaaacgtttttatgattaagacgtggggaaaggtgcattgttttttttttttttttaaaggcactaagacaaattatctttgttaattattttggggtataggtgggagcagaaaggcaaattagctagtatcattttcttttttaatttttgacttttcacaaatgagtaaataagagcaacctatttttcaagcagattgcacatttttttgcagctttaatggaatattgggtgaatcagaggggtaagagctattttcattgccacaaagtgctttgatgatgtaatacctaataaagggtaggatgaatatttcacaataaatgtttgtttgcact

SEQ ID NO: 3 - protein, 530 aa, Homo sapiens
MDDLQSQNLSMDMTDSPPALANNRLENGMAQLITTEAWNINSTDLVKKALVTVPAPSILNPPAESQSGMALKVAATVLQPLCLGESPVVMPIHMQVEGSSAPELNPNGNATYVMTTQGPVQLPVVLEQHVFQHLNSPLVLPQEAPCSSSTIHNNLFQGAEDPEAQPQLLDLRIPSQPQEPTLPFEAVLQNLFPSQGTLGPPPCQPPPGYAPVPPQPFSSPLSPLVPPATLLVPYPVIVPLPVPVPIPIPIPVPQSSESKFSSSFPKPPSSFGLHPFKGTQTPLEKDELKPFDILQPKEYFQLSRHTVIKMGSENEALDLSMKSVPWLKAGEVSPPIFQEDAALDLSVAAHRKSEPPPETLYDSGASVDSSGHTVMEKLPSGMEISFAPATSHEAPAMMDSHISSSDAATEMLSQPNHPSGEVKAENNIEMVGESQAAKVIVSVEDAVPTIFCGKIKGLSGVSTKNFSFKREDSVLQGYDINSQGEESMGNAEPLRKPIKNRSIKLKKVNSQEIHMLPIKKQRLATFFPRK

SEQ ID NO: 4 - protein, 529 aa, Mus musculus
MDDLQSQNLSMDMTDSPPTLANNRLENRMAQLITTEAWNINSTDLVKKALVTVPAPSILNPPAESQSGMALKVAATVLQPLCLGESPVVMPIHMQVEGSSAPELNPNGNATYVMTTQGPVQLPVVLEQHVFQHLNSPLVLPQEAPCSSNAIHNNLFQGAEDSEAQPQLLDLRIPSQPQEPALPFEAVLQNLFPTQGSLGPPPCQPPPGYAPVPPQPFNSPLSPLVPPATLLVPYPVIVPLPVPVPIPIPVPVPQSTESKFSPTFPKPPSSFGLHSFKGTQTTLEKDELKPLDILQSKEYFQLSRHTVIKMGSENEALDLSMKSVPWLKAGEASPPVFQEDAALDLSLAAHRKAEAPPETLYNNSGSADIQGHTILEKLSSGMEMPFAPAKCSEASAMMESHSSNSNGTEMVSQPSHPGSELKAENNIEILSESQAAKVIVSVEDAVPAIFCGKIKGLSGVSTKNFSFKREDSVLQGYDINSQGEESLGNAEPLRKPIKNRSIKLKKVNSQEIHMLPIKKQRLATFFPRK

SEQ ID NO: 5 - DNA, 5030 bp, Homo sapiens
  source 1..5030: mol_type = unassigned DNA
tttctgcggagggattacgctgacgaaagagacctgcttgcgcgtcgctgttccgtggtccgcgcgagcgtggtcgggagccgctggttcctggggtgacccgcggaggtgggagagggaagggcttccgaagccggcgggggtgccatggaccctctccgccggcgcggccttcacagctgggccgcgccgggcatccgtagtccgctctcccaaagcctcggtggagctgaagctgccacagagtgcatgttcacaaagggtcatcacacacggagctgcccctccctgtctccctagagcccatcttcgaggccaggggcttttctaccaggattctggggtgtttctcctcctttcctccctcccagatcttctcacggtaaggggagcagcgaaagcgcagggactttgcattccacgacccgttctgactagtcaacagccgatctgtccctgctgctctaattccagctgccctgccttgttttaacttcagagaaagggggagttctcatttgataagtttaagcctttgctttcgtaggaaggtcatgtggcttaagggacatcgtgaccgcgtatgctatttctgcctgtgcattctaaatcttgggggcagcatattccagaagtccttttggtcgattggttctgtgtcctaggataacaatttgtagtttctgaccatttctttacagaaaaaccacaattggtattttggactgcggggttttttagtggtctcaaactaaatgattatattctggaataatgctgacaatttggatagggtggtgtggaggaaacaagtctcgtgtagaagaaattatttagtaaaaaggattttagtttttggtacttctgaatgcaaatggccaaggaatccagcagtttgttggggtttctccagacaaaaataggctgaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtttatcctttcatatagttagctaataaaaagcttttgtctacacaccctgcatatcataatgggggtaaagttaagttgagatagttttcatccataactgaacatccaaaatcttgatcagttaagaaatttcacatagcccacttacatttacaaactgaagagtaatcaatctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagactgccatattaaattttttgttgacattagtctcagtgaagactatgaaaatgctggctatagatgtcttttcccatttatctaaatatggactgctcaggaaacgagactttccattacaagtatttttaattaattgggccagcttttcaaacaaagatgccacattcaaaatagggtatattttcctatattacggtttgcccctttataaatccaagtagataggaagaaagaagacaaactttgcatctcagtatgaattattcaatttatttgaatgatttttctttacaaaacaaactcattcattagtcatgtttatctgcttaggagtttagggaacaatttggcaattttgtggttttcgagattatcgttttcttaaagtgccagtattttaaaatagcgttcttgtaattttacacgcttttgtgatggagtgctgttttgttatataatttagacttggattctttccatttgcatttgtttatgtaatttcaggaggaatactgaacatctgagtcctggatgatactaataaactaataattgcagaggttttaaatactagttaaatggctttcacttaagaacttaagattttgttacatatttttaaatcttgtttctaataatacctcttagcagtaccttttaaataagtataagggatggcaaagtttttccctttaaaaatactcactttatgcttataaataggttaatgggctgataaaaggttttgtcaaacattgcaagtattcggtgctatatataaaggaggaaaaactagttttactttcagaatgatttaaacaagatttttaaaaacaagatacatgcaagcgaacagcagggttagtgataggctgcaattgtgtcgaacatcagattttttgttaagaggagcaaatgactcaatctgatttagatggaagtttctactgtatagaaatcaccattaatcaccaacattaataattctgatccatttaaaatgaattctggctcaaggagaatttgtaactttagtaggtacgtcatgacaactaccatttttttaagatgttgagaatgggaacagtttttttagggtttattcttgaccacagatcttaagaaaatggacaaaacccctcttcaatctgaagattagtatggtttggtgttctaacagtatcccctagaagttggatgtctaaaactcaagtaaatggaagtgggaggcaatttagataagtgtaaagccttgtaactgaagatgattttttttagaaagtgtatagaaactattttaatgccaagatagttacagtgctgtggggtttaaagactttgttgacatcaagaaaagactaaatctataattaattgggccaacttttaaaatgaagatgctttttaaaactaatgaactaagatgtataaatcttagtttttttgtattttaaagataggcatatggcatattgattaacgagtcaaatttcctaactttgctgtgcaaaggttgagagctattgctgattagttaccacagttctgatgatcgtcccatcacagtgttgttaatgtttgctgtatttattaattttcttaaagtgaaatctgaaaaatgaaatttgtgtgtcctgtgtacccgaggggtaatgattaaatgataaagataagaaaagcgcccatgtaacacaaactgccattcaacaggtatttcccttactacctaaggaattgtaaccattgctcagacattgtaggatttaactatgttgaaaactacaggagaggccgggcgcagtggctcacgcctgtaatcccagcactttgggaggccaaggcgggcagatcacgaggtcaggagattgagaccatcctggctaacgtggtgaaaccccgcctctactaaaaatacaaaaaattagccaagcgtggtgctgggcgcctgtagtcccagtaactcaggaggctgaggcaggagaatggcgtgaacccgggaggcggaggttgcagtgagccgagattgtgccactgcactccagcctgggtgacagagcaagactccatctcaaaaaaaaaaaaaaaacacaggagagacaactggtttttgaatgaaatacatgggtactgccttgcttgacatcacatagtccttgatgaaagttcacatttaggtctgcttggtacaatacgcctcctaaaaaggtccttgatgaaagttcacatttaggtctgcttggtacaacacgcctcctgaaagggtctgatagctttcagtagcagtaagacacttgcatgtgatggtaaggtatctgcaaatttgcacacaccgtacacagcttaagtcttagaattaacttgctaaaatgtgagcctttggtaattaggctgttttattagggagtgtgataatatttgaatttcttttcatatttgtgctttgtgtcattttcaaatgacccttgaaatgtattttaaaagtagataaaagccagaaagtga

SEQ ID NO: 6 - DNA, 2855 bp, Mus musculus
  source 1..2855: mol_type = unassigned DNA
cgcgctggctggagagtaatgttacagagcggagagagtgaggaggctgcgtctggctcccgctctcacagccattgcagtacattgagctccatagagacagcgccggggcaagcgcgagccggacgggcactgggcgactctgtgcctcgcggaggaaaatcaactaaacatgggcaaaggagatcctaaaaagccgagaggcaaaatgtcctcatatgcattctttgtgcaaacttgccgggaggagcacaagaagaagcacccggatgcttctgtcaacttctcagagttctccaagaagtgctcagagaggtggaagaccatgtctgctaaagaaaaggggaaatttgaagatatggcaaaggctgacaaggctcgttatgaaagagaaatgaaaacctacatcccccccaaaggggagaccaaaaagaagttcaaggaccccaatgcacccaagaggcctccttcggccttcttcttgttctgttctgagtaccgccccaaaatcaaaggcgagcatcctggcttatccattggtgatgttgcaaagaaactaggagagatgtggaacaacactgcagcagatgacaagcagccctatgagaagaaagctgccaagctgaaggagaagtatgagaaggatattgctgcctacagagctaaaggaaaacctgatgcagcgaaaaagggggtggtcaaggctgaaaagagcaagaaaaagaaggaagaggaagatgatgaggaggatgaagaggatgaggaagaggaggaagaagaggaagacgaagatgaagaagaagatgatgatgatgaataagttggttctagcgcagtttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtctgggggttgtaaattggcatggaaatttaaagcaggttcttgttggtgcacagcacaaattagttatatatggggacagtagtttggttttttgtttttttttttttttcttttggttttctttttgggttttatttttttcatcttcagttgtctctgatgcagcttatacgaagataattgttgttctgttaactgaataccactctgtaattgcaaaaaaaaaattgcggctgttttgttgacattctgaatgcttctaagtaaatacaattttttttattagtattgttgtccttttcataggtctgaaagttttcttctcaaggggaagctagtcttttgctttgcccattttgggtcacatggattattagtgtgttatctttcatctagttagctggaagagagcttttgtccacatgccctgccattgtggtagggtaacattttcatccatagttgaagaatctcctaaatcgtgatagttggataagagatattatataacctacttggcaaagcaaggagtgatcaatactgtcacaccgtgggactattaggatcaagcaatctgaacgtctgtccttgaaggactgatagaaaagtaccttctaatccttacacgaggactctcctttaaccgccattactgtgtaatgacagttatattttgcagtttcccctactaaagaagacctgagaatgtatccccaaaagtgtgagcttaaaatacaagactgctgtactatttgttgaccttagtcccagcgaaggctatcacaagaacgctggctgtaaagcctttgcccttctatctagatatggattgctcaggaaacttgactgtttaaaggtatttttaattacttgagccagcttttaaaattatgccacatttaaaatgaagggtatattttcctatactgtggtttgtccctttatgaatcagatacaagaggataaactttgcatattagtaccatttgtccaatacatttgctttttctttataaaacccaaactcattcattaatcaggtttaatctgcttagtttagggaacaatttggcaattttgtggatttttttttgagattatcgttctcttaaagtgccagtgttttaaatagcgttcttgtaatttcacgcgcttttgtgatggagtgctgttatataattttgacttgggttctttacatttgcgttgttaatgtaatttgaggaggaatactgaacatgagtcctggatgatactaataaactaataattacagaggttttaaatattagttaaatgactttcacttaagaatttaagcttttggtcacactttataatagtgccttatagtataaacaactgaaaggctctttcccattaacaacccttgatgctggggccagtgagatagtgggtaaaaaggcagttggctgccaaccctgacaaccgatggcaaaaggagggaaccagcttccaaaatgctttgaccaaatgctccctccattcatgaacacagttttaaaatgttaaataggctagagggcagtaaaaacaggtttttttatcgagcatccctaatctatacatatgaggagccataatctgaatgttaagtgaaaagcgaggttggtcttaaagattgcacgtgtgttcttaagcctgtagaggacctccgcaggccgtaatggtctcgattaccaacttaagaacaagtgactggcttggaaacttgtactgttgctttagaactaccattgtggacatctgttgttagtaagtgatccatttaaaagtgaactctgcctcaa

SEQ ID NO: 7 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 8 - protein, 215 aa, Mus musculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 9 - DNA, 4198 bp, Homo sapiens
  source 1..4198: mol_type = unassigned DNA
cggccgaggggcgcccgagccgccgcggcggtcgcctggaaaagtttccccgccagggctccccaggggtcggcactcttcaccctcccgagccctgcccgtctcggccccatgcccccaccagtcagccccgggccacaggcagtgagcaggcacctgggagccgaggccctgtgaccaggccaaggagacgggcgctccagggtcccagccacctgtcccccccatggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaacgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcatgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccctccagggttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctgatgagcccactgcccaccccctcaagccaatcccctccttgggcactttttaattcaccaaagtatttttttatcttgggactgggtttggaccccagctgggaggcaagaggggtggagactgcttcccatcctaccctcgggcccacctggccacctgaggtgggcccaggaccagctggggcgtggggagggccgtaccccaccctcagcaccccttccatgtggagaaaggagtgaaacctttagggcagcttgccccggccccggccccagccagagttcctgcggagtgaagaggggcagcccttgcttgttgggattcctgacccaggccgcagctcttgcccttccctgtccctctaaagcaataatggtcccatccaggcagtcgggggctggcctaggagatatctgagggaggaggccacctctccaagggcttctgcaccctccaccctgtcccccagctctggtgagtcttggcggcagcagccatcataggaagggaccaaggcaaggcaggtgcctccaggtgtgcacgtggcatgtggcctgtggcctgtgtcccatgacccacccctgtgctccgtgcctccaccaccactggccaccaggctggcgcagccaaggccgaagctctggctgaaccctgtgctggtgtcctgaccaccctcccctctcttgcacccgcctctcccgtcagggcccaagtccctgttttctgagcccgggctgcctgggctgttggcactcacagacctggagcccctgggtgggtggtggggaggggcgctggcccagccggcctctctggcctcccacccgatgctgctttcccctgtggggatctcaggggctgtttgaggatatattttcactttgtgattatttcactttagatgctgatgatttgtttttgtatttttaatgggggtagcagctggactacccacgttctcacacccaccgtccgccctgctcctccctggctgccctggccctgaggtgtgggggctgcagcatgttgctgaggagtgaggaatagttgagccccaagtcctgaagaggcgggccagccaggcgggctcaaggaaagggggtcccagtgggaggggcaggctgacatctgtgtttcaagtggggctcgccatgccgggggttcataggtcactggctctccaagtgccagaggtgggcaggtggtggcactgagcccccccaacactgtgccctggtggagaaagcactgacctgtcatgcccccctcaaacctcctcttctgacgtgccttttgcacccctcccattaggacaatcagtcccctcccatctgggagtccccttttcttttctaccctagccattcctggtacccagccatctgcccaggggtgccccctcctctcccatccccctgccctcgtggccagcccggctggttttgtaagatgctgggttggtgcacagtgatttttttcttgtaatttaaacaggcccagcattgctggttctatttaatggacatgagataatgttagaggttttaaagtgattaaacgtgcagactatgcaaaccaggaaaaaaa

SEQ ID NO: 10 - DNA, 4171 bp, Mus musculus
  source 1..4171: mol_type = unassigned DNA
ggtttagccagaactggaaagctactgcctgacgtgacagggtcctcaccggggcaccagcagccaccagggaagcagaggcaggcaagatggatcttggattctctggagctagatgatctggcccttaccctgtcctccagccctgcccatctcggccccatgcccccaccagtcagccccgggaccctggcactgagcaggcacgtggcagccaaggccctgtgaccaggccagggagacaggcgctctggggttccagtcacctgcccccccatggagctgagatcctggttgctatgggtggtcgcagcagcaggagccgtggtcctgctggcagctgatgctcaaggccagaagatcttcaccaacacctgggccgtgcacattcctggaggcccagctgtggctgatagggtggcgcagaagcatggcttccacaacctgggccagatcttcggtgactattaccacttctggcacagagcagtgacaaagcggtccctgtcgcctcaccgcccgcggcacagccggctacagagggagcctcaagtaaagtggctggagcagcaggtagccaagcgaagagccaagagggacgtgtatcaggagcccacggaccccaagttcccccagcagtggtacctgtctggtgtcactcagcgagacctgaatgtgaaggaggcctgggcccagggcttcacaggccatggcattgtggtctccatcctggatgacggcattgagaagaatcatcccgacctagcaggcaattatgaccctggagccagttttgacgtgaatgaccaggaccccgacccacagcctcggtacacacagatgaatgacaacaggcatggcactcgctgtgccggggaagtggcagcagtggccaacaatggtgtctgtggcgtaggtgtagcttacaatgcccgaattggaggggtgcggatgttggatggcgaggtgactgatgcagtagaggcacgttcgctgggcctgaatcccaaccacatccacatctacagcgccagctggggccctgaggacgacggcaagaccgtggatggaccagcccggctcgctgaggaggccttctttcggggagttagccagggccgaggagggctgggctccatctttgtctgggcctcagggaatgggggccgggaacatgacagctgcaactgtgacggctacaccaacagcatctatacactgtccatcagcagcgccacacagttcggcaatgtgccctggtacagtgaggcctgctcctccacactggccaccacctacagcagcggcaaccagaatgagaagcagatcgtgacaactgacctgaggcagaagtgcacagaatctcacacaggcacctcggcttctgcccccttggcagctggtatcattgctctcaccctggaggccaacaagaacctcacctggcgggacatgcagcacctggtagtgcagacctcaaagccagcccacctcaacgctgatgattgggctaccaacggcgtgggccggaaagtgagccattcgtatggctacgggctgttggatgcaggtgccatggtggctctggcccagaactggacaacagtggccccccagcggaagtgcattgttgaaatcctggtggaacccaaggacatcggcaaacggctagaggtgcgcaaggcggtgacagcatgcctgggtgagcccaaccacatcaccaggctggaacacgttcaggcccggctcaccctgtcttacaatcgccgcggtgacctggctatccacctcatcagccccatgggcacccgctccaccctgttagctgccagaccacatgactactctgctgatgggtttaatgattgggctttcatgacaacccattcctgggatgaggatccggctggcgaatgggtcctagagattgaaaataccagtgaagccaacaactatgggacgctgaccaagttcactctcgttctgtatggcacagcccccgaggggctctctacacctccagaaagcagcggctgcaagaccctcacatccagccaggcctgcgtggtgtgtgaggaaggttactctctgcaccagaaaagctgtgtccagcactgcccaccaggcttcatcccccaagtccttgatacacactacagcactgagaatgatgtggagatcatccgtgccagcgtctgtaccccctgccacgcctcatgtgccacttgccagggcccggcccccacagactgcctcagctgccccagccatgcctccctggacccagtggagcagacctgctcccggcaaagccagagcagccgggagtctcggcctcagcagcagcctcctgcactgcgccctgaggtggagatggagccccggctgcaggctgggttggcctctcacctgcccgaggtcctggccggcctcagctgccttatcatcgtgctcatctttggcatcgtcttcctgttcctgcatcggtgttcgggcttcagcttccggggagtgaaagtgtacaccatggaccgcggcctcatctcctacaaggggctgccccctgaagcctggcaggaggagtgcccatcggactcagaggaggacgagggccggggcgagaggaccgccttcatcaaagaccagagcgccctttgaaaagcccactgcccaccctatcaaatcgatcccttcctcgggcactttttaattcaccaaagtatttttttatcttgcgactggatttggaccctagctgggaggcgagaggggcagagactgcttcttaccccacccttgggcctacctgtttgcctgaggtgggatcccaggagcctcacccctaccacccctccatgtggaggaaagaggaaatctttagggcagcttccttggcccagctggacttcccgcagagtgaagaggggcggcctctctctgagtccctgaccaggctgcagccctgccccttcccttccctgcccctcagaaagcaataatggttcccatccaggcaacagggaggctggccaggaggtacttgaagaaggcagccacctctccaagggcttttgcatcccctgaccctctctggctcacaggtgagcctccgaagaagctgcagtcttaggaagggaccaaagcaaagcaggcccctcccagtgtgcctgtactttcttgctctcagcctgcctctgcactccaggcctccacctccgtggaagccaggctgagctaggccagcacgggccggccgagctaaagctctgggggccttgctggtgtctgaccatcctttatggcaccctccccccttgccagggccctgttttctgagcccggggctgcctgggctgttggcagttacagtacaggagcccctgggtgggtggtggggaggcacggtggcccagccttctcctcccgcctcccacccgatgctgctttcccctgtggggatctcaggagctgtttgaggatatattttcactttgtgattattcactttagatgctgattcttttttttttttttttttgtatttttaatgggggtagcagctggacccttgaccttctcatgcccacacccaccctgcccttcccatggctgcccagccctggggtatagggaataactaagccccatgtcctgaagaggcaggccagcccggtgggctctaagaaagggggttgtggtgggaggggtaggctgacatcatctgtttcaaatggggttgtgccaagggcttatgggtcgctggctctccaagtgccaggggtgggcaggtggcaccaccaagccctttctaacactgtgccctggtggagaaagcattggcctatgatgccccccaaaccccctcttctgatgtgccttttgcacccttcccattaggacaatcagtcccctcccacttgggctcccccccccccattttcttttccttagcccttctcagtacccagcctacctgtccaggggtgccccttgctctcctgcccacccctcacggccatccaggctggttttgtaagatgctgggttggtgcacagtgattttttttttccttgtaatttaaacaggcctggcattgttggttctatttaatggacacgagataatgttagaggttttaaagtgattaaacgtgcagactatgcaaaccaa

SEQ ID NO: 11 - protein, 794 aa, Homo sapiens
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 12 - protein, 793 aa, Mus musculus
MELRSWLLWVVAAAGAVVLLAADAQGQKIFTNTWAVHIPGGPAVADRVAQKHGFHNLGQIFGDYYHFWHRAVTKRSLSPHRPRHSRLQREPQVKWLEQQVAKRRAKRDVYQEPTDPKFPQQWYLSGVTQRDLNVKEAWAQGFTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNADDWATNGVGRKVSHSYGYGLLDAGAMVALAQNWTTVAPQRKCIVEILVEPKDIGKRLEVRKAVTACLGEPNHITRLEHVQARLTLSYNRRGDLAIHLISPMGTRSTLLAARPHDYSADGFNDWAFMTTHSWDEDPAGEWVLEIENTSEANNYGTLTKFTLVLYGTAPEGLSTPPESSGCKTLTSSQACVVCEEGYSLHQKSCVQHCPPGFIPQVLDTHYSTENDVEIIRASVCTPCHASCATCQGPAPTDCLSCPSHASLDPVEQTCSRQSQSSRESRPQQQPPALRPEVEMEPRLQAGLASHLPEVLAGLSCLIIVLIFGIVFLFLHRCSGFSFRGVKVYTMDRGLISYKGLPPEAWQEECPSDSEEDEGRGERTAFIKDQSAL

SEQ ID NO: 13 - DNA, 1478 bp, Homo sapiens
  source 1..1478: mol_type = unassigned DNA
cttcctcggctggatttaaggttgccgctagccgcctgggaatttaagggacccacactaccttcccgaagttgaaggcaagcggtgattgtttgtagacggcgctttgtcatgggacctgtgcggttgggaatattgcttttcctttttttggccgtgcacgaggcttgggctgggatgttgaaggaggaggacgatgacacagaacgcttgcccagcaaatgcgaagtgtgtaagctgctgagcacagagctacaggcggaactgagtcgcaccggtcgatctcgagaggtgctggagctggggcaggtgctggatacaggcaagaggaagagacacgtgccttacagcgtttcagagacaaggctggaagaggccttagagaatttatgtgagcggatcctggactatagtgttcacgctgagcgcaagggctcactgagatatgccaagggtcagagtcagaccatggcaacactgaaaggcctagtgcagaagggggtgaaggtggatctggggatccctctggagctttgggatgagcccagcgtggaggtcacatacctcaagaagcagtgtgagaccatgttggaggagtttgaagacattgtgggagactggtacttccaccatcaggagcagcccctacaaaattttctctgtgaaggtcatgtgctcccagctgctgaaactgcatgtctacaggaaacttggactggaaaggagatcacagatggggaagagaaaacagaaggggaggaagagcaggaggaggaggaggaagaggaggaagaggaagggggagacaagatgaccaagacaggaagccaccccaaacttgaccgagaagatctttgacccttgcctttgagcccccaggaggggaagggatcatggagagccctctaaagcctgcactctccctgctccacagctttcagggtgtgtttatgagtgactccacccaagcttgtagctgttctctcccatctaacctcaggcaagatcctggtgaaacagcatgacatggcttctggggtggagggtgggggtggaggtcctgctcctagagatgaactctatccagccccttaattggcaggtgtatgtgctgacagtactgaaagctttcctctttaactgatcccacccccacccaaaagtcagcagtggcactggagctgtgggctttggggaagtcacttagctccttaaggtctgtttttagacccttccaaggaagaggccagaacggacattctctgcgatctatatacattgcctgtatccaggaggctacacaccagcaaaccgtgaaggagaatgggacactgggtcatggcctggagttgctgataatttaggtgggatagatacttggtctacttaagctcaatgtaacccagagcccaccatatagttttataggtgctcaattttctatatcgctattaaacttttttctttttttctaaaaa

SEQ ID NO: 14 - DNA, 1781 bp, Mus musculus
  source 1..1781: mol_type = unassigned DNA
aaagggcccgctgccttccagaagttggtctgaaggcaaagggtggttgtatgtgtggactgcgttttatcatgggacctgtgcggctggagatactgcttttcatcctggcagcgtacggtgcttgggctgaggcgacaaaagaggaggaagatgacacagaacgtttgcccagcaaatgcgaagtgtgtaagctgctgagcatggagctccaggaagcgttgagtcgaactggccgatcccgagaagtgctggagctgggtcaggtgctagacactggcaagaggaagagacacgtgccttacagcctctctgagacgcggctagaagaggctttggagaatttgtgcgagcggatcctggattacaatgttcatgctgaacgcaagggctcactaagatacgccaagggtcaaagtcagactatggcaacactgaaaggcctggtccagaagggagtgaaagtggatctgggaatccctttggagctgtgggacgagcccagtgtggaggtcacattcctcaagaagcagtgtgagacgatgctggaggagtttgaagatgttgtaggggactggtacttccaccatcaggagcagcccctccagcattttctctgtgagcggcatgtgcttccagcctctgaaactgcttgtctacgggaagcttggactggaaaggagaagatcagtgatgggcaggaggaggcagatgacgaagaggaggaagaagaagaagagataaccaagacttcaggcaatcccaagcatgacccagaagatctttgacccttacttttgaaccccaaggatgggtcgtggagagttttctaaactcttgtgctctcccgtccttccccgcccctaggcttctaatgtcttcttggtatgatctcggctatgatcctggggacccagaatggcaacaggaaaggagaagggtaggggggaagaagcagaagcaagttttgccttcagggttgaaccacatgcagccacttaattgaacaagtatgtggtgacagttttgaaacttttctctacgttttactggtcttcacctgcaccgattggtcaatagtggtgtgagctgtgggaaagtcactttgctttttaaagggtctattttaaaccctttcaagaggccagagaagagcgagcagcgcaattggtagagtgcctgcctagtatggacagtgatgggagttgaaccctgatgtggtggcataccgctgtaatctcagcactcaaagaagcgttggcaggaatatcagaataccatccagctacttggcagttcgggaccaaccagagctgaaccctgtcttaaagtaagaagccaaaaagagaccctcaagaaccaatatacactgcaaacatgaggccgtataccatcaatctgtgaataagaacaggatgctggaagagagtgaagcaatactcgggttagatggggtagacatgtgggctatatttcctatttcacaggtgccatattttctatattgctattaaaccttttatatatatatcagctgagtcccatttcaaacatgcacacattatcgatgagggagcacccagacagtggggccattggcctgctatctattagttgctaatggtgttcttataattatccaccccaaggatactggttggggttgggaaaacaactgtaccactaggacttggaaaagaggatcaagagctcagtcttcctcagctatgtagcgagctcaagaccatcttggtctccttgagcccctgtatcaggaaaat

SEQ ID NO: 15 - protein, 248 aa, Homo sapiens
MGPVRLGILLFLFLAVHEAWAGMLKEEDDDTERLPSKCEVCKLLSTELQAELSRTGRSREVLELGQVLDTGKRKRHVPYSVSETRLEEALENLCERILDYSVHAERKGSLRYAKGQSQTMATLKGLVQKGVKVDLGIPLELWDEPSVEVTYLKKQCETMLEEFEDIVGDWYFHHQEQPLQNFLCEGHVLPAAETACLQETWTGKEITDGEEKTEGEEEQEEEEEEEEEEGGDKMTKTGSHPKLDREDL

SEQ ID NO: 16 - protein, 245 aa, Mus musculus
MCGLRFIMGPVRLEILLFILAAYGAWAEATKEEEDDTERLPSKCEVCKLLSMELQEALSRTGRSREVLELGQVLDTGKRKRHVPYSLSETRLEEALENLCERILDYNVHAERKGSLRYAKGQSQTMATLKGLVQKGVKVDLGIPLELWDEPSVEVTFLKKQCETMLEEFEDVVGDWYFHHQEQPLQHFLCERHVLPASETACLREAWTGKEKISDGQEEADDEEEEEEEEITKTSGNPKHDPEDL

SEQ ID NO: 17 - DNA, 2392 bp, Homo sapiens
  source 1..2392: mol_type = unassigned DNA
tgtgtatctgggtgagtttccaacatcagatagatcttacaggtcccagcctgtagacatcttttactccaatttcctgaatagatagctttattccttcaaggtaatatagtgcggtggcttctggctgagatgtttgctgttgttttcttcatcttgtctttgatgacttgtcagcctggggtaactgcacaggagaaggtgaaccagagagtaagacgggcagctacacccgcagcagttacctgccagctgagcaactggtcagagtggacagattgctttccgtgccaggacaaaaagtaccgacaccggagcctcttgcagccaaacaagtttgggggaaccatctgcagtggtgacatctgggatcaagccagctgctccagttctacaacttgtgtaaggcaagcacagtgtggacaggatttccagtgtaaggagacaggtcgctgcctgaaacgccaccttgtgtgtaatggagaccaggactgccttgatggctctgatgaggacgactgtgaagatgtcagggccattgacgaagactgcagccagtatgaaccaattccaggatcacagaaggcagccttggggtacaatatcctgacccaggaagatgctcagagtgtgtacgatgccagttattatgggggccagtgtgagacggtatacaatggggaatggagggagcttcgatatgactccacctgtgaacgtctctactatggagatgatgagaaatactttcggaaaccctacaactttctgaagtaccactttgaagccctggcagatactggaatctcctcagagttttatgataatgcaaatgaccttctttccaaagttaaaaaagacaagtctgactcatttggagtgaccatcggcataggcccagccggcagccctttattggtgggtgtaggtgtatcccactcacaagacacttcattcttgaacgaattaaacaagtataatgagaagaaattcattttcacaagaatcttcacaaaggtgcagactgcacattttaagatgaggaaggatgacattatgctggatgaaggaatgctgcagtcattaatggagcttccagatcagtacaattatggcatgtatgccaagttcatcaatgactatggcacccattacatcacatctggatccatgggtggcatttatgaatatatcctggtgattgacaaagcaaaaatggaatcccttggtattaccagcagagatatcacgacatgttttggaggctccttgggcattcaatatgaagacaaaataaatgttggtggaggtttatcaggagaccattgtaaaaaatttggaggtggcaaaactgaaagggccaggaaggccatggctgtggaagacattatttctcgggtgcgaggtggcagttctggctggagcggtggcttggcacagaacaggagcaccattacataccgttcctgggggaggtcattaaagtataatcctgttgttatcgattttgagatgcagcctatccacgaggtgctgcggcacacaagcctggggcctctggaggccaagcgccagaacctgcgccgcgccttggaccagtatctgatggaattcaatgcctgccgatgtgggccttgcttcaacaatggggtgcccatcctcgagggcaccagctgcaggtgccagtgccgcctgggtagcttgggtgctgcctgtgagcaaacacagacagaaggagccaaagcagatgggagctggagttgctggagctcctggtctgtatgcagagcaggcatccaggaaaggagaagagagtgtgacaatccagcacctcagaatggaggggcctcgtgtccagggcggaaagtacagacgcaggcttgctgagggcctctggacacaggctggaccagatgctgtggatgtcgacccctgcactgactattggataaagacttctttcaactaagagaagatgcaaatcagcacacttttttctttgttctgccagcttccaggcctaagactaggttttgctgtctacagccaactattctattagttacaaaactcaatcattttattcagcaactggatgttgactgttaactagaagctctgtcctacttacagcactttggatcatcaaaaaaataaagtaaaatagaaaactgagaaaactcaatccatgaccagggagaacttacaggatgttagagacaaaacaagcagacacctgaaacaatcaacgcccaataaaacaaagtaggatgaaaattctcttagttctttgataacaatttgttcactcatagaaacattattaattggtagggtaagcagacactctgaaacaatgagaaaaatactaaaaattgacttgagttatttc

SEQ ID NO: 18 - DNA, 3498 bp, Mus musculus
  source 1..3498: mol_type = unassigned DNA
gaactgggggacttttgaccattttaaacatccggtagacaatttgggtcccagcctggatatatctatggcttctaagtaccgataccggagcctcttgcagccaagcaagtttggaggaaccatctgcagtggtgatatctgggatgaggccagctgtgacagccccacaccttgtctaaggcaagcacagtgtggacaggatttccagtgtagagagacaggtcgctgcctgaaacgccacctggtgtgtaacggagataacgactgcctggatggctctgatgagtccgactgtgaagatgtcagggtcactgaggatgattgccatcagtatgaaccaatcccaggatcagaaagggcagccttggggtacaacatcctgactcaagaggaagctcagagtgtttacgatgctaagtattatgggggccagtgtgaaactgtgtacaatggagactggagaaagctgcggtacgaccccacctgtgagcgcctctactatggggaggatgagaaatactttcggaagccctacaacttccttaagtaccactttgaagccctggcggatacttcaatctcctcagagttttatgatgatgcgaatgatcttttttttcacataaagaatggcaagtcccactcagctggtgtaaccgttggcgtagccccagtaaaaagtcctgtctcaatagaggtgactggatccgggtccaaagcatcttcattcctgaacaagttaaacaagtataatgagaagagatatggcttcatgagagtgtctacaaagatacagactgcacagtttaagatgagaaggaacaatattgtgctggatgaaggaatgctgcagtcattaatggagctcccagagcagttcaactatggcatgtacgccaagttcatcaatgactatggcacccactacatcacctctggaaccatgggtggcatttatgaatatgttatggtgcttgacaaagagaaaatgaaaactgaaggtactactgtggatgaagtccaaaaatgcattggcggtggcattggcataggaattaaggatagtacaattgaaggagtaggtatatcaggagagttttgtgaaaactctggagatggtgaccgtgacataaggaagaaaatcacaggagtggaggacatcatttctcgggtgcaaggtggaagttctgtctggggcagtgtcttgacacacaacagcagtgccattacatatcagtcctgggggagatcgttaaagtataaccctgttgttatcgattttgagatgcagcctatctaccagctactgcggcacacaaacttgggacccctggagacgaaacgtcagaacctgcgacgggctttggaccagtacctgatggaatttaatgcctgtcgttgtgggccctgcttcaacaatggggaacccatacttgacggcaccaactgccggtgtcagtgtagtatgggccggcagggcctggcctgtgaacgcacggtgatagaggggctcaaagacttcaaagccgctggccactggagctgctggagttcctggtcagagtgcagaggaggttcccaggagaggaggagacagtgcaacaacccaccacccaagaatggaggtaccccatgcctagggaggaacctgcagacccaggcctgctgagtacctctggatgcagactgggcaaggaggctgcagatgctgttacctgtctgggttgctggttaggactgcttgctgctctgagtaaatgcaaacccacaaaggctattctcttaatatactcatgttttgttttgttttttctagtcataattcttagcctctatcaataacctggctctaaagctgcctctttgttccagggagttgttgccctttacttcccagaaccacctttctatcctgttctattcaggatgcttaacagtaaacgggctatggtttagatacatcagaatgtattcctcacaattctggaagcatatactgagagatgaaggttccagaaggtctagggtctggagaggctgtgttccattttatatatgtccttttgttatgttatcacatggcagaggggcctctagggcctcttttgtaagggcactagtcctactcatgagaactttgcctttatgactttgtcatctctcaaagtccagcctcctctttccacactgggtagagggcttccatgtggagaacttagaaatcccagcattcagaccccagaccatcccctgtgattctcttcttggtaagcaatcaaatttgaagacagaaagtggcctggttttattcagcacagtatcttcagatgctgaggctacgcactgcagactgtaggggagttcaacagcaatggttgctggacaagcagacagaccaagagtcagttcatgggtgaggtctgtattggccaacctcccagaactgtaggtggacttggaagggtcaatcaataaagatcaacgtccattccctgaccttcccgaaaagagccagtcacaccagccaacctagtgtaccagtgcactctaaataatgtacatgttcagcacagttgccaagcgacctaattaaggtttctgttgctatgagaaaacatcacaagcaaaagcaactggggagaaaaggaataacttcagcttgcatgcctcactcacagtctatcatgaagggaagtcagggcaggaattcaaggcaggaacctggaggcaggaactgaagcagcgatcatggaggggcactgctggccaacttcctcctcatggttttctcagcctgctctcttggatcacccaataccacctgcccagggatagcaacacccagagtgagctgagtcctccaacatcaatcactaatttttaaaaaaatcataatacagacttgctcagaggccagtctagtgttttttgttttgtttgttttttttccttaattgaagttcccccttccaaaatgtgtcacattgacatacaactagataaatcaacagcccaggtttatccctggcttctcttcaaagttgatagactatgtagatatgaagccttcagaaacagataaccaactgtatacacaaaaaagaggaggctcttgggccactgggagtaaattattaacatcaacatatgtagcatccaagtgactgctgagggaggacaagtcagaaaatgcagtctgcacacgttatatatacatcagaggcaacaagcacggtgcctatctgaacctggatggactttttaacagatcaacatggaggagacttttgaaatagaaatccagcctgatgcaaaacctgacagtgagaaagaacgtggggactgggaaaaatgacgaaagacaaagaaagacggtgtatgaaacgtgttccagtgcctaccacatggcaaattattagcatcatcattgttattcccattgttaaaacgtgattcagaggtttacctaaataaacttggctcttctatctgaaa

SEQ ID NO: 19 - protein, 584 aa, Homo sapiens
MFAVVFFILSLMTCQPGVTAQEKVNQRVRRAATPAAVTCQLSNWSEWTDCFPCQDKKYRHRSLLQPNKFGGTICSGDIWDQASCSSSTTCVRQAQCGQDFQCKETGRCLKRHLVCNGDQDCLDGSDEDDCEDVRAIDEDCSQYEPIPGSQKAALGYNILTQEDAQSVYDASYYGGQCETVYNGEWRELRYDSTCERLYYGDDEKYFRKPYNFLKYHFEALADTGISSEFYDNANDLLSKVKKDKSDSFGVTIGIGPAGSPLLVGVGVSHSQDTSFLNELNKYNEKKFIFTRIFTKVQTAHFKMRKDDIMLDEGMLQSLMELPDQYNYGMYAKFINDYGTHYITSGSMGGIYEYILVIDKAKMESLGITSRDITTCFGGSLGIQYEDKINVGGGLSGDHCKKFGGGKTERARKAMAVEDIISRVRGGSSGWSGGLAQNRSTITYRSWGRSLKYNPVVIDFEMQPIHEVLRHTSLGPLEAKRQNLRRALDQYLMEFNACRCGPCFNNGVPILEGTSCRCQCRLGSLGAACEQTQTEGAKADGSWSCWSSWSVCRAGIQERRRECDNPAPQNGGASCPGRKVQTQAC

SEQ ID NO: 20 - protein, 534 aa, Mus musculus
MASKYRYRSLLQPSKFGGTICSGDIWDEASCDSPTPCLRQAQCGQDFQCRETGRCLKRHLVCNGDNDCLDGSDESDCEDVRVTEDDCHQYEPIPGSERAALGYNILTQEEAQSVYDAKYYGGQCETVYNGDWRKLRYDPTCERLYYGEDEKYFRKPYNFLKYHFEALADTSISSEFYDDANDLFFHIKNGKSHSAGVTVGVAPVKSPVSIEVTGSGSKASSFLNKLNKYNEKRYGFMRVSTKIQTAQFKMRRNNIVLDEGMLQSLMELPEQFNYGMYAKFINDYGTHYITSGTMGGIYEYVMVLDKEKMKTEGTTVDEVQKCIGGGIGIGIKDSTIEGVGISGEFCENSGDGDRDIRKKITGVEDIISRVQGGSSVWGSVLTHNSSAITYQSWGRSLKYNPVVIDFEMQPIYQLLRHTNLGPLETKRQNLRRALDQYLMEFNACRCGPCFNNGEPILDGTNCRCQCSMGRQGLACERTVIEGLKDFKAAGHWSCWSSWSECRGGSQERRRQCNNPPPKNGGTPCLGRNLQTQAC

SEQ ID NO: 21 - DNA, 2016 bp, Homo sapiens
  source 1..2016: mol_type = unassigned DNA
acacttgtggcttccgtgcacacattaacaactcatggttctagctcccagtcgccaagcgttgccaaggcgttgagagatcatctgggaagtcttttacccagaattgctttgattcaggccagctggtttttcctgcggtgattcggaaattcgcgaattcctctggtcctcatccaggtgcgcgggaagcaggtgcccaggagagaggggataatgaagattccatgctgatgatcccaaagattgaacctgcagaccaagcgcaaagtagaaactgaaagtacactgctggcggatcctacggaagttatggaaaaggcaaagcgcagagccacgccgtagtgtgtgccgccccccttgggatggatgaaactgcagtcgcggcgtgggtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactgaaaaatatggagtggcaatatagaaacacgaactttagctgcatcctccaagaatctatctgcttatgcagtttttcagagtggaatgcttcctagaagttactgaatgcaccatggtcaaaacggattagggcatttgagaaatgcatattgtattactagaagatgaatacaaacaatggaaactgaatgctccagtcaacaaactatttcttatatatgtgaacatttatcaatcagtataattctgtactgatttttgtaagacaatccatgtaaggtatcagttgcaataatacttctcaaacctgtttaaatatttcaagacattaaatctatgaagtatataatggtttcaaagattcaaaattgacattgctttactgtcaaaataattttatggctcactatgaatctattatactgtattaagagtgaaaattgtcttcttctgtgctggagatgttttagagttaacaatgatatatggataatgccggtgagaataagagagtcataaaccttaagtaagcaacagcataacaaggtccaagatacctaaaagagatttcaagagatttaattaatcatgaatgtgtaacacagtgccttcaataaatggtatagcaaatgttttgacatgaaaaaaggacaatttcaaaaaaataaaataaaataaaaataaattcacctagtctaaggatgctaaaccttagtactgagttacattgtcatttatatagattataacttgtctaaataagtttgcaatttgggagatatatttttaagataataatatatgtttaccttttaattaatgaaatatctgtatttaattttgacactatatctgtatataaaatattttcatacagcattacaaattgcttactttggaatacatttctcctttgataaaataaatgagctatgtattaa

SEQ ID NO: 22 - DNA, 2857 bp, Mus musculus
  source 1..2857: mol_type = unassigned DNA
gcagaagcagcgacgcctcctcagtggaactgacttgcaatagcgagctttctctgctgcacatttgtggcttctgtggacatattagtaaccagcggttttagctcccagtctcacagagttgccagagaggttagaagtcatttgaaaagcctattagccgaatctttctgatccagaaggccagctggcttctcctgagctactttcaattcgcagcaaccactgatcctggtccaggtgactggggaagacgctgagggtataaacccaaacattgaacctgaagacccagcgcaaagtagaaactgaaagtaccctgcttagtctgccggcagatcctacggaagttatggcaaagccagagcgcctgggtggccggtgatgcatgcggcccctcttgggatggatggaccaggcgtggcgtgggtgagaggagtcagctgcctgaactgccctgcccagcaccggtttgcggccacccggtggatgaccggggtcctgggagtgattatgggtggtgagagccggctcctgctgcagtcccagtcatcatgactacacccacctcccgcagaccatgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtatataaacaggacatgtagtaacaacctccaagaatctactggttcatatacttggagaggttgaaacccttccagaagttcctggatgcctcctgctcaaataagccaagcagctgagaaatctacagtgaggtatgagatgatggacacagaaatgcagctgactgctgccgtcagcatatacatataaagatatatcaactatacagatttttgtaatgcaatcatgtcaactgcaatgcttttaaaaccgttccaaatgtttctaacactacaaagtctacaaaaagcaaggctatgaagattcagagtcaccactgttttcttagcaaaatgatggtatggttaaacattcattggtgaaccactgggggagtggaactgtcctgttttagactggagatactggagggctcacggtgatggataatgctcttgaaaacaagagtctatcttaaagcagcagcaaaaagaagcttaaggcacttaaggcatcaacaaatgtagttaaatatgaatgtataacacataacttcagtaaagagcatagcagatatttttaaataaaagtatttttaaagatagaaatgcacttattccaaagatactgaaccttagtattcagtcgcttttgacacttgtgtataataaagcttatataactgaattttcaatttgaaaagtatatttttaaaagaataatatatgctagacttttaattaatgtatatgtttaattttggcattctgtctgtctctctgtctctctctctctctctctctctctctacctatctatctatatatataattttcatatactaccaattgcgtactttggatagtgtctctttttaacctaaatgacctttattaacactgtcaggttcccttactctcgagagtgttcattgctgcactgtcatttgatcccagttttattgaacacatatcctttaacacactcacgtccagatttagcaggagactaggaccctataactttgttaagagagaaaacactaatttcttgttttatagtagggtcttattcgtatctaaggcaggctaggattgcagacatgagccaatatgcttaattagaaacattctttttatgttaaactcatgtcttttacaagatgcctacatatatcctatgtatatgcctgtttaaatccttttttgtaaggtctgctgtcttccttcagttgtaatggaaagaaacactatgttgtagaggccaaatttctgaaagtgataagggtttgcttgtactgaattctcattctccttgctttttccagccacgtgagcatctagctatctatacgctggatgtatttgaccgatgcctgctccactggcacattgcatgtgtggtagccatgccttcttgcttctccttttccccaacccctataatgctctactcagtggtacagatagctgggattatcacaattttgagagaaacaccaattgtttaaagtttgtttcataatcaccatttgcccagaaaacagttctctcaacttgtttgcaacatgtaataatttaagaaactcaattttgttaatggactttcgataacttccttagatatcccacatctcctacgtgtcagtcctttgtcctgaggaactggtaaaatgggtaagcccttagctagcgaactgaaggcattcgcatgtgtaagataatctctatacctgcaaggctgtctggatggctccctaccaatattgaacaatattctgattttggcaaaataaaggataatattttaaaatgcttattaaaagttttaaaaattt

SEQ ID NO: 23 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 24 - protein, 113 aa, Mus musculus
MISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 25 - DNA, 2996 bp, Homo sapiens
  source 1..2996: mol_type = unassigned DNA
gcgcacacccgcgcacccgcggccgcaggagggcccagcgacgccgccgcgccagctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacggagttagacgaa

SEQ ID NO: 26 - DNA, 2791 bp, Mus musculus
  source 1..2791: mol_type = unassigned DNA
agagggcccagcgccactgcagccgcgccacctcccagggccgggccagcccaggcgtccgcgctctcggggtggactccccccgctgcgcgctcaagccggcgatggctcctctcggatacctcttagtgctctgcagcctgaagcaggctctgggcagctacccgatctggtggtccttggctgtgggaccccagtactcctctctgagcactcagcccattctctgtgccagcatcccaggcctggtaccgaagcagctgcgcttctgcaggaactacgtggagatcatgcccagcgtggctgagggtgtcaaagcgggcatccaggagtgccagcaccagttccgaggccggcgttggaactgcaccaccgtcagcaacagcctggccatctttggccctgttctggacaaagccacccgggagtcagcctttgtccatgccatcgcctccgctggagtagctttcgcagtgacacgctcctgtgcagagggatcagctgctatctgtgggtgcagcagccgcctccagggctccccaggcgagggctggaagtggggcggctgtagtgaggacattgaatttggaggaatggtctctcgggagtttgccgatgccagggagaaccggccggatgcccgctctgccatgaaccgtcacaacaatgaggctgggcgccaggccatcgccagtcacatgcacctcaagtgcaaatgccacgggctatctggcagctgtgaagtgaagacctgctggtggtcgcagccggacttccgcaccatcggggatttcctcaaggacaagtatgacagtgcctcggagatggtggtagagaaacaccgagagtctcgtggctgggtggagaccctgaggccacgttacacgtacttcaaggtgccgacagaacgcgacctggtctactacgaggcctcacccaacttctgcgaacctaaccccgaaaccggctccttcgggacgcgtgaccgcacctgcaatgtgagctcgcatggcatagatgggtgcgacctgttgtgctgcgggcgcgggcataacgcgcgcactgagcgacggagggagaaatgccactgtgttttccattggtgctgctacgtcagctgccaggagtgcacacgtgtctatgacgtgcacacctgcaagtaggagagctcctaacacgggagcagggttcattccgaggggcaaggttcctacctgggggcggggttcctacttggaggggtctcttacttggggactcggttcttacttgagggcggagatcctacctgtgagggtctcatacctaaggacccggtttctgccttcagcctgggctcctatttgggatctgggttcctttttaggggagaagctcctgtctgggatacgggtttctgcccgagggtggggctccacttggggatggaattccaatttgggccggaagtcctacctcaatggcttggactcctctcttgacccgacagggctcaaatggagacaggtaagctactccctcaactaggtggggttcgtgcggatgggtgggaggggagagattagggtccctcctcccagaggcactgctctatctagatacatgagagggtgcttcagggtgggccctatttgggcttgaggatcccgtgggggcggggcttcaccccgactgggtggaacttttggagacccccttccactggggcaaggcttcactgaagactcatgggatggagctccacggaaggaggagttcctgagcgagcctgggctctgagcaggccatccagctcccatctggcccctttccagtcctggtgtaaggttcaacctgcaagcctcatctgcgcagagcaggatctcctggcagaatgaggcatggagaagaactcaggggtgataccaagacctaacaaaccccgtgcctgggtacctcttttaaagctctgcaccccttcttcaagggctttcctagtctccttggcagagctttcctgaggaagatttgcagtcccccagagttcaagtgaacacccatagaacagaacagactctatcctgagtagagagggttctctaggaatctctatggggactgctaggaaggatcctgggcatgacagcctcgtatgatagcctgcatccgctctgacacttaatactcagatctcccgggaaacccagctcatccggtccgtgatgtccatgccccaaatgcctcagagatgttgcctcactttgagttgtatgaacttcggagacatggggacacagtcaagccgcagagccagggttgtttcaggacccatctgattccccagagcctgctgttgaggcaatggtcaccagatccgttggccaccaccctgtcccgagcttctctagtgtctgtctggcctggaagtgaggtgctacatacagcccatctgccacaagagcttcctgattggtaccactgtgaaccgtccctcccccctccagacaggggaggggatgtggccatacaggagtgtgcctggagagcgcggaaagaggaagagaggctgcacacgcgtggtgactgactgtcttctgcctggaactttgcgttcgcgcttgtaactttattttcaatgctgctatatccacccaccactggatttagacaaaagtgattttctttttttttttttcttttctttctatgaaagaaattattttagtttatagtatgtttgtttcaaataatggggaaagtaaaaagagagaaaaaaaaaaaaaaaaaaaaaaaaaaaaaaaaaaaaaaaaaa

SEQ ID NO: 27 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 28 - protein, 352 aa, Mus musculus
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 29 - DNA, 1919 bp, Homo sapiens
  source 1..1919: mol_type = unassigned DNA
atgctcgccgcctccatcttccgtccgacactgctgctctgctggctggctgctccctggcccacccagcccgagagtctcttccacagccgggaccgctcggacctggagccgtccccactgcgccaggccaagcccattgccgacctccacgctgctcagcggttcctgtccagatacggctggtcaggggtgtgggcggcctgggggcccagtcccgaggggccgccggagacccccaagggcgccgccctggccgaggcggtgcgcaggttccagcgggcgaacgcgctgccggccagcggggagctggacgcggccaccctagcggccatgaaccggccgcgctgcggggtcccggacatgcgcccaccgcccccctccgccccgccttcgcccccgggcccgccccccagagcccgctccaggcgctccccgcgggcgccgctgtccttgtcccggcggggttggcagccccggggctaccccgacggcggagctgcccaggccttctccaagaggacgctgagctggcggctgctgggcgaggccctgagcagccaactgtccgtggccgaccagcggcgcattgtggcgctggccttcaggatgtggagcgaggtgacgccgctggacttccgcgaggacctggccgcccccggggccgcggtcgacatcaagctgggctttgggagaggccggcacctgggctgtccgcgggccttcgatgggagcgggcaggagtttgcacacgcctggcgcctaggtgacattcactttgacgacgacgagcacttcacacctcccaccagtgacacgggcatcagccttctcaaggtggccgtccatgaaattggccatgtcctgggcttgcctcacacctacaggacgggatccataatgcaaccaaattacattccccaggagcctgcctttgagttggactggtcagacaggaaagcaattcaaaagctgtatggctcctgtgagggatcatttgatactgcgtttgactggattcgcaaagagagaaaccaatatggagaggtgatggtgagatttagcacatatttcttccgtaacagctggtactggctttatgaaaatcgaaacaataggacacgctatggggaccctatccaaatcctcactggctggcctggaatcccaacacacaacatagatgcctttgttcacatctggacatggaaaagagatgaacgttatttttttcaaggaaatcaatactggagatatgacagtgacaaggatcaggccctcacagaagatgaacaaggaaaaagctatcccaaattgatttcagaaggatttcctggcatcccaagtcccctagacacggcgttttatgaccgaagacagaagttaatttacttcttcaaggagtcccttgtatttgcatttgatgtcaacagaaatcgagtacttaattcttatccaaagaggattactgaagtttttccagcagtaataccacaaaatcatcctttcagaaatatagattccgcttattactcctatgcatacaactccattttctttttcaaaggcaatgcatactggaaggtagttaatgacaaggacaaacaacagaattcctggcttcctgctaatggcttatttccaaaaaagtttatttcagagaagtggtttgatgtttgtgacgtccatatctccacactgaacatgtaataagaaaaagtaggaaatggagggtcataggacttcgctaaaattctgatactgtttacaaagaaaaccggattttcagtagctgaagaaaatatggcactgtaagttaaaacccaatgggaaaagccttagttgaacttttaaaatacttgatttaaaaacaattgctcaggcaaaacataattcataaactaaaaggtttaaaaaaa

SEQ ID NO: 30 - DNA, 1858 bp, Mus musculus
  source 1..1858: mol_type = unassigned DNA
agtcccagtgaagccatgcttgctgcctctgtcttgcgtctgacactgccgctctgctggctagttgcaccccagcccacccagcctgagaggctcttccacagccgggaccgttcggacctggagccatctcccctgagccaggccaagcccattgctgacctccatgatgctcagagcttcctattgaaatatggctggtcagagatacctagcccaaaggagtcagcaggagtccctgtgggattcaccttggctcaagctgtacgtagattccagaaggcgaacagactgccagccagtggggaattggactcacctacactggccgctatgaacaagccacgctgtggggttcctgacacacggctgccccctcgggctgccctgccaaccccaccagccctgctgacctctttaggtctccggcccagggccagacaaaagcgctttcttcaaatgctgttccccaagccagatgggcaacaggaggatgcctcagacactggggccagcagggccttctccaagaagacactgacctggaggctggtgggagatgcctacagcagccaactctctggggatgagcagaggtacatctttagactggccttcaggatgtggagtgaggtgacaccactggacttccgggaggaccgcactgcccctggcaccatggtggacataaagctgggttttgggagaggccggcacctgggctgtccacgggtgtttgacgggagtggacaggagtttgcacatgcctggcgcctgggtgagatccactttgatgacgacgagcacttcactcctctctccagtgacacaggcatcagccttctcaaagtagctgtccatgaaattggccacgtcctcggattgcctcacacctacagggtgggatccataatgcaaccaaactacattccccaggagcctgcctttgagttggactgggcagacagaaaagcaattcaaagactatatggttcgtgcaaggggtcatttgatacagtgtttgactggatccgcaaggagaggaaccaatatggtgaggtgagggtgagattcaacacgtacttcttccgcaacagctggtactggctctacgaaaaccgcaacaacaggactcgctacggggaccccctccaaatcctcactggctggcgtgggatccccacgcagagcatcgatgcctacgtccatgtctggtcatggggaaaagacgagcgctactttttcaaaggaagccaatactggagatatgacagtgagaatgaccaggcacacacagaagacgaacaaggaaggagctaccctaaactgatctcagaaggattccctggcatccccagccccctggacacggccttttacgatcggaggcagcagttaatttacttcttcaaggagtctctagtatttgcatttgatgtcaaccgaaaccaagtacttaattcttatccaaagaagatgagccaagtgtttccagcaataatgcctcaaaaccacccattcagaaacctcgattcagcgtactactcctatgcacacaactccatcttcttcttcaaaggcaactcgtactggaaagtagttagtgacaaggacaaacagcagaacacccggctgcctctgaatggcttatttccaaaaaaacctgtttccgagaagtggtttgatgtttgtgatgttcacacctctaccctgaacatgtaaagaagaaaatcaagactaggacttaggaacagttttaagggaacgtgtcagccttctctgcaaagaaaacaaacctgattcttaatagctaaagcaaatatggcttcctaagctaaaccaaacaagaaagcattac

SEQ ID NO: 31 - protein, 569 aa, Homo sapiens
MLAASIFRPTLLLCWLAAPWPTQPESLFHSRDRSDLEPSPLRQAKPIADLHAAQRFLSRYGWSGVWAAWGPSPEGPPETPKGAALAEAVRRFQRANALPASGELDAATLAAMNRPRCGVPDMRPPPPSAPPSPPGPPPRARSRRSPRAPLSLSRRGWQPRGYPDGGAAQAFSKRTLSWRLLGEALSSQLSVADQRRIVALAFRMWSEVTPLDFREDLAAPGAAVDIKLGFGRGRHLGCPRAFDGSGQEFAHAWRLGDIHFDDDEHFTPPTSDTGISLLKVAVHEIGHVLGLPHTYRTGSIMQPNYIPQEPAFELDWSDRKAIQKLYGSCEGSFDTAFDWIRKERNQYGEVMVRFSTYFFRNSWYWLYENRNNRTRYGDPIQILTGWPGIPTHNIDAFVHIWTWKRDERYFFQGNQYWRYDSDKDQALTEDEQGKSYPKLISEGFPGIPSPLDTAFYDRRQKLIYFFKESLVFAFDVNRNRVLNSYPKRITEVFPAVIPQNHPFRNIDSAYYSYAYNSIFFFKGNAYWKVVNDKDKQQNSWLPANGLFPKKFISEKWFDVCDVHISTLNM

SEQ ID NO: 32 - protein, 415 aa, Mus musculus
MLAASVLRLTLPLCWLVAPQPTQPERLFHSRDRSDLEPSPLSQAKPIADLHDAQSFLLKYGWSEIPSPKESAGVPVGFTLAQAVRRFQKANRLPASGELDSPTLAAMNKPRCGVPDTRLPPRAALPTPPALLTSLGLRPRARQKRFLQMLFPKPDGQQEDASDTGASRAFSKKTLTWRLVGDAYSSQLSGDEQRYIFRLAFRMWSEVTPLDFREDRTAPGTMVDIKLGFGRGRHLGCPRVFDGSGQEFAHAWRLGEIHFDDDEHFTPLSSDTGISLLKVAVHEIGHVLGLPHTYRVGSIMQPNYIPQEPAFELDWADRKAIQRLYGSCKGSFDTVFDWIRKERNQYGEVRVRFNTYFFRNSWYWLYENRNNRTRYGDPLQILTGWRGIPTQSIDAYVHVWSWGKDERYFFKGICI

SEQ ID NO: 33 - DNA, 2159 bp, Homo sapiens
  source 1..2159: mol_type = unassigned DNA
aaaaagcgccccgccccgcgctcctctcttgactttgagcgtccggcggtcgcagagccaggaggcggaggcgcgcgggccagcctgggccccagcccacaccttcaccagggcccaggagccaccatgtggcgatgtccactggggctactgctgttgctgccgctggctggccacttggctctgggtgcccagcagggtcgtgggcgccgggagctagcaccgggtctgcacctgcggggcatccgggacgcgggaggccggtactgccaggagcaggacctgtgctgccgcggccgtgccgacgactgtgccctgccctacctgggcgccatctgttactgtgacctcttctgcaaccgcacggtctccgactgctgccctgacttctgggacttctgcctcggcgtgccacccccttttcccccgatccaaggatgtatgcatggaggtcgtatctatccagtcttgggaacgtactgggacaactgtaaccgttgctggcaggctgggaaccacagcgccttctggggcatgaccctggatgagggcattcgctaccgcctgggcaccatccgcccatcttcctcggtcatgaacatgcatgaaatttatacagtgctgaacccaggggaggtgcttcccacagccttcgaggcctctgagaagtggcccaacctgattcatgagcctcttgaccaaggcaactgtgcaggctcctgggccttctccacagcagctgtggcatccgatcgtgtctcaatccattctctgggacacatgacgcctgtcctgtcgccccagaacctgctgtcttgtgacacccaccagcagcagggctgccgcggtgggcgtctcgatggtgcctggtggttcctgcgtcgccgaggggtggtgtctgaccactgctaccccttctcgggccgtgaacgagacgaggctggccctgcgcccccctgtatgatgcacagccgagccatgggtcggggcaagcgccaggccactgcccactgccccaacagctatgttaataacaatgacatctaccaggtcactcctgtctaccgcctcggctccaacgacaaggagatcatgaaggagctgatggagaatggccctgtccaagccctcatggaggtgcatgaggacttcttcctatacaagggaggcatctacagccacacgccagtgagccttgggaggccagagagataccgccggcatgggacccactcagtcaagatcacaggatggggagaggagacgctgccagatggaaggacgctcaaatactggactgcggccaactcctggggcccagcctggggcgagaggggccacttccgcatcgtgcgcggcgtcaatgagtgcgacatcgagagcttcgtgctgggcgtctggggccgcgtgggcatggaggacatgggtcatcactgaggctgcgggcaccacgcggggtccggcctgggatccaggctaagggccggcggaagaggccccaatggggcggtgaccccagcctcgcccgacagagcccggggcgcaggcgggcgccagggcgctaatcccggcgcgggttccgctgacgcagcgccccgcctgggagccgcgggcaggcgagactggcggagcccccagacctcccagtggggacggggcagggcctggcctgggaagagcacagctgcagatcccaggcctctggcgcccccactcaagactaccaaagccaggacacctcaagtctccagccccactaccccaccccactcctgtattcttttttttttttttttagacagggtcttgctccgttgcccaggttggagtgcagtggcccatcagggctcactgtaacctccgactcctgggttcaagtgaccctcccacctcagcctctcaagtagctgggactacaggtgcaccaccacacctggctaatttttgtattttttgtaaagaggggggtctcactgtgttgcccaggctggtctcgaactcctgggctcaagcggtccacctgcctccgcctcccaaagtgctgggattgcaggcatgagccactgcacccagccctgtattcttattcttcagatatttatttttcttttcactgttttaaaataaaaccaaagtattgataaaaaaaaaaaaaaaaa

SEQ ID NO: 34 - DNA, 1954 bp, Mus musculus
  source 1..1954: mol_type = unassigned DNA
acttgactcggtctggcagtctcgagcttggaggcggaggcgcgcagggcagcctgggtccagcccacacccctcaccaggaggcaccatgtggggatgttggctggggctgctcttgctgctgctggctggccaggctgccctggaggcccggcggagtcgttggcgcagggagctggcgccagggctgcacctgcggggcatccgggacgccggtggcagatactgccaagagcaggacatgtgctgtcgaggccgtgctgacgagtgtgccctgccctacctgggagccacctgttactgtgacctcttctgcaaccgcaccgtctctgactgctgccccgacttctgggacttctgcctcgggattccaccccccttccctcccgtccaagggtgcatgcatgggggccggatctacccagtcttcggaacctactgggacaactgcaatcgatgcacctgccatgagggagggcattgggagtgcgaccaggagccgtgtctagtggacccagacatgattaaagccatcaaccgaggcaactacgggtggcaggctgggaaccacagtgccttctggggcatgaccctggatgagggcattcgctaccgcctgggcacaatccgcccatcctccactgtcatgaatatgaatgagatttatacggtgctgggccaaggggaagtgctacccactgcctttgaagcttcagagaagtggcccaacctgatccacgagccattggaccagggcaattgtgcaggttcctgggctttctccacagcagctgtcgcatctgatcgcgtctctatccattctttgggacacatgacacccatcctatcaccgcaaaacctgctgtcctgtgatacccaccaccagcagggctgccgaggtgggcgtcttgatggcgcttggtggttcctgcggcgccgcggggtggtgtctgacaactgttacccattctccggccgtgagcagaacgaggccagccccactcctcgttgtatgatgcacagccgcgccatggggcggggcaagcgccaagccacttcccgctgccccaatggtcaggttgattccaacgacatctaccaggtcacgcctgcctaccgcctgggctctgatgagaaggagatcatgaaggagctaatggaaaacggccctgttcaagcactcatggaagtacacgaggacttcttcttgtaccagcgtggcatctacagccacacacctgtaagccaggggaggccggagcagtaccgcagacatgggactcactcagtcaagatcactgggtggggagaagagacgctgccagacggaaggaccattaagtactggactgctgccaactcgtggggcccatggtggggtgaaaggggccacttccggatcgtgcgtggcaccaacgagtgcgacatcgagaccttcgtgctgggcgtctggggtcgcgtgggaatggaggacatggggcaccactgagtctcagccactaggcgaggtgggatccacagccacagaagaggccttgggggccatgcccgatgaagccttgtgtgcacttcgggaccaggtgctaatctctacagactcagatccgcgcgtgcgcgctaaggcagaatcccacctaggagacaaagatgcaccaggctggcggaagcccccagatatcacagccggaactgggaagggccctgtttggaaactgcagggagtatagacagattccaggtccctggtcagccaggccaagaccacaggagctaagacaccccaacctcatcaccctcctacccaccctctctctcatcttctttttgatgaattctgtccatctccctagcctccattttggttgtacctttccattctcagtactgctttcttattctttaaagatatttatttttcttttcattaaaataaaaccaaagtattgataattac

SEQ ID NO: 35 - protein, 436 aa, Homo sapiens
MWRCPLGLLLLLPLAGHLALGAQQGRGRRELAPGLHLRGIRDAGGRYCQEQDLCCRGRADDCALPYLGAICYCDLFCNRTVSDCCPDFWDFCLGVPPPFPPIQGCMHGGRIYPVLGTYWDNCNRCWQAGNHSAFWGMTLDEGIRYRLGTIRPSSSVMNMHEIYTVLNPGEVLPTAFEASEKWPNLIHEPLDQGNCAGSWAFSTAAVASDRVSIHSLGHMTPVLSPQNLLSCDTHQQQGCRGGRLDGAWWFLRRRGVVSDHCYPFSGRERDEAGPAPPCMMHSRAMGRGKRQATAHCPNSYVNNNDIYQVTPVYRLGSNDKEIMKELMENGPVQALMEVHEDFFLYKGGIYSHTPVSLGRPERYRRHGTHSVKITGWGEETLPDGRTLKYWTAANSWGPAWGERGHFRIVRGVNECDIESFVLGVWGRVGMEDMGHH

SEQ ID NO: 36 - protein, 466 aa, Mus musculus
MWGCWLGLLLLLLAGQAALEARRSRWRRELAPGLHLRGIRDAGGRYCQEQDMCCRGRADECALPYLGATCYCDLFCNRTVSDCCPDFWDFCLGIPPPFPPVQGCMHGGRIYPVFGTYWDNCNRCTCHEGGHWECDQEPCLVDPDMIKAINRGNYGWQAGNHSAFWGMTLDEGIRYRLGTIRPSSTVMNMNEIYTVLGQGEVLPTAFEASEKWPNLIHEPLDQGNCAGSWAFSTAAVASDRVSIHSLGHMTPILSPQNLLSCDTHHQQGCRGGRLDGAWWFLRRRGVVSDNCYPFSGREQNEASPTPRCMMHSRAMGRGKRQATSRCPNGQVDSNDIYQVTPAYRLGSDEKEIMKELMENGPVQALMEVHEDFFLYQRGIYSHTPVSQGRPEQYRRHGTHSVKITGWGEETLPDGRTIKYWTAANSWGPWWGERGHFRIVRGTNECDIETFVLGVWGRVGMEDMGHH

SEQ ID NO: 37 - DNA, 1484 bp, Homo sapiens
  source 1..1484: mol_type = unassigned DNA
ctactttgtgttctttccctaccaactaagcttgcctaatttgcttcagaattggaagagggaattgcagcaggaaaatatgtgaagagtttttaaacccacaaattcttcttactttagaattagttgttacattggcaggaaaaaataaatgcagatgttggaccatgttggaaaccttgtcaagacagtggattgtctcacacagaatggaaatgtggcttctgattctggtggcgtatatgttccagagaaatgtgaattcagtacatatgccaactaaagctgtggacccagaagcattcatgaatattagtgaaatcatccaacatcaaggctatccctgtgaggaatatgaagtcgcaactgaagatgggtatatcctttctgttaacaggattcctcgaggcctagtgcaacctaagaagacaggttccaggcctgtggtgttactgcagcatggcctagttggaggtgctagcaactggatttccaacctgcccaacaatagcctgggcttcattctggcagatgctggttttgacgtgtggatggggaacagcaggggaaacgcctggtctcgaaaacacaagacactctccatagaccaagatgagttctgggctttcagttatgatgagatggctaggtttgaccttcctgcagtgataaactttattttgcagaaaacgggccaggaaaagatctattatgtcggctattcacagggcaccaccatgggctttattgcattttccaccatgccagagctggctcagaaaatcaaaatgtattttgctttagcacccatagccactgttaagcatgcaaaaagccccgggaccaaatttttgttgctgccagatatgatgatcaagggattgtttggcaaaaaagaatttctgtatcagaccagatttctcagacaacttgttatttacctttgtggccaggtgattcttgatcagatttgtagtaatatcatgttacttctgggtggattcaacaccaacaatatgaacatgagccgagcaagtgtatatgctgcccacactcttgctggaacatctgtgcaaaatattctacactggagccaggcagtgaattctggtgaactccgggcatttgactgggggagtgagaccaaaaatctggaaaaatgcaatcagccaactcctgtaaggtacagagtcagagatatgacggtccctacagcaatgtggacaggaggtcaggactggctttcaaatccagaagacgtgaaaatgctgctctctgaggtgaccaacctcatctaccataagaatattcctgaatgggctcacgtggatttcatctggggtttggatgctcctcaccgtatgtacaatgaaatcatccatctgatgcagcaggaggagaccaacctttcccagggacggtgtgaggccgtattgtgaagcatctgacactgacgatcttaggacaacctcctgagggatggg

SEQ ID NO: 38 - DNA, 2701 bp, Mus musculus
  source 1..2701: mol_type = unassigned DNA
ggagcaggaggagctaggcagcattgtgacattctaagcaatgtttgcagcctacaccgtgttctttcattcctgttcttcttctgactctagatgtagttgttacatccttagcaacaaagcaaatgccgatgttggaccatgtcagaaatcttgtcaagagtgtggactgtttcgcacagagtggagatatggcttctgattctggtagcgtatttactccaaagaaatgtgaactcgggacatttgcccacgaaagctgcggatccagaagcattcatgaatgttagcgaaatcatcaaacacaagggttatcccagtgaggagtatgaagttgcaacggaagatgggtacatcctttctgtgaacagaatccctcggggacagacacggttaaagaaggaaggatccaggccagtggtgttactgcaacatggtcttcttggggatgctagcaactggatttccaacctgcccaacaacagcctgggttttattctggcagatgcaggttttgatgtgtggatgggaaacagcagaggaaacacctggtctcggaagcacaagaccctctccatagatcaagatgagttctgggctttcagttatgatgaaatggctaggtttgaccttccagctgtgataaactttatcttacagaaaacgggccagaaaaaggtctattatgtcggctactcacagggcaccaccatgggctttattgcattttccacaatgccagagctagctcataaaatcaaaatgtattttgccttagctcctatagccactgttaaatatgcaagaagtcctggtaccaaatttctgctgctgccagatatgatgatcaaggtattatttggcagacaagagtttttataccagactagatttttcagacagctttttatttacctttgtggccagatgattcttgaccaaatctgcagcaacatcatcttactcctgggaggttttaacacaaacaatatgaacatgagcagagcaaatgtgtatgtcgcccatacacctgctggaacgtctgtgcagaacattctccactggagccaggccgtgaattctggggaacttcgtgcctttgactggggaagtgagaccaaaaatcaggagaaatgtaatcagccaactcctataaggtacaaagttcgagatatgatggtcccaacagcaatgtggactggaggtcaagactggctttcaaatccagatgatgtgaaaacattactttctgaagtaaccaacctcatctaccacaagaacattcctgagtgggctcatgtggatttcatctgggggctggatgcccctcagcgtgtttacaatgaaattatacatctgatgaagcaggagcccaaccttccccagggaacctgcagggtcaaattgtgaagctcctgcaagtcttagaacaaccccatgcggaatgggcagggccatgggaggatggatgctcaaagaaatgtacagacttggaaacctgctatatcagttagtcagaaggacagaggagatacccagatgccagaagacctcctggtccaaattaaagcttcatttgtttcttcatttgacacgaaagctgatacttacactttttccagttggttaaactcagttggtgaacacaggttttctgtgctttctatctttgtcatggtgcaatgtagtttttacttacccacaagatgtatctagactgttatcattgcatcattcaacctttccaacataatttttgtttttgaaagtcgaatttttagagcattaaaagcaataattggggcagggatcaaaattgtaaatctttggattgttgacttggataaaatgaagtggaccttgccatcacattgccataaagacaagacacttctttaagaagccaggctatttggtcatcaactgaaaactgaaatcattgtgggtaggtggagtctaactaaacaatcaagttttaaagttggatctcaaagcttattttgtaggatatttatgagttttccaataaattaattatgtagctcccatgtcatgtctgtgtatcagtactaagattttcatctgctattagaccagctgctccaaatgagtaaaatgaaataaactgttgtgtattttatcctttaatcaatgccgaatcactcttagataatctccaaactcttgcatagatctagatttatgtccttggaaaactctttaattttcacacaatttcaaatatgacagtagtaaattatgagtattatttggcattctactctgaagacaaataaaagctctcctgtgcctgttggagttccagttgagaatatacagttgtttgttaaacatacattagactgtgaagttctttttttcccccagagttgcctggtaagttagtccaatgttaccaaatcttaggtaaattacttagtaatatttagggacttcaggaccccatgtgtacccttagggtctatatatagcttagtatacatgtccagatttgatttttgttgaacctcttgtaaaaaatgactgttattataaatgtatcagtaaattaatacgtcagttgtttttatttatcctaccttgttctccaataatgtcacagacagaccaaaattagattaccagaggataatgcctagctattgtacgttaagttgaaaataaatcacaaaggagtctt

SEQ ID NO: 39 - protein, 423 aa, Homo sapiens
MLETLSRQWIVSHRMEMWLLILVAYMFQRNVNSVHMPTKAVDPEAFMNISEIIQHQGYPCEEYEVATEDGYILSVNRIPRGLVQPKKTGSRPVVLLQHGLVGGASNWISNLPNNSLGFILADAGFDVWMGNSRGNAWSRKHKTLSIDQDEFWAFSYDEMARFDLPAVINFILQKTGQEKIYYVGYSQGTTMGFIAFSTMPELAQKIKMYFALAPIATVKHAKSPGTKFLLLPDMMIKGLFGKKEFLYQTRFLRQLVIYLCGQVILDQICSNIMLLLGGFNTNNMNMSRASVYAAHTLAGTSVQNILHWSQAVNSGELRAFDWGSETKNLEKCNQPTPVRYRVRDMTVPTAMWTGGQDWLSNPEDVKMLLSEVTNLIYHKNIPEWAHVDFIWGLDAPHRMYNEIIHLMQQEETNLSQGRCEAVL

SEQ ID NO: 40 - protein, 422 aa, Mus musculus
MSEILSRVWTVSHRVEIWLLILVAYLLQRNVNSGHLPTKAADPEAFMNVSEIIKHKGYPSEEYEVATEDGYILSVNRIPRGQTRLKKEGSRPVVLLQHGLLGDASNWISNLPNNSLGFILADAGFDVWMGNSRGNTWSRKHKTLSIDQDEFWAFSYDEMARFDLPAVINFILQKTGQKKVYYVGYSQGTTMGFIAFSTMPELAHKIKMYFALAPIATVKYARSPGTKFLLLPDMMIKVLFGRQEFLYQTRFFRQLFIYLCGQMILDQICSNIILLLGGFNTNNMNMSRANVYVAHTPAGTSVQNILHWSQAVNSGELRAFDWGSETKNQEKCNQPTPIRYKVRDMMVPTAMWTGGQDWLSNPDDVKTLLSEVTNLIYHKNIPEWAHVDFIWGLDAPQRVYNEIIHLMKQEPNLPQGTCRVKL

SEQ ID NO: 41 - DNA, 863 bp, Homo sapiens
  source 1..863: mol_type = unassigned DNA
gttcccagaagctccccaggctctagtgcaggaggagaaggaggaggagcaggaggtggagattcccagttaaaaggctccagaatcgtgtaccaggcagagaactgaagtactggggcctcctccactgggtccgaatcagtaggtgaccccgcccctggattctggaagacctcaccatgggacgcccccgacctcgtgcggccaagacgtggatgttcctgctcttgctggggggagcctgggcaggaaatacacagtacgcctgggagaccacagcctacagaataaagatggcccagagcaagaaatacctgtggttcagtccatcccacacccctgctacaacagcagcgatgtggaggaccacaaccatgatctgatgcttcttcaactgcgtgaccaggcatccctggggtccaaagtgaagcccatcagcctggcagatcattgcacccagcctggccagaagtgcaccgtctcaggctggggcactgtcaccagtccccgagagaattttcctgacactctcaactgtgcagaagtaaaaatctttccccagaagaagtgtgaggatgcttacccggggcagatcacagatggcatggtctgtgcaggcagcagcaaaggggctgacacgtgccagggcgattctggaggccccctggtgtgtgatggtgcactccagggcatcacatcctggggctcagacccctgtgggaggtccgacaaacctggcgtctataccaacatctgccgctacctggactggatcaagaagatcataggcagcaagggctgattctaggataagcactagatctcccttaataaactcacaactctctggttcaaaaaaaaaa

SEQ ID NO: 42 - DNA, 919 bp, Mus musculus
  source 1..919: mol_type = unassigned DNA
gacacaccgaagggaagtccgggggcctcttccaccgagtccgagtgaccccgccccttgcattctggaagaggccaccatgggacgccccccaccctgtgcaatccagccgtggatccttctgcttctgttcatgggagcgtgggcagggctcaccagagctcagggctccaagatcctggaaggtcgagagtgtataccccactcccagccttggcaggcagccttgttccagggcgagagactgatctgtgggggtgtcctggttggagacagatgggtcctcacggcagcccactgcaaaaaacagaagtactccgtgcgtctgggtgatcatagcctccagagcagagatcagccggagcaggagatccaggtggctcagtctatccagcatccttgctacaacaacagcaacccagaagatcacagtcacgatataatgctcattcgactgcagaactcagcaaacctcggggacaaggtgaagccggtccaactggccaatctgtgtcccaaagttggccagaagtgcatcatatcaggctggggcactgtcaccagccctcaagagaactttccaaacaccctcaactgtgcggaagtgaaaatctattcccagaacaagtgtgagagagcctatccagggaagatcaccgagggcatggtctgtgctggcagcagcaatggagctgacacgtgccagggtgactcaggaggccctctggtgtgcgacgggatgctccagggcatcacctcatggggctcagacccctgtgggaaacccgagaaacctggagtctacaccaaaatctgccgctacactacctggatcaagaagaccatggacaacagggactgatcctggtgtgtgtgtgtggggggggttgtcaataaacaccaccattggctggctcct

SEQ ID NO: 43 - protein, 139 aa, Homo sapiens
MLLQLRDQASLGSKVKPISLADHCTQPGQKCTVSGWGTVTSPRENFPDTLNCAEVKIFPQKKCEDAYPGQITDGMVCAGSSKGADTCQGDSGGPLVCDGALQGITSWGSDPCGRSDKPGVYTNICRYLDWIKKIIGSKG

SEQ ID NO: 44 - protein, 260 aa, Mus musculus
MGRPPPCAIQPWILLLLFMGAWAGLTRAQGSKILEGRECIPHSQPWQAALFQGERLICGGVLVGDRWVLTAAHCKKQKYSVRLGDHSLQSRDQPEQEIQVAQSIQHPCYNNSNPEDHSHDIMLIRLQNSANLGDKVKPVQLANLCPKVGQKCIISGWGTVTSPQENFPNTLNCAEVKIYSQNKCERAYPGKITEGMVCAGSSNGADTCQGDSGGPLVCDGMLQGITSWGSDPCGKPEKPGVYTKICRYTTWIKKTMDNRD

SEQ ID NO: 45 - DNA, 1624 bp, Homo sapiens
  source 1..1624: mol_type = unassigned DNA
gatattcttcaaggaaaatgaaccccacactaggcctggccatttttctggctgttctcctcacggtgaaaggtcttctaaagccgagcttctcaccaaggaattataaagctttgagcgaggtccaaggatggaagcaaaggatggcagccaaggagcttgcaaggcagaacatggacttaggctttaagctgctcaagaagctggccttttacaaccctggcaggaacatcttcctatcccccttgagcatctctacagctttctccatgctgtgcctgggtgcccaggacagcaccctggacgagatcaagcaggggttcaacttcagaaagatgccagaaaaagatcttcatgagggcttccattacatcatccacgagctgacccagaagacccaggacctcaaactgagcattgggaacacgctgttcattgaccagaggctgcagccacagcgtaagtttttggaagatgccaagaacttttacagtgccgaaaccatccttaccaactttcagaatttggaaatggctcagaagcagatcaatgactttatcagtcaaaaaacccatgggaaaattaacaacctgatcgagaatatagaccccggcactgtgatgcttcttgcaaattatattttctttcgagccaggtggaaacatgagtttgatccaaatgtaactaaagaggaagatttctttctggagaaaaacagttcagtcaaggtgcccatgatgttccgtagtggcatataccaagttggctatgacgataagctctcttgcaccatcctggaaataccctaccagaaaaatatcacagccatcttcatccttcctgatgagggcaagctgaagcacttggagaagggattgcaggtggacactttctccagatggaaaacattactgtcacgcagggtcgtagacgtgtctgtacccagactccacatgacgggcaccttcgacctgaagaagactctctcctacataggtgtctccaaaatctttgaggaacatggtgatctcaccaagatcgcccctcatcgcagcctgaaagtgggcgagcaaaggtggacttgatgcataaaggaaggattcaccctaaccaagaatgagggtgcccagtgtcagaggcccaggagagacaggctgccttggtgtggtccagtttgacctacacgatacagaaaacctctgtgcagcctccgagcaggggactttctacctcttccagcataggctgggtgccaggagtctggtttgctgtctgcccccactctgcctgcagcactgcctgcctgggctgaggtcattcatgtgggcgcataacagaagtgacagactggtgtgttggcaagaatgatcacagaagggaagtcctagttcttggccctgcttttcccagacactgactgtgtggctggagcaaataccttcttcctgttgggcttccactgcccatttgtgcaatgatgaggttgccctggatgcccccagaagacccttctattttgggcaatactttgatgttcatattggaaatgatagaactggctcgaaagttttatgtatagtcattctcccttgcatttgtttagtcaataaatacttactgggaa

SEQ ID NO: 46 - DNA, 3690 bp, Mus musculus
  source 1..3690: mol_type = unassigned DNA
ggtgggagatgaggaggatccttcaggggaagtggtgtggccacggctcctggaagcccattgactgactccagcagcttcaattggccacactctgcctcttcaggaggccctggaggtataaataataaaacaaaccaggcaggcccgtttggccctgtggatggcgcctggacccactgataatccccaaggacaatgactcgcatgctggacctgggcctgtttctggcaggcctcctcactgtgaaaggtctcctgcaggacagagatgctccagatatgtatgattctccagtcagggtccaagagtggaggggcaagaaggatgcccggcagcttgctcgacacaacatggaatttggcttcaagctactgcagaggctggccagcaatagccctcagggaaacatcttcctatccccactgagcatctccactgccttctccatgctgtctctcggggcccagaactccacactggaggaaatccgggagggcttcaacttcaaagagatgtcaaactgggacgtccatgcggctttccactaccttctccacaagctcaaccaggagacggaggacacaaagatgaacttaggcaatgctttatttatggatcagaagctgaggccccaacagaggttcctgaacctggctaagaatgtgtatgatgcggacatggtgctcactaacttccaggacttagaaaacactcagaaggatatcaacagatacatcagtcagaaaacccacagcagaatcaaaaacatggtaaaaagcatagacccaggcaccgtgatgattctcacaaattatatttactttcgaggcaggtggcagtatgaattcgacccaaaacagaccaaggaggaggagttcttcatagagaaaggcaagaccgtgaaggtacccatgatgttccaaaggggcttgtacgacatggcctatgacagccagctctcctgcaccatcttggaaatcccctaccggggaaacatcacagccacatttgtccttcctgacaatggcaagctgaagctgctggagcaggggttgcaggcagacatctttgccaagtggaaaagccttctctcaaaaagagtggtggatgtgtgggtgcccaagctccgcatctcgtctacttacaacatgaagaaggttctgtcccgcctgggcatcagcaaaatctttgaggagaatggtgacctcacccggatctcatctcatcgaagcctgaaggtgggagaggctgtgcacaaggctgaactgaagatggatgagaaaggcatggaaggggctgcaggctctggagcccagacattgcccatggagaccccacgacatatgaagctagacagacccttcctcatgatgatctatgagaatttcatgccttccatggtcttcttggcaaggatttatgaccctagtgggtaataaaggagcattccctgttggcttcagaccccacccatttccaagggttgcacctccaaaacctctccaaacaatgccacccaccagggaccaagtgttatggagtcccaacacggcccagcagcccagcttcctctagggactttgtgctctgtacccatttatagcctctcccaagccatcagcactcctggaccaaatgccaacctttactcccactatgcccacatctccttcatccctgaaggcagggagtgtctgtgatgtgataaactccacctcccctgacccatctgctagagtcaggtagatgctcagagggaaatgggagccacagacagtgaaccgagcctgggtagaagaagacattgctaggaaagccacgggtctcattttggaacctctctgcctccctttctgacgtgaggattgccctgctccttgatgaggcgttgaggaaatcggtgccaattccttgcagaattcgttttccattgtgagttccaggctgagaactctggcatgcgtgttacatggctcacctcccagagtgtccaggtagggttggtgctagcccagctagacctccgggaacacggtggaagtgacccagcattagaagaaggctgccttggggtgaagctggagatcgctgccagatttgttcatttctttcggtcacaggaggaagattctaaggttcctcgggcctctggtcaaagacgggttctactattgaacctttggaaaatgggggtctctacctgtctcagaaaactgaccctgtggtgtcaacttactgccactagggggcaccaagggccagaccaatggtgtgggtctggagcgcagagggatgcctttatccctctggctgtgaatagcctgtgcaggccttgttgggggagggacactcagacgggcttggtacctttaaagcagcaattgtcacttcttgcctagcagttctcagaacaccgtgaccttcgtgctttgttcactaaagctcaataggcaaccagaatgagcaagccttgtggaggaaatgaagcttagagaggcgactcggcttcctgtggcgttagagtaagcttagcatagcagtactggagtttgtggtaggaagctctctcgttgctatacatctctgctccagccaatgagaggccagcaatcacctctttccgccatccgacccctatgggatgaggatcctcggaggccaggcctgtggagaggtctgggaacgaccgggtgagcagaaagtttcagagtcagctgcttacctgggtggaagtaggagttggggagaaaccagtggatcagaggctgagctccccttgagcaatttactacatcacagtttataagtcgtggtgctgctcatgaggcacggccacgttagccactgatccgctgagtggcccagctggagccagagccaatgcatgtaccgagggaatagtgatggatgcctccggggaccaggtagactagaagcattccgtgagattctaccttaagaccctggttaccgtcttctgtcacctcctccagtctgagatggccagcgtcttcctacggatatcaactctaaatatctagccccccttccctcccccacagtcccgtgggaatgccctccttctcctgatgagtccctactcgccctccgttcttacctcttcacaggcttccatcctcttcctcggtatcactcaagcagcctttcttccaggaaatggcccacttccaagctcattagtgtctgtgtgtctgtcccctctccttccacttgagctgaagtcagtcagagcacaccatgggctacagttaagctcatgtctacagtctccactaaccatagaaacggctccctgaggtacaggaaagcacatggcaggacagtcagatcctatttcaggaccccagaaaggaaaccaacacaattataacccctgattccaattatagggctcagtcagatagtgtttgcccagtatgtgtgaagccatgggtataaaaataccgtcttaaaaatgaaggaaaaaaaaaaggaagaagttaaaagtacaatattttcccaggatagttaacaagagacaaaatacaaacctgtcttccaaacagaaattggacataagccctaacttttaaaaatgactgttgcttagcataaatatttcccaatatggttcctgaccttcacaaccacatgttcaatactcacaataaaaatcacatatctgc

SEQ ID NO: 47 - protein, 355 aa, Homo sapiens
MNPTLGLAIFLAVLLTVKGLLKPSFSPRNYKALSEVQGWKQRMAAKELARQNMDLGFKLLKKLAFYNPGRNIFLSPLSISTAFSMLCLGAQDSTLDEIKQGFNFRKMPEKDLHEGFHYIIHELTQKTQDLKLSIGNTLFIDQRLQPQRKFLEDAKNFYSAETILTNFQNLEMAQKQINDFISQKTHGKINNLIENIDPGTVMLLANYIFFRARWKHEFDPNVTKEEDFFLEKNSSVKVPMMFRSGIYQVGYDDKLSCTILEIPYQKNITAIFILPDEGKLKHLEKGLQVDTFSRWKTLLSRRVVDVSVPRLHMTGTFDLKKTLSYIGVSKIFEEHGDLTKIAPHRSLKVGEQRWT

SEQ ID NO: 48 - protein, 413 aa, Mus musculus
MTRMLDLGLFLAGLLTVKGLLQDRDAPDMYDSPVRVQEWRGKKDARQLARHNMEFGFKLLQRLASNSPQGNIFLSPLSISTAFSMLSLGAQNSTLEEIREGFNFKEMSNWDVHAAFHYLLHKLNQETEDTKMNLGNALFMDQKLRPQQRFLNLAKNVYDADMVLTNFQDLENTQKDINRYISQKTHSRIKNMVKSIDPGTVMILTNYIYFRGRWQYEFDPKQTKEEEFFIEKGKTVKVPMMFQRGLYDMAYDSQLSCTILEIPYRGNITATFVLPDNGKLKLLEQGLQADIFAKWKSLLSKRVVDVWVPKLRISSTYNMKKVLSRLGISKIFEENGDLTRISSHRSLKVGEAVHKAELKMDEKGMEGAAGSGAQTLPMETPRHMKLDRPFLMMIYENFMPSMVFLARIYDPSG

SEQ ID NO: 49 - DNA, 2203 bp, Homo sapiens
  source 1..2203: mol_type = unassigned DNA
atctctgaagcgccacttctcagaaacacagagctttagctccgccaaaatgaaacactcattaaacgcacttctcattttcctcatcataacatctgcgtggggtgggagcaaaggcccgctggatcagctagagaaaggaggggaaactgctcagtctgcagatccccagtgggagcagttaaataacaaaaacctgagcatgcctcttctccctgccgacttccacaaggaaaacaccgtcaccaacgactggattccagagggggaggaggacgacgactatctggacctggagaagatattcagtgaagacgacgactacatcgacatcgtcgacagtctgtcagtttccccgacagactctgatgtgagtgctgggaacatcctccagctttttcatggcaagagccggatccagcgtcttaacatcctcaacgccaagttcgctttcaacctctaccgagtgctgaaagaccaggtcaacactttcgataacatcttcatagcacccgttggcatttctactgcgatgggtatgatttccttaggtctgaagggagagacccatgaacaagtgcactcgattttgcattttaaagactttgttaatgccagcagcaagtatgaaatcacgaccattcataatctcttccgtaagctgactcatcgcctcttcaggaggaattttgggtacacactgcggtcagtcaatgacctttatatccagaagcagtttccaatcctgcttgacttcaaaactaaagtaagagagtattactttgctgaggcccagatagctgacttctcagaccctgccttcatatcaaaaaccaacaaccacatcatgaagctcaccaagggcctcataaaagatgctctggagaatatagaccctgctacccagatgatgattctcaactgcatctacttcaaaggatcctgggtgaataaattcccagtggaaatgacacacaaccacaacttccggctgaatgagagagaggtagttaaggtttccatgatgcagaccaaggggaacttcctcgcagcaaatgaccaggagctggactgcgacatcctccagctggaatacgtggggggcatcagcatgctaattgtggtcccacacaagatgtctgggatgaagaccctcgaagcgcaactgacaccccgggtggtggagagatggcaaaaaagcatgacaaacagaactcgagaagtgcttctgccgaaattcaagctggagaagaactacaatctagtggagtccctgaagttgatggggatcaggatgctgtttgacaaaaatggcaacatggcaggcatctcagaccaaaggatcgccatcgacctgttcaagcaccaaggcacgatcacagtgaacgaggaaggcacccaagccaccactgtgaccacggtggggttcatgccgctgtccacccaagtccgcttcactgtcgaccgcccctttcttttcctcatctacgagcatcgcaccagctgcctgctcttcatgggaagagtggccaaccccagcaggtcctagaggtggaggtctaggtgtctgaagtgccttgggggcaccctcattttgtttccattccaacaacgagaacagagatgttctggcatcatttacgtagtttacgctaccaatctgaattcgaggcccatatgagaggagcttagaaacgaccaagaagagaggcttgttggaatcaattctgcacaatagcccatgctgtaagctcatagaagtcactgtaactgtagtgtgtctgctgttacctagagggtctcacctccccactcttcacagcaaacctgagcagcgcgtcctaagcacctcccgctccggtgaccccatccttgcacacctgactctgtcactcaagcctttctccaccaggcccctcatctgaataccaagcacagaaatgagtggtgtgactaattccttacctctcccaaggagggtacacaactagcaccattcttgatgtccagggaagaagccacctcaagacatatgaggggtgccctgggctaatgttagggcttaattttctcaaagcctgacctttcaaatccatgatgaatgccatcagtccctcctgctgttgcctccctgtgacctggaggacagtgtgtgccatgtctcccatactagagataaataaatgtagccacatttactgtg

SEQ ID NO: 50 - DNA, 2074 bp, Mus musculus
  source 1..2074: mol_type = unassigned DNA
accaaagttcacattgaaacctgctgctgaccttctctggtctcatctctgccacaccactgctcagaggtacagagcagctttagctctgcagaaatgaaacatccactctgcactcttctttctctcatcacatttatgtgtattgggagcaaaggccttgctgagcagctaactaatgaaaacctgaccacgtccttcctgcctgctaacttccacaaagaaaacacagtcaccaatgactggatcccagagggggaagaggatgaggactacctggacctggagaagctgcttggtgaagatgacgactacatttatatcatcgatgccgtttccccgacagactcagaatcaagtgctggcaacatcctgcagcttttccaaggcaagagccggatccagcgtcttaatatcctcaatgcaaagtttgccttcaacctttaccgagtcctgaaggaccaggctaccacatctgataacctcttcatagcacctgttggcatttctactgccatggggatgatctccttaggtctgaggggagagacccatgaagaagtacactcagttctacacttcagagactttgtcaatgctagcagcaagtacgaggttaccaccattcacaatctctttcgaaagctgacccatcgtctattcaggaggaattttgggtacacacttcggtcagttaatggcctttatattcagaagcagtttcccatacgggaggacttcaaagctgctatgagagagttttactttgctgaggcccaggaggctaacttcccagatcctgccttcatatcaaaggcaaacaaccacattctgaaactcaccaagggccttataaaagaagctctggagaatatagatcctgctactcagatgctgattctgaactgcatctacttcaaaggaacttgggtgaataaattcccagtagaaatgacgcacaaccacaacttccggctgaatgagagagaagtagtcaaagtctccatgatgcaaactaaagggaatttccttgcggcaaatgaccaggagctagactgtgacattctccagctggagtacgtagggggcatcagcatgcttattgtagttccacggaagctatcgggaatgaagactcttgaagcacagcttacaccccaggtggtggagagatggcaaaaaagcatgacaaacagaactcgagaggtacttctgccaaagtttaagcttgagaagaattacaacctggtggaggtccttaagtccatgggaatcacaaagctcttcaacaagaatggcaatatgtcaggcatctcagaccagaggatcgccattgacctgttcaagcaccaaagtaccatcacagtgaatgaagagggtacccaagctgcagctgtaactacagtggggttcatgccgctgtctacccaagtccgattcactgtcgaccgtcccttcctgttcctagtgtatgagcaccgaaccagctgtctgctcttcatgggaaaagtgaccaaccctgccaagtcttaatggcagtggtctgggcacctgatgtagtttggggacactctgtattcctgttttcattctaacaaagaggacagagctattttgctatgataatctctgtggtttatgataataatctgaacagcaaacagtgccattaagtgcacagatgttaatgcaactaatggttgtgccagctattccctagaggtcctagacacacagactgccttagcccccacacctctgtccctaggagaatgaatgggcagagtggagctgacactctctcctcaggcagcctcctcagcctttattcgtcaacggcctctgctcagccaggcccttccagacaaaaggcactgaggaacttgcaccccttccagggagggtgtccaagtagcaccgttcctgcccagtgggaaagcaatcccaacatgtctgagatgttcggacttttaaattcatgtaaatatctcctcttctgctgtgtgtacataggactacatagtctatcctagagataaataaacaaccatatttaccatgtaaaaaaaaaaaa

SEQ ID NO: 51 - protein, 499 aa, Homo sapiens
MKHSLNALLIFLIITSAWGGSKGPLDQLEKGGETAQSADPQWEQLNNKNLSMPLLPADFHKENTVTNDWIPEGEEDDDYLDLEKIFSEDDDYIDIVDSLSVSPTDSDVSAGNILQLFHGKSRIQRLNILNAKFAFNLYRVLKDQVNTFDNIFIAPVGISTAMGMISLGLKGETHEQVHSILHFKDFVNASSKYEITTIHNLFRKLTHRLFRRNFGYTLRSVNDLYIQKQFPILLDFKTKVREYYFAEAQIADFSDPAFISKTNNHIMKLTKGLIKDALENIDPATQMMILNCIYFKGSWVNKFPVEMTHNHNFRLNEREVVKVSMMQTKGNFLAANDQELDCDILQLEYVGGISMLIVVPHKMSGMKTLEAQLTPRVVERWQKSMTNRTREVLLPKFKLEKNYNLVESLKLMGIRMLFDKNGNMAGISDQRIAIDLFKHQGTITVNEEGTQATTVTTVGFMPLSTQVRFTVDRPFLFLIYEHRTSCLLFMGRVANPSRS

SEQ ID NO: 52 - protein, 478 aa, Mus musculus
MKHPLCTLLSLITFMCIGSKGLAEQLTNENLTTSFLPANFHKENTVTNDWIPEGEEDEDYLDLEKLLGEDDDYIYIIDAVSPTDSESSAGNILQLFQGKSRIQRLNILNAKFAFNLYRVLKDQATTSDNLFIAPVGISTAMGMISLGLRGETHEEVHSVLHFRDFVNASSKYEVTTIHNLFRKLTHRLFRRNFGYTLRSVNGLYIQKQFPIREDFKAAMREFYFAEAQEANFPDPAFISKANNHILKLTKGLIKEALENIDPATQMLILNCIYFKGTWVNKFPVEMTHNHNFRLNEREVVKVSMMQTKGNFLAANDQELDCDILQLEYVGGISMLIVVPRKLSGMKTLEAQLTPQVVERWQKSMTNRTREVLLPKFKLEKNYNLVEVLKSMGITKLFNKNGNMSGISDQRIAIDLFKHQSTITVNEEGTQAAAVTTVGFMPLSTQVRFTVDRPFLFLVYEHRTSCLLFMGKVTNPAKS

SEQ ID NO: 53 - DNA, 3719 bp, Homo sapiens
  source 1..3719: mol_type = unassigned DNA
gccgagccgggactgcgctagcccgccgcgctctgggctgcccgagcgagcgttcggacctcgcaccccgcgcgccccgcgccgccgccgccgccggcttttgttgtctccgcctcctcggccgccgccgcctctggaccgcgagccgcgcgcgccgggaccttggctctgcccttcgcgggcgggaactgcgcaggacccggccaggatccgagagaggcgcgggcgggtggccgggggcgccgccggccccgccatggagctccgggcccgaggctggtggctgctatgtgcggccgcagcgctggtcgcctgcgcccgcggggacccggccagcaagagccggagctgcggcgaggtccgccagatctacggagccaagggcttcagcctgagcgacgtgccccaggcggagatctcgggtgagcacctgcggatctgtccccagggctacacctgctgcaccagcgagatggaggagaacctggccaaccgcagccatgccgagctggagaccgcgctccgggacagcagccgcgtcctgcaggccatgcttgccacccagctgcgcagcttcgatgaccacttccagcacctgctgaacgactcggagcggacgctgcaggccaccttccccggcgccttcggagagctgtacacgcagaacgcgagggccttccgggacctgtactcagagctgcgcctgtactaccgcggtgccaacctgcacctggaggagacgctggccgagttctgggcccgcctgctcgagcgcctcttcaagcagctgcacccccagctgctgctgcctgatgactacctggactgcctgggcaagcaggccgaggcgctgcggcccttcggggaggccccgagagagctgcgcctgcgggccacccgtgccttcgtggctgctcgctcctttgtgcagggcctgggcgtggccagcgacgtggtccggaaagtggctcaggtccccctgggcccggagtgctcgagagctgtcatgaagctggtctactgtgctcactgcctgggagtccccggcgccaggccctgccctgactattgccgaaatgtgctcaagggctgccttgccaaccaggccgacctggacgccgagtggaggaacctcctggactccatggtgctcatcaccgacaagttctggggtacatcgggtgtggagagtgtcatcggcagcgtgcacacgtggctggcggaggccatcaacgccctccaggacaacagggacacgctcacggccaaggtcatccagggctgcgggaaccccaaggtcaacccccagggccccgggcctgaggagaagcggcgccggggcaagctggccccgcgggagaggccaccttcaggcacgctggagaagctggtctccgaagccaaggcccagctccgcgacgtccaggacttctggatcagcctcccagggacactgtgcagtgagaagatggccctgagcactgccagtgatgaccgctgctggaacgggatggccagaggccggtacctccccgaggtcatgggtgacggcctggccaaccagatcaacaaccccgaggtggaggtggacatcaccaagccggacatgaccatccggcagcagatcatgcagctgaagatcatgaccaaccggctgcgcagcgcctacaacggcaacgacgtggacttccaggacgccagtgacgacggcagcggctcgggcagcggtgatggctgtctggatgacctctgcagccggaaggtcagcaggaagagctccagctcccggacgcccttgacccatgccctcccaggcctgtcagagcaggaaggacagaagacctcggctgccagctgcccccagcccccgaccttcctcctgcccctcctcctcttcctggcccttacagtagccaggccccggtggcggtaactgccccaaggccccagggacagaggccaaggactgactttgccaaaaatacaacacagacgatatttaattcacctcagcctggagaggcctggggtgggacagggagggccggcggctctgagcaggggcaggcgcagaggtcccagccccaggcctggcctcgcctgcctttctgccttttaattttgtatgaggtcctcaggtcagctgggagccagtgtgcccaaaagccatgtatttcagggacctcaggggcacctccggctgcctagccctccccccagctccctgcaccgccgcagaagcagcccctcgaggcctacagaggaggcctcaaagcaacccgctggagcccacagcgagcctgtgccttcctccccgcctcctcccactgggactcccagcagagcccaccagccagccctggcccaccccccagcctccagagaagccccgcacgggctgtctgggtgtccgccatccagggtctggcagagcctctgagatgatgcatgatgccctcccctcagcgcaggctgcagagcccggccccacctccctgcgcccttgaggggccccagcgtctgcagggtgacgcctgagacagcaccactgctgaggagtctgaggactgtcctcccacagacctgcagtgaggggccctccatgcgcagatgaggggccactgacccacctgcgcttctgctggaggaggggaagctgggcccaaaggcccagggaggcagcgtgggctctgccaatgtgggctgcccctcgcacacagggctcacagggcaggccttgctggggtccagggctgttggaggaccccgagggctgaggagcagccaggacccgcctgctcccatcctcacccagatcaggaaccagggcctccctgttcacggtgacacaggtcagggctcagagtgaccctcagctgtcacctgctcacagggatgctggtggctggtgagaccccgcactgcagacgggaatgcctaggtcccttcccgacccagccagctgcagggcacggggacctggatagttaagggcttttccaaacatgcatccatttactgacacttcctgtccttgttcatggagagctgttcgctcctcccagatggcttcggagggccgcagggcccaccttggaccctggtgacctcctgtcactcactgaggccatcagggccctgccccaggcctggacgggccctccttccctcctgtgccccagctgccaggcggccctggggaggggtggtgtggtgttgggaaggggtcctgcagggggaggaggacttggagggtctgggggcagctgtcctgaaccgactgaccctgaggaggccgcttagtgctgctttgcttttcatcaccgtcccgcacagtggacggaggtccccggttgctggtcaggtccccatggcttgttctctggaacctgactttagatgttttgggatcaggagcccccaacacaggcaagtccaccccataataaccctgccagtgccagggtgggctggggactctggcacagtgatgccgggcgccaggacagcagcactcccgctgcacacagacggcctaggggtggcgctcagaccccaccctacgctcatctctggaaggggcagccctgagtggtcactggtcagggcagtggccaagcctgctgtgtccttcctccacaaggtccccccaccgctcagtgtcagcgggtgacgtgtgttcttttgagtccttgtatgaataaaaggctggaaaccta

SEQ ID NO: 54 - DNA, 3608 bp, Mus musculus
  source 1..3608: mol_type = unassigned DNA
tggggccgggcgggcggcgggggaggcgccgagccggggctgcgcgttcgcccgccgcgctctaggcggcccgactgagcggccggacctcgcgcctcgcgcctagctccccgcgccgcagccgcagccggcttttgttgtctccgcctcctcgactgccaccgcctctggaccacgagcgtcgcgcgctgggaccttggctctgcccttcgcgcacactgggctgagcgggccggccgggaccgagggaggcgcgcaccggcggccagggagcctcgggccccgccatggaactccggacccgaggctggtggctgctgtgcgcggccgccgcgctggtcgtctgcgcccgcggggaccccgccagcaagagccggagctgcagcgaagtccgccagatctacggggctaagggctttagcctgagcgatgtgcctcaggcagagatctcgggtgagcacctgcggatctgcccccagggctacacttgctgtactagtgagatggaggagaatttggccaaccacagccgaatggagctggagagcgcactccatgacagcagccgcgccctgcaggccacactggccacccagctgcatggcatcgatgaccacttccagcgcctgctgaatgactcggagcgcacactgcaggaggctttccctggggcctttggggacctgtatacgcagaacactcgtgccttccgggacctatatgctgagctgcgcctctactaccgtggggccaacctgcaccttgaggagacgctggccgagttctgggcacggctgctggagcgcctcttcaagcagctgcacccccagctgctgcctgatgactacctggactgcctgggcaagcaggcggaggcactgcggccgtttggagatgcccctcgagaactgcgcctgcgggccacccgtgcctttgtggctgcacgttcctttgtgcagggcctgggtgtggccagtgatgtagtccggaaggtggcccaggtacctctggccccagaatgttctcgggccatcatgaagttggtctactgtgctcattgccggggagtcccgggcgcccggccctgccccgactattgccgaaatgtgctcaaaggctgccttgccaaccaggccgacctggatgccgagtggaggaacctcctggactccatggtgctcatcactgacaagttctggggcccgtcgggtgcggagagtgtcattggcggtgtgcacgtgtggctggcggaggccatcaacgccctccaggacaacaaggacacactcacagctaaggtcatccaggcctgtggaaaccccaaggtcaatccccacggctctgggcccgaggagaagcgtcgccgtggcaaattggcactgcaggagaagccctccacaggtactctggaaaaactggtctctgaggccaaggcccagctccgagacattcaggacttctggatcagcctcccagggacactgtgcagtgagaagatggccatgagtcctgccagtgacgaccgctgctggaatggaatttccaagggccggtacctaccagaggtgatgggtgacgggctggccaaccagatcaacaaccctgaggtggaagtggacatcaccaagccagacatgaccatccgccagcagattatgcagctcaagatcatgaccaaccgtttacgtggcgcctatggcggcaacgacgtggacttccaggatgctagtgatgacggcagtggctccggcagcggtggcggatgcccagatgacacctgtggccggagggtcagcaagaagagttccagctcccggacccccttgacccatgccctccccggcctgtcagaacaggagggacagaagacctcagctgccacctgcccagagccccacagcttcttcctgctcttcctcgtcaccttggtccttgcggcagccaggcccaggtggcggtaactgccccctatccccaaggactgtcttggccaaaacatgcaacagaccatatttaattcccttggccttttgaggtccagggcaggataaggagacagtagctctgagtgctggggcagggtgcatgggggggtcctggcctcctgggtctggccacgcctgtcaccctagcttttaattttgtatcaggtcagctgcgagccagtgtccccaaaagccatgtatttcagggacctcaggggcacctctggctgcacactcctcccctaccctcctgcaccacccccagaagctcatgaggccaaccagaagggcggctgttagctacaacccaacggagacctcaagtgagcctgtgccttcctctcctgcctcttcccgctggggactccctaccagaccccatgggccacagatgtcagaaattgaggcccatcctgcagctccccaggaagcctgcaagggatgccagtatgttgctgtccaggctctggcagggccttcaagtttatgcatgataccttctactcagaagaagctctgcagccaggatccccacacacagcagaccaggaccctgtaccactgtggagctcagtgaatctggttctcaaaaaaagacccactgtgggattctctagtatgacgtaggtgaggtgggaggtggcagctggcacagccatactacgctcaactctgccacactgtttgagggtatgttccgtcccctcagatgggctcacaaagactggccttggtggtccacatgaggagcctcacttcaggggaacagccagctacatctagcccctaccccacttagaaaggtcacctggccatggggatgctgggtggctggtgaaactttagctcagggtcaatgagacctggcactgcaggtcaagaggatgcctgggccccttcctgacccaggcagctctgcaggagctggggcagcattgacagtcaagggcttttacagacatgcacattcagaccctcagtgtccttgtccactgagcgctacatctccatcttccttgacagcactggagaggctcagggcaccacttggaatctggtttcctcctatggcctcgagacctgggggcacctggcttaccaaggctgtcaaggctccatcttcccttccagggcctaggggatgctgagttgctatatccaggaaggcagtggtcctgacatgcagaaggcctggaaaaaaggctgcttaccgcttttttttttttttaattttaattttttttttttaactttcttttagtcactgggttagagaggctattgccagcacagcaccaaggcctgcccagtagtcaggtcccctggtccaggatgggtattctccataattggtctgtgggtttgttgggcccatgccctgagcacactcaacccagccacagccctgctagtgccactcaagcaggggcactgggagctagacctgggctctgagatgagtgcctaggtgtcaggacagcagcacccatgctgtacagggcagccgggacagtggccagggtactgggtcctttctctgtcactcggtgtcagtggataatgtgtgttctttgagtccttgtatgaattaaaggctggggacctacagtggacatgacctca

SEQ ID NO: 55 - protein, 558 aa, Homo sapiens
MELRARGWWLLCAAAALVACARGDPASKSRSCGEVRQIYGAKGFSLSDVPQAEISGEHLRICPQGYTCCTSEMEENLANRSHAELETALRDSSRVLQAMLATQLRSFDDHFQHLLNDSERTLQATFPGAFGELYTQNARAFRDLYSELRLYYRGANLHLEETLAEFWARLLERLFKQLHPQLLLPDDYLDCLGKQAEALRPFGEAPRELRLRATRAFVAARSFVQGLGVASDVVRKVAQVPLGPECSRAVMKLVYCAHCLGVPGARPCPDYCRNVLKGCLANQADLDAEWRNLLDSMVLITDKFWGTSGVESVIGSVHTWLAEAINALQDNRDTLTAKVIQGCGNPKVNPQGPGPEEKRRRGKLAPRERPPSGTLEKLVSEAKAQLRDVQDFWISLPGTLCSEKMALSTASDDRCWNGMARGRYLPEVMGDGLANQINNPEVEVDITKPDMTIRQQIMQLKIMTNRLRSAYNGNDVDFQDASDDGSGSGSGDGCLDDLCSRKVSRKSSSSRTPLTHALPGLSEQEGQKTSAASCPQPPTFLLPLLLFLALTVARPRWR

SEQ ID NO: 56 - protein, 557 aa, Mus musculus
MELRTRGWWLLCAAAALVVCARGDPASKSRSCSEVRQIYGAKGFSLSDVPQAEISGEHLRICPQGYTCCTSEMEENLANHSRMELESALHDSSRALQATLATQLHGIDDHFQRLLNDSERTLQEAFPGAFGDLYTQNTRAFRDLYAELRLYYRGANLHLEETLAEFWARLLERLFKQLHPQLLPDDYLDCLGKQAEALRPFGDAPRELRLRATRAFVAARSFVQGLGVASDVVRKVAQVPLAPECSRAIMKLVYCAHCRGVPGARPCPDYCRNVLKGCLANQADLDAEWRNLLDSMVLITDKFWGPSGAESVIGGVHVWLAEAINALQDNKDTLTAKVIQACGNPKVNPHGSGPEEKRRRGKLALQEKPSTGTLEKLVSEAKAQLRDIQDFWISLPGTLCSEKMAMSPASDDRCWNGISKGRYLPEVMGDGLANQINNPEVEVDITKPDMTIRQQIMQLKIMTNRLRGAYGGNDVDFQDASDDGSGSGSGGGCPDDTCGRRVSKKSSSSRTPLTHALPGLSEQEGQKTSAATCPEPHSFFLLFLVTLVLAAARPRWR

SEQ ID NO: 57 - DNA, 2757 bp, Homo sapiens
  source 1..2757: mol_type = unassigned DNA
gcattgcagactgcggagtcagacggcgctatgtacgccctcttcctcctggccagcctcctgggcgcggctctagccggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaggatcagcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaactaggaggaggaatattccatcttggcagaaaccacagcattggtttttttctacttgtgtgtctgggggaatgaacgcacagatctgtttgactttgttataaaaatagggctcccccacctcccccatttctgtgtcctttattgtagcattgctgtctgcaagggagcccctagcccctggcagacatagctgcttcagtgccccttttctctctgctagatggatgttgatgcactggaggtcttttagcctgcccttgcatggcgcctgctggaggaggagagagctctgctggcatgagccacagtttcttgactggaggccatcaaccctcttggttgaggccttgttctgagccctgacatgtgcttgggcactggtgggcctgggcttctgaggtggcctcctgccctgatcagggaccctccccgctttcctgggcctctcagttgaacaaagcagcaaaacaaaggcagttttatatgaaagattagaagcctggaataatcaggctttttaaatgatgtaattcccactgtaatagcatagggattttggaagcagctgctggtggcttgggacatcagtggggccaagggttctctgtccctggttcaactgtgatttggctttcccgtgtctttcctggtgatgccttgtttggggttctgtgggtttgggtgggaagagggccatctgcctgaatgtaacctgctagctctccgaaggccctgcgggcctggcttgtgtgagcgtgtggacagtggtggccgcgctgtgcctgctcgtgttgcctacatgtccctggctgttgaggcgctgcttcagcctgcacccctcccttgtctcatagatgctccttttgaccttttcaaataaatatggatggcgagctcctaggcctctggcttcctggtagagggcggcatgccgaagggtctgctgggtgtggattggatgctggggtgtgggggttggaagctgtctgtggcccacttgggcacccacgcttctgtccacttctggttgccaggagacagcaagcaaagccagcaggacatgaagttgctattaaatggacttcgtgatttttgttttgcactaaagtttctgtgatttaacaataaaattctgttagccaga

SEQ ID NO: 58 - DNA, 2667 bp, Mus musculus
  source 1..2667: mol_type = unassigned DNA
gtttgggcggggcccgcgcctgcgcactggcgtctgcggcgaggttggggggtagatttatattcgtctggtcagctgacgttctgcattgcagcctgcgaagtgaagcggcgccatgtacgccctcgccctcttcgccagccttctggccaccgctctgaccagccctgtccaagacccgaagacatgctctgggggctcagcagtgctgtgcagagatgtgaagacggcggtggactgtggggccgtgaagcactgccagcagatggtctggagcaagcccacagcgaaatcccttccttgcgacatatgcaaaactgttgtcaccgaagctgggaacttgctgaaagataatgctacgcaggaggagatccttcattacctggagaagacctgtgagtggattcatgactccagcctgtcggcctcgtgcaaggaggtggttgactcttacctgcctgtcatcctggacatgattaagggcgagatgagcaaccctggggaagtgtgctctgcgctcaacctctgccagtcccttcaggagtacttggccgagcaaaaccagaaacagcttgagtccaacaagatcccggaggtggacatggcccgtgtggttgcccccttcatgtccaacatccctctcctgctgtaccctcaggatcacccccgcagccagccccaacctaaggctaacgaggacgtctgccaggactgtatgaagctggtgtctgatgtccagactgctgtgaagaccaactccagctttatccagggcttcgtggaccacgtgaaggaggattgtgaccgcttggggccaggcgtgtctgacatatgcaagaactacgtggaccagtattccgaggtctgtgtccagatgttgatgcacatgcaacccaaggaaatctgtgtgctggctggcttctgtaatgaggtcaagagagtgccaatgaagactctggtccctgccaccgagaccattaagaacatcctccctgccctggagatgatggacccctatgagcagaatctggtccaggcccacaatgtgattttatgccagacctgtcagtttgtgatgaataagttttctgagctgattgtcaataatgccactgaggagctcctagttaaaggtttgagcaacgcatgcgcactgctccccgatcctgccagaaccaagtgccaggaggtggtgggaacatttggcccctccctgttggacatctttatccatgaggtaaaccccagctctctgtgcggtgtgatcggcctctgtgctgcccgcccggagttggtggaggcacttgagcagcctgcgccagccattgtatctgcactgctcaaagagcccacaccgccaaagcagcccgcacagcccaagcagtcggcattgcccgcccatgtgcctcctcagaagaatggtgggttctgtgaggtgtgcaagaaactggtcctctatttggaacataacctggagaaaaacagcaccaaggaggaaatcctggccgcacttgagaagggctgcagcttcctgccagacccttaccagaagcagtgcgatgactttgtggctgagtatgagcccttgctattggagatcctcgtggaagtgatggatcctggatttgtgtgctcgaaaattggagtttgcccttctgcctataagctgctgctgggaaccgagaagtgtgtctggggccctagctactggtgtcagaacatggagactgccgcccgatgcaatgctgtcgatcattgcaaacgccatgtgtggaactagtttcccagctgcagaagtcacctacttgtgggtctagggtaatgaacacatagatctatttgacttaataagtaggaaccccctttgcccttcccccatctcctctcccttactgtagcatttctgtcatgtaagaggtgctgacagccacttccgtgtcccctttctgctcgaaggatgaggataccttgggcatcagctccccggctgcccttttcacccacctgctggaggggggtggtgagccagagggcaggagcattttctgagccctttcttggtgtgtgggggatctatggccatctcctaccatgagggagctacccagcttcctgtggtaccaaggagttattttggatgattagaagcacagaatgatcaggcctttagagcgatggaatggccattgtcatagcacagagatttcagaagcacctgcaggtggcttgcttgggatgttgctgtccctgggtcagccttccattctgctttcctgtcttcccgtctgccttgttggggttctgtggggtagggtggggaggggaaacttgtgaatgtaacttgcctgtgccgtgtgacggtcacgtgggcctggtcttttgtgtgtgaggcccttgaccgtgtggcctctgcctggctgtttggggtcctgcacggctttcccaccacctgtagctcttgttgacctgcctgttcacctcatgagtgaagcgtctgcctggcagtgggccatgaactgaggggtctctgtgtagagtagaagcttcctgtgcctccggttgccaggagacagcctgtgcagttaaatggacctagattttgttttgcactaaagtttctgtgacttaataaagttctgttaaccaacagaaaaaaaaaaaaaaaaa

SEQ ID NO: 59 - protein, 527 aa, Homo sapiens
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQDQQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 60 - protein, 554 aa, Mus musculus
MYALALFASLLATALTSPVQDPKTCSGGSAVLCRDVKTAVDCGAVKHCQQMVWSKPTAKSLPCDICKTVVTEAGNLLKDNATQEEILHYLEKTCEWIHDSSLSASCKEVVDSYLPVILDMIKGEMSNPGEVCSALNLCQSLQEYLAEQNQKQLESNKIPEVDMARVVAPFMSNIPLLLYPQDHPRSQPQPKANEDVCQDCMKLVSDVQTAVKTNSSFIQGFVDHVKEDCDRLGPGVSDICKNYVDQYSEVCVQMLMHMQPKEICVLAGFCNEVKRVPMKTLVPATETIKNILPALEMMDPYEQNLVQAHNVILCQTCQFVMNKFSELIVNNATEELLVKGLSNACALLPDPARTKCQEVVGTFGPSLLDIFIHEVNPSSLCGVIGLCAARPELVEALEQPAPAIVSALLKEPTPPKQPAQPKQSALPAHVPPQKNGGFCEVCKKLVLYLEHNLEKNSTKEEILAALEKGCSFLPDPYQKQCDDFVAEYEPLLLEILVEVMDPGFVCSKIGVCPSAYKLLLGTEKCVWGPSYWCQNMETAARCNAVDHCKRHVWN

SEQ ID NO: 61 - skipped

SEQ ID NO: 62 - DNA, 1185 bp, Mus musculus
  source 1..1185: mol_type = unassigned DNA
ggggaccatctgtcttgcttctatctgggacatacttctgacactgagtccccaagaacctgtatcctggacaatcctgggcacacccacatcttttggggggctctcataaacgtcctgtgagtggttctcataggagagaacctggtttttctggagccaagatgctgatccagctgtgcctgaccctcttctttcttgggtgctccattgctggaaccccagcacctgggcctgagggcgttctgatgggcatcgtggggggtcatagtgccccacaggggaagtggccatggcaggtcagcctgaggatctatagatactactgggccttctgggtgcacaactgtgggggctccatcatccacccacagtgggtgctgactgctgcccactgcattcgtgagagagatgccgacccatcagtctttcggatccgtgttggggaggcgtacctctatgggggcaaggagctgctgagtgtgagccgggtcatcatccacccagactttgtccacgctggcctgggttcagatgtggctctgctccagctggcagtgtctgtacaatcctttcctaatgtcaagccagtcaagctgccctctgagtctcttgaggtcaccaagaaggatgtgtgctgggtgaccggctggggtgcagtgagcacacacaggtcgctgcctcctccctaccgcctacagcaggtgcaggtaaagataattgacaactcactttgtgaggagatgtaccacaatgccaccaggcaccgcaatcgtggccagaaactgatcctaaaggacatgttatgtgcaggcaaccagggccaagattcctgctatggtgactcaggtggccctctggtctgcaatgtgacaggctcctggaccctggtgggagtggtgagctggggctatggctgtgccctcagggacttccctggggtctatgcacgtgtacagtccttcctgccctggatcacgcagcagatgcagaggttctcctgagcccagctgctggcctcgtggccatcagatgaggataggtcggctttggtcacgattccagagtggacggccagggtttaggctctggagtgtaggtgaccagggcaccgagcattagctactgaaggacatggctcagtttcctggcttcctaaataaagatatgtttgttgcacaaaaa

SEQ ID NO: 63 - skipped

SEQ ID NO: 64 - protein, 279 aa, Mus musculus
MLIQLCLTLFFLGCSIAGTPAPGPEGVLMGIVGGHSAPQGKWPWQVSLRIYRYYWAFWVHNCGGSIIHPQWVLTAAHCIRERDADPSVFRIRVGEAYLYGGKELLSVSRVIIHPDFVHAGLGSDVALLQLAVSVQSFPNVKPVKLPSESLEVTKKDVCWVTGWGAVSTHRSLPPPYRLQQVQVKIIDNSLCEEMYHNATRHRNRGQKLILKDMLCAGNQGQDSCYGDSGGPLVCNVTGSWTLVGVVSWGYGCALRDFPGVYARVQSFLPWITQQMQRFS

SEQ ID NO: 65 - DNA, 1262 bp, Homo sapiens
  source 1..1262: mol_type = unassigned DNA
agttcacagctgccctgttgcagggaggcggtggcccttctgttgctagaccgagcctgtgggatataccaaggcagaggagcccatagccatgaggagcctcggggccctgctcttgctgctgagcgcctgcctggcggtgagcgctggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaagtttctctatcacaaatccaaatggaacataaccatggagtcctatgtggtccacaccaactatgatgagtatgccattttcctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgagagtccggagggctgtgctaccccaagaagaggaaggatcagggggtgggcaactggtaactgaagtcaccaagaaagaagattcctgccagctgggctactcggccggtccctgcatgggaatgaccagcaggtatttctataatggtacatccatggcctgtgagactttccagtacggcggctgcatgggcaacggtaacaacttcgtcacagaaaaggagtgtctgcagacctgccgaactgtggcggcctgcaatctccccatagtccggggcccctgccgagccttcatccagctctgggcatttgatgctgtcaaggggaagtgcgtcctcttcccctacgggggctgccagggcaacgggaacaagttctactcagagaaggagtgcagagagtactgcggtgtccctggtgatggtgatgaggagctgctgcgcttctccaactgacaactggccggtctgcaagtcagaggatggccagtgtctgtcccggggtcctgtggcaggcagcgccaagcaacctgggtccaaataaaaactaaattgtaaactcctgaaa

SEQ ID NO: 66 - DNA, 1284 bp, Mus musculus
  source 1..1284: mol_type = unassigned DNA
tcatttgcctccggatccttccccagcctcagtttgtagctgccactgccggggaagtaatccctgctcccacactgagctgtgagagagacatcaaagaccaaagaccacagagccatgcagggtctcaggaccctgttcctgctgctgactgcctgcctcgcttcgagggctgaccctgcgtcaacactgccagatatccaggttcaggagaacttcagtgagtcccggatctatggaaaatggtacaacctggcggtgggatccacctgcccgtggctgagccgcattaaggacaagatgagcgtgagcacgctggtgctgcaggagggggcgacagaaacagagatcagcatgaccagtactcgatggcggagaggtgtctgtgaggagatcactggggcgtaccagaagacggacatcgatggaaagttcctctaccacaaatccaaatggaacataaccttggaatcctatgtggtccacaccaactatgacgaatatgccattttccttaccaagaagtccagccaccaccacgggctcaccatcactgccaagctctatggtcgggagccacagctgagggacagccttctgcaggagttcaaggatgtggccctgaatgtgggcatctctgagaactccatcatttttatgcctgacagaggggaatgtgtccctggggatcgggaggtggagcccacatcaattgccagagcccggcgggcagtgctgccccaagagagtgaggggtcagggactgagccactaataactgggaccctcaagaaagaagactcctgccagctcaattactcagaaggcccctgcctagggatgcaagagaggtattactacaacggcgcttccatggcctgcgagacctttcaatatgggggttgcctaggcaacggcaacaacttcatctctgagaaggactgtctgcagacatgtcggaccatagcggcctgcaatctccccatagtccaaggcccctgccgagccttcataaagctctgggcatttgatgcagcacaagggaagtgcatccaattccactacgggggctgcaaaggcaacggcaacaaattctactctgagaaggaatgcaaagagtactgtggagtccctggtgatgggtacgaggaactaatacgcagttgaaggtgccagtctgcaagccagagggtagccactgtttgtcacagcgcagtccagcttagatgatctggacccaaataaaacaagttgtcacttcctagcaaaaaaaaaaaaaaaaaa

SEQ ID NO: 67 - protein, 352 aa, Homo sapiens
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 68 - protein, 349 aa, Mus musculus
MQGLRTLFLLLTACLASRADPASTLPDIQVQENFSESRIYGKWYNLAVGSTCPWLSRIKDKMSVSTLVLQEGATETEISMTSTRWRRGVCEEITGAYQKTDIDGKFLYHKSKWNITLESYVVHTNYDEYAIFLTKKSSHHHGLTITAKLYGREPQLRDSLLQEFKDVALNVGISENSIIFMPDRGECVPGDREVEPTSIARARRAVLPQESEGSGTEPLITGTLKKEDSCQLNYSEGPCLGMQERYYYNGASMACETFQYGGCLGNGNNFISEKDCLQTCRTIAACNLPIVQGPCRAFIKLWAFDAAQGKCIQFHYGGCKGNGNKFYSEKECKEYCGVPGDGYEELIRS

SEQ ID NO: 69 - DNA, 2011 bp, Homo sapiens
  source 1..2011: mol_type = unassigned DNA
gctgctgctgcttgtcccaagaccaagtcgtaatagcaacttcccttcctcagctgcctgaactttttttttcccttgtagctggagagaagtgtcacattttgctcactctcaaccttcctcgcccacccccttcccggagaacctgtgcggtgtgtagagggtgctgtgagccacctccagcctcgggtggctgcttaagtaactttcaactcctctcttcttaacactatgaagtgtctcgggaagcgcaggggccaggcagctgctttcctgcctctttgctggctctttttgaagattctgcaaccggggcacagccacctttataacaaccgctatgctggtgataaagtgataagatttattcccaaaacagaagaggaagcatatgcactgaagaaaatatcctatcaacttaaggtggacctgtggcagcccagcagtatctcctatgtatcagagggaacagttactgatgtccatatcccccaaaatggttcccgagccctgttagccttcttacaggaagccaacatccagtacaaggtcctcatagaagatcttcagaaaacactggagaagggaagcagcttgcacacccagagaaaccgaagatccctctctggatataattatgaagtttatcactccttagaagaaattcaaaattggatgcatcatctgaataaaactcactcaggcctcattcacatgttctctattggaagatcatatgagggaagatctctttttattttaaagctgggcagacgatcacgactcaaaagagctgtttggatagactgtggtattcatgcaagagaatggattggtcctgccttttgtcagtggtttgtaaaagaagctcttctaacatataagagtgacccagccatgagaaaaatgttgaatcatctatatttctatatcatgcctgtgtttaacgtcgatggataccattttagttggaccaatgatcgattttggagaaaaacaaggtcaaggaactcaaggtttcgctgccgtggagtggatgccaatagaaactggaaagtgaagtggtgtgatgaaggagcttctatgcacccttgtgatgacacatactgtggcccttttccagaatctgagccggaagtgaaggctgtagctaacttccttcgaaaacacagaaagcacattagggcttatctctcctttcatgcatatgctcagatgttactgtatccctattcttacaaatatgcaacaattcccaattttagatgtgtggaatctgcagcttataaagctgtgaatgcacttcagtcagtatacggggtacgatacagatatggaccagcctccacaacgttgtatgtgagctctggtagctcaatggattgggcctacaaaaatggaataccttatgcatttgctttcgaactacgtgacactggatattttggatttttactcccagagatgctcatcaaacccacctgtacagaaactatgctggctgtgaaaaatatcacaatgcacctgctaaagaaatgtccctgagacagcccaaggctcaggtcaactgccataggattctgagcaaggcctacttggccctggatagaaattgttttcaaagagaagggcagctgcttagagtgaacatgtctatggactttaaaaagaccccacgcaatttgactttgtggcaatagaaaacagtaaaaaacagggcatagcctagtttgttataagaaaaagcatccattttctatccttttagagtcttatttgattatggtgggagggaatgttttcaaatttcccatttctcaagaaatgttcatattaattgaggatttcccttcaataaatctcatgtcctcaattaggatctaggctagtaatgatgtttaattacttggttctcattggaaatggaaaattatgaaatggaaaaaaacaactatgcttcttaatgtaataaacataggccgggcgcggtggctcacacctgtaattccag

SEQ ID NO: 70 - DNA, 1815 bp, Mus musculus
  source 1..1815: mol_type = unassigned DNA
gcttccaggctgggctgggctgcatttcctcacaagacccagtcttaatagcaactccctagctgcctaaagtgtcacattttgctcattcccaagccttctcccccacccctttcccagacaacttgctggtgagcagggggtgttgtaagccacctccagcctcttcctttggtgactgctgttaagcaactttcagctcctctcattgcaatagtatgaacttcctcgggaatcccaggagccacacagcagcttttctgccggtttgctggctccttttgaatattctgaaacctgggcactgccactcttacgacaaccgctatgctggtgataaagtgataagactcatccccaaatccgaagaggaagcacttgcactcaagaacatataccaccaacttaagggtcctcatagaagatcttcagaaagctgtggaaaatgaaaacagcttgcaaacccagagaaacagaagatccctgtctgaatacaactacgaggtgtatcactccttagaagatattcagagttggttgcaccatctgaaccaaactcagccaggcctcgttcgagtgttctctattggaagatcatacgaaggaagacctctctttattatgcagctgggcagaaaatcacgagcctacaaaagagctgtctggatagactgcggcattcacgcaagagaatggatcggtcctgccttttgtcagtggtttgtaagagaagccattctgacctacaagactgacccagccatgaaaaaaatgttgaatcatctgtatttctatatcatgcctgtattcaatgtcgatggataccatttcagctggacacatgatagattctggagaaagacccggtcaagagactctaagtttcgctgtcgtggagttgatgccaatcgcaactggaaagtgaaatggtgcgatgaaggagcctccgcacacccttgtgatgatacatactgtggtcctttcccagaatctgaaccagaagtgaaggctgtagctaacttccttcgaaaacacagaaagcgcatcagggcgtacctgtcttttcatgcctatgctcagatgttgctatatccttattcttacaaatatgcaacaatccccaattttagctgtgtggaatttgcagctcacaaagctgtgaaggcacttcggtcggtacatgggatccgatacagacatggccccgcctcccaaacactgtatgtaagctctggtaactcaatggattgggcttataaaaatggaataccctatgcatttgcttttgaactgcgagacactgggcattttggatttctactgccagagatgctcattaaacccacctgcacagagaccatgctggctgtgaagaatatcacaatgcacctgctaaagaaatgtccttgagaggaacagtcatctagtctaccaacacagagaaagcagattctgatcaaaaggcaccttggcaaacataactgtttagactggaaggatccatggacctaaaaggatctttcatgcaattcgacactttgcaaagactgaaaagtagggcacagcctactttggtatgagaagaaagtccactttatatctctttagagtcctatttgattacaagggggagggaatgttttcaaattcctttgtctcaaaaggtgtacaaattaattgaggatttctcttctctcaaccactccatccttgattaagacctatgctaccaacgacattgaattacttggttcttatttgaaatggaaacaatcaactgtgtttcttaataaatacataacttttcaaaaaact

SEQ ID NO: 71 - protein, 437 aa, Homo sapiens
MKCLGKRRGQAAAFLPLCWLFLKILQPGHSHLYNNRYAGDKVIRFIPKTEEEAYALKKISYQLKVDLWQPSSISYVSEGTVTDVHIPQNGSRALLAFLQEANIQYKVLIEDLQKTLEKGSSLHTQRNRRSLSGYNYEVYHSLEEIQNWMHHLNKTHSGLIHMFSIGRSYEGRSLFILKLGRRSRLKRAVWIDCGIHAREWIGPAFCQWFVKEALLTYKSDPAMRKMLNHLYFYIMPVFNVDGYHFSWTNDRFWRKTRSRNSRFRCRGVDANRNWKVKWCDEGASMHPCDDTYCGPFPESEPEVKAVANFLRKHRKHIRAYLSFHAYAQMLLYPYSYKYATIPNFRCVESAAYKAVNALQSVYGVRYRYGPASTTLYVSSGSSMDWAYKNGIPYAFAFELRDTGYFGFLLPEMLIKPTCTETMLAVKNITMHLLKKCP

SEQ ID NO: 72 - protein, 262 aa, Mus musculus
MQLGRKSRAYKRAVWIDCGIHAREWIGPAFCQWFVREAILTYKTDPAMKKMLNHLYFYIMPVFNVDGYHFSWTHDRFWRKTRSRDSKFRCRGVDANRNWKVKWCDEGASAHPCDDTYCGPFPESEPEVKAVANFLRKHRKRIRAYLSFHAYAQMLLYPYSYKYATIPNFSCVEFAAHKAVKALRSVHGIRYRHGPASQTLYVSSGNSMDWAYKNGIPYAFAFELRDTGHFGFLLPEMLIKPTCTETMLAVKNITMHLLKKCP

SEQ ID NO: 73 - DNA, 516 bp, Homo sapiens
  source 1..516: mol_type = unassigned DNA
acactttgtatggcaagtggaaccactggcttggtggattttgctagatttttctgatttttaaactcctgaaaaatatcccagataactgtcatgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctgttgagcaccttgtggaggggctaaggaagtgtgtaaatgagctgggaccagaggcttctgaagctgtgaagaaactgctggaggcgctatcacacttggtgtgacatcaagataaagagcggaggtggatggggatggaagatgatgctcctatcctccctgcctgaaacctgttctaccaattatagatcaaatgccctaaaatgtagtgacccgtgaaaaggacaaataaagcaatgaataca

SEQ ID NO: 74 - DNA, 615 bp, Mus musculus
  source 1..615: mol_type = unassigned DNA
agtacctctgaggaaaagcagccatcaggtgacagcgagcagaactattgacaccgtgattttgttgggctttctgactgcattccaaagtcccggaaaacatcacaggcaccagctatgaagctggtatctatctttctgctggtgaccattggtatttgtggttattctgccactgcccttctcatcaaccgtctccctgttgttgacaaattacctgtacctttggacgacattattccctcatttgatcccttgaagatgcttctgaaaaccctgggcatttctgtagaacatctggtgacaggactgaagaagtgtgtggacgagctgggaccagaggcttccgaggccgtgaagaagcttctggtaattataatttgttcttatttccctgggagaagcctttgctacgtgaacaacctcccatcatttgaggctctttcacacctggtataaaatcttcataaagagatttaaggaaggagtatgaaaagaaggatttgctcactctggctggctggatctctcattctatcatttgtaaactgaatgtcccagagtttaaggagtctagaaaagtatgaataaagcaatgaaaagaaaaaaaaaaaa

SEQ ID NO: 75 - protein, 93 aa, Homo sapiens
MKLVTIFLLVTISLCSYSATAFLINKVPLPVDKLAPLPLDNILPFMDPLKLLLKTLGISVEHLVEGLRKCVNELGPEASEAVKKLLEALSHLV

SEQ ID NO: 76 - protein, 113 aa, Mus musculus
MKLVSIFLLVTIGICGYSATALLINRLPVVDKLPVPLDDIIPSFDPLKMLLKTLGISVEHLVTGLKKCVDELGPEASEAVKKLLVIIICSYFPGRSLCYVNNLPSFEALSHLV

SEQ ID NO: 77 - skipped

SEQ ID NO: 78 - DNA, 644 bp, Mus musculus
  source 1..644: mol_type = unassigned DNA
gtgaaatcacctctgtgctgtcaaagcattgcagacctctgtatagacagatataccatggtgtctcacaagacccttcatagcatgtcctggatgctactgtgttgcctgatgtccctttcttgggtacaaggtgaacaatcccagaaaaaactgtcttctccacgcatcagctgtccccaagaagcccaagcttatggctcctattgctatttactgattctggaaccacagacctgggctaatgcagaggggagtttgcccaatgaaaatggatggaagtggagcagctctgaccctctgaccttctataactgggagataccaccctccatgtctgcacaccacggctactgtgcagctttgtctcaggcctcaggttatcagaagtggagagattattattgtgacacaatatttccctatgtctgcaaattcaagggttaggccagttctgatttcaactgcctgaaagtatcctgaagatcacatagacaaaggagcaagcatgatggctcaccaagaaagtccttctcacaccccgacaccgaattcctcatctcatctctgctgttcttccataagtgtattctctggggactctggcctaaggattcggagaacaataataaaatttagtcaat

SEQ ID NO: 79 - skipped

SEQ ID NO: 80 - protein, 129 aa, Mus musculus
MVSHKTLHSMSWMLLCCLMSLSWVQGEQSQKKLSSPRISCPQEAQAYGSYCYLLILEPQTWANAEGSLPNENGWKWSSSDPLTFYNWEIPPSMSAHHGYCAALSQASGYQKWRDYYCDTIFPYVCKFKG

SEQ ID NO: 81 - skipped

SEQ ID NO: 82 - DNA, 503 bp, Mus musculus
  source 1..503: mol_type = unassigned DNA
cctctccaccatgaaggggacacttcttctgctggccttgctgatgattggagagctgggcttccatacaacagaagcatgtgttcctttctttgctggatatgctggtgttatctcaggaagcaggctgtggttgtatcatgaacttagcgcattcaatggtacacctaaggaaacagtggcctatgaaaaaatccaggactgctacaaagaacaaggagttaaatcccaaacactggaaccacaaattttggcctctatacttgtcaccccagaatgcctgcagtactattccgaagaaacctttacgaaaattaaggatgcattaaaaaaaatatcacaacattagaagttaatgggttataggcaacccatatttgcaacatgtctaatctgagcaccatcttgctgatctgtcctttttctctcctgaagttggattcctgacacctgtgccatcatcctgcactctcttaggctgattctaataaatgcatgcagct

SEQ ID NO: 83 - skipped

SEQ ID NO: 84 - protein, 112 aa, Mus musculus
MKGTLLLLALLMIGELGFHTTEACVPFFAGYAGVISGSRLWLYHELSAFNGTPKETVAYEKIQDCYKEQGVKSQTLEPQILASILVTPECLQYYSEETFTKIKDALKKISQH

SEQ ID NO: 85 - DNA, 670 bp, Homo sapiens
  source 1..670: mol_type = unassigned DNA
aggtggcagcatggattctaggcaggctgctgcactgctggtgctgctgctgttaatagacgggggctgtgctgaagggccaggaggccagcaggaagaccagctctccgcggtgagtgtgtgtcccatccccatatcaccattgcctctacttcggttgagacttgtgctctaggttctgatactttctctggctgccaaggttgtcattaggtcctcacatctgaggaaatggttccgcagcctcctaccacttgcccctggaagccagtcccttccccttgtgacttacgtgtccagggtatttgcccatcttccttccctgatacccccttggcacaggaggaagacagcgaacccctcccaccacaggatgcccagacctctgggtcactgttgcactacctgctccaggcaatggagagacctggccggagccaagccttcctgtttcagccccagaggtttggcagaaatacccagggatcctggaggaatgaatggctgagtccccgggctggagaggggctgaattcccagttctggagcctggctgcccctcaacgctttgggaagaagtgacatgtcatcccttgatatgtctgcatgcaaggtccacacccaaaagtgtcaatgtttgccccccaaataaaattgtctggcttctgaa

SEQ ID NO: 86 - DNA, 451 bp, Mus musculus
  source 1..451: mol_type = unassigned DNA
ggcagatggcagcatggactccaagtgggctgctctgctgctgctgctactgctgctgctgaattggggccacactgaagaggcagggagctggggtgaagaccaagtctttgcaggagaagataagggaccccacccaccacagtatgcccacattccagacaggatccagactcctgggtccctctttcgtgttctgctccaggccatggacacacctagaaggagcccagccttcctgtttcagccccagaggtttggcagaagtgcatgggggtcctggagcaaggaacagctaaatccgcaggccagacagttctggagcctggccgctcctcagcgctttgggaagaagtagcatcgtcagctgtgatgcctgcatgcaaaaccacttccccatgttccctgtgtgcccccaaataaaaatggtccggctggcttcagaaaaaaa

SEQ ID NO: 87 - protein, 116 aa, Homo sapiens
MVPQPPTTCPWKPVPSPCDLRVQGICPSSFPDTPLAQEEDSEPLPPQDAQTSGSLLHYLLQAMERPGRSQAFLFQPQRFGRNTQGSWRNEWLSPRAGEGLNSQFWSLAAPQRFGKK

SEQ ID NO: 88 - protein, 114 aa, Mus musculus
MDSKWAALLLLLLLLLNWGHTEEAGSWGEDQVFAGEDKGPHPPQYAHIPDRIQTPGSLFRVLLQAMDTPRRSPAFLFQPQRFGRSAWGSWSKEQLNPQARQFWSLAAPQRFGKK

SEQ ID NO: 89 - DNA, 1317 bp, Homo sapiens
  source 1..1317: mol_type = unassigned DNA
cttcctgacccaggggctccgctggctgcggtcgcctgggagctgccgccagggccaggaggggagcggcacctggaagatgcgcccattggctggtggcctgctcaaggtggtgttcgtggtcttcgcctccttgtgtgcctggtattcggggtacctgctcgcagagctcattccagatgcacccctgtccagtgctgcctatagcatccgcagcatcggggagaggcctgtcctcaaagctccagtccccaaaaggcaaaaatgtgaccactggactccctgcccatctgacacctatgcctacaggttactcagcggaggtggcagaagcaagtacgccaaaatctgctttgaggataacctacttatgggagaacagctgggaaatgttgccagaggaataaacattgccattgtcaactatgtaactgggaatgtgacagcaacacgatgttttgatatgtatgaaggtgataactctggaccgatgacaaagtttattcagagtgctgctccaaaatccctgctcttcatggtgacctatgacgacggaagcacaagactgaataacgatgccaagaatgccatagaagcacttggaagtaaagaaatcaggaacatgaaattcaggtctagctgggtatttattgcagcaaaaggcttggaactcccttccgaaattcagagagaaaagatcaaccactctgatgctaagaacaacagatattctggctggcctgcagagatccagatagaaggctgcatacccaaagaacgaagctgacactgcagggtcctgagtaaatgtgttctgtataaacaaatgcagctggaatcgctcaagaatcttatttttctaaatccaacagcccatatttgatgagtattttgggtttgttgtaaaccaatgaacatttgctagttgtatcaaatcttggtacgcagtatttttataccagtattttatgtagtgaagatgtcaattagcaggaaactaaaatgaatggaaattcttaaagggaatgatgtgattcaagctggaaagagggttgggagaaacagcttgtccaggtggagctatgttatgatcagatcgaagtgtgacccctgtgtggtccagacagccctgcagagagaaaacctttattccattatcaccaagcacctcctagtttccgacagtcatctccttctgctgggagaattagcagcagttcagggggcttatgttatgtccttgttcaactcaacttgagctcttgaactcctcctgtgggcctgtgaatgtattcattcattccacaactctgggtg

SEQ ID NO: 90 - DNA, 972 bp, Mus musculus
  source 1..972: mol_type = unassigned DNA
ataatctatgtgcagctcaagcaggtaggccagctttcataggtccctggggattgcagccaagagctgagtagaaagagagcagtttctggaagatgcgtccagttgctacaggcatcttcaaggcactagtgtttattttctcctccctgtgcgcctggtattctgggtacctgctcgcggagctcattcctgacgtgcccctgtccagcactctctacaacatccgaagcattggagagagacctgttctcaaagccccagcccccaaaagacaaaaatgtgaccattggtccccatgtcctcctgacacctatgcctaccggctgctcagtggtggtggccgcgacaagtatgccaagatctgctttgaggatgaagtgctaataggagagaagacggggaatgtggcaagagggataaacattgctgtcgtcaactatgagacaggaaaagtgatagcgacaaagtactttgatatgtatgaaggtgataactccgggccaatggccaagttcattcagagcactccttcaaaatccctgctgttcatggtgactcatgatgatggaagttccaaactgaaggctcaagcaaaggatgccatagaagcccttggaagcaaagaaatcaagaacatgaagttcagatcaagctgggtgtttgttgcagcaaagggctttgagctcccttcagaaatcgagagagaaaaaatcaaccactcagatcaatccaggaacagatatgcaggctggccagcggagatccagatcgaaggatgcatacccaaagggctgagataacggtgcttggacctcaataaacacgttttgtacagatatactcagctgaatgtcacaggagtctgatcctttgaagtccagctgtgaaggaaagtgttgacggggatttggcagtttgttgtaaaccactaagacactgttaatcacacggatcctaagcagctgatgg

SEQ ID NO: 91 - protein, 235 aa, Homo sapiens
MRPLAGGLLKVVFVVFASLCAWYSGYLLAELIPDAPLSSAAYSIRSIGERPVLKAPVPKRQKCDHWTPCPSDTYAYRLLSGGGRSKYAKICFEDNLLMGEQLGNVARGINIAIVNYVTGNVTATRCFDMYEGDNSGPMTKFIQSAAPKSLLFMVTYDDGSTRLNNDAKNAIEALGSKEIRNMKFRSSWVFIAAKGLELPSEIQREKINHSDAKNNRYSGWPAEIQIEGCIPKERS

SEQ ID NO: 92 - protein, 235 aa, Mus musculus
MRPVATGIFKALVFIFSSLCAWYSGYLLAELIPDVPLSSTLYNIRSIGERPVLKAPAPKRQKCDHWSPCPPDTYAYRLLSGGGRDKYAKICFEDEVLIGEKTGNVARGINIAVVNYETGKVIATKYFDMYEGDNSGPMAKFIQSTPSKSLLFMVTHDDGSSKLKAQAKDAIEALGSKEIKNMKFRSSWVFVAAKGFELPSEIEREKINHSDQSRNRYAGWPAEIQIEGCIPKGLR

SEQ ID NO: 93 - DNA, 5682 bp, Homo sapiens
  source 1..5682: mol_type = unassigned DNA
cctccgaggctggcgatcggcggagctcccacctccgcttacagctcgctgccgccgtcctgccccgcgcccccaggagacctggaccagaccacgatgtggaaacgctggctcgcgctcgcgctcgcgctggtggcggtcgcctgggtccgcgccgaggaagagctaaggagcaaatccaagatctgtgccaatgtgttttgtggagccggccgggaatgtgcagtcacagagaaaggggaacccacctgtctctgcattgagcaatgcaaacctcacaagaggcctgtgtgtggcagtaatggcaagacctacctcaaccactgtgaactgcatcgagatgcctgcctcactggatccaaaatccaggttgattacgatggacactgcaaagagaagaaatccgtaagtccatctgccagcccagttgtttgctatcagtccaaccgtgatgagctccgacgtcgcatcatccagtggctggaagctgagatcattccagatggctggttctctaaaggcagcaactacagtgaaatcctagacaagtattttaagaactttgataatggtgattctcgcctggactccagtgaattcctgaagtttgtggaacagaatgaaactgccatcaatattacaacgtatccagaccaggagaacaacaagttgcttaggggactctgtgttgatgctctcattgaactgtctgatgaaaatgctgattggaaactcagcttccaagagtttctcaagtgcctcaacccatctttcaaccctcctgagaagaagtgtgccctggaggatgaaacgtatgcagatggagctgagaccgaggtggactgtaaccgctgtgtctgtgcctgtggaaattgggtctgtacagccatgacctgtgacggaaagaatcagaagggggcccagacccagacagaggaggagatgaccagatatgtccaggagctccaaaagcatcaggaaacagctgaaaagaccaagagagtgagcaccaaagagatctaatgaggaggcacagaccagtgtctggatcccagcatcttctccacttcagcgctgagttcagtatacacaagtgtctgctacagtcgccaaatcaccagtatttgcttatatagcaatgagttttattttgtttatttgttttgcaataaaggatatgaaggtggctggctaggaagggaagggccacagccttcatttctaggagtgctttaagagaaactgtaaatggtgctctggggctggaggctagtaaggaaactgcatcacgattgaaagaggaacagacccaaatctgaacctcttttgagtttactgcatctgtcagcaggctgcagggagtgcacacgatgccagagagaacttagcagggtgtccccggaggagaggtttgggaagctccacggagaggaacgctctctgcttccagcctctttccattgccgtcagcatgacagacctccagcatccacgcatctcttggtcccaataactgcctctagatacatagccatactgctagttaacccagtgtccctcagacttggatggagtttctgggagggtacacccaaatgatgcagatacttgtatactttgagccccttagcgacctaaccaaattttaaaaatactttttaccaaaggtgctatttctctgtaaaacacttttttttggcaagttgactttattcttcaattattatcattatattattgttttttaatattttattttcttgactaggtattaagcttttgtaattatttttcagtagtcccaccacttcataggtggaaggagtttggggttcttcctggtgcaggggctgaaataacccagatgcccccaccctgccacatactagatgcagcccatagttggcccccctagcttccagcagtccactatctgccagaggagcaagggtgccttagaccgaagccaggggaagaagcatcttcataaaaaactttcaagatccaaacattaatttgtttttatttattctgagaagttgaggcaaatcagtattcccaaggatggcgacaagggcagccaagcagggcttaggatatcccagcctaccaatatgctcattcgactaactaggagggtgagttggccctgtctcttcttttttctggacctcagtttcctcagtgagctggtaagaatgcactaaccttttgatttgataagttataaattctgtggttctgatcattggtccagaggggagataggttcctgtgatttttccttcttctctatagaataaatgaaatcttgttactagaacaagaaatgtcagatggccaaaaacaagatgaccagatttgatctcagcctgatgaccctacaggtcgtgctatgatatggagtcctcatgggtaaagcaggaagagagtgggaaagagaaccaccccactctgtcttcatatttgcatttcatgtttaacctccggctggaaatagaaagcattcccttagagatgaggataaaagaaagtttcagattcaacagggggaagaaaatggagatttaatcctaaaactgtgacttggggaggtcagtcatttacagttagtcctgtgtctttcgacttctgtgattattaaccccactcactaccctgtttcagatgcatttggaataccaaagattaaatccttgacataagatctcatttgcagaaagcagattaaagaccatcagaaggaaattatttaggttgtaatgcacaggcaactgtgagaaactgttgtgccaaaaatagaattccttctagtttttcttgttctcatttgaaaggagaaaattccactttgtttagcatttcaagcttttatgtatccatcccatctaaaaactcttcaaactccacttgttcagtctgaaatgcagctccctgtccaagtgccttggagaactcacagcagcacgccttaatcaaaggttttaccagcccttggacactatgggaggagggcaagagtacaccaatttgttaaaagcaagaaaccacagtgtctcttcactagtcatttagaacatggttatcatccaagactactctaccctgcaacattgaactcccaagagcaaatccacattcctcttgagttctgcagcttctgtgtaaatagggcagctgtcgtctatgccgtagaatcacatgatctgaggaccattcatggaagctgctaaatagcctagtctggggagtcttccataaagttttgcatggagcaaacaaacaggattaaactaggtttggttccttcagccctctaaaagcatagggcttagcctgcaggcttccttgggctttctctgtgtgtgtagttttgtaaacactatagcatctgttaagatccagtgtccatggaaacattcccacatgccgtgactctggactatatcagtttttggaaagcagggttcctctgcctgctaacaagcccacgtggaccagtctgaatgtctttcctttacacctatgtttttaagtagtcaaacttcaagaaacaatctaaacaagtttctgttgcatatgtgtttgtgaacttgtatttgtatttagtaggcttctatattgcatttaacttgtttttgtaactcctgattcttccttttcggatactattgatgaataaagaaattaaagtgatggttttattggtttcctttcccccaattaaggccaaataaagtcgtgagaacattacccatttacttcctaagagtggatttgagtttcaggagccttaaaaaggactgaagagtttctcctgggactacagcttggatggaaagtttactcggggactggtagaacaagtttctttgaaaggatcatggatagtgggtctggagtagaaggtgatgacaagtcagaaagtgtcttagtcccttcaagttgctataacaaaatgcctcagactgggtaattataggcaacagaaatgtattgctcacagttctgaaggctgggaagcctaagatcaaggtaccagcagattcagtgtctgatgacggcttgttgaagcttgcctgcttcaaagaatgcacctttttgatgcatccccacatggtgaaaagggtgaacaaactcccttaggcctcttttataagggcacttacccattcatgagggttaaactttgatgacctaatcatctcccaagagcctcacctctcaataccattgctttggggattcaattttaacatatgaattttgggggacataaacattcaaaccatagcaggtgcattcctgtattgggaagacattaaattaaatgactcgtaaatcccccatatgatttaggagactaaatggcatccagtgaggctgactgcagtagcctgtgagaaggcaaataaacatgggaaggtagtacagggaacatggaatcattgccagggacaggctaaataaccttaatggcacttccttgatacacacattccatgggggtaaacagttccaagtgggctgaacgttagctggttaaaacctcttttgcttggggacacatgactaacgctttccaacctgccattttcatatgtatcatgcactctccacccacaataattctgcctcccaaaagagcatattatagtcaatgaggtttaaggtttcttcggttcagtcatgtggaagttttgctttcctaaaattatagctggatcactttgattggatttgggatattacccaaatgctaatcaaatattaaaaggcaatcttttttgttatgtgattgctacagattactgctcttacagtttgattcttcaaattttcattctcagaagggaatcttaaacccagtttgaagggtaccttgtccacaaccacttttaaattttggctaacttcccaaccttccaccatgttctgggaaaactttttctgctgttatagtatgtatgtattcaggaagtgcaacattattaaactaagcataattcagaagctaagtttcctttcatcaaaatttaatattacgaattgtgcaaagaagtatgttactttgatcagctttattttagagcttctttatcaagttatcatcagtcctcttgaattatttctgaaataaggggaaggaaggaagggaaaaaatatgaatgcatagatagatctaagcagatgccttcagagccctggaataatcctagctgggatatgggacatgaaaacaactctggcctactgctgctgggggctgttgcatggtgtgacataggaagtaaaatgttgctgtatgaataggaaggagggtcttcctgtcaatgagaattaatgttttcctaatgatttaacttttttactgaaaaaataatatgtgtacatggtaaaaatccaaattgtatcacagaaaatacagtgaataaaaccatcctcttaagagggtgtgtacatttataaatttgcctatgattgcctataaagtcaaagaagttgcaccagtatgcatgactgccaaaagtacataaactgagcatatccatttccctacattcttgcaaacattgtgccttcgaactttttggcctctgatagaggaaaaatggtaactccttgttttattttttatattcattattccttttgtaagattaaatatcatctcatgaataa

SEQ ID NO: 94 - DNA, 3609 bp, Mus musculus
  source 1..3609: mol_type = unassigned DNA
ttcctcggagcctggtgataagcgacgctcccaccttcgcctctaactcgctgccgccaccctgcccagtgtcctccggagtcccggacccgagcacgatgtggaaacgatggctggcgctctcgctggtgaccatcgccctggtccacggcgaggaggaacctagaagcaaatccaagatctgcgccaatgtgttttgtggagctggcagggaatgtgccgtcacagagaagggggagcccacgtgcctctgcattgagcaatgcaaacctcacaagaggcctgtgtgtggcagtaatggcaagacctacctcaaccactgtgaacttcatagagatgcctgcctcactggatccaagatccaggttgattatgatgggcactgcaaagaaaagaagtctgcgagtccatctgccagcccagttgtctgctatcaagctaaccgcgatgagctccgacggcgcctcatccagtggctggaagctgagatcattccagatggctggttctctaaaggcagtaactacagtgagatcctagacaagtactttaagagctttgataatggcgactctcacctggactccagtgaattcctgaaattcgtggagcagaatgaaacagccatcaacatcaccacttatgcagatcaggagaacaacaaactgctcagaagcctctgtgttgacgccctcattgaactgtctgatgagaacgctgactggaaactcagcttccaagagttcctcaagtgcctcaacccatccttcaaccctcctgagaagaagtgtgccctggaggacgaaacctatgcagatggagctgagactgaggtggactgcaatcgctgtgtctgttcctgtggccactgggtctgcacagcaatgacctgtgatggaaagaatcagaagggggtccagacccacacagaggaggagaagacaggatatgtccaggaactccagaagcaccagggcacagcagaaaagaccaagaaggtgaacaccaaagagatctaagaagaggcacagagcaccgtgtccggagcccagcgcctcctcttcagcgctgagcccagtacacacagagtctgcagcaatcaccaaatcactagtatttgcttgtatggcagcgaatcttattttgtttgttttgcaataaaggaaatgagggtggccagcctagcgagggaaggccacaaccttcacctgtaggaatgctttaagagaaactaaaggacaccttgggacgagaggcaactaaggaaacagcatcgggttggcagaggagcagaggcaggtttgaatgaagcctttctggggtcacagcagctgcgaggagaatacaggaaaagcatagagaaacattgaactagccctgctggaggaagtggggggagctttgtagggaggaaccctgctgctttgacccttgtcaccactgtcagcatgacagacctgcagcaagtctgcttctccttttggtcccaacaatcacctgaacacacagccgcccaactagttacctgtgtccctcagccttgcatggagtttcctggaggaggtgtttaaatgatgcagacacttatgtacttcaagcgccatggagacctaaccaaatttttaaaatacatttttcttttttttttttttttgttaaccaaaggtgctatttctctgtaaaagacttttttccaagctgacttcattcctcagttattaccgttatattattgttgttttttaatatttcattttttgactagatattaagcttttgtaattattttttcattagtcctactatttcagaagtgaaggtgaagggggtttgggcatttttccaggtacagggaactctgtaacacaaacagcccataccctgtcacatattagaccggttgcagttcggagctgcaccccaacccagagcttctagaaaatcagctccatgccacgaagcacaagaggcccctcagacagaagccacaggacaaagcatcttcatagacagctgttgagatccaaacagttaatttgcttttgtttcttgtaagaagttccaaggatggacgctcaggctatcccagcctgccagcctgctgtgatctgtggctaactggcagagtcagccactgtggtccttagctgctcctgtttctaggtgtcagtttacttagtaaactggtaagaatgaatcttggaatttaataaatggtagtttgtggtttagccaactggtccagagggagctaccttctccttaggatagatgaatctacaccataagaaaaaccagccaggaatagcatggatgggttttgctttggttgaaatgatcctagcaggtgactgggtatgaggacttcatggtcactctgcccaggaagagagcgtgaaggacaactagcagcttccttagggatggtacacatgagtgtgatctctggagtcagaggtttccccacacacatgatgataaaacttttcagatttagagcggttaaaactggagatcgaatctggattgagaatcagcactgggggagaaactgttattgaaagtcaatcctttctttgagcctccgaataaactatggagattttcctgcataggaaagtgtggaatgttgagctattgagatgggagtggaattcgtcctaaatagtttttcctggtctcatctgaacaagacaatttggctctgcctagtgttctgtgccctccctttcaaaagctctgagccccgctcatgcagtccagatttcatccccctctccaagtgccttggagagctcacagcagcaatgccatcatcaaaagttttgctgctgggaagaaaaaaaaaaaaaaaaaagaaaaaaaaagttttgctggttcttggaggtgatgggagagggcaggagtatcaatttgttcaagtagctgcagtgtctgttggccaatcatctttagaaccctgtaacactgagtaccagagggcaagcccacattcctcttctgctacttcagtgtaactagggcagctgttgtctgaagcaccatagattcaggtgatcctccatagattcaggtgatcctccatagattcaggtgatccttgaaccgttcatgaaaagctgctaagtagcctggtttgggagacttgggttgagatttgcatgggaccaacacgcaggattacatcttcttcagttcctagagtcctctagaagcctatagaacaagacggcaagctctctgggcttgctctggggttttgttctgtggttttatgttgttcttgttgttttattaacatcagtgtctcttaagatccaggacccagggaggtcttttcacatacatataccaggactcttgggtactactgtcagtcttggggaagcaggctcctccatcggcaaccaaatccatgtgagcactccgaatgccttggttagtcctatgacctaaatagttaaacttcagaaaatggtttcaacagatttctgtgccgagtggttttgaaattgcatttgtatttgctgtgcttctgtattccatttaactgggttttttgttacttctgatttttgttactattaataaataaaaataaaataaaat

SEQ ID NO: 95 - protein, 308 aa, Homo sapiens
MWKRWLALALALVAVAWVRAEEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 96 - protein, 306 aa, Mus musculus
MWKRWLALSLVTIALVHGEEEPRSKSKICANVFCGAGRECAVTEKGEPTCLCIEQCKPHKRPVCGSNGKTYLNHCELHRDACLTGSKIQVDYDGHCKEKKSASPSASPVVCYQANRDELRRRLIQWLEAEIIPDGWFSKGSNYSEILDKYFKSFDNGDSHLDSSEFLKFVEQNETAINITTYADQENNKLLRSLCVDALIELSDENADWKLSFQEFLKCLNPSFNPPEKKCALEDETYADGAETEVDCNRCVCSCGHWVCTAMTCDGKNQKGVQTHTEEEKTGYVQELQKHQGTAEKTKKVNTKEI

SEQ ID NO: 97 - DNA, 1616 bp, Homo sapiens
  source 1..1616: mol_type = unassigned DNA
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaaaaaaaaaaaaaaaaaaa

SEQ ID NO: 98 - DNA, 1475 bp, Mus musculus
  source 1..1475: mol_type = unassigned DNA
agccaccagcagctcacactgaagaagcatccttgcttgggtttgcagtcttctgcggcaggcattctcggaggaaaccagccaaggtaagcctgcagtggcccatgaggctgcagttctcctggctgaattctgagggactaactacgaccatgagattggcagtgatttgcttttgcctgtttggcattgcctcctccctcccggtgaaagtgactgattctggcagctcagaggagaagaagctttacagcctgcacccagatcctatagccacatggctggtgcctgacccatctcagaagcagaatctccttgcgccacagaatgctgtgtcctctgaagaaaaggatgactttaagcaagaaactcttccaagcaattccaatgaaagccatgaccacatggacgacgatgatgacgatgatgatgacgatggagaccatgcagagagcgaggattctgtggactcggatgaatctgacgaatctcaccattcggatgagtctgatgagaccgtcactgctagtacacaagcagacactttcactccaatcgtccctacagtcgatgtccccaacggccgaggtgatagcttggcttatggactgaggtcaaagtctaggagtttccaggtttctgatgaacagtatcctgatgccacagatgaggacctcacctctcacatgaagagcggtgagtctaaggagtccctcgatgtcatccctgttgcccagcttctgagcatgccctctgatcaggacaacaacggaaagggcagccatgagtcaagtcagctggatgaaccaagtctggaaacacacagacttgagcattccaaagagagccaggagagtgccgatcagtcggatgtgatcgatagtcaagcaagttccaaagccagcctggaacatcagagccacaagtttcacagccacaaggacaagctagtcctagaccctaagagtaaggaagatgataggtatctgaaattccgaatttctcatgaattagagagttcatcttctgaggtcaactaaagaagaggcaaaaacacagttccttactttgcatttagtaaaaacaagaaaaagtgttagtgagggttaagcaggaatactaactgctcatttctcagttcagtggatatatgtatgtagagaaagagaggtaatattttgggctcttagcttagtctgttgtttcatgcaaacaccgttgtaaccaaaagcttctgcactttgcttctgttcttcctgtacaagaaatgcaaacggccactgcattttaatgattgttattcttttatgaataaaatgtatgtagaaacaagcaaatttactgaaacaagcagaattaaaagagaaactgtaacagtctatatcactatacccttttagttttataattagcatatattttgttgtgattattttttttgttggtgtgaataaatcttgtaacgaatgtaagga

SEQ ID NO: 99 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 100 - protein, 311 aa, Mus musculus
MRLQFSWLNSEGLTTTMRLAVICFCLFGIASSLPVKVTDSGSSEEKKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPNGRGDSLAYGLRSKSRSFQVSDEQYPDATDEDLTSHMKSGESKESLDVIPVAQLLSMPSDQDNNGKGSHESSQLDEPSLETHRLEHSKESQESADQSDVIDSQASSKASLEHQSHKFHSHKDKLVLDPKSKEDDRYLKFRISHELESSSSEVN

SEQ ID NO: 101 - DNA, 1501 bp, Homo sapiens
  source 1..1501: mol_type = unassigned DNA
cttagatattaaactgataggataagatataaaataatttaagattgctgatatatgttttaaaattaattatttgctcaagcatttgtgacaatttacagttctaattgaggttttaaatttagtagtttgtaggtattttaagttttgcccctgaattctttataggtgctgataagcctttggtaagttttactccatgaaagactattactgaaaaaaacgtaatctcaataaaagaactttaataagcttgactaaatatttagaaagcacattgtgttcagtgaaactttgtatataatgaatagaataataaaagattatgttggatgactagtctgtaattgcctcaaggaaagcatacaatgaataagttattttggtacttcctcaaaatagccaacacaatagggaaatggagaaaatgtactctgaacaccatgaaaagggaacctgaaaatctaatgtgtaaacttggagaaatgacattagaaaacgaaagcaacaaaagagaacactctccaaaataatctgagatgcatgaaaggcaaacattcactagagctggaatttccctaagtctatgcagggataagtagcatatttgaccttcaccatgattatcaagcacttctttggaactgtgttggtgctgctggcctctaccactatcttctctctagatttgaaactgattatcttccagcaaagacaagtgaatcaagaaagtttaaaactcttgaataagttgcaaaccttgtcaattcagcagtgtctaccacacaggaaaaactttctgcttcctcagaagtctttgagtcctcagcagtaccaaaaaggacacactctggccattctccatgagatgcttcagcagatcttcagcctcttcagggcaaatatttctctggatggttgggaggaaaaccacacggagaaattcctcattcaacttcatcaacagctagaatacctagaagcactcatgggactggaagcagagaagctaagtggtactttgggtagtgataaccttagattacaagttaaaatgtacttccgaaggatccatgattacctggaaaaccaggactacagcacctgtgcctgggccattgtccaagtagaaatcagccgatgtctgttctttgtgttcagtctcacagaaaaactgagcaaacaaggaagacccttgaacgacatgaagcaagagcttactacagagtttagaagcccgaggtaggtggagggactagaggacttctccagacatgattcttcatagagtggtaatacaatttatagtacaatcacattgctttgattttgtgtatatatatatttatctgtgttttaagattgtgcatattgaccacaattgtttttattttgtaatgtggctttatatattctatccattttaaattgtttgtatgtcaaaataaattcattaatatggttgattcttcaaaaaaaaaaaaaaaaaaaaaaaaaaaaa

SEQ ID NO: 102 - DNA, 664 bp, Mus musculus
  source 1..664: mol_type = unassigned DNA
agatccctgtgtgccctccaccatggttcacagacagctccctgaaacggtgttgctgctcttggtttcttccactatcttctccctagaaccgaaacggattcccttccaattgtggatgaacagagaaagcctacaactactgaaacctttgccaagctcgtcagtccagcagtgtctagcacacaggaagaatttcctgcttcctcagcagcctgtgagtcctcaccagtaccaagagggacaggtgctggctgttgtgcacgagatccttcagcagatcttcacgctcctccagacacatgggactatgggcatttgggaggaaaaccatatagaaaaagtcttagctgcgcttcaccggcagctggaatacgtggagtcactgggtggactgaacgcagcgcagaagagtgggggctcgagtgcgcagaaccttaggttacagattaaagcatacttcaggaggatccacgattacttggaaaaccaaaggtacagcagctgtgcctggatcattgtccagacagaaatccaccgctgtatgttctttgtgttcaggttcacaacatggctgagcagacaagacccagacccttgaacactgagaagcaagagccaacaggggatttgaaagacataggttggtggcggagtgggagga

SEQ ID NO: 103 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 104 - protein, 192 aa, Mus musculus
MVHRQLPETVLLLLVSSTIFSLEPKRIPFQLWMNRESLQLLKPLPSSSVQQCLAHRKNFLLPQQPVSPHQYQEGQVLAVVHEILQQIFTLLQTHGTMGIWEENHIEKVLAALHRQLEYVESLGGLNAAQKSGGSSAQNLRLQIKAYFRRIHDYLENQRYSSCAWIIVQTEIHRCMFFVFRFTTWLSRQDPDP

SEQ ID NO: 105 - skipped

SEQ ID NO: 106 - DNA, 1125 bp, Mus musculus
  source 1..1125: mol_type = unassigned DNA
atctgctcgattatataggaacaggaaagtaactgaatataaaactcctgtgaagcagatctgcagacataggactcatcctgcttaggaactcctaacgatggcactgctattgagtcaacctcacttctcaggtccactcctgctgctggtggtgtcaaatttgctgttgtgggaaaaagctgcatcaaatcttccatgtgttgcagaagagggtggttgctggaacccacttttggaaacatttaacagtgccacccagaaagctgaaaccctccataatcttgctgaccaattatatgtagaactttattacaaccaattctcatctggacaattttgggattttagttctcaaataattagacaggataagactgttgtcagagctggaagttactgccattcaagtctcacgaaccccccaaatacaggtgtacacataaatattgaaatcgcatcatatttaaaaactttgatcaattttgtgggttcctggatcagccctctattccatctagtgattgaactaagtgcaacaaaagatgtccctgaaactatcctctcaaaagccaaggagatcgaagagaacaacagacaaatcctgagtgaccttaggtggatactcaccaaggtctctcctgcggcagagatgacagaagaatttccccactgggaatatctttcattcttaaaatcaagtgacaaaaataataagtttttggcaatgtttaacctttcctactgcatagatcatgattcaaagtacattttgcttcagctcagattattgaagtgtctcataactgggaaagattgctaagtgcacatacatttaccatgtctgctttgaagatgctccacaaaagttcattgctgagcagttgtgtttaattgtatacatttgaaatgcttgtgtggatataatgagtctctttaaaaaaataaaataaaaacattctctttagaaatatccacatctaaaatgtctagccatcttttgttcattgaatagaatataaatcaataaactcagtttgcctcaatcaaatatatcaccaacaatacatgttaatctagcaaataatgctaaagtttatttaaataaaatcacatttatttcac

SEQ ID NO: 107 - skipped

SEQ ID NO: 108 - protein, 240 aa, Mus musculus
MALLLSQPHFSGPLLLLVVSNLLLWEKAASNLPCVAEEGGCWNPLLETFNSATQKAETLHNLADQLYVELYYNQFSSGQFWDFSSQIIRQDKTVVRAGSYCHSSLTNPPNTGVHINIEIASYLKTLINFVGSWISPLFHLVIELSATKDVPETILSKAKEIEENNRQILSDLRWILTKVSPAAEMTEEFPHWEYLSFLKSSDKNNKFLAMFNLSYCIDHDSKYILLQLRLLKCLITGKDC

SEQ ID NO: 109 - DNA, 6646 bp, Homo sapiens
  source 1..6646: mol_type = unassigned DNA
acacttccccctcttctgcagcagacggactgagttcctctaatccctgtgttccttctcccccatctttctaaaacccttctctgagagaggaataactatagcttcagggataatatagctttaaggaaacttttggcagatgtggacgtcgtaacatctgggcagtgttaacagaatcccggaggccgggacagaccaggagccactcgttctaggaatgttaaagtagaaggttttttccaattgatgagaggagcagagaggaaggagaaagaggaggagagagaaaaagggcacaaaataccataaaacagatcccatatttctgcttcccctcacttttagaagttaattgatggctgacttctgaaagtcactttcctttgccctggtacttcaggccatatacatcttttcttgtctccataatcctccctttcaaggatggccagtcagctaactcaaagaggagctctctttctgctgttcttcctaactccggcagtgacaccaacatggtatgcaggttctggctactatccggatgaaagctacaatgaagtatatgcagaggaggtcccacaggctcctgccctggactaccgagtcccccgatggtgttatacattaaatatccaggatggagaagccacatgctactcaccgaagggaggaaattatcacagcagcctgggcacgcgttgtgagctctcctgtgaccggggctttcgattgattggaaggaggtcggtgcaatgcctgccaagccgtcgttggtctggaactgcctactgcaggcagatgagatgccacgcactaccattcatcactagtggcacttacacctgcacaaatggagtgcttcttgactctcgctgtgactacagctgttccagtggctaccacctggaaggtgatcgcagccgaatctgcatggaagatgggagatggagtggaggcgagcctgtatgtgtagacatagatccccccaagatccgctgtccccactcacgtgagaagatggcagagccagagaaattgactgctcgagtatactgggacccaccgttggtgaaagattctgctgatggtaccatcaccagggtgacacttcggggccctgagcctggctctcactttcccgaaggagagcatgtgattcgttacactgcctatgaccgagcctacaaccgggccagctgcaagttcattgtgaaagtacaagtgagacgctgcccaactctgaaacctccgcagcacggctacctcacctgcacctcagcgggggacaactatggtgccacctgtgaataccactgtgatggcggttatgatcgccaggggacaccctcccgggtctgtcagtccagccgccagtggtcaggttcaccaccaatctgtgctcctatgaagattaacgtcaacgtcaactcagctgctggtctcttggatcaattctatgagaaacagcgactcctcatcatctcagctcctgatccttccaaccgatattataaaatgcagatctctatgctacagcaatccacctgtggactggatttgcggcatgtgaccatcattgaactggtgggacagccacctcaggaggtggggcgcatccgggagcaacagctgtcagccaacatcatcgaggagctcaggcaatttcagcgcctcactcgctcctacttcaacatggtgttgattgacaagcagggtattgaccgagaccgctacatggaacctgtcacccccgaggaaatcttcacattcattgatgactacctactgagcaatcaggagttgacccagcgtcgggagcaaagggacatatgcgagtgaacttgagccagggcatggttaaagtcaagggaaaagctcctctagttagctgaaactgggacctaataaaaggaggaaatgttttcccacagttctagggacaggactctgaggtgggtgagtttgacaaatcctgcggtgtttccaggcatccttttaggactgtgtaatagtttccctagaagctaggtagggactgaggacaggccttgggcagtgggttgggggtagaagttcttcctttcctaacccgggcccctgcccagctctccaaagtctttcagaaaagtaaatcctaaattcagtgatgaactggctgttttaactggttcctctaccctgaggtgacagcagagggcagcacgagcacaacagaaaccagcttacactttaagcaagtgtagctggattttccttacaggatgaggggtgggtagcagggtacaaaacatccttttttttttttttttgagacggagtcttgccttgcgcccaggctggagtgcagtggcgcgatcttggctcaccgcaagctccgtctcctgggttcatgccattctcctgcctcagcctcccgagaagctgggactacaggcgcccaccaccacacccggctaacgttttgtatttttagtagagacgggttttgaccatgttagccaggatggtctctatctcctgacctcgcgatccacccgcctcggcctcccaaagtgctgggattacaggcgtgagccaccacgcccagcccaaaacattcttgaacttgtttgtcatttccccttctagggcactgggcagcaaatgttatctgaaccattgcttcactgttagaaaaaccctgcagtgggagagagcttggtaagatgtatccaatcagagcagaatgttctctggggccctccagctggctcacgggcaagaaccccttcttcctgaggcctcccgtttctgtagcagaaggcaaacgattctctcccttcctcagaggagtccccagcttgttacttaggaaacttcaaaaccaccactgcaggtcgtagggaaagaaaagaaaaacagactcagcctcatgtattaacttcttcagatacttgtccccttttgagagatgagcacagagcaggtgggagctatagcagcacaaaatcaagccttaccaggcctaaaattcgaggtggagccacacacagatccctgctctgactgcagtccctctcaatggtgtgccagtgtctgaaagacaagtgttctctcccttgctgctgagagccttctttcatacagtcatggaacccagagtcatagctaagactcagcagcctctctccttcagctttactcagggggtcatcagtggatgacttgaggggagaaggaagcaggctgggtggggaggcagagctcataatgctcaagcactatgtgccaggtgctgttctaaacactctactgattgactccgaagccttcacaagaaccctatgaggtaggtactgttattaaccccattttacagatgaggaaattgaagctgagtgagattaggtaatttgcctaagatcagacagctagtaagtggcagagtcaggatttcaacaccagcagcctggctctagaggcaatgttcttcttagacattatgccatcctgacgctacttcttttcctactttccatttaaagacaactgaaagctgtaagcaaggagaaactttccatgttctataatcctacccagacacaaggctcagagtgggctcaggaggaaagagggaaatggaaatgaaagggtctcaacctgagaaaattcagtcttgaaagcggagtgatgggacataaatcatgagcccagaacaaattgggggagcagtgagtcagagagaatctccttccactgggattttagtggaaatctcatttttaaaatgcaatataaaaaattagggagttgctgggtagaatggaacatggaggaaatgttcaaagttggagcaaaaaggtcccttctccctcttccctccagggaaatatgggaactgtgagaggcagcaggcaagttatgggcagaggcagatacaatgagttcatgtgggaaatttctggtcagactagctctgtggttccaaaggcaaagactgcaccttgggcgagatgaatgcccttcgttgtctttatcactaacacttcaaatggggcaagccaggacatactagcaggtcaaaggcccaaacagcggaggcctactacctcctgaacagagctccacctggacctgactcaaggaacatttatatgcaactgaggagcttttgtttcatttcattttgaatagtcaagtcagtcttgctttctttaggtaaagttttacagtaacctcctttgtagtcctttgaagatgaggatttgagaaatcccaactcctaaagtaggggagagacagcagcagaatatgacccacagccagacccaaaggggctagcccgacccctgtatatattgtacctctctctctgcccacctctcccccacttctccccactgccccattctctcttatcctgcctattcctctcccctatttctctgtctctctctctctcacatacacacacactcactcacaagcagtacagtcactaagttggctaggtaatatccaaagcattctgattgtcctactttttgtaaattgaacttatttaagatattttagaagagatgagtcacgtcttcctttgacccttcacccataacacacataccattctcacaggaggccacatggtgcagtagagtcagactttgattcaaagcccaactctgtcacgtgacagtaggcttgagtttcctcatctgtaaaaagggaagatttcctacttcataaagtggttggagaaattaaagaaatagtatatgaagccttgcacataaaaagcatttgatatatattaatgcccttttgcacaagaggtcaagaattgcttgggttctacctagaaatcaagagccttatttctgagctgggttacttggcacctgtgttatttctctctcaaaactacataatggtaggaggagtaaaagaaaaagtccccgcctcccagagaaattgaaggatccagaaagaagtgcttatagcagggaatgtctcccctcacctcctgtatgaacatctctctccttcctccagtataggagggtctgcaaagctaaaggaagagttcctttgttacaagatagaaaagtggtgccactttaaagatgatttctcagtgttctccttagataccaaatacaaaggacgagggatcaagctcagcgaaagtatcaggcatttaaggtatcaggcagcaatgcggggaaaggtgaattttcttcaatcagcataggatggttagggaagagcatttatcactttggttcttatccttcaagccaggggaaaagcaacagtgaggacatcagagacaaaagcatttatagaactaacaaacacaaacgtttgacaagtgagaaagctttattaaagcacacatacatgtcaggggggtgggaaacaaaagagcaagttacagcccgggatcccaagttatgccttccattacaattgcaatccacaccaaatcaatctttgaaaacattcctccattcggttcatacatacagtagaaaccactgtggctgcccttaatccagtgtgcttataggaaatcagttagcagctgactctgttgaaagggatttcctctctgctatacactccttccacacacaactttgggtagaaacgcttatgagaatagaaaggtcagtttgatcaatgagtaccagaagatgaaacttagctcactagacccttctttgaaaagcctcaacacactacacccatgtaatatgtaggtcaaatttaggaaacaatagcaaaaacaggaactgaaagctcatcaagcaggaagtcagaataacagggagactctgtctacattcccttagagcccttaacagtccccagcctggagaaacactgggccaattgagataacacttggctcttctggttgacacagtgggatgtagaccatgctctgtttgataacacaccccagacgcaaacactaagacagatgtggtacatgaggatgaatatatctcatctttcattcgagaaatgaggagctctctctccaatgactgcagtatcagtttaaaatgcagaaaattcagcatgggctgtgagacactttaccctcagtcactctggtcctctgagatcagctagaagctctcactgggcatcctcgtgatttagtcaaaatacattatctaattaaagtgcttttcaaaagcagctccattataagagtcccccaaaggtctagtaaagatatctagaactggaaaaagcttgggaattctgttagcatttcaaagaaaagcattagaccaaagcaaggtggaaactgcttaaaaactagaaaagttaaatttaaaagccaagttccagaagtttatccaggtagaaaataagttaaaagcataaattggagaggagaaagaggaaggaactactcacctctccccaacattcctgtctttactaaccaaccctgcttcccagagccttaaaacccagactggtcctaatgcgaaaggtagccagggcttcctagggaagtaggaacaatgatagcaagacagaagataaaaagttgttttaaaagctgtttttaagttaagtgaaccttataaagacataagaaaaaatggactctgaaaataaactagattatacacagaaa

SEQ ID NO: 110 - DNA, 2871 bp, Mus musculus
  source 1..2871: mol_type = unassigned DNA
actccacccagagcttgaaaccagtgacagtcacacttcccctcttctgcagcagacagcactagctcctctaatcctcttgcttccccctcccccaaccatttcttggggaataacaaatatagctttggggataatatagctttaagacgacttttggcaaatgtaaatgtcctaacatctgggcagtgttaccagaatcccggaggccctgacagaccaggagccactggttctaggaatgttaaagtacaagggctttttcccacccccgactgactgatgagaggagcagagagcaaagaaaaagaagagagatgaacgagaccacaaaaatcataaaataaaaagcagatgtcatatgcctgcttcctctcacttttgggagttcatagctggttctgctctgaaagtcattcccctttatcctggtacttctggccacatagccctagtcttgtcttctgaagcttccctgtcgacgatgatgaccagtccactgactcagagaggtgctctctcactgctgctcctcctaatgcccgcagtgacaccaacatggtacgcaggctcaggttactctccagatgaaagctacaatgaagtatatgcagaagaggtccccgctgcccgtgcccgtgccctggactacagagtcccccgatggtgttacacattgaatatccaggatggagaagccacatgctactcaccaaggggaggaaattatcacagtagcctaggcacacgttgtgagctctcctgtgaccggggctttcgattgattggacggaagtcagtgcaatgtttgccaagccggcgttggtctggaactgcctactgcaggcagataaggtgccacacactgccattcatcactagtggcacctatacgtgcaccaatggaatgctgcttgactcccgttgtgactatagctgttctagtggctaccacctagaaggagatcgcagccgcatctgcatggaagatgggcgatggagtggaggcgagcctgtatgtgtagacatagatccacccaagatccgttgtcctcactctcgtgagaaaatggcagagccagagaaactaactgctcgagtatactgggacccacccttagtgaaagattctgctgatggtaccatcaccagggtgacacttcggggtccagagcctggttctcacttccctgaaggagaacatgtgattcgttatactgcctatgaccgagcctacaaccgggccagctgcaaattcattgtaaaagtacaagtgagacgctgtcccattctgaagccaccacagcacggctacctcacctgcagctcagcgggggacaactatggtgccatctgtgaataccactgtgatggtggttatgaacgccaggggaccccatcccgagtctgccagtcaagtcgacagtggtcaggaacaccacctgtctgtactcctatgaagattaatgtcaatgttaactcggctgctggcctcctggatcagttctatgagaaacagcgactcctcatagtctctgctcctgatccctccaatcggtactacaaaatgcaaatctctatgctgcagcaatccacctgtggactcgacctgcggcatgtgaccattattgagctggtgggacagccacctcaggaggtggggcgcatccgggagcaacaactatcagcaggcatcattgaggagctcaggcaattccagcgcctcactcgttcctacttcaacatggtgttaattgacaagcagggtattgaccgggaacgctatatggaacctgtcacccctgaggaaatcttcacattcatcgatgactacctgctgagcaatgaggagctggcacggagagtggagcagagggacctatgcgagtgaccttgagtcagggtgtggctgaagcacctgtcctagggagcttaaactaaggaggaaatgtcgtgtcctcccccccccacacacacacacacacactgttgggagcagccctcgggtgggggtgaggtttgccaaatctaggacagttccaggcagcattttagggcagaatattcatttccctaagcaagtctccacttcgggtagcactggaggagcctatgaaactgaggacatgccctgggtggtgagttgtagaccgaaggccttcttcacctgcctggagcctctctctctagactcttcccaaagcctttcagataagtaaataccaaattcattcctttacggtgttgtaaatggttcctctaccctacaataacagcagggggcagcattgcaacagacaaacaagacactttgaccaagtataaatagatttccctcacagttagttatgtgaggaacaggagagaggagactaggatctacaaaacattcttgaagctgctcgtcatcctctaggatgctggccttaaaaacaatgttgcttgagccatttcttcatcaagagttagaaaaacattttctccagggggagtttgggaagcaacgtctagccatagctggctgttccctcctagatgctccaactagcttgtaggcaagaacttctaataactgaggtgtttagtaccctggtgacagctcttccttagaggattctgcagtctgtgaacaacattacctctaagaattagggagatggctccgttggtgaagtgcttgctatcaagcactgagacccgagtgattcccagaacccatgtaaaatagccagatgtggtggtgtgcacttggaatcccagcagagaggtagacataaacagatctgtggggatcactgcttagcaagccaagtctaatgatgagtaccaggtcatgtaagaaacactatctcaaaaaccatggtaaatggc

SEQ ID NO: 111 - protein, 465 aa, Homo sapiens
MASQLTQRGALFLLFFLTPAVTPTWYAGSGYYPDESYNEVYAEEVPQAPALDYRVPRWCYTLNIQDGEATCYSPKGGNYHSSLGTRCELSCDRGFRLIGRRSVQCLPSRRWSGTAYCRQMRCHALPFITSGTYTCTNGVLLDSRCDYSCSSGYHLEGDRSRICMEDGRWSGGEPVCVDIDPPKIRCPHSREKMAEPEKLTARVYWDPPLVKDSADGTITRVTLRGPEPGSHFPEGEHVIRYTAYDRAYNRASCKFIVKVQVRRCPTLKPPQHGYLTCTSAGDNYGATCEYHCDGGYDRQGTPSRVCQSSRQWSGSPPICAPMKINVNVNSAAGLLDQFYEKQRLLIISAPDPSNRYYKMQISMLQQSTCGLDLRHVTIIELVGQPPQEVGRIREQQLSANIIEELRQFQRLTRSYFNMVLIDKQGIDRDRYMEPVTPEEIFTFIDDYLLSNQELTQRREQRDICE

SEQ ID NO: 112 - protein, 468 aa, Mus musculus
MMTSPLTQRGALSLLLLLMPAVTPTWYAGSGYSPDESYNEVYAEEVPAARARALDYRVPRWCYTLNIQDGEATCYSPRGGNYHSSLGTRCELSCDRGFRLIGRKSVQCLPSRRWSGTAYCRQIRCHTLPFITSGTYTCTNGMLLDSRCDYSCSSGYHLEGDRSRICMEDGRWSGGEPVCVDIDPPKIRCPHSREKMAEPEKLTARVYWDPPLVKDSADGTITRVTLRGPEPGSHFPEGEHVIRYTAYDRAYNRASCKFIVKVQVRRCPILKPPQHGYLTCSSAGDNYGAICEYHCDGGYERQGTPSRVCQSSRQWSGTPPVCTPMKINVNVNSAAGLLDQFYEKQRLLIVSAPDPSNRYYKMQISMLQQSTCGLDLRHVTIIELVGQPPQEVGRIREQQLSAGIIEELRQFQRLTRSYFNMVLIDKQGIDRERYMEPVTPEEIFTFIDDYLLSNEELARRVEQRDLCE

SEQ ID NO: 113 - DNA, 1515 bp, Homo sapiens
  source 1..1515: mol_type = unassigned DNA
gggactctggtgggtctaggcgcggatcggacccaagcaggtcggcggcggcggcaggagagcggccgggcgtcagctcctcgacccccgtgtcgggctagtccagcgaggcggacgggcggcgtgggcccatggccaggcccggcatggagcggtggcgcgaccggctggcgctggtgacgggggcctcggggggcatcggcgcggccgtggcccgggccctggtccagcagggactgaaggtggtgggctgcgcccgcactgtgggcaacatcgaggagctggctgctgaatgtaagagtgcaggctaccccgggactttgatcccctacagatgtgacctatcaaatgaagaggacatcctctccatgttctcagctatccgttctcagcacagcggtgtagacatctgcatcaacaatgctggcttggcccggcctgacaccctgctctcaggcagcaccagtggttggaaggacatgttcaatgtgaacgtgctggccctcagcatctgcacacgggaagcctaccagtccatgaaggagcggaatgtggacgatgggcacatcattaacatcaatagcatgtctggccaccgagtgttacccctgtctgtgacccacttctatagtgccaccaagtatgccgtcactgcgctgacagagggactgaggcaagagcttcgggaggcccagacccacatccgagccacgtgcatctctccaggtgtggtggagacacaattcgccttcaaactccacgacaaggaccctgagaaggcagctgccacctatgagcaaatgaagtgtctcaaacccgaggatgtggccgaggctgttatctacgtcctcagcacccccgcacacatccagattggagacatccagatgaggcccacggagcaggtgacctagtgactgtgggagctcctccttccctccccacccttcatggcttgcctcctgcctctggattttaggtgttgatttctggatcacgggataccacttcctgtccacaccccgaccaggggctagaaaatttgtttgagatttttatatcatcttgtcaaattgcttcagttgtaaatgtgaaaaatgggctggggaaaggaggtggtgtccctaattgttttacttgttaacttgttcttgtgcccctgggcacttggcctttgtctgctctcagtgtcttccctttgacatgggaaaggagttgtggccaaaatccccatcttcttgcacctcaacgtctgtggctcagggctggggtggcagagggaggccttcaccttatatctgtgttgttatccagggctccagacttcctcctctgcctgccccactgcaccctctcccccttatctatctccttctcggctccccagcccagtcttggcttcttgtcccctcctggggtcatccctccactctgactctgactatggcagcagaacaccagggcctggcccagtggatttcatggtgatcattaaaaaagaaaaatcgcaaccaa

SEQ ID NO: 114 - DNA, 1421 bp, Mus musculus
  source 1..1421: mol_type = unassigned DNA
ccccactgcagtgaccagggagccaccgatcttcagtaaccccagactaagctccccgctcccagtgcctggttcgccgaacgaggtgggcagacggctcgggaccatgactagagctggcatggagcggtggcgcgaccggctggcactggtgacgggagcctcggggggcatcggtgcggccgtggcccgggcattagtccagcagggactgaaggttgtgggttgtgcccgcaccgttggcaacatcgaggagctggctgctgaatgtaagagtgcaggctaccccgggactttgatcccctacagatgtgacctgtcaaatgaggaggacatcctctccatgttctcagctgtccgatcccagcacagtggcgtggatatctgcatcaacaatgccggcatggcccggcctgacaccctgctctcgggcagcaccagcggatggaaggacatgttcaatgtgaatgtgctggccctcagcatctgcactcgggaggcttatcagtccatgaaggagcggaacatagacgacgggcacatcattaacatcaacagcatgtgtggccaccgagtcccaccccagtctgtgatccatttctatagtgcgactaagtatgccgtcactgcactgacagagggactcaggcaagagcttctggaggcccagacccatatccgggccacgtgtatctctccaggcttggtagagacacagttcgccttcaaactccatgacaaggacccgggggaagcagctgccacctatgaacacataaagtgtctcagaccagaggacgtggctgaggctgtcatctacgttcttagcacacccccacatgttcaggttggtgacatccagatgaggcccacagagcaggtgacctagtgttctgtggggagatcctaccttctccacccttcacggctcgcctcctgcctctggattttaggtgttgatttctggactctgcagcttactttctatgtccaccccactaagggatagacagcttgtcagactattcccattacaaatgtgaaaaactgggccgggaagagaagacagaatctcgactgttctatctaacttcttctgagtcctcttggggctccgtaacctttgctctcctgtgcctcttccttttgacctaggaaagggatgtacctagaaggggcattgctctatcctcttacaccctaccccccaccccttagggttggagcagcagagacctcatcttttacctgtgtggttgtccagggttccaggttcccttcccaaccctcctgcctcccccccccccagctctccagccaagttttggccctcaccctggggtccttaccacggctacagagtatcagggtctgtctcctactacatttcactgtaacaattaaagagagagattacaacc

SEQ ID NO: 115 - protein, 260 aa, Homo sapiens
MARPGMERWRDRLALVTGASGGIGAAVARALVQQGLKVVGCARTVGNIEELAAECKSAGYPGTLIPYRCDLSNEEDILSMFSAIRSQHSGVDICINNAGLARPDTLLSGSTSGWKDMFNVNVLALSICTREAYQSMKERNVDDGHIININSMSGHRVLPLSVTHFYSATKYAVTALTEGLRQELREAQTHIRATCISPGVVETQFAFKLHDKDPEKAAATYEQMKCLKPEDVAEAVIYVLSTPAHIQIGDIQMRPTEQVT

SEQ ID NO: 116 - protein, 260 aa, Mus musculus
MTRAGMERWRDRLALVTGASGGIGAAVARALVQQGLKVVGCARTVGNIEELAAECKSAGYPGTLIPYRCDLSNEEDILSMFSAVRSQHSGVDICINNAGMARPDTLLSGSTSGWKDMFNVNVLALSICTREAYQSMKERNIDDGHIININSMCGHRVPPQSVIHFYSATKYAVTALTEGLRQELLEAQTHIRATCISPGLVETQFAFKLHDKDPGEAAATYEHIKCLRPEDVAEAVIYVLSTPPHVQVGDIQMRPTEQVT

SEQ ID NO: 117 - DNA, 722 bp, Homo sapiens
  source 1..722: mol_type = unassigned DNA
acttccggtctcccccaaacctgccaccgacggccacttccgtttccccgatagtatttggggatctcgaagcgatacttccggctccccccaggtccccaagctttacttttgtggggcacgacgagaaagtccgcagccccaaacattcccagaggtccacttgggccagtggtactttatcgcaggggcagctcccaccaaggaggagttggcaacttttgaccctgtggacaacattgtcttcaatatggctgctggctctgccccgatgcagctccaccttcgtgctaccatccgcatgaaagatgggctctgtgtgccccggaaatggatctaccacctgactgaagggagcacagatctcagaactgaaggccgccctgacatgaagactgagctcttttccagctcatgcccaggtggaatcatgctgaatgagacaggccagggttaccagcgctttctcctctacaatcgctcaccacatcctcccgaaaagtgtgtggaggaattcaagtccctgacttcctgcctggactccaaagccttcttattgactcctaggaatcaagaggcctgtgagctgtccaataactgacctgtaacttcatctaagtccccagatgggtacaatgggagctgagttgttggagggagaagctggagacttccagctccagctcccactcaagataataaagataatttttcaatcctca

SEQ ID NO: 118 - DNA, 764 bp, Mus musculus
  source 1..764: mol_type = unassigned DNA
agagtagaccgagcggccatcagagaacagttaaggcggaggtcccctcaactgcagagcaccaactctcatcccgagatgttccaccaagtttgggcagctctgctctctctctatggccttctctttaactccatgaatcagtgccctgagcacagtcaactaactgcgctgggaatggacgacacagagaccccagagccccacctgggcctgtggtactttattgcgggagcagcctccaccacggaagagttggcaacttttgatccggtggacaatattgtcttcaacatggctgccggctctgccccaaggcagctccagcttcgtgctaccatccgcacgaaaagtggggtctgtgtgccccggaagtggacataccgattgactgaagggaaaggaaacatggaactcagaactgaagggcgcccagacatgaaaacagacctgttctccagctcgtgcccaggaggaatcatgctgaaagagacgggccagggctaccagcgctttctcctctacaatcggtcaccacaccctccagagaagtgtgtggaggaattccagtctctgacctcttgcttggacttcaaagccttcttagtgactcccaggaatcaagaggcctgcccgctgtccagcaagtgaccagtgacttcacccgtgtcctagacagacaagataggagcccagcttcgagtggagaagccagcgactcctgccccctctcgtggataataaagatgatttgtcagtcctca

SEQ ID NO: 119 - protein, 116 aa, Homo sapiens
MAAGSAPMQLHLRATIRMKDGLCVPRKWIYHLTEGSTDLRTEGRPDMKTELFSSSCPGGIMLNETGQGYQRFLLYNRSPHPPEKCVEEFKSLTSCLDSKAFLLTPRNQEACELSNN

SEQ ID NO: 120 - protein, 190 aa, Mus musculus
MFHQVWAALLSLYGLLFNSMNQCPEHSQLTALGMDDTETPEPHLGLWYFIAGAASTTEELATFDPVDNIVFNMAAGSAPRQLQLRATIRTKSGVCVPRKWTYRLTEGKGNMELRTEGRPDMKTDLFSSSCPGGIMLKETGQGYQRFLLYNRSPHPPEKCVEEFQSLTSCLDFKAFLVTPRNQEACPLSSK

SEQ ID NO: 121 - DNA, 2512 bp, Homo sapiens
  source 1..2512: mol_type = unassigned DNA
attggtcgcggccgggaagcgtgggaggaggcccagcggcgagctggctttctcctggccacttgccggggtgaggggcagccggaggagtccgagaggaagcggaggcgcgagctggaggcggcggctcccgtcggcctccggcaggactgagcgctgggaggccggagcatattaatgaaaagtgccataaactgaaaaaccaaacatgagggtagcaggtgctgcaaagttggtggtagctgtggcagtgtttttactgacattttatgttatttctcaagtatttgaaataaaaatggatgcaagtttaggaaatctatttgcaagatcagcattggacacagctgcacgttctacaaagcctcccagatataagtgtgggatctcaaaagcttgccctgagaagcattttgcttttaaaatggcaagtggagcagccaacgtggtgggacccaaaatctgcctggaagataatgttttaatgagtggtgttaagaataatgttggaagagggatcaatgttgccttggcaaatggaaaaacaggagaagtattagacactaaatattttgacatgtggggaggagatgtggcaccatttattgagtttctgaaggccatacaagatggaacaatagttttaatgggaacatacgatgatggagcaaccaaactcaatgatgaggcacggcggctcattgctgatttggggagcacatctattactaatcttggttttagagacaactgggtcttctgtggtgggaagggcattaagacaaaaagcccttttgaacagcacataaagaacaataaggatacaaacaaatatgaaggatggcctgaagttgtagaaatggaaggatgcatcccccagaagcaagactaatggaaatgtggagagaattgaagaaagcgcactttcactcttaatgggagagctataaatggcagagctatgtgtaaatattttaagagcatgcagccatcttggtgtgtgcatgagtattgtctcttttgatatcaggattatttattgctaacgtaaatagatagcattgtaaataatcatcacaatgatcaaatcactgaaccatgtctccgcacatttccctaaaagtacaatgtttagactgctatggtaatacatattttaaattctaaaagcatacacaatgtgtaactgaatggtttgtgaaaaatatattgatatatatactagttgctatgaaaatatcatggaataatagggattttagggtggatactttattttcttttatgtttctatatgttgcgttgtgatgacattatcttttaaattaaaaagagatttggctagttgtgtgtgtaatgttactttacagtccgactctcctgatgtacctcttttcatgatctttttctttccttcccaagaaactgaggaatgtttaatatgaaaacatacatcggatatgtgaaaagcacaacaaaattcttaatgtacacagtaaaaaagtaaatatataaatgtagatggcatttaggaccacagcttgctggatttgtgttagctatgggaataacttgattttgtataagctatttagagtgaggctggaggtggcagcttcacagaactggagaaccaggccaagtcccctccccaacctaattaggtcattcaggacagctaagtcagtatatttagagcaatactagcatacgtttttcttaattgttatcagcattgaccaagtggtttggaaggaggcatgctttaatatcacaataattttgatttgtaaaccaagaaattaatcctgtgtttatctaacttcataatagcaattattgcccgaagctatagtggcatatttacaaaagttcttattactgggcggactgataacatttaaaaaataattgtgtttgaccccaaatgactttatacccaattctacataaaaatatagaagatctatctttttttgttaccttcagatgttcactaaataactcagtttttaagcagaagttttcagggcattaaatatatgttgtgtatgaagtatctcaaactggaacataaatttagtgatcaaactgccattcacagtgtaaggcagcacttaaatttcgaacctaaagtttagatgcattgtataaaaaaacctaaaagcagtatctgttatttagctgtaaaccaagttggaagctattcggataatttcttaaatattgatgaactttggagtactgtttcttccttcaaactgaatgtaattaattcatgaataaatgcaccttatatgtttaaacaatctttgtatacttttgggatttttggtgcttatatgctaaatcacattcagcatgtgtattttgacatttaaaatacttccctcaattctgtaaattaaaagaatagttattttacagttccagggattgtgaaataaatgttgcagttttttaaaataatgaaaataaatactcttggttttgctttgtgaaaaaaaaaaaa

SEQ ID NO: 122 - DNA, 2739 bp, Mus musculus
  source 1..2739: mol_type = unassigned DNA
cgcgatcggcgcgctcgggaggaggcggcggcggtggcacctcccgtcggcctccgacaggactgacggcggcgcgaccgcagacgggaccttcttttggagacgtgctagagagtgagaaggcaaaaatgagggtagcaggagctgcaaagttggtagtggccgtggcagtattcttactgaccttctatgttatttctcaagtatttgaaattaaaatggatgcaagtttaggaaatctatttgctcgatccgcgctggactcagccattcgttctacgaaacctccgaggtacaagtgtgggatctcaaaggcgtgcccagagaagcattttgcttttaagatggctagtggagcagccaatgtcgtgggacccaagatctgcctggaggacaatgttttgatgagtggtgtgaagaataatgtcggaagaggaatcaatattgccttggtaaatgggaaaacaggggaagtaatagacaccaaattttttgacatgtggggaggagatgtggcaccattcattgagtttttgaagaccatacaagacggaacagtagtgctaatggctacatacgatgatggagcaaccaaactcacggatgaggcacggcggctcattgctgaactgggcagcacttcgatcaccagtcttggtttccgagataactgggtcttctgtggtgggaagggcattaagacaaagagtccctttgaacagcacataaagaacaataaggaaacgaacaagtacgagggatggcctgaggtggtggagatggaaggatgtatcccccagaagcaagactgacgggaaggagcgagaggcgaggaggaacgcactttgactctgcaggagagtggtcaggcagccgctctgtgtacatatttcagtagcatgtggccgtccagggtgtgcatgagcaagaccgtctctgtcgattccaggattatttattgctaacagaaatggctacctttttgtaaatagtcttcattgagccaatcactgaactatttgtaagcacatttccctgaaagtacaacgtgtagacgacaatggcaataatgtattttaattctaaaagcatattcagggggtaaccacgcaggttgtacaggacgccgatatttatcctggttgctgggaaaccctatcatggaataatgtggcttttagggaggagattttgttcgtatacgtgtctctgtataaaaatctggcatgttatggcagtacccttcaaatgaaagagatttttggctagttgtctgtgtactgtcaccttacagtccaactcctgttgtatcttttcacagtctttatttttgaccttccaaagaaatccagaaataaaatgtttcacacaaaaatttacatcagatttgtgaaaagcacaacagtttttttgttgttgttattttttttaataacagtgaagaggaatattaatgtaggtgtcattcagagctcattttctcaattctgtgttagcggtgtgaattctgtgatgctgtgtaagctgtgtagagtgagcctaggggtaggattccacagaactgggggcccaggccgagtcccttcctcgacctaatcaggtcattcaagacaactgggtcagtacatttagggtaatacaggcatttttctgctttcatcgaccacatgattttaggacaggaatacttgactctcatagccttgatttgtaaaccaagaaattctgttatttatttctaactttgtgatagcagctgttgcctaaacgtgtagcagaagccttgaaaagtcccgattattgttttttttagggcatttgatgtctattatataggtgacatacatccacttcaaatgcaacataaacaagtgaaatgttcatgctgccctttgtgggatgaggccacacgtaaaatctgaacctaaaatttaggtggtttgtataaaaatctaagagcagtatttgctattttgttaaaagctagaagcagcccaggtgtccccttcttgactgttactgacctgcgctactgtttctctcctcaaattgaatgtagttcatgaggagataaataatcgtctgtgctcagagaggttttgtagactttaaatgtagagttctgctgctcctgtactaatacatccagcatggatggtttagcttttctttgtttgtttgttttttgtttgattttttttttttttttttacagttctagggatgggaaaataaacattgcagggttattttgttttttaatactgaatataagtactcttggtttgacattgtgaattttttatttttcatttttgtatttgacaaagcactatacctgcagaacatcccctgaaatccttccttttttttttttttttttttttttggatacatcttaacatataaaaattaagtttttgccattcaacatagtagtatcatatggcctggatatatttgaatgtgtatgaagtaaagttgcttgagaacaggctaaccctttgagagatgggctttccatttgcataagctgctgggagggagaagactggttctgtaactgctgtggcccagtggggtcttttggcctggattctgtcttgtactggttacaccggctcaactcgggaagcaataatcgactgttttatgttcacatcagccttagaagtggaacagtgctcacttactgttgacaacaagttttctttattaaaatcaaaaatctttaacact

SEQ ID NO: 123 - protein, 227 aa, Homo sapiens
MRVAGAAKLVVAVAVFLLTFYVISQVFEIKMDASLGNLFARSALDTAARSTKPPRYKCGISKACPEKHFAFKMASGAANVVGPKICLEDNVLMSGVKNNVGRGINVALANGKTGEVLDTKYFDMWGGDVAPFIEFLKAIQDGTIVLMGTYDDGATKLNDEARRLIADLGSTSITNLGFRDNWVFCGGKGIKTKSPFEQHIKNNKDTNKYEGWPEVVEMEGCIPQKQD

SEQ ID NO: 124 - protein, 227 aa, Mus musculus
MRVAGAAKLVVAVAVFLLTFYVISQVFEIKMDASLGNLFARSALDSAIRSTKPPRYKCGISKACPEKHFAFKMASGAANVVGPKICLEDNVLMSGVKNNVGRGINIALVNGKTGEVIDTKFFDMWGGDVAPFIEFLKTIQDGTVVLMATYDDGATKLTDEARRLIAELGSTSITSLGFRDNWVFCGGKGIKTKSPFEQHIKNNKETNKYEGWPEVVEMEGCIPQKQD

SEQ ID NO: 125 - DNA, 2177 bp, Homo sapiens
  source 1..2177: mol_type = unassigned DNA
gcactcatttgcccaagacgataactcacaaaacgccctgtggcccgactgtcacgactgtagttcgccaaccaagggcatttcctggaaatgcaaggggacacgtatctgtgacactaatggccgttctgacgtgcgttacgcccctggaaggacttggaaatactgaacttgagtgagcactgggaggatggagaccctcatcctgggaggagtcactcctcccgcagccatcacagcctgacgccccctctccccagaagtgcttcttaggcccacccaaaagggcaatggacctctctgtatgcaagccagagcagtctactgtttgtgtatatttgggttgtttactttctttttaacacttaaatgttaattgacttcaatcatttttaaaattataaatagtgtcgctatagacttttttgtacttattaagtactgacctgcatgatcttcgtgaagcagtgcttctgttaccagaaaggggtccggatccgaccccaagagagagttcttagagctcacgcaagaaaaaatccatgggagagcacagtgcaaagccaaagcaagtgtattaagaaagtaaagaaacaatgggtactccattgttagagcagcagcttgggctgctggactagggatacttgtatttattgcttgatgatgtgctaaacaagggtggattattcatgagttttccaggaaagaggtgggcaatttcaatttctcggagctgagggcttcccagctttttgaaccatacagggtaacttctgaaccttaccatggcatttgtaaattgccatggcactgatgagcgtttctcttagcgtgctaatgcatataattagcatataaccgagaggtgaagccagctgggcttctggatcaggtggggccttggagaacttttctgtatagctgaaggattgtaaatacaccaatcagtgctctgtgtctagctaaaggtttgtaaatgcaccagtcagcactctgtaaaaacggaccaaacagcactctgtaaaatagaccaatcagcgctctgtaaaatggacaaatcagcaggatgtgggcggggccaaataagggaataaaaacgggccacccgaacaaccagcggcaaccgcgctggggtctacttcccaggtgtgggatgtttgttcttttgctctttgggtctgcactacctttatgagctgcaacactcaccctgaaggtctgcagcttcactcttgaagccagcgagaccacaaacccactgggaggaacgaacaactccagacacgccgtctttaggagctgtaacactcactgtgaaggtctgcagcttcactcctgaagccagcgaaatcatgaacccaccgggaggaacaaacaactccagcacgactccagacgcaccgcctttatgagctgtaacacacaccgcgagggtctgcagcttcactcctgaagccagccagaccaggaacccaccagaaggaaccaattccggacacataacaagcagcagtgaggacaccaaagccaagccacgccacccgggcccgcccccaggacacggagtttaaggaccgcgtctcccgagatgccagagggacctgcgtccgcactgccccagtgtgacccgcatacctaccggccccgaccccgaaccgcgtggcgtccgcccgagccctggcgctgctgggtctcctgctgctgccgctgctcgcagagcccaggaaactctgcccgcagcttatgacggtcattaaccagtttctgaccaaggacaaggacacctacatggacactgtcaacagataccacctcacggagccggaaagaaacacatcctctaaactcaaggactgcgtgaccgacacaatgacccccgaggagacagaggccgtcgtgcagcaactggaagaaatcaacaaccagtgtgccgacacgatactgaagtaacaccatccataggcacctcgggttcctgtccaggctgcctgtcccaaccatgagaatctgggcccagggccccaccctccctagctcccgccctgctgctgcctctactctctctcctgctgtgctgattggggcaagcctgggaacggctgccccccacctccccacccaggcctgtcccctgcgggacaatgctgcctaataaactcacctgcatccatcca

SEQ ID NO: 126 - DNA, 425 bp, Mus musculus
  source 1..425: mol_type = unassigned DNA
acctgccaaaattaccatgaagctcactggtgctctggtgatcctcggggctaccctgctcctcctaacttcagggggagattgtggcatttgtccagctataaaagaggatgttcatctatttcttttcgggaccccagaagagtatgttaattatgtggagaaatacaaagatgaccctgaaacactggaaaatactgaaaaactcaagatatgtgtcgataggacgttgacgaaggaaaacaaggaacatgcggctgcttttatcgaaaaaatagaatccagtccgttatgttgatggctacatctttgctctcaaggaagcctcttgcctgattacctacatagatgcaacaagccctacccattccctcaggtgtctaagaacaggaatccaacaataaaagccttgcaatgcaaaaaaa

SEQ ID NO: 127 - skipped

SEQ ID NO: 128 - protein, 93 aa, Mus musculus
MKLTGALVILGATLLLLTSGGDCGICPAIKEDVHLFLFGTPEEYVNYVEKYKDDPETLENTEKLKICVDRTLTKENKEHAAAFIEKIESSPLC

SEQ ID NO: 129 - DNA, 5496 bp, Homo sapiens
  source 1..5496: mol_type = unassigned DNA
ctgtggcttgccccagagctgatccttgtctttgtccacttctcagcgaggatggcacttcagggagcccttcccttactatcgcagagagagcaggccctccccagtcatgtccaacccagaactctgttttgttttcttcatagccctagcatcacagaaaatcaccctgtgcattcatggatgtccacgggggcaagggctttgtgttgcttaacccagcatcctgaaccgtgtttgttgaatgaatacagaaccccgtttgctctgggagagcacagaaaacagtcttctatcatatatcatagccagctgcaaacagcagatggcttcccatatcccagagagtaagaaccagagagagagagaaagagagagagtttgggtctttctcctctgtgcctgctctctccagagaaactggaggggtagcagttagcattcccccgctggttccaccaagcacagtcaaggtctctaggacatggccacccctcacctgtggaagcggtcctgctggggtgggtgggtgttagttggttctggtttgggtcagagacacccagtggcccaggtgggcgtggggccagggcgcagacgagaaggggcacgagggctccgctccgaggacccagcggcaagcaccggtcccgggcgcgccccagcccacccactcgcgtgcccacggcggcattattccctataaggatctgaacgatccgggggcggccccgccccgttaccccttgcccccggccccgccccctttttggagggccgatgaggtaatgcggctctgccattggtctgagggggcgggccccaacagcccgaggcggggtccccgggggcccagcgctatatcactcggccgcccaggcagcggcgcagagcgggcagcaggcaggcggcgggcgctcagacggcttctcctcctcctcttgctcctccagctcctgctccttcgccgggaggccgcccgccgagtcctgcgccagcgccgaggcagcctcgctgcgccccatcccgtcccgccgggcactcggagggcagcgcgccggaggccaaggttgccccgcacggcccggcgggcgagcgagctcgggctgcagcagccccgccggcggcgcgcacggcaactttggagaggcgagcagcagccccggcagcggcggcagcagcggcaatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccatacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatctctatttttttaggaaaacaaaaatgaaaaactactccatttgaggattgtaattcccacccctcttgcttcttccccacctcaccatctcccagaccctcttccctttgcccttctcctccaatacataaaggacacagacaaggaacttgctgaaaggccaaccatttcaggatcagtcaaaggcagcaagcagatagactcaaggtgtgtgaaagatgttatacaccaggagctgccactgcatgtcccaaccagactgtgtctgtctgtgtctgcatgtaagagtgagggagggaaggaaggaactacaagagagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatggggacataagctaatttttttacaggacacagaattctgttcaatgctgttaaatatgccaatagtttaatctcttctattttgttgtcgttgcttgtttgaagaaaatcatgacattccaagttgacatttttttttcattttaattaaaatttgaaattctgaacaccgtcagcaccctctcttccctatcatgggtcatctgacccctgtccgtctccttgtccctgcttcatgtttgggggcctttctttaactgccttcctggcttagctcagatggcagatgagagtgtagtcaagggcctgggcacaggagggagagctgcagagtgtcctgcctgccttggctggagggacacctctcctgggtgtggagacagcttggttccctttccctagctccctggtgggtgaatgccacctcctgagatcctcacctcttggaattaaaattgttggtcactggggaaagcctgagtttgcaaccagttgtagggtttctgttgtgtttttttttttttttttgaaataaaactataatataaattctcctattaaataaaattattttaagttttagtgtcaaaagtgagatgctgagagtaggtgataatgtatattttacagagtgggggttggcaggatggtgacattgaacatgattgctctctgtctcttttttcagcttatgggtatttatcttctattagtatttgtatcttcagttcattccactttaggaaacagagctgccaattgaaacagaagaagaaaaaaaaaaaaagcagcagacaacacactgtagagtcttgcacacacacaagtgcccaggcaaggtgcttggcagaaccgcagagtgggaagagagtaccggcatcgggtttccttgggatcaatttcattaccgtgtacctttcccattgtggtcatgccatttggcagggggagaatgggaggcttggccttctttgtgaggcagtgtgagcagaagctgatgccagcatgtcactggttttgaagggatgagcccagacttgatgttttgggattgtccttattttaacctcaaggtctcgcatggtggggcccctgaccaacctacacaagttccctcccacaagtggacatcagtgtcttctctgtgaggcatctggccattcgcactccctggtgtggtcagcctctctcacacaaggaggaacttgggtgaaggctgagtgtgaggcacctgaagtttccctgcggagtcgataaattagcagaaccacatccccatctgttaggccttggtgaggaggccctgggcaaagaagggtctttcgcaaagcgatgtcagagggcggttttgagctttctataagctatagctttgtttatttcacccgttcacttactgtataatttaaaatcatttatgtagctgagacacttctgtatttcaatcatatcatgaacattttattttgctaaatcttgtgtcatgtgtaggctgtaatatgtgtacattgtgtttaagagaaaaatgaaacccacatgccgccattttcctgaatcaaattctgcagtggaatggagaggaaaatacttctaggcaagcagctagactggtgaattgggggaaatagaaggaactagtaactgagactcctccagcctcctccctattggaatcccaatggctcctggagtaggaaaaaagtttaaactacattcatgttcttgttctgtgtcactcggccctgggtagtctaccatttacttcaccccaagtcctgctgcccatccagttgggaagccatgattttcctaagaatccagggccatgggagatacaattccaagttctcgcttcctcctttgggcatctcttctgcctcccaatcaaggaagctccatgctcaggctctcagctctcgggccagtgctctgctctgtccagggtaggtaatactgggagactcctgtcttttaccctcccctcgttccagacctgcctcatggtggcaacatggttcttgaacaattaaagaaacaaatgactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatgaattttatattccgtgaatgtatattgtcttgtaatgttgcataatgttcactttttatagtgtgtcctttattctaaacagtaaagtggttttatttctatcacaaaaaaaaaaa

SEQ ID NO: 130 - DNA, 4681 bp, Mus musculus
  source 1..4681: mol_type = unassigned DNA
gcgccccccggccccgccccctttttggagggccgatgaggtaatgcggcgctgccattggtcggagggggcggaccccgagctcccgaggggctgggtcccctcgcgcccctatatcactctgcagccctggactctcgctcacagtgggcagcgggcagccgccagcgccaagaatcttcttctcccgcttctccgcttcccgatccttctccgggaggccactcgttggctccgcggactcgtgttccagcgacccttggccacttagtcctgtcccgccgggctacttggaaggcacttccccggagctcatcgttgcccaccgtgcacagtgcccggttaaacccagcgagtgagctcggactgtagcatcagcgctagcgctcggcaactttgaagaaaagagcggcagtccccgcagcggaccacaacagctaccatgactccctggcttgggcttgtcgtgctcctgagctgctggagccttgggcactggggcgcggaagcgtgcacatgctctcccagccatccccaggatgccttctgcaactccgacatcgtgatccgggccaaagtggtgggaaagaagctggtgaaggaggggccctttggcactctggtctacactattaagcagatgaagatgtaccgaggcttcagtaagatgccccacgtgcagtacattcacacggaagcctctgaaagtctttgtggcctcaagctagaagtcaacaaataccagtacctgctgacagggcgcgtgtatgaaggcaagatgtacacaggactgtgcaactttgtggagaggtgggaccacctcacactgtcccagcgcaagggcctcaattaccgctaccacctgggttgcaattgcaagatcaagtcctgctactacttgccttgttttgtgacctccaagaatgagtgtctctggaccgacatgctctccaattttgggtaccctggctatcagtccaaacactacgcctgcatccggcagaagggtggctactgcagctggtaccgaggatgggctcccccagacaagagcatcagcaacgccacagacccctgaacccagaccttccccacctcacctccttcccatcccgccgagcgtcccagacactaactcttcccagatgatgacaatgaaattagtgcctgttttcttgcaaatttagcactggggacacttaaagtctctgctgtctatggagttgatttggaaataccttcctggccccgcccctaccccttctttttggttttgacatcacccatttccaactggggatctctggtgccaagccagaaagaacgaggcatgtatacacctcttcttcatgatatataatctatatttttttaggaaaacagaaattgaaaaatttatcccattggggcatttaaagctactcttctttcttcttcctctcttgaccacatcctagacccagttccatatacacttcttcatcccaatacacaggaaacatggataaggaagttggcagaagtccaactgtttcccaatcagtcaaaggcagcaagcagacagacgccaggtatttgaaaggtcttctacacagaggatatacaactgtatgtctgggtcagacagtctgtctatgtgtgcatgtcagagggagagaagagacagactgtaggagtgggtctcacagttatccagccctgtgatacttgggttgaccccatgtttcagatcctggaacaagcatccaagagtaacagaactttctgagctggcaaaggcttaaacatctgcctgggttcagcaggcaacctgcccgtctgtacaaaaaactcaaagggctgagatgaacgtctttcctctcctgcctcccatgagacagtctcctaggaattcttggggaagtttggctggaagcttgccaactgtaagttggagaaacacacacctgacactatccacagatatagtcaagtagatttggatagagaatactatttccaaagtagtgtttagctcatctagggggatgtgtttatatatacatttgcatatacccacgggggattatgagctaattttttttataggacacagaattctattcaatgctgttaaatatgcaaatagtttaatctttttttcttttgttgtttgatgggaaattgtgacattccaagttgacttttttttattatctgaattaaaatttgaaactctgaccacactcagtaccctactgtccctataactggggttatctgaccatccatctcccttctttgttctgctttctgttcggggtgcccttcacttaattgccttactggctggctgacggcaggggagtgtgagtgttaggttagggccttctcatgaaaggggcagttgcacagtggaatgactcctctcctggatctggggacagcatacacttcttccccagctcagtaatgggtgaagtcacttccggaggtggtcacctcatagaatgaacatgattggttactgagggagccaggctctgccgtccaaatgtaggccttttgttgtgttgtatttttgtttttaaataaaagtacagtataaattttcctattaaataagattattttaagttttagtgtcaacattgcaatgccaatagtaggtgagagtgtattgcacagagttggagatagtaaggtggtgacatttaacacagttactctgtcttttttcagattatgggtatttatcctctattagtatcatatcgttttcattccactttagaaacacagttaccagttgagagagagagagagagagagagagagagagagagagagagagagagagaacaagttctagtctgtccccaccccacacataccactgctttgtccaggtgtttgatagaagagcagggcaggaagacagttcaggagatgggtgtcaagttttctcttggttgatttcattcccttgcatctttcccctgtgctgacagggggcagaaggcacaaccatttccatggtgtgccgagagaaggagctgaggtcagcaagccagtggttcttaagagctgaacctctggctttactgttggaagttgtctatttcaacctcagagtttcctaatatggcgctcctgatctatcttactcaagtgccctcccatatgtataccccaacatcttttccatcttaatccagcatcgggccattctcaatccctggtaatcacctcccttttacatggacagacttgaggtgaggcctaagtatgaggcacctgaagataaaaccaatgggcagaaccaatggccccagtcggcttttgaggtcttggtgagaagtactggccacgaaagggtcttgtgtaaagttacatcatcggagagcggtttttgagctttcaataagctatagctttgtttatttcacctgttcacttactgtataatttaaaatcatttatgtagctgagacactttgatacttcaattatatcgtgaatgttttattttgctaaatcatgtgccatgtgtaggctgttgtgtgtacactgtgtctaagagaagaagaaaagaaaaaaaaaagaaagaaacccacatgccattttttttttctgattcaaattctacagtagaatggagggcagaattactgggacacaatcaccactcagctagaatggagattcaaggaaatggaaggtactagaaacagactcctccagcctcctccccatagaatcctaagggtgggaagaagaagaagaaaaaaagcaaaagcaaaacaaaacagaaatcaaaagtacactcaagttcttgtcctgtatcaaatatccctaggtcatctgccacccactttccaagacctttctcctactcaaccaggaagtcatggtttctgcagtccagagccatggcaactgagattctaagctctctttgtctcatccaggcaacttttctgtctcccaaccaaggaattcctctgctcaggctcctgttgcccaggatagctctcttctatagccaggatgtggagcttaaggagactccaatgtttactttctccagtcctagctctgccatataaacactgtctcatgttgtgcttcttaaacgacaaagaacatagtgacttttggggatagatctgtctgggagataatgtgagtccaggaggcattactcttccctgcctcacatcaaggtgccattcaggtagttggctcccagctacttaacgataaaaatatcactttcgtatttgtatcagagaaacttaaaaaaaaaataaagtctgatggtagaaatccaatgaaatgggagaaggcaggagagagaggtctcagtagatctaagtcggctgtttgggttgaggaggagagaacccgaggaaatgaccatgctctggggacaggggtaccttagattctctgtctatttttcctctagcaagccagctgtgctgaagcccagtagagatgaagagacaccagccagagaaaaccccctctagaaggaatgtatttgttgctaaattttgtagcactgtttacagttttcctccatgttatttatgaattttatattccgtgaatgtatattgtcttgtaatgttgcatagtgttcactttttatagctgtcctttattctaaacagtaaagtggttttatttctatcac

SEQ ID NO: 131 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 132 - protein, 211 aa, Mus musculus
MTPWLGLVVLLSCWSLGHWGAEACTCSPSHPQDAFCNSDIVIRAKVVGKKLVKEGPFGTLVYTIKQMKMYRGFSKMPHVQYIHTEASESLCGLKLEVNKYQYLLTGRVYEGKMYTGLCNFVERWDHLTLSQRKGLNYRYHLGCNCKIKSCYYLPCFVTSKNECLWTDMLSNFGYPGYQSKHYACIRQKGGYCSWYRGWAPPDKSISNATDP

SEQ ID NO: 133 - DNA, 5846 bp, Homo sapiens
  source 1..5846: mol_type = unassigned DNA
acagtgttcgcggctgcaccgctcggaggctgggtgacccgcgtagaagtgaagtacttttttatttgcagacctgggccgatgccgctttaaaaaacgcgaggggctctatgcacctccctggcggtagttcctccgacctcagccgggtcgggtcgtgccgccctctcccaggagagacaaacaggtgtcccacgtggcagccgcgccccgggcgcccctcctgtgatcccgtagcgccccctggcccgagccgcgcccgggtctgtgagtagagccgcccgggcaccgagcgctggtcgccgctctccttccgttatatcaacatgccccctttcctgttgctggaagccgtctgtgttttcctgttttccagagtgcccccatctctccctctccaggaagtccatgtaagcaaagaaaccatcgggaagatttcagctgccagcaaaatgatgtggtgctcggctgcagtggacatcatgtttctgttagatgggtctaacagcgtcgggaaagggagctttgaaaggtccaagcactttgccatcacagtctgtgacggtctggacatcagccccgagagggtcagagtgggagcattccagttcagttccactcctcatctggaattccccttggattcattttcaacccaacaggaagtgaaggcaagaatcaagaggatggttttcaaaggagggcgcacggagacggaacttgctctgaaataccttctgcacagagggttgcctggaggcagaaatgcttctgtgccccagatcctcatcatcgtcactgatgggaagtcccagggggatgtggcactgccatccaagcagctgaaggaaaggggtgtcactgtgtttgctgtgggggtcaggtttcccaggtgggaggagctgcatgcactggccagcgagcctagagggcagcacgtgctgttggctgagcaggtggaggatgccaccaacggcctcttcagcaccctcagcagctcggccatctgctccagcgccacgccagactgcagggtcgaggctcacccctgtgagcacaggacgctggagatggtccgggagttcgctggcaatgccccatgctggagaggatcgcggcggacccttgcggtgctggctgcacactgtcccttctacagctggaagagagtgttcctaacccaccctgccacctgctacaggaccacctgcccaggcccctgtgactcgcagccctgccagaatggaggcacatgtgttccagaaggactggacggctaccagtgcctctgcccgctggcctttggaggggaggctaactgtgccctgaagctgagcctggaatgcagggtcgacctcctcttcctgctggacagctctgcgggcaccactctggacggcttcctgcgggccaaagtcttcgtgaagcggtttgtgcgggccgtgctgagcgaggactctcgggcccgagtgggtgtggccacatacagcagggagctgctggtggcggtgcctgtgggggagtaccaggatgtgcctgacctggtctggagcctcgatggcattcccttccgtggtggccccaccctgacgggcagtgccttgcggcaggcggcagagcgtggcttcgggagcgccaccaggacaggccaggaccggccacgtagagtggtggttttgctcactgagtcacactccgaggatgaggttgcgggcccagcgcgtcacgcaagggcgcgagagctgctcctgctgggtgtaggcagtgaggccgtgcgggcagagctggaggagatcacaggcagcccaaagcatgtgatggtctactcggatcctcaggatctgttcaaccaaatccctgagctgcaggggaagctgtgcagccggcagcggccagggtgccggacacaagccctggacctcgtcttcatgttggacacctctgcctcagtagggcccgagaattttgctcagatgcagagctttgtgagaagctgtgccctccagtttgaggtgaaccctgacgtgacacaggtcggcctggtggtgtatggcagccaggtgcagactgccttcgggctggacaccaaacccacccgggctgcgatgctgcgggccattagccaggccccctacctaggtggggtgggctcagccggcaccgccctgctgcacatctatgacaaagtgatgaccgtccagaggggtgcccggcctggtgtccccaaagctgtggtggtgctcacaggcgggagaggcgcagaggatgcagccgttcctgcccagaagctgaggaacaatggcatctctgtcttggtcgtgggcgtggggcctgtcctaagtgagggtctgcggaggcttgcaggtccccgggattccctgatccacgtggcagcttacgccgacctgcggtaccaccaggacgtgctcattgagtggctgtgtggagaagccaagcagccagtcaacctctgcaaacccagcccgtgcatgaatgagggcagctgcgtcctgcagaatgggagctaccgctgcaagtgtcgggatggctgggagggcccccactgcgagaaccgattcttgagacgcccctgaggcacatggctcccgtgcaggagggcagcagccgtacccctcccagcaactacagagaaggcctgggcactgaaatggtgcctaccttctggaatgtctgtgccccaggtccttagaatgtctgcttcccgccgtggccaggaccactattctcactgagggaggaggatgtcccaactgcagccatgctgcttagagacaagaaagcagctgatgtcacccacaaacgatgttgttgaaaagttttgatgtgtaagtaaatacccactttctgtacctgctgtgccttgttgaggctatgtcatctgccacctttcccttgaggataaacaaggggtcctgaagacttaaatttagcggcctgacgttcctttgcacacaatcaatgctcgccagaatgttgttgacacagtaatgcccagcagaggcctttactagagcatcctttggacggcgaaggccacggcctttcaagatggaaagcagcagcttttccacttccccagagacattctggatgcatttgcattgagtctgaaagggggcttgagggacgtttgtgacttctggcgactgccttttgtgtgtggaagagacttggaaaggtctcagactgaaatgtgaccaattaaccagcttgtttgatgatgggggaggggctgagtgtgcaatgggcccaggtctggaggggccacgtaaaatcgttctgagtcgtgagcagtgtccacctgaagggtcttcctttcaaaagaggctgcggccagagactgtggctcatgcctgtaatcccagcactttggaggctgaggcaggtggatcacccgaggtcaggagtttgagaccagcctggccaacgtggtgaaagtttgtctttactaaaaatacaaaaggtagccgggggtggtggtggatgcctataatcccagctactcgggaggctgagacaggagaatggcttgaacctgggaggcggaagttacagtgagccgagatctcaccactgcactccagcctgggcaacaagagtaaaaatctgtctcaaaaagaaaaaaatgtacttaggaggggttaattgtggcgtgtttatggaattctttccttattctccttttagtggggcaaagagaagtaagattcttaaactcaaaaatataggataaagaaacttacagagattttgctttttaaagcattgatcttacgctgtctaggttttaattttgttttgctttgcttttctacacagtttttaaagaaatatttcaagaaatgttggttatttattaaacagggatatttgtacctatgtggcaaagaggcatatttggaatattctctggcaaactagatacttacttccctatcgctgcagtatttaggaattacttcttctccttggttgtgttgtttagagttggattttctgtagaaatctttctagagctctgatgtgactccagacactttatcgtttttcttttttttgagacggagttttgctcttgttgcccaggctagagtgcagtggtacaatcttggctcactgcaacctttgcctcccaggttgaagtgattctcatgcctcagcctcttgagtagctgggattagaggcaggtgccaccatgcctagctaatttttgtatttttagttagagacagggtttcaccatgttggccaggctagtctcgaacttctgacctcaggtgatccccctgccttggcctcccaaagttctgggattacaggggtgaaccactgtgcctggcccatttttctttataaatattgtaacataatgttttatagacaaacattcaagggtactttggctttaagaacttcaggatttctggtgctagaaaagcgcttgaagcagtatcaccaagattttagatattaaaaagtctggtgtaccagacattgagtcataatcatctatattcaagggatactttcattgataactttgttattatgctgcccttcacagaagacaacgtccggggcaggatcacatgctccctagcagatgctgatcagtgatgtcatagaaattacatgaatgcattgtctttaaatagcagtttaaccatgttataatgtaggctttttgtcttgttccgggctggtatttgggtgccctgattgaattacttggatttaaacagcaaactgtgggctctcgacttacatagtaagggcctttactgtttctttgtaggaaatgggtttctcgcctttgaaacattttttccccttttgtagtgacagtgccactaaatagttcagcttttgtcagtcccccaggaaagtgctatcctatggcctaactagccaagccttttttctttcatttaaaagaaattagctttaatttttacctttaattacttattcaaataagacagaaatatattttccttgcaataattaaaacattgcatataggccataaatttccttattttctctgaacgatcctgattccagtcatcttgttgaataccctagttctaataattgactcttgcttttctagagaaatatttccaaatgatgctagttttgtctcttcctttcaaagttgtataccacttctttttcttgtcattttgcattgcctgggacctccagaataatgtttcatgaagtagcatgtatccatatctggttcttgactttttcatcataataattgttttctatgggttacttatcagtttaagaatgcttaattcctagatgaactaagagtgtttattacatgttgagatttatggtatgctttttcttcctcaagataatgcattttttgtattatctgttaatgtgataggttatccatttgtgtattttcaatcattgaacaacccttgatttttttggataaactctatttggtcattatgcatcattctataaaccctgctgaatttttcatttgccaacatcttatttcagattcttttaatctgtgtccaacaatgagatttgttttcttttgcaatttgttttgaatttttggtatcagagctatactaaccttataatggaaaatacatatttctcaaacttttacactgatatattcatagtatttttttataatttgaaaaatcttgtcagtatctgtattaaggcctccatttcagttctgctatttcatattgccttaggttgtctatttgtctctttaatgaacccgattttgattttgtcatttttaaaataaacacatttatgctataaa

SEQ ID NO: 134 - DNA, 3885 bp, Mus musculus
  source 1..3885: mol_type = unassigned DNA
ttgccagtgagcgagcgagcgtgtggaaccccaggcctgagcccgcgcccctacctgcccagccaagccgagtgccgccggctttgttcgctttgtcctcgcgcacctaagcggccggcctggaagaacgccatcccggagagcgcacgcggcgtcgcaccaggtctaacaacatgcctccacttctgcttctaccagccatctacatgctcctgttcttcagagtgtccccgaccatctctcttcaggaagtgcatgtgaaccgggagaccatggggaagatcgctgtggccagcaaattaatgtggtgctcagccgcggtcgacatcctgtttctgttagatggctctcacagcatcgggaaggggagcttcgagaggtccaagcgcttcgccatcgctgcctgtgatgccctggacatcagccctggcagggtcagagtcggagccttgcagtttggttccactcctcatctggaattccccttggactccttctcaactcgacaggaagtgaaggaaagcatcaaggggatagttttcaaaggtgggcgcaccgagacgggcctagccctgaaacgcctgagcagagggttccccggaggcagaaatggctctgtgccccagattcttatcatcgtcacggatggcaagtcccaggggcccgtggctctcccggctaagcagctgagagaaaggggcatcgtcgtgtttgccgtaggagtccgttttcccaggtgggacgagctgctcacgctggccagtgagccgaaggaccggcatgtgctgttggctgagcaagtggaggatgccaccaatggcctcctcagcaccctcagcagctccgcactctgcaccactgctgatccagactgcagggtggaacctcatccctgtgagcggaggacgctggagaccgtcagggagctcgctggcaatgccttgtgctggagaggatcaaggcaagcagacactgtgctggctctgccctgtcccttctacagctggaagagagtgttccagacacaccctgccaactgctacagaaccatctgtccaggcccctgtgactcccagccctgccaaaatggaggcacgtgcattccagaaggtgtggataggtaccactgtctctgcccactggcattcggaggggaagtcaactgtgccccgaagctgagcctggaatgcagaatcgatgtcctcttcctgctggacagttctgcaggcaccacattggggggcttccggagggccaaggcctttgtcaagcgctttgtgcaggccgtgctgagggaggactcccgagcccgcgttgggatagccagttatggcaggaatctaatggtggcggtgcccgtcggggagtaccagcatgtgccggacctgatcaggagccttgacagcattcccttcagcggtggcccgaccctaaccgggagtgccttgctccaggtggcagagcacggctttgggagtgccagcaggactggtcaggacaggccacgcagagtagtagttctgctcactgaatcacgctcccaggatgaggtgtctgggccagcagctcacgcaagggctcgggagctactcctcctgggcgtgggcagtgagatcctgcaggcggagctggtgaagatcaccggtagcccgaagcatgtgatggtccacacagaccctcaggacctgttcagccaaatcccagagctgcagaggaggctatgcagccagccacggccaggctgccaggcacagtcactggacctggtcttcctgttggatgcctctgcctctgtgggacgtgagaactttgcccaaatgcagagcttcatcaggaaatgcaccctccggtttgatgtgaatcctgatgtgacacaggttggcctggtggtatatggcagccgggtgcagactgccttcgggctagacacccatcccacccgggctgccgtgttgcgggccatgagccaggctccctacctagggggtgtgggttctgctggtacagccttgctgcacattgaggacaaggtgatgacggtccagaggggtgcccgccccggtgtccccaaggccgtggtgatgctcacaggcgggagtggggcagaagatgcagctgtccctgcccagaagctgagaggcaatggcatctcagtcttggttatgagcgtgggagccgtcctcagggaggcagtacggaggcttgccggtccacgcgactccctgatccatgtggcagcttacacagaccttccgtaccatcaggacatgctcatcgagtggctatgtagagaagccaggctgccagtcaacctctgcaagccgagcccatgcatgaatgaaggcacctgtgtcctgaagaacggcagttaccgatgcgagtgccggggtggctgggaaggcccccactgtgagaaccgcatcttgagaggagatgcccccatggcacgtagcttccaccaagagcctgcaggactacaagggccaaccccctcgcagcaagctcccaagcatcttagaatcggaaaggccctgtcctccgccaagtaaatctcccgggggccctggagcatcagcttccagctctgccacaggggacactgcttgggacagtagcagctgtcgctatggtgatgttgggaacgactactgcgtcagcatttatcccctgaatccgtttgtaccttgctgaggtaagctgcctgtcatttttcctgaggataagaggcaggccagagagatctgacgacctgcttgagggtcatcacagatgctagacagatgttcctgaggtggtagcgctttgaagaagactctgtcagagctcaccgcctggtgattggtgcaaaaagtgggtttctccctgttccaaaagacagctcctgtggatttcatttgcaaagggattatgtgtcatttgtgacgttttctggtgaccagatctgtgtgtgggagagacctgggaaggtctcagacagccatttgaccaattactcagtttgcacgtaggggagggacatgtgtgaacagccgtgtacgattggtctgggcgctgagcaacggcctccagaggggcttcctttcagagattactcaggagagagtggcagtgagtgctgccctcctcttagtggtggcaatgatgggcagacttctcaaactcagcaagggagggatgaggggttgcccaggaggtttgctttcaaagcttactgtgctgctctgttttaatgtgcatctcacgcccactcggtaatgtctcaagacagattggttggttgttcgtccatcaggactgtatccgctggcaacgaggcttctttggaatatctttgggaaactaggtacttaactgcagcgctaacacatcagtctccccccacaccccttccccaccctgcccctttcttgtgctgtttgaagctgggttttcagaagaagccatgtgtagcgctctgggagatgagaagcattggtaagggtgtgtatctttgtgcagccaatgctgtaaactctgtttgtgttctgtgtgccaggctggctggctgaggtgctactccacgtctgcaggatgctctccccgtctctgacccacattttcttccttcacatttatacactggggatgtaaactttacgtattgttatatttcaatcatcgaaccaatatgtagacattgtcacagcagaatagtgctgatgtcttcgtggaacaccaggacttcccaaacacccgtgcagctgtagtcacagtaccttcctgtaaactcctgtcctggtctgttatattctactttactctgggttgtcagtaccatgttcaaggagtcagaatttggtcttgatggtttttaaacataaaataaaacatttatgtccttaaaaa

SEQ ID NO: 135 - protein, 755 aa, Homo sapiens
MPPFLLLEAVCVFLFSRVPPSLPLQEVHVSKETIGKISAASKMMWCSAAVDIMFLLDGSNSVGKGSFERSKHFAITVCDGLDISPERVRVGAFQFSSTPHLEFPLDSFSTQQEVKARIKRMVFKGGRTETELALKYLLHRGLPGGRNASVPQILIIVTDGKSQGDVALPSKQLKERGVTVFAVGVRFPRWEELHALASEPRGQHVLLAEQVEDATNGLFSTLSSSAICSSATPDCRVEAHPCEHRTLEMVREFAGNAPCWRGSRRTLAVLAAHCPFYSWKRVFLTHPATCYRTTCPGPCDSQPCQNGGTCVPEGLDGYQCLCPLAFGGEANCALKLSLECRVDLLFLLDSSAGTTLDGFLRAKVFVKRFVRAVLSEDSRARVGVATYSRELLVAVPVGEYQDVPDLVWSLDGIPFRGGPTLTGSALRQAAERGFGSATRTGQDRPRRVVVLLTESHSEDEVAGPARHARARELLLLGVGSEAVRAELEEITGSPKHVMVYSDPQDLFNQIPELQGKLCSRQRPGCRTQALDLVFMLDTSASVGPENFAQMQSFVRSCALQFEVNPDVTQVGLVVYGSQVQTAFGLDTKPTRAAMLRAISQAPYLGGVGSAGTALLHIYDKVMTVQRGARPGVPKAVVVLTGGRGAEDAAVPAQKLRNNGISVLVVGVGPVLSEGLRRLAGPRDSLIHVAAYADLRYHQDVLIEWLCGEAKQPVNLCKPSPCMNEGSCVLQNGSYRCKCRDGWEGPHCENRFLRRP

SEQ ID NO: 136 - protein, 791 aa, Mus musculus
MPPLLLLPAIYMLLFFRVSPTISLQEVHVNRETMGKIAVASKLMWCSAAVDILFLLDGSHSIGKGSFERSKRFAIAACDALDISPGRVRVGALQFGSTPHLEFPLDSFSTRQEVKESIKGIVFKGGRTETGLALKRLSRGFPGGRNGSVPQILIIVTDGKSQGPVALPAKQLRERGIVVFAVGVRFPRWDELLTLASEPKDRHVLLAEQVEDATNGLLSTLSSSALCTTADPDCRVEPHPCERRTLETVRELAGNALCWRGSRQADTVLALPCPFYSWKRVFQTHPANCYRTICPGPCDSQPCQNGGTCIPEGVDRYHCLCPLAFGGEVNCAPKLSLECRIDVLFLLDSSAGTTLGGFRRAKAFVKRFVQAVLREDSRARVGIASYGRNLMVAVPVGEYQHVPDLIRSLDSIPFSGGPTLTGSALLQVAEHGFGSASRTGQDRPRRVVVLLTESRSQDEVSGPAAHARARELLLLGVGSEILQAELVKITGSPKHVMVHTDPQDLFSQIPELQRRLCSQPRPGCQAQSLDLVFLLDASASVGRENFAQMQSFIRKCTLRFDVNPDVTQVGLVVYGSRVQTAFGLDTHPTRAAVLRAMSQAPYLGGVGSAGTALLHIEDKVMTVQRGARPGVPKAVVMLTGGSGAEDAAVPAQKLRGNGISVLVMSVGAVLREAVRRLAGPRDSLIHVAAYTDLPYHQDMLIEWLCREARLPVNLCKPSPCMNEGTCVLKNGSYRCECRGGWEGPHCENRILRGDAPMARSFHQEPAGLQGPTPSQQAPKHLRIGKALSSAK

SEQ ID NO: 137 - DNA, 1054 bp, Homo sapiens
  source 1..1054: mol_type = unassigned DNA
gccaaaacagtgggggctgaactgacctctcccctttgggagagaaaaactgtctgggagcttgacaaaggcatgcaggagagaacaggagcagccacagccaggagggagagccttccccaagcaaacaatccagagcagctgtgcaaacaacggtgcataaatgaggcctcctggaccatgaagcgagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgagactttctgaggttgtgctttatttctgctgcgtcgtgggagagggcgggagggtgtcaggggagagtctgcccaggcctcaagggcaggaaaagactccctaaggagctgcagtgcatgcaaggatattttgaatccagactggcacccacgtcacaggaaagcctaggaacactgtaagtgccgcttcctcgggaaagcagaaaaaatacatttcaggtagaagttttcaaaaatcacaagtctttcttggtgaagacagcaagccaataaaactgtcttccaaagtggtcctttatttcacaaccactctcgctactgttcaatacttgtactattcctgggttttgtttctttgtacagtaaacattatgaacaaacaggca

SEQ ID NO: 138 - DNA, 458 bp, Mus musculus
  source 1..458: mol_type = unassigned DNA
atcctgtgcagtggtcctgaagcttgcctgctgccatggagaccagagccctctggctaatgctgttggtggtcctggttgctgggtcctctgggatagctgcagattacgttggcctgtctccaagccaatgtatggtgccggcaaatgtcagagtggactgtggctacccctctgtcacatcggagcagtgtaacaaccgtggctgctgctttgactccagtatcccaaatgtgccctggtgcttcaaacctctgcaggagacagaatgcacattttgaagctgtccaggctccaggaagggagctctgcaccctggactcctgctgctgatggtggccaagggtagcaagcatccccgatctgctccctgctgcaggccaataaaggagccaggagtcctgaagaataaagacctcacagccaacacaaggctgatctgattgctgagacctcta

SEQ ID NO: 139 - protein, 94 aa, Homo sapiens
MKRVLSCVPEPTVVMAARALCMLGLVLALLSSSSAEEYVGLSANQCAVPAKDRVDCGYPHVTPKECNNRGCCFDSRIPGVPWCFKPLQEAECTF

SEQ ID NO: 140 - protein, 81 aa, Mus musculus
METRALWLMLLVVLVAGSSGIAADYVGLSPSQCMVPANVRVDCGYPSVTSEQCNNRGCCFDSSIPNVPWCFKPLQETECTF

SEQ ID NO: 141 - DNA, 3503 bp, Homo sapiens
  source 1..3503: mol_type = unassigned DNA
cccgccagcgccgcaatcccgtcactgtattgtcctcctcggcaccgagcgatctgatccgccgagccagaaagacacccgatgggggagagaagcaaagtggaaaaaaaccgacgtccctgctctccagaagggagaccccagcggcgccgacgagccctgcgccctccgcggccgcccggcgcgagctcccgggcggggcgctgtccagcgcgcacagagttaaaggcatcatcgcagccattagagggaaaagttgcggcgccgcgagcccggcgaagtcctcagcccgccccgcttgcccactccccacttcccgagccggctccgtgtttagggagggcagtgatcacgcaagccggagcggcgggctgacgttggacgagctgccaggtagctgaaagcaggcagccaggcagccgagacacttcccagcgattccagcctgggctccgcaggaagcctcgctgaatcccagccagctggttctaaccttccagaatcgcaatcccttctccccacagccagccctcgccaagcaagcagcaggatgtttgcagtgtcgcgcccagggctctgagactgagcctgccatccactcgcacgcctttctttcagggcttttcggctgttggctacactgatgtgacccccctccctttttggaatgatggggatctttttggtgtatgttggatttgttttcttttccgttttatatgtacaacaagggctttcttctcaagcaaaatttaccgagtttccgcggaacgtgacggcgaccgaggggcagaatgtggagatgtcctgcgccttccagagcggctccgcctcggtgtatctggagatccaatggtggttcctgcgggggccggaggacctggatcccggggccgagggggccggcgcgcaggtggagctcttgcccgacagagacccggacagcgacgggaccaagatcagcacagtgaaagtccaaggcaatgacatctcccacaagcttcagatttccaaagtgaggaaaaaggatgaaggcttatatgagtgcagggtgactgatgccaactacggggagcttcaggaacacaaggcccaggcctatctgaaagtcaatgccaacagccatgcccgcagaatgcaggccttcgaagcctcgcccatgtggctgcaggatatgaagccccgcaagaacgtctccgcagccatccccagcagcatccatggctctgccaaccaacgaacgcactccacctccagccctcaagtggtagccaaaatccccaaacaaagtccacaatcagtgcatgccaagacattcatgagcaccagagccaagctggcgagctaattaaatatatatatgtatgtgtttaaaacaacctaataaggagcgttggaaatcttcaagatgagcgaatatcttataacttcttccgcaggtgcgaggatagctacaagccatggactttctgtcctgcttcttgtttgtggctttgtgaagggtgctttgctttaatctaaagtgctgacttttttccaatatcactttctcttcttaaagcaaagagcaaatcgcctgtaaaatctacggagcggacagcaaagttgaccctaaactccaagcaccaccctgcacccactgtactctaattcactacacaaggagcgcctgcttccggaagcataaatgaagaggctatcacatgctttgttgatcatattttctttggcaaaacactgatcttttattttaagagaattaacgtgaagtgatagaacgttttctaatagcaagatctattttttcccttttctttcggcgactaaaatcatctcactgactgctcaagggttggcctgaatgtcatcaggatagggaatatttactatggataccactaatttcctactaaaggacccagcatcttcaggaagcaaacagagatcaaaaggctacagaacagaagctatcatcagacaaaaccccctttttaggggaagaacaaaactatcaacactgcaagtcaacaaggaggacattactttaaggaataacatgagaaagaaatttttttattttcccctttttttcttggatttttcttctttttcttgattacagttcttgttcaatggtggcaagtgctggttatggccaatctccgtcaatcctaggaggtttatagaactacattttgagtgttctatatttcagtgtattttaattcattttgcaattcctgtgtatgcaaacctgataaattctgtaaattgcttatagtatgtgtgctataacttcaaagtagatgtactgcgaccctcatgcaagctgatttttatcattatatatataaatatatacttaagaatatctgtgtgttgggccaatgaccaactttttttgacgaagcatttgtttttcgttgataattcatacactgcgtgaattttgtaattcattgtttcacttccacaggtttgacagctgcagatggtctctattgtcctctgcttcattctggaaagtgcatattcaaaattgtatcagtctctgatcgatgaattaattttgttatgtctgtgctaagttggaatttactatgttcctttatacatggtgtttattgaggtttgagagtctcttgactgtgaagaatgtacactgtctggttttgacagctatttctgtattattatcattataatcttgtcaaatagaaatgttgcttctagaaaatttgcctaggaggcgaatgtggggaaggtgaagcactcaccataattccctaaattcatgttagaacatttcttgctatggttaaaaatgctgcttcctttgacctttatgaatttctgtacctttgtcattctgttacctttgtcattctgttatttgcgctaattatattttaatgtctcctaattaaaaattaaaatttggttgttggctaaatacaatatgcaaaagatgatggcaggtccccgactaaagaagattaatgtgttcagttttcactggttaagtgaattataattttaaatattccattcttatttatgtctttatgataattcagactattttattttgaacctgttttctactctggcaaagaaagataggcagaactattagtgttccatatacattatggaatagataaagcttgagagataaatgacctaagttttccttccagagagactcttccattttctctcattacaaaaccagaagatcagctatgtggggccatcagctcccagcctaaggtcctataacccgaagcttgaaggcaatcagtacctctgcttttcaaaggtaaatgcattcattttctttgattcagttataagcaagtatatattttcataatattcttgacattacctgagaaataatacttcatgctaaatcttttactgccacttttctcattcatttggtttatcatttcagtttaatgagaaaaattaataaaggttttgattgcactatttta

SEQ ID NO: 142 - DNA, 3489 bp, Mus musculus
  source 1..3489: mol_type = unassigned DNA
aggtagcggaagcagccagccggcggcggagacacttcacggcgtggcaacccgggtctgtgccttgaagcctccggatcgcagccagctcggtccatccctcactagtcgcaatcccctgtgtccaagctactctttgctatgagcggcagcatgcgtgcagtatcgcgccccaggctctgagagcagcctgcggacacgcttgcctatctgtctttttaggttttggggctctgggctacacggatgtgccccactcccttggcatgatggggatctttttggcgtctgttggatttatgttcttttccgtgttatatgtacaacaagggctttcttctcaagcaaaatttaccgagttgccgagaaatgtgactgctaccgaagggcaaaatgtggagatgtcctgtgctttccaaagcggctctgcttcagtgtacctggagatccagtggtggttccttcgggggccagaggacctggagcaaggcacggaggctgcaggctcgcaggtggagctcttacccgacagagacccggacaacgatgggaccaagattagtacagtgaaagtccaaggcaatgatatctcccacaagcttcagatatccaaagtgagaaaaaaggatgaaggtttatacgagtgcagggtgactgacgctaactacggggagcttcaggaacacaaggcccaggcctatctgaaagtcaatgccaacagccatgctcggaggatgcaggcctttgaagcctcacctatgtggctgcaagacacgaagcctcgaaagaacgcatcatcggtggttcccagcagcgtccacaactctgccaaccaacgaatgcactccacctccagccctcaagcggtagccaaaatccccaagcaaagtccacaatcagcaaagagcaaatcgcctgtaaaatctacggagcggacagcaaagttgaccctatactccaagcaccattctgcacccctgtactctagttatctacacaaggagcatcagcttccggaagcataagtgaagacactgtcacacgctttattgataatattttctttgggaagttgctgatcttttatttcaagagaattaatgggaagagataggacattttccaattacaagaccaatttttttccttttatttcaacaaataaaacctgcatttcactgactgctcaggagttggcctgaatgacatcagtatactaaatatttccatggattccaccaatttcctaacgagggacacctaatcttcaagaagcaaacaaagatggaaaacctaagaaccacaaactgtctcatacagcaccccagctgaggaacaaaacaaatagctaaatgctgaccatggcaaatcaacatcagacaactttattttacatatggaataatcaaagaaagtttttttttacttcctttttgccccctggaatttatcttggagtttcccttttttccttgattgccgttttcgttcaatggtagcaagtgccaattatggccaatccttgtcaatcctggaaggtttatattcatatacattgagtgtggtatatatcaatgtattttaattcatttggcaatttctgtataggcaaacctggcaaattctgtaaattgcttatagtatgtgtgatatgacttcaaggtagataggctatgatgctcatgcaagctgactttcttcattctatatacaaatatattcatgagcatatattaggccaccaacttcttttcctaaagaattatttttcatttgtacctcatgtattttgtgaattttgtagtatatttctctgttccactagtttgaccgctacagtttgtctctgttgtcctctacttccttctggaaaaatttaaaattgtgtatgtctctgataaatgaattaattttgttgtgtgtatgctatgttggaatttgctgtgttcttttaaacatgtatttattaaggtttggggatcttgagttgagtctgaagaatgcacacctggtttttgacagagttcctcatgttaccaatattctatctcagagaaagaaagacaccaagtgggaaaactaagaagacattttgacttcccaagatcctggaagagcacttcacactctgactaaataatgttgctttttttgttcttcaagacttttttgtagctttgtctttctgttagttgctgctaattatattttaatgtctactaattaaaaattaaaatgtgattgttggctgaatacaatatgcaaatgactgcaaagcccatactgaagaaaatagatgtttaatcttcactcaataattataattttaaatagttcatcattattttttgaccttatgatattttgtttagacctgttctaattacatctttctctggcaaagaaagatagaacaatcaatacattccctcttacagtatggaatggttgtggcttaagaaagaatgcatccagatggtcttccagagagattattttattttcattataaaaccagaaaccatatatgtaggaatggttcattcctaatgtaaggccataaattgtagcttgaaggcaaggaatacatttgtttttttatggtaaaggactggcctctgacatgcacttataagcaatgtgaatattttcataatatgcttgacattctcctttaacaaatattgttttatggtaaatctttccttgccattttccttctttcatttgattcattatttcattctaatgaagaaaataaaggtttaattatgatactttattaacatacaaatgtattttctttctaagttaaatatctgaaagttgtataaaatgatggtagagaaatattactcattcggtttctttgagctttaagaatcccatacattgcagtatatattagaatactgatttaacatcaaactgggggggaaaatcatgtattatacttttactcaatgtctaggtaatgaattcagctaattttacagcaagccaaatgtgtacccgtatcagtaatgttcaccatgcttgtaataaaagggtatatgctagttttggaagaatgctcattagattcattgtatcagtgtccaaaataataaagacctgtttatcactgtgtgaccaaagtccaccccaccctccactttgtaaaaagaataccgcaaaccacctgtttgctccaaactccaaaagagtatggaactttaaatattagagcagaaaatacagcagatcaatatgcttggatactccattatattcctttgggaatataactttttatgtaaccatgacaatggaagtggatagaaatgcaaaggagaagtatctggctttctcaaccttgttttcaagtttatacagtattttttttataagagagacttatagaaagctgaaattgcttactttgtttcttaaagacttatagacagcctaaaaagcaaagaaagcaaataaaaatactcctgaacaaag

SEQ ID NO: 143 - protein, 226 aa, Homo sapiens
MMGIFLVYVGFVFFSVLYVQQGLSSQAKFTEFPRNVTATEGQNVEMSCAFQSGSASVYLEIQWWFLRGPEDLDPGAEGAGAQVELLPDRDPDSDGTKISTVKVQGNDISHKLQISKVRKKDEGLYECRVTDANYGELQEHKAQAYLKVNANSHARRMQAFEASPMWLQDMKPRKNVSAAIPSSIHGSANQRTHSTSSPQVVAKIPKQSPQSVHAKTFMSTRAKLAS

SEQ ID NO: 144 - protein, 252 aa, Mus musculus
MMGIFLASVGFMFFSVLYVQQGLSSQAKFTELPRNVTATEGQNVEMSCAFQSGSASVYLEIQWWFLRGPEDLEQGTEAAGSQVELLPDRDPDNDGTKISTVKVQGNDISHKLQISKVRKKDEGLYECRVTDANYGELQEHKAQAYLKVNANSHARRMQAFEASPMWLQDTKPRKNASSVVPSSVHNSANQRMHSTSSPQAVAKIPKQSPQSAKSKSPVKSTERTAKLTLYSKHHSAPLYSSYLHKEHQLPEA

SEQ ID NO: 145 - DNA, 3924 bp, Homo sapiens
  source 1..3924: mol_type = unassigned DNA
actcggtgcgccttccgcggaccgggcgacccagtgcacggccgccgcgtcactctcggtcccgctgaccccgcgccgagccccggcggctctggccgcggccgcactcagcgccacgcgtcgaaagcgcaggccccgaggacccgccgcactgacagtatgagccgcacagcctacacggtgggagccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagccccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcactccctattttgtaaacatatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgatagtgactatgtcagtctaatctcttgtttgccaaggttcctaaattaattcacttaaccatgatgcaaatgtttttcattttgtgaagaccctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacagaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacctcagaggctgaaattcctaatacctttcctttatcgtggttatagtcagctcatttccattccactatttcccataatgcttctgagagccactaacttgattgataaagatcctgcctctgctgagtgtacctgacagtagtctaagatgagagagtttagggactactctgttttagcaagagatattttgggggtctttttgttttaactattgtcaggagattgggctaaagagaagacgacgagagtaaggaaataaagggaattgcctctggctagagagtagttaggtgttaatacctggtagagatgtaagggatatgacctccctttctttatgtgctcactgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcttcaagcctgaggttttatatacaaactccctgaatactctttttgccttgtatcttctcagcctcctagccaagtcctatgtaatatggaaaacaaacactgcagacttgagattcagttgccgatcaaggctctggcattcagagaacccttgcaactcgagaagctgtttttatttcgtttttgttttgatccagtgctctcccatctaacaactaaacaggagccatttcaaggcgggagatattttaaacacccaaaatgttgggtctgattttcaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccagcaaacactgaatttctcttgttgttttaactctgccacaagaatgcaatttcgttaacggagatgacttaagttggcagcagtaatcttcttttaggagcttgtaccacagtcttgcacataagtgcagatttggctcaagtaaagagaatttcctcaacactaacttcactgggataatcagcagcgtaactaccctaaaagcatatcactagccaaagagggaaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagatatttgtggtcttgatcatacctattaaaataatgccaaacaccaaatatgaattttatgatgtacactttgtgcttggcattaaaagaaaaaaacacacatcctggaagtctgtaagttgttttttgttactgtaggtcttcaaagttaagagtgtaagtgaaaaatctggaggagaggataatttccactgtgtggaatgtgaatagttaaatgaaaagttatggttatttaatgtaattattacttcaaatcctttggtcactgtgatttcaagcatgttttctttttctcctttatatgactttctctgagttgggcaaagaagaagctgacacaccgtatgttgttagagtcttttatctggtcaggggaaacaaaatcttgacccagctgaacatgtcttcctgagtcagtgcctgaatctttattttttaaattgaatgttccttaaaggttaacatttctaaagcaatattaagaaagactttaaatgttattttggaagacttacgatgcatgtatacaaacgaatagcagataatgatgactagttcacacataaagtccttttaaggagaaaatctaaaatgaaaagtggataaacagaacatttataagtgatcagttaatgcctaagagtgaaagtagttctattgacattcctcaagatatttaatatcaactgcattatgtattatgtctgcttaaatcatttaaaaacggcaaagaattatatagactatgaggtaccttgctgtgtaggaggatgaaaggggagttgatagtctcataaaactaatttggcttcaagtttcatgaatctgtaactagaatttaattttcaccccaataatgttctatatagcctttgctaaagagcaactaataaattaaacctattctttc

SEQ ID NO: 146 - DNA, 1639 bp, Mus musculus
  source 1..1639: mol_type = unassigned DNA
actaggtgcgcccttcgcagacctggagacccagagtaccgtggtagcggcgctctccttcgtcttcctcccgcgtcgagcccaagtggctcgagccgcggtctcacgcagcgccactcgtccacagcgaagaacctgaggacccacggaagtgacagaatgaatcgcaccgcatacactgtgggagcgttgcttctcctcctggggaccctactgccaacagctgaggggaaaaagaaaggttcccaaggagccattccgcctcctgacaaggctcagcacaatgactctgagcagacccagtccccaccacaacctggctccaggacccgggggcggggccaggggcggggcaccgccatgcctggagaggaggtgcttgagtccagccaagaggccctgcacgtgacagagcgcaagtatctgaagcgagattggtgcaaaactcagcccctgaagcagaccatccacgaggagggctgcaacagccgcactatcatcaaccgcttctgttatggccagtgcaactccttctacatccccaggcacatccgaaaggaggaagggtcctttcagtcttgctccttctgcaagcccaagaagttcaccaccatgatggtcacactcaactgtcctgagctacagccacccaccaagaagaaaagggtcacacgcgtgaagcagtgccgttgcatatccatcgacttggattaagtcaaagcgggcacattcagcctgtcatagccatgctgagagagccacacccaaaccacccgattcctacttggcttaaacctagaggccagaagaaccagcagttgcttcctggctggaggctgcttatgcatagtgtatgcgcatgagtgtgcatgggtgcctgtgggtgtttccaaacaccagccggaaacagcctttgctagaaggcacttcctgttactctgcttcagatggtcggaaatgcccacaccactggacccaaacatccacaggggcagggctgtagttggctttgtcattgtgttccatgtgcctcctgggcaccaggatttcacttgagaatgaatactaatgggggaggtaactctgagggctgcattagactcggaactgttcagtgctcgccctatgctcccatagcccatccctttctttgctctccctgacatctcagtcgtagcccatgttcctaaattaattcacttgaccgcgggtgtaagtcttttgtcttgtgaagaaccttcagaatgtggggagacacgtggtgatggcaaacgggacagaggactgacgcaggaacggtcaggctgaggaccagtctgggccagtgaaattcagtagtgagatgtctagagtttaaaagttgtttcccaaaacaatattagtcttgtttttagcaaaagggttttcctgatatttaaaagaacccagacacacagaggaaaaatataatcagcaaaaaaacaaaacaaaacaaaataacacaacaataacaacaacaacaaacaaaaacccaattctctgtgccagcttctgtgacctactgatactagctgtaactgatactagctgttaagggtgaaatgctgaccactcctgttttaagaaccaagtgaaattaaaaaagaaaatgtggcctcct

SEQ ID NO: 147 - protein, 114 aa, Homo sapiens
MSRTAYTVGALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 148 - protein, 184 aa, Mus musculus
MNRTAYTVGALLLLLGTLLPTAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 149 - DNA, 624 bp, Homo sapiens
  source 1..624: mol_type = unassigned DNA
atgctgccttgcctcgccctgcttctcctgatggagctgtccgtgtgcactgtggcaggtgatggtggagaggaacagacactcagcactgaagcagagacctgggaaggcgctggccccagcattcagctccagctgcaggaggtgaagacgggcaaggcaagccagttctttgggctgatggggaagcgagtgggagcatatcagctggaacacacgttccagggcctcctgggcaagagaagcctgttcacagaaggcagagaggatgaggcccaaggttcagagtaaaagcccccaccacagacttcccagaggacacggtgccgcttcttcctacctggatgtcacagctgacaagccggcaggccaactctcttctctgtgtctcctgtcctcatcgctggcacttcacacaaggcccacactgaacccactgggcttcttcctggactctcagtgtcaagcagcagtcctgcataaatgcacagcttcgccgtagcaagctgcactgactctgccctccctcacactcagagttggcatctcactgcacagcagtgaggagactcgcacactctgtatcctgtgcctagcacacagtaggcactcaataaatgagtg

SEQ ID NO: 150 - DNA, 1262 bp, Mus musculus
  source 1..1262: mol_type = unassigned DNA
gagaggctgtcccatgaagcagtgcaaaggtggggtgaggcataggaaggaagcagagagtctaggacacatctcagccacatacaagtggccccgtggggaccggagcctgctggaggaggtggcatcaggctgagtggggctccctggacagagaggcacctgctcaccatgttgcctctccttgccctgcttctcctgatcgggccatcagtgtgcactacagcaggagacagagaggaactggcttttggtgcagaggcagagtcctgggtgaccgtgaacctgaagggaatccccgtccccagcattgaacttaagcttcaggagttgaagagaagcaggactcgccagttctatggtctgatggggaagcgggtgggagggtatcagctgggacgtatagtgcaggatctccttggcacgagaggtttgtccatagaagggacctgcagacaggcggcgagtcaacagagggcacgacccggagcagtgaccagagaaagcctccagagtcgagaggaagatgaggctcccctaaccaccagcaacgtgtagcactctgccacccatctctccccagaacatcacagctgaggagcctcagccaacagtcctgtcttttgtcctcaacttgggtatttcctgccaggccctcgtcacactctgcattttctcccaggactcactcttggcatctggtagcaacgacacacaaagcacggctgcctcctcacaagctggactgacccagctctcccttgtccctacagcaaagcttcacactctgtatcccaggcctagcacacagtagggctcaatcaacgacgagttagcagtaataggtagcttcctcagccatgcagggtagagggtgggatgtcagaaaagcagagaccaacattacacagcccaggtctaatgtctaatccttgggtgggaagagagcttggggcctggctaaaacggcaatagaaacaagaaaaagaccctggtggagaagacgctgcatgtattgtatttgggggcggggtcaggagccttcttccctttagattcctgatgttgctaccaacagacatctccctctgtgctgaggctatggctcagtgggctaaggcgattgtggccaagcctgacaacttgagttcaatccccaggacccacaaagtggaagaaaaaaattgtccttttacctctgcatgtgtcatggcatatgttacacaaaataaaaagacagtttggaaaagaaaaaaaaaaaa

SEQ ID NO: 151 - protein, 96 aa, Homo sapiens
MLPCLALLLLMELSVCTVAGDGGEEQTLSTEAETWEGAGPSIQLQLQEVKTGKASQFFGLMGKRVGAYQLEHTFQGLLGKRSLFTEGREDEAQGSE

SEQ ID NO: 152 - protein, 128 aa, Mus musculus
MLPLLALLLLIGPSVCTTAGDREELAFGAEAESWVTVNLKGIPVPSIELKLQELKRSRTRQFYGLMGKRVGGYQLGRIVQDLLGTRGLSIEGTCRQAASQQRARPGAVTRESLQSREEDEAPLTTSNV

SEQ ID NO: 153 - DNA, 2436 bp, Homo sapiens
  source 1..2436: mol_type = unassigned DNA
acagtgctccggatcctccaatcttcgctcctccaatctccgctcctccacccagttcaggaacccgcgaccgctcgcagcgctctcttgaccactatgagcctcctgtccagccgcgcggcccgtgtccccggtccttcgagctccttgtgcgcgctgttggtgctgctgctgctgctgacgcagccagggcccatcgccagcgctggtcctgccgctgctgtgttgagagagctgcgttgcgtttgtttacagaccacgcaaggagttcatcccaaaatgatcagtaatctgcaagtgttcgccataggcccacagtgctccaaggtggaagtggtagcctccctgaagaacgggaaggaaatttgtcttgatccagaagccccttttctaaagaaagtcatccagaaaattttggacggtggaaacaaggaaaactgattaagagaaatgagcacgcatggaaaagtttcccagtcttcagcagagaagttttctggaggtctctgaacccagggaagacaagaaggaaagattttgttgttgtttgtttatttgtttttccagtagttagctttcttcctggattcctcactttgaagagtgtgaggaaaacctatgtttgccgcttaagctttcagctcagctaatgaagtgtttagcatagtacctctgctatttgctgttattttatctgctatgctattgaagttttggcaattgactatagtgtgagccaggaatcactggctgttaatctttcaaagtgtcttgaattgtaggtgactattatatttccaagaaatattccttaagatattaactgagaaggctgtggatttaatgtggaaatgatgtttcataagaattctgttgatggaaatacactgttatcttcacttttataagaaataggaaatattttaatgtttcttggggaatatgttagagaatttccttactcttgattgtgggatactatttaattatttcactttagaaagctgagtgtttcacaccttatctatgtagaatatatttccttattcagaatttctaaaagtttaagttctatgagggctaatatcttatcttcctataattttagacattctttatctttttagtatggcaaactgccatcatttacttttaaactttgattttatatgctatttattaagtattttattaggagtaccataattctggtagctaaatatatattttagatagatgaagaagctagaaaacaggcaaattcctgactgctagtttatatagaaatgtattcttttagtttttaaagtaaaggcaaacttaacaatgacttgtactctgaaagttttggaaacgtattcaaacaatttgaatataaatttatcatttagttataaaaatatatagcgacatcctcgaggccctagcatttctccttggataggggaccagagagagcttggaatgttaaaaacaaaacaaaacaaaaaaaaacaaggagaagttgtccaagggatgtcaattttttatccctctgtatgggttagattttccaaaatcataatttgaagaaggccagcatttatggtagaatatataattatatataaggtggccacgctggggcaagttccctccccactcacagctttggcccctttcacagagtagaacctgggttagaggattgcagaagacgagcgggagcggggagggcagggaagatgcctgtcgggtttttagcacagttcatttcactgggattttgaagcatttctgtctgaatgtaaagcctgttctagtcctggtgggacacactggggttgggggtgggggaagatgcggtaatgaaaccggttagtcagtgttgtcttaatatccttgataatgctgtaaagtttatttttacaaatatttctgtttaagctatttcacctttgtttggaaatccttcccttttaaagagaaaatgtgacacttgtgaaaaggcttgtaggaaagctcctccctttttttctttaaacctttaaatgacaaacctaggtaattaatggttgtgaatttctatttttgctttgtttttaatgaacatttgtctttcagaataggattctgtgataatatttaaatggcaaaaacaaaacataattttgtgcaattaacaaagctactgcaagaaaaataaaacatttcttggtaaaaacgtatgtatttatatattatatatttatatataatatatattatatatttagcattgctgagctttttagatgcctattgtgtatcttttaaaggttttgaccattttgttatgagtaattacatatatattacattcactatattaaaattgtacttttttactatgtgtctcattggttcatagtctttattttgtcctttgaataaacattaaaagatttctaaacttca

SEQ ID NO: 154 - DNA, 1656 bp, Mus musculus
  source 1..1656: mol_type = unassigned DNA
agtttcctgcctgaaggaagagagagaacagataaaaggggtgcagtgggtttgagaacaccatagtgctctgtagagccccaatctccacacctcctccagcatatcccgagctggtgctccctggtccgggatcttgtccacaatgagcctccagctccgcagctccgcccgcatccccagcggttccatctcgccattcatgcggatggcgccgctggcatttctgttgctgttcacgctgccgcagcatctagctgaagctgccccttcctcagtcatagccgcaacggagctgcgttgtgtttgcttaaccgtaactccaaaaattaatcccaaattgatcgctaatttggaggtgatccctgcaggtccacagtgccctacggtggaagtcatagctaaactgaaaaaccagaaggaggtctgtctggatccagaagctcctgtgataaagaaaatcattcagaaaatattgggcagtgacaaaaagaaagctaagcggaatgcactcgcagtggaaagaacggccagtgttcaatagaaagatttctgaggactctgaccccagtgaagataagaagaaagggctgattctctccacccacggattttctttatgaactccctgctttgatgagaaaagggaaaccattgtccctgaagcttcctgctcatctcacaagtgtatttagcaaaaatatttcctattctttcctatctgtcacactatcggaaattgtgatactttagtgtgtaagtcaagaatcattggttgttaacctttcaaaatgtcttgaactgaaggtgactattgcattgtctccggaatatgtatatgtatatgtatatgtatatgtatatgtatatgtatatgtatatgtatatgtatatgtatatgtatatgtatatgtatatgatgtatatgtatatgtatatgtgtgtgtatatatatatatatatcctttgtaatgttcatgagaaggcaatgctgtcatactctctctcataaaatggggacctcttttactgttgtttcttgggtgtgttaagagtgttcttacgtctgagcctgggctggcatataacttttatggttttagaaagctgctgtgtcatgcagaaacctatgtagaacacagttccttatttagaatttctaaatgtgtaagttctagaaggatcaatgtgttctcatcttataattttagacatttgatgtcttcgtagtatggcataatgtcatgacttactcaacaaactgtatactatttattaattattaactagtttattagtattactatttgggccactaactatatattttagatagtatatattttagataggtgaagaaggtaagaagcaaggaaattccttacctagttttcataaaaaacatttaaaagttatttctagggagagaaactttagcaaatatctatactcttagagttttgaaaacatacttaacatgtttgaatatggattgctgagatatttagatgtctgtagtgtgtcttttaaagagtgtgctttgttataaggagattaatgaattcatcacatccattattttaagcctgtacatcttatagtgtctcacttctttatattactcttttgaggaaaataaagtagctctagattcca

SEQ ID NO: 155 - protein, 114 aa, Homo sapiens
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 156 - protein, 132 aa, Mus musculus
MSLQLRSSARIPSGSISPFMRMAPLAFLLLFTLPQHLAEAAPSSVIAATELRCVCLTVTPKINPKLIANLEVIPAGPQCPTVEVIAKLKNQKEVCLDPEAPVIKKIIQKILGSDKKKAKRNALAVERTASVQ

SEQ ID NO: 157 - DNA, 3207 bp, Homo sapiens
  source 1..3207: mol_type = unassigned DNA
ggcccacagaggagcacagctgtgtttggctgcagggccaagagcgctgtcaagaagacccacacgcccccctccagcagctgaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagcctaatcagcccaccatgttctccactgaaaaatcctctttctttggggtttttctttctttcttttttgattttgcactggacggtgacgtcagccatgtacaggatccacaggggtggtgtcaaatgctattgaaattgtgttgaattgtatgctttttcacttttgataaataaacatgtaaaaatgtttcaaaaaaataataaaataaataaatacgaagaatatgtcaggacagtcaaaaaaaaaaaaaaaaaa

SEQ ID NO: 158 - DNA, 3024 bp, Mus musculus
  source 1..3024: mol_type = unassigned DNA
gagcacagctggatcaggctgcagcagagccccgagagctttgtgaaggaggaccgccgcacacccgcctccggcagcacacagccaaccacagctgagcgacagccaacaagagccaatcacaaggcacctttgaatactcaggatgcagatgtcttcagcccttgcttgcctcatcctgggcctggttctggtctctgggaaagggttcactttacccctccgagaatcccacacagcccatcaggccaccgacttcggagtaaaagtgtttcagcaggtggtccaggcctccaaagaccggaatgtggtcttctctccctatggcgtgtcctcggtgctggctatgctgcagatgaccacagcggggaaaacccggcggcagatccaagatgctatgggattcaaagtcaatgagaagggcacagctcatgccctccgccagctctccaaggagctcatggggccgtggaacaagaatgagatcagtactgcggatgccatctttgtccagcgggacctagagctggtccagggcttcatgccccacttcttcaagctcttccagactatggtgaaacaggtggacttctcagaagtggaaagagccagatttatcatcaatgactgggtggaaaggcataccaaaggtatgatcagtgacttactggccaagggggctgtagacgagctgacacgcctggtgctggtgaatgccctctacttcagtggccaatggaagactcctttcttagaggccagcacccaccagcgcctcttccacaagtctgatggcagcaccgtctctgtgcccatgatggctcagagcaacaagttcaactacactgagttcaccacccccgatgggctcgagtatgacgttgtggaactgccctaccagggcgacaccctcagcatgttcatcgctgcaccctttgagaaagatgtgcacctctccgccctcaccaacatcttggatgctgaactcatcagacaatggaagggcaacatgaccaggctgccccgcctcctcatcctgcctaagttctctctggagactgaagtggacctcagagggcccctggagaagttgggcatgcctgacatgtttagtgcaaccctggccgacttcacaagtctttccgaccaagagcagctctctgtagcacaggcactgcaaaaggtcaggatcgaggtaaacgagagcggcacagtggcgtcttcctccacagcctttgtcatctcagcccgcatggcccccacggagatggttatagaccgatcctttctctttgtggttcggcacaacccgacagagacaatcctcttcatgggccaagtgatggagccttgacagtgggaagagacgccttcatttgggacgaaactggagatgttataagcagaaactctgaagaaaaggattatttaaaggactctatggggagaaagagaaggcaactcctccttaccccccacactggtaatctttccaaccagcatcccagacctcggactcttgaagggaaaagagtctaactccctcctccctagggattcctaccccacaaaggtctcatggaccatagaactcacagtacctggatctgcccagcatgccctttggacccagttcccaccgaggccccagcagagtggagggcacaacactttcattcagcaaaatcgtttgtgttccagtcacactgtgggcacctcttgcatcgcctgccattgctgtggagggtggccatgggccaaaggaaaaagcactgtcctatctcaaggtccactgtggaaatgtccaccttgcccacctccaaggggcaacggatagacagatcaaatggtggcccaatagcgagccttctccctgctccctcccttgacacagcttgcttatgttatttcagagtgtaggtgacttgtttacacagcttttttcgacccacaaacttttttcatttggaaagggtgtaagaaaagtcggacgtgtgtgtgcctggctcttcgtccccagtctcccagtgggggggccctggggagattccaggggtgtgattgaatatttatctcttgctcttgtatgtttgttggggagaagaagcacttttaaggaaaatgcttcttatttaaaccgtggcatacggcatcccatttggggtctgcatccctgtatgtcaggggtgcatcactccacaaacctgcccctctgggtagcctcgtgatggggctcacactgccgcctagtggcagccgaacacacccttacccggtccctccctccctcccccccccccccccccccccccgtggctcttttccttagggaccttgccaaggtgatgcttggcaacccacgttaaaggaaggggggaaaaaagattagatggaagagagagagatttgagagagggcaaagtggtttcaaatttttccaaggcattcagaagcagagagggaaaaggggctgtgtgacctaacaggacagaactttctccaattactgggtgagtcagagctgcactggtgactcacttcaatgtgtcatttccggctgctgtatgtgagcagtggacacgtgggggggcgggggggggatgaaagagacagcagctcctggtcaaccaccttagttagataatcttttttgaaagcttcctagctggaggtatgatcagaaaaccaatttactgaaaaactgcacaagaaggtaccgtgaatgtaatttcctagcaggccactctgcatctgttatgtctccaccggaaaaaaaataatcatgttggtgtttttgcttttctctctctccctctttctctctgatttttttttcctctcttttcattatgcactggacagccacacaccgtgtacccatagggccccaaatgtggggtcacatggtcttgaattttgttggttacatatgcctttttgttgttgtttgtcttcacttttgatatataaacaggtaaatatgttttttaaaaaatactaaatatagagaatatgcaaacaaaa

SEQ ID NO: 159 - protein, 402 aa, Homo sapiens [sequence omitted]

SEQ ID NO: 160 - protein, 402 aa, Mus musculus
MQMSSALACLILGLVLVSGKGFTLPLRESHTAHQATDFGVKVFQQVVQASKDRNVVFSPYGVSSVLAMLQMTTAGKTRRQIQDAMGFKVNEKGTAHALRQLSKELMGPWNKNEISTADAIFVQRDLELVQGFMPHFFKLFQTMVKQVDFSEVERARFIINDWVERHTKGMISDLLAKGAVDELTRLVLVNALYFSGQWKTPFLEASTHQRLFHKSDGSTVSVPMMAQSNKFNYTEFTTPDGLEYDVVELPYQGDTLSMFIAAPFEKDVHLSALTNILDAELIRQWKGNMTRLPRLLILPKFSLETEVDLRGPLEKLGMPDMFSATLADFTSLSDQEQLSVAQALQKVRIEVNESGTVASSSTAFVISARMAPTEMVIDRSFLFVVRHNPTETILFMGQVMEP

SEQ ID NO: 161 - DNA, 2619 bp, Homo sapiens
  source 1..2619: mol_type = unassigned DNA
gccgactgggtcccctgccgctgtcgccaccatggctccgcaccgccccgcgcccgcgctgctttgcgcgctgtccctggcgctgtgcgcgctgtcgctgcccgtccgcgcggccactgcgtcgcggggggcgtcccaggcgggggcgccccaggggcgggtgcccgaggcgcggcccaacagcatggtggtggaacaccccgagttcctcaaggcagggaaggagcctggcctgcagatctggcgtgtggagaagttcgatctggtgcccgtgcccaccaacctttatggagacttcttcacgggcgacgcctacgtcatcctgaagacagtgcagctgaggaacggaaatctgcagtatgacctccactactggctgggcaatgagtgcagccaggatgagagcggggcggccgccatctttaccgtgcagctggatgactacctgaacggccgggccgtgcagcaccgtgaggtccagggcttcgagtcggccaccttcctaggctacttcaagtctggcctgaagtacaagaaaggaggtgtggcatcaggattcaagcacgtggtacccaacgaggtggtggtgcagagactcttccaggtcaaagggcggcgtgtggtccgtgccaccgaggtacctgtgtcctgggagagcttcaacaatggcgactgcttcatcctggacctgggcaacaacatccaccagtggtgtggttccaacagcaatcggtatgaaagactgaaggccacacaggtgtccaagggcatccgggacaacgagcggagtggccgggcccgagtgcacgtgtctgaggagggcactgagcccgaggcgatgctccaggtgctgggccccaagccggctctgcctgcaggtaccgaggacaccgccaaggaggatgcggccaaccgcaagctggccaagctctacaaggtctccaatggtgcagggaccatgtccgtctccctcgtggctgatgagaaccccttcgcccagggggccctgaagtcagaggactgcttcatcctggaccacggcaaagatgggaaaatctttgtctggaaaggcaagcaggcaaacacggaggagaggaaggctgccctcaaaacagcctctgacttcatcaccaagatggactaccccaagcagactcaggtctcggtccttcctgagggcggtgagaccccactgttcaagcagttcttcaagaactggcgggacccagaccagacagatggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctgaggaggggcagggcccacccatgtcaccggtcagtgccttttggaactgtccttccctcaaagaggccttagagcgagcagagcagctctgctatgagtgtgtgtgtgtgtgtgtgttgtttcttttttttttttttacagtatccaaaaatagccctgcaaaaattcagagtccttgcaaaattgtctaaaatgtcagtgtttgggaaattaaatccaataaaaacattttgaagtgtg

SEQ ID NO: 162 - DNA, 2640 bp, Mus musculus
  source 1..2640: mol_type = unassigned DNA
gtgccctgcccaccctgccgctcgcacagcgcaccccgccccaaggccaggaaccagctgagcacacagctggactgagccgtcgctgtcgccgtcaccagctctgctgcatcaggtaccaccagcttctcttccagtgcttcagccgctgtcatagcatctcctctcggagtggctctctccttgtgcagcctgtaagcccagcactatggtggtggagcaccccgaattcctgaaggcagggaaggagcctggcctgcagatctggcgtgtggagaagtttgacctggtgcctgtgccccccaacctctatggagacttcttcacgggtgatgcctatgtcatcctaaagactgtgcagctgaggaatgggaatctgcagtatgacctccactattggctgggcaatgaatgcagccaggatgagagcggggctgctgccatctttactgtgcaactggatgactacctgaacggccgggctgtacagcaccgtgaggttcagggctttgagtcgtccaccttctccggctacttcaagtctggacttaagtacaagaaaggaggtgtggcatctggattcaaacacgtggtacccaatgaggtggtggtccagaggctcttccaggtcaaaggacgccgtgtagtccgtgctactgaggtacctgtgtcctgggacagtttcaacaatggcgactgcttcattctggacctgggaaacaatatctatcagtggtgtggctctggcagcaacaaatttgaaaggctgaaggccacacaggtgtccaagggcatccgggacaacgagaggagtggccgtgctcaagtacacgtgtctgaagagggaggagagccagaagccatgctgcaggtgctgggccccaagccagctctgcctgaaggtaccgaggacacagccaaggaagatgcagccaaccgaaggctggccaagctctacaaggtctccaacggtgcaggtagcatgtcagtctccctagtggctgatgagaaccccttcgcccagggcgccctgagatctgaggactgcttcatcctggaccatggcagagatgggaaaatctttgtttggaaaggcaagcaggccaacatggaggagcggaaggctgccctcaaaacagcctctgacttcatctccaagatgcagtaccccaggcagacccaggtttcagttctcccagagggcggtgagacccctctctttaagcagttcttcaagaactggcgggacccagaccagacagacggccccggcctgggctacctctccagccacattgccaacgtggagcgcgtacctttcgatgctgctacgctgcacacctccaccgccatggccgctcagcacggcatggatgatgatggaactggccagaaacagatctggagaattgaaggttccaacaaggtgccagtggaccctgccacatacgggcagttctatggaggcgacagctacatcattctgtacaactaccgccacggtggccgccagggacagatcatctacaactggcagggtgcccagtctacccaggatgaggttgctgcttctgccatcctgactgcccagctggatgaggagctgggaggaactcctgtccagagccgagtggtccaaggcaaagagcctgcacacctcatgagcttgtttggcgggaagcccatgatcatctacaagggtggcacctcccgtgatggtgggcagacagctcctgccagtatccgcctcttccaagtgcgagccagcagctctggagccaccagggctgtggaggtgatgcctaagtctggtgctctgaactccaacgatgcctttgtgctgaaaaccccctccgctgcctacctgtgggtgggcgcaggagccagtgaggcggagaagaccggcgcccaggagcttctgaaggtgcttcggtcccagcatgtgcaggtggaagaaggcagtgagccagatgccttctgggaggctctgggcgggaagactgcctaccgcacatcccccaggcttaaggacaagaagatggatgcccatcctcctcgactctttgcctgctccaacaggatcggacgctttgtgatcgaagaggttcctggcgagcttatgcaggaagacctggctactgatgacgtcatgctcctggacacctgggaccaggtctttgtctgggttggaaaagactcccaggaagaggaaaagacggaagccttgacttctgctaagcggtacatcgagacagatccagcaaatcgggacaggcggacccccatcacagtcgttaggcagggctttgagcctccttccttcgtgggctggttcctcggctgggatgacaactactggtcggtggatcctttggaccgggccttggctgagctggctgcctgagtaaggaccaaagccatcaatgtcaccaatcagtgcctttgagggttgtccatctcccaaagacatcatatggcaagcaggaaaactatgatgtgtgcgcgcgtgtttttgtttttgttttttacggtagccaaaacaagccctgtggaaactcagggtctttacagaattgcttcaaatgtctgtactttggaaatgaaagccaataaaagctttttgaagtgtgttctccccag

SEQ ID NO: 163 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 164 - protein, 731 aa, Mus musculus
MVVEHPEFLKAGKEPGLQIWRVEKFDLVPVPPNLYGDFFTGDAYVILKTVQLRNGNLQYDLHYWLGNECSQDESGAAAIFTVQLDDYLNGRAVQHREVQGFESSTFSGYFKSGLKYKKGGVASGFKHVVPNEVVVQRLFQVKGRRVVRATEVPVSWDSFNNGDCFILDLGNNIYQWCGSGSNKFERLKATQVSKGIRDNERSGRAQVHVSEEGGEPEAMLQVLGPKPALPEGTEDTAKEDAANRRLAKLYKVSNGAGSMSVSLVADENPFAQGALRSEDCFILDHGRDGKIFVWKGKQANMEERKAALKTASDFISKMQYPRQTQVSVLPEGGETPLFKQFFKNWRDPDQTDGPGLGYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSRDGGQTAPASIRLFQVRASSSGATRAVEVMPKSGALNSNDAFVLKTPSAAYLWVGAGASEAEKTGAQELLKVLRSQHVQVEEGSEPDAFWEALGGKTAYRTSPRLKDKKMDAHPPRLFACSNRIGRFVIEEVPGELMQEDLATDDVMLLDTWDQVFVWVGKDSQEEEKTEALTSAKRYIETDPANRDRRTPITVVRQGFEPPSFVGWFLGWDDNYWSVDPLDRALAELAA

SEQ ID NO: 165 - DNA, 513 bp, Homo sapiens
  source 1..513: mol_type = unassigned DNA
accagtcacggaccctggacccagcgcacccgcaccatggccggccccagcctcgcttgctgtctgctcggcctcctggcgctgacctccgcctgctacatccagaactgccccctgggaggcaagagggccgcgccggacctcgacgtgcgcaagtgcctcccctgcggccccgggggcaaaggccgctgcttcgggcccaatatctgctgcgcggaagagctgggctgcttcgtgggcaccgccgaagcgctgcgctgccaggaggagaactacctgccgtcgccctgccagtccggccagaaggcgtgcgggagcgggggccgctgcgcggtcttgggcctctgctgcagcccggacggctgccacgccgaccctgcctgcgacgcggaagccaccttctcccagcgctgaaacttgatggctccgaacaccctcgaagcgcgccactcgcttcccccatagccaccccagaaatggtgaaaataaaataaagcaggtttttctcctcta

SEQ ID NO: 166 - DNA, 555 bp, Mus musculus
  source 1..555: mol_type = unassigned DNA
aaccatcacctacagcggatctcagactgagcaccatcgccatggcctgccccagtctcgcttgctgcctgcttggcttactggctctgacctcggcctgctacatccagaactgccccctgggcggcaagagggctgtgctggacctggatatgcgcaagtgtctcccctgcggcccgggcggcaaaggacgctgcttcggaccaagcatctgctgcgcggacgagctgggctgcttcgtgggcaccgccgaggcgctgcgctgccaggaggagaactacctgccttcgccctgccagtctggccagaagccctgcgggagcggaggccgctgcgccgccacaggcatctgctgcagcccggatggctgccgcacagaccccgcctgcgaccctgagtctgccttctcggagcgctgagcccactttctgggaatacctttagcgcgcttccttcgttccccatggccactgccagaaaaaaaaaaaaaaaagaaaagaaaagaaaagaaaagaaaaataaagtagatttcctcttcaaaaaaaaaaaaaaaaa

SEQ ID NO: 167 - protein, 125 aa, Homo sapiens
MAGPSLACCLLGLLALTSACYIQNCPLGGKRAAPDLDVRKCLPCGPGGKGRCFGPNICCAEELGCFVGTAEALRCQEENYLPSPCQSGQKACGSGGRCAVLGLCCSPDGCHADPACDAEATFSQR

SEQ ID NO: 168 - protein, 125 aa, Mus musculus
MACPSLACCLLGLLALTSACYIQNCPLGGKRAVLDLDMRKCLPCGPGGKGRCFGPSICCADELGCFVGTAEALRCQEENYLPSPCQSGQKPCGSGGRCAATGICCSPDGCRTDPACDPESAFSER

SEQ ID NO: 169 - DNA, 1641 bp, Homo sapiens
  source 1..1641: mol_type = unassigned DNA
gaagggagccgggatcagccaggggccagcatgagccggagggagggaagtctggacccccagactgattcctcagtctcacttcttccccacttggaggccaagatccgtcagacacacagccttgcgcacctcctcaccaaatacgctgagcagctgctccaggaatatgtgcagctccagggagaccccttcgggctgcccagcttctcgccgccgcggctgccggtggccggcctgagcgccccggctccgagccacgcggggctgccagtgcacgagcggctgcggctggacgcggcggcgctggccgcgctgcccccgctgctggacgcagtgtgtcgccgccaggccgagctgaacccgcgcgcgccgcgcctgctgcgccgcctggaggacgcggcgcgccaggcccgggccctgggcgccgccgtggaggccttgctggccgcgctgggcgccgccaaccgcgggccccgggccgagccccccgccgccaccgcctcagccgcctccgccaccggggtcttccccgccaaggtgctggggctccgcgtttgcggcctctaccgcgagtggctgagccgcaccgagggcgacctgggccagctgctgcccgggggctcggcctgagcgccgcggggcagctcgccccgcctcctcccgctgggttccgtctctccttccgcttctttgtctttctctgccgctgtcggtgtctgtctgtctgctcttagctgtctccattgcctcggccttctttgctttttgtgggggagaggggaggggacgggcagggtctctgtcgcccaggctggggtgcagtggcgcgatcccagcactgcagcctcaacctcctgggctcaagccatccttccgcctcagcttccccagcagctgggactacaggcacgcgccaccacagccggctaattttttatttaattttttgtagagacgaggtttcgccatgttgcccaggctggtcttgaactccggggctcaagcgatcctcccgcttcagcctccctaagtgctgggattgcaggcgtgagccactttcccagcctctctttgctttgcctgccccgttctcttaactcttggaccctcctcgtctgcatggtaactccgtctgagtctaccattttcttgctctccctccttccttgggcctgcctcagttccctttggcctccccctttacccagctcttggggtgtctctgttttttccatccccacttcctgccttctcgtggccctgtgtgagcacatgtgtacatctcagccttatctcaaggaggtgacaccttctctccttgtccccatctggctgtctctctgtgcttccctggccaggggcgtgcctgctggtcctatggggggaaggctactctgcatctcagccaccttcctcaggctcactccacctacatccccagtctgccacaccccatccctttgggcctcagccctgtccctttgatgtcctcctttccttcagcccctctgccctgtccctgcacacctccactgcctcccagcagatctgtggagacaaaacaagcaacaccttaaaagagttttatttctgaaaataaattaattttttgtaaataaaatgtttaaaaataaaaaa

SEQ ID NO: 170 - DNA, 1403 bp, Mus musculus
  source 1..1403: mol_type = unassigned DNA
cgggtgagttgggagaaagagcaggcgcaaggaattgaggcttcagggccttgggttcaggggttagatcaccgaatatatatggaagaccaccagactgactcctcaatctcattcctaccccatttggaggccaagatccgccagacacacaaccttgcccgcctcctgaccaaatatgcagaacaacttctggaggaatacgtgcagcaacagggagagccctttgggctgccgggcttctcaccaccgcggctgccgctggccggcctgagtggcccggctccgagccatgcagggctaccggtgtccgagcggctgcggcaggatgcagccgccctgagtgtgctgcccgcgctgttggatgccgtccgccgccgccaggcggagctgaacccgcgcgccccgcgcctgctgcggagcctggaggacgcagcccgccaggttcgggccctgggcgccgcggtggagacagtgctggccgcgctgggcgctgcagcccgcgggcccgggccagagcccgtcaccgtcgccaccctcttcacggccaacagcactgcaggcatcttctcagccaaggtgctggggttccacgtgtgcggcctctatggcgagtgggtgagccgcacagagggcgacctgggccagctggtgccagggggcgtcgcctgagagtgaatactttttcttgtaagctcgctctgtctcgcctctttggcttcaaattttctgtctctccatctgtgtcctgtgtgttcttgggctgtccctatctttctgcatttgtgtggtctctctcttctgctctcctctctgcagggagcttcttttttccaacagtttctcgttttgtctctctccagtcttgaacacttttgtctccgagaggtctctttttgtttccttgtctcttggttctttctttgcttgcttgcttgcttgcttgcttgttgttgagacagggtctcaccatatagctctggatggcctggaacttgctatgtaggccaggctggcctccagctcatagagatccacttgcctccgactcccaatttccccatctgtctccctgtgatccatatgggtatgtgtaacccttactttgtctcatggaggtgacaatttttctcccttcagtttctttgttctttactgaccagaaaagtgcctacttgtcccctggtggcaaggccattcaccttaggaccttcccaccagttcctttgtaggcaaatccctccccctttgaggtccttccctttcataccgccctaggctggtcaatggagagagaaaggcagaaaaacatctttaaagagttttatttgagaataaattaatttttgtaaataaaatgtttaacaataaaactaaacttttatgaaaaaaaaaaaaaaaaa

SEQ ID NO: 171 - protein, 200 aa, Homo sapiens
MSRREGSLDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 172 - protein, 196 aa, Mus musculus
MEDHQTDSSISFLPHLEAKIRQTHNLARLLTKYAEQLLEEYVQQQGEPFGLPGFSPPRLPLAGLSGPAPSHAGLPVSERLRQDAAALSVLPALLDAVRRRQAELNPRAPRLLRSLEDAARQVRALGAAVETVLAALGAAARGPGPEPVTVATLFTANSTAGIFSAKVLGFHVCGLYGEWVSRTEGDLGQLVPGGVA

SEQ ID NO: 173 - DNA, 909 bp, Homo sapiens
  source 1..909: mol_type = unassigned DNA
ggacgggcccggcgcgggagggagcgaagcagcgcgggcagcgagcgagtgagcgcgcggcggcccctggtccgcccggccgcggccgatctaggggctgggggctggaggcgggggtgggggtctgagctgcgtcctgggctcgaggcgtcccccggggagtcgcctcttagcggatgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttcc

SEQ ID NO: 174 - DNA, 878 bp, Mus musculus
  source 1..878: mol_type = unassigned DNA
agtgagcgagtgagcgcacgcagtggctgtggccccagtcccttcaggcggctgctctgccaccaagggggctgaggtgggggtgggggtacgctgagacatcggttccaagtcctccctccgtctcccccttgtcggtccgacgttttgggcctggaaagtgggacaagatgcagcaccgaggcttcttccttctcgcccttcttgccctcttggtggtcacgtccgcggtggccaaaaaaaaagagaaggtgaagaagggcagcgagtgttcggagtggacctgggggccctgcacccccagcagcaaggactgcggcatgggcttccgcgagggtacctgtggggcccagacccagcgcgtccattgcaaggtgccctgcaactggaagaaggaatttggagccgactgcaaatacaagtttgagagctggggggcgtgtgatgggagcactggcaccaaagcccgccaagggaccctgaagaaggcgcggtacaatgcccagtgccaggagaccatccgcgtgactaagccctgcacctccaagaccaagtcaaagaccaaagccaagaaaggaaaaggaaaggactaagtcaggaggccagagagcctccggcctcgcctggagcctgaacggagccctcctctcccacaggcccaagatataacccaccagtgccttttgtcttcctgtcagctctgtcaatcacgcctgtcctctcacgcccacaccaagtgcccaaagtggggagggacaagagattctggaaagtgagcctccccataccctcttttgttctccccaccctgatacttgttattaagaaatgaataaaataaactcacttttttccaataaaagcttcttttttaatat

SEQ ID NO: 175 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 176 - protein, 140 aa, Mus musculus
MQHRGFFLLALLALLVVTSAVAKKKEKVKKGSECSEWTWGPCTPSSKDCGMGFREGTCGAQTQRVHCKVPCNWKKEFGADCKYKFESWGACDGSTGTKARQGTLKKARYNAQCQETIRVTKPCTSKTKSKTKAKKGKGKD

SEQ ID NO: 177 - DNA, 734 bp, Homo sapiens
  source 1..734: mol_type = unassigned DNA
aggttcagctgggagacccctcacacacaggtgaaggcgtggctgtagaacctcagaccccctggtctcctcaggaatgaaggtcattgccatcctcaccctcctcctcttctgctcccccacccacagcagtttctggcagtttcagaggagggtcaaacacatcacggggcgaagtgccttcttctcatattacggatatggctgctactgtgggcttggggataaagggatccccgtggatgacactgacagcccctcatctccctctccctacgagaagctgaaggagttcagctgccagcctgtgttgaacagctaccagttccacatcgtcaatggcgcagtggtttgtggatgcacccttggtcctggtgccagctgccactgcaggctgaaggcctgtgagtgtgacaagcaatccgtgcactgcttcaaagagagcctgcccacctatgagaaaaacttcaagcagttctccagccagcccaggacagctgcccccaccctgcaaaaaagcctgggctagcctgctggaggatgagagcccacctggatcagttgtctcagctgatttcagacacgtgagagagagctcagcgagactcagcttgtagctgactacagatgtgtgagggaacctggctgagaccaaaacaactgtccagctgagcccaggctaaactgccaacatgcagaattgtgagctaaataaaggctgctgttctaagtca

SEQ ID NO: 178 - DNA, 1618 bp, Mus musculus
  source 1..1618: mol_type = unassigned DNA
ggactagggcacggagggcggaggggcagaggtggttcacggattccaggctcaggaccttggtgtggccctgccccatggaggccacagggccctgggcctggacccctgccattggaactcttcttcctgacctggcctgactcttgtgtctcctggggaatggtctaactatatacagtcagcaactccagccacttcaccgcgtttcccaacagctttgagttttggaggccggcagcctgactgctttctcagaagctatggtccactacctcagccattctgttggagccaaactggcaaatgaaggtgaaatccccgaaccgtggacctcctggtcacctcgggaatgaagggcattgccatcttccttgtcttcatcttctactggacaacctccaccctcagcagtttctggcagttccagaggatggtcaaacacgtcacagggcgcagcgccttcttctcctattacggatatggctgctactgtgggcttgggggcaaagggctccctgtggatgccacagacaggtgctgctgggctcatgactgctgctaccacaagcttaaggaatatggctgccaacccatcttgaatgcctaccagttcaccattgtcaatgggacggtgacctgtggatgcaccgtggccagcagctgcctctgcgggcagaaggcctgtgagtgtgacaaacagtctgtgtactgcttcaaggagaacctggccacctacgagaaggccttcaagcagctcttcccgaccaggcctcagtgtggcagggacaaactccagtgctaggaaggccaccctcatccctccatcacctgctctagtcccgagccccttctgaagagctttgctgtctggggagtgtcttttcagtcattaagggactggaggtggagaattcctgccctggaaacaaaccgtgggtatctggccattaagagatacagtctgtcagtaggaaacaaggacataagggagcagggatggggggcatcctgcaatcccagcatttgagacatggaggcaggagaggaaggagaggcaggaggccaggggcaacctccactatacggtaagttcaaggctagcctgagctactttagaccctgccttgaaaaattttttaaacattttttaaaggaaaataaaacaggggaacacaggggttgagctgaaaggtgctctcaaacccttctccccagaagagcagagagccccaggatccagcctaaactcccctgtaccctctatcctggtcaggatgtgtgtctgactggggaaccaagtcatctacccaggtcaggctgggaagtgagccgggatcctagaaaacacagcccaggagcctcgcctggaatctcctcatttgcaccaggtctgacctctccagcaggatgcaggatgcacccctgtctcagaaggtggggcactatcagaaaaatggtgtgcaccagtgccacaaagatgtcactggttaagatggcatcaagaaatggaaagaggatgttcgggacagggggtccaaggcacccaaaatccttaccccaatttagaagcctttggtcctgtaagacttaattctagtaaacgaggaaggtctaa

SEQ ID NO: 179 - protein, 150 aa, Homo sapiens
MKVIAILTLLLFCSPTHSSFWQFQRRVKHITGRSAFFSYYGYGCYCGLGDKGIPVDDTDSPSSPSPYEKLKEFSCQPVLNSYQFHIVNGAVVCGCTLGPGASCHCRLKACECDKQSVHCFKESLPTYEKNFKQFSSQPRTAAPTLQKSLG

SEQ ID NO: 180 - protein, 150 aa, Mus musculus
MKGIAIFLVFIFYWTTSTLSSFWQFQRMVKHVTGRSAFFSYYGYGCYCGLGGKGLPVDATDRCCWAHDCCYHKLKEYGCQPILNAYQFTIVNGTVTCGCTVASSCLCGQKACECDKQSVYCFKENLATYEKAFKQLFPTRPQCGRDKLQC

SEQ ID NO: 181 - DNA, 3411 bp, Homo sapiens
  source 1..3411: mol_type = unassigned DNA
attcgccctggtctctgttccctttcgtactcaaagctcgtgcatccagggaggggaaaccggagatagggtcttcgggccccgggcagaccctctgtgccgctgcaaaccgttgcagcctgaggctgtcaggtcctcccccagacacctgcggaccctccctctcctggcttcccgtctggtcatggcgagattctgggtctgcgtagccggtgctggcttctttcttgcatttttggttttgcattcgcgtttttgtggctctccagttttgaggaactttacttttgcagtttcctggagaactgagaaaattctttaccggctggatgtgggttggcctaagcacccagaatattttaccggaacaacattttgtgttgcagttgactccctcaatggattggtttacataggtcaaagaggggataacatcccaaagatattagtgttcacagaggatggatatttcctacgagcctggaattatacagttgacacacctcatggtatatttgcagccagtactctatatgaacaatccgtctggatcacggatgtaggaagtggattctttggtcatactgttaaaaaatacagttcttttggtgatcttgttcaagtcttgggtactccaggcaaaaaaggcactagtttgaatcctttgcagtttgataacccagcagaattatatgtagaggacacaggagatatttacattgtggatggagatggaggattgaataacagattgatcaaactgtcccaagatttcatgatcctttggctgcatggagaaaatgggacagggcctgctaagttcaacatacctcacagtgttacacttgattcagctggtcgggtgtgggttgctgaccgaggaaataaaagaatccaagtatttgataaagacactggggagtggttaggagcatggaataattgtttcacagaagagggaccttcttcagtcaggtttactcctgatgggaagtacttgattgtggcccagctgaatcttagcaggctctcagtcgtagcagcacccccagtgggaagcattggggagtgttctgtgatcagcacaatccaactagcagatcaagttttgccacatctcctagaagtcgacagaaagactggagcagtctatgtagcagaaattggagcaaaacaagtacaaaaatatgtccctttgaatagctatgttccttcatttggttcataatgtttctttcctgggaatatttcaagtggcagttcagattctcaattcactaagtgcttaaaaatgatgttcaagcacaagaatttatttttctagtataaaagatctagtatcagaaagatttgtttttgtatcattaagaatcttatattttgttgccctcttgggacttagttttatttgtaagtgcataaggatattttaatgaaaggaaagtaactaaaaaatggggttgggaagagggactaaggtggtaacctcattatttgccctggtagactgattctccctgggtaaaaaaaatgggaataaaaatgagcttgcatgataatttattaaatttcatgtgaagaactccagacctccagattgtgcaactaacataaaatgagctgcttgagagattgtaaataagatgaactattgattaatttgagtacccacagagtgctgtgtcttgacgacttaaaaatgaaaaagcaggattgccttttgagtagcttgcagtctagtggggagacaagcaggcaaacagtcacaacacagcaaaagcgaccttggagcatagtgggacttttggagtaggagtgctgcatttgactgagggaatcatggatacttcgcaggagaagtgaatttgagctcagacttgaaaactgaggaggagcttaccaagggacaaggaggagaaaacaataatttccaagtaaagaaggtataaaaagttagaagtgtactgtaaactttgataggcttttaggccttttttaaagcccaacttggcttctgtccattacctataagatatttaatgtcagtcagcctttaaatgtaggaataaaatggctggcatctaagcactttagtaaaagaggtttttacaaataactaaggattgtagagcttccttctctttttttttctttttctttcttttgttttacatgaactcaacttattcctaacatttgtctacctcaaagaaatttcaagattatttagataacatggatatgtgccaaatcctttgagctgttaagatgataatttcctgctttcctcctacatcttctcctcccactccctcctttggtgtgaatattggcttcccaattaagacctttttttttttttttccagtttgttttagcttattataggttttggaggaactttgccattttgtaatctttcaaatcattcttcacccttcctcacatcagcttcctgcttttcccagtgttttactgtaaattgtgtagcatatgacaaatcttgagctgactttcctcttcacctgttatggctggagtattttccagacctgaagggactcacacttgttttgatacttggatcacatctccgtgaggttaggaaggtaaatctaccaacaggaagccctgtactctgtattccaaggccattggtaaatgtgttggtgccactgatcggactgtatgaccttaaacaagtcaccttagttttcagtgaaatgggaatatcattgtctcctctttcatgaatgctgtgagaatcagatgtgcaacaggtacagacttgccctttgaaatctatacctctggatacattagaggcattttattaacaaaggcccttctaaatgtgctatttatttgacaataactatcagatttgccttaattttgtgtttatagcatttatcaaaacgtatcctcatagactttatgcagattaatatggtcaattgatttggataaaagaaagtaatttcagggtttgtttttaagccaggacaagaagtgcaaatgcctctttgaagcaatttaggctaaactgattttgaaatttcaaaatgttttattttactttgttttattaagccaggacaagaagtgcaaatgcctctttgaagcaattcaggctaggtaaaccgattttgccatttcaaaacgttttattttactttgttttatatcagagtcttataaaacctgctgcaaatatttctgaatgtctttgtaaaagtgtttgttagtgtacctgtgattatagtacttcacttttttcctttggattaattggttaaatgaatgagaaatgtgttatgttttttactaaaaagtataaattaaaattttggaaagaaaaggcaatattatctggctccccaattaaagtttgattttattgtcacaaaa

SEQ ID NO: 182 - DNA, 1560 bp, Mus musculus
  source 1..1560: mol_type = unassigned DNA
gacacccggaattagtcttcggcggttctccggtcgagtttcccggtgctttagtctgcctctgccctagagccggcttcaggcctcgctgactttcgcctcccagaccctcgcgatggcgagagcctgggtctgcctggcgggcgccgccttcttcctttcctgtctggttttgcattcgcgcttctgtggctcgctggtttcaaggacattttcttttcacgtttcctggagaatggaggaccctcttttccggctggatttgggttggcccaagaattcggaatacttcactggtgcaacattttgtgttgcagttgattccctcaacgggttagtttacgtagcccaaagaggggataacatccccaaggtattagtgttctcagaggatggctatttcctacgagcctggaattatacagttgacacaccccatggtatgtttgtctccggcacaccatttgaacagtccgtctggatcacggacgtaggaagcggaccctacggccatactgttaaaaaatataattccctgggtgatcttgttcaagtcttgggaaccccaggcaagaagggcactggcttgaacccgctgcagtttgacaaccctgcagaattatatgtagatgacacaggggagatgtacattgtggatggagatgggggactgaataacagactggtcaaactatcccaagatttcatgatcctctggctgcgtggagagaatgggacaggacctgccaagttcaacatacctcacagcgttacacttgacgcagttggtcgggtgtgggttgcggaccgaggaaataagaggctccaagtatttgataaggacactggagagtggttaggagcatgggacaactgtttcacagaggaggggccttctgctgtgaggttcaccccagatgggaagtacttgattgtggcccagctgaacctcagtaggctgtcagtgttgcttgccccaccctccggaagcatcggagactgctctgtggtcagcactatccagctggcagatcaggttctgccacatcttttggaagtcgacagaaagactggcgcagtgtatgtagcagaaattggggcaaaacaaatacaaaaatacatcccgtggcacagccgcacccctgccttcggcgcttagtgcttccggctgggaggagcttcgggtagcacttcagagccacagggtcattttcacctcttctgcaagaaaagtgtgttgctgggtcatgaagagtcatgttttctcgtggttttgggacttggttttacaactaagtgtgtttctttctggtggccctgggagttgaactcatcacttcctaagtgctaggcggctgccctagcaggggctacatcttcacagaagcaccttttaaaggaagaaatgcagttctaaaatggggccatgacgggaactgtggttgtaaccaagctgcttgcctgcatcgattaactctctctgggttcctgggtgacttcatatgatgagtggaaactactagttggtttagcctatgtgtattagactgtggaagagat

SEQ ID NO: 183 - protein, 347 aa, Homo sapiens
MARFWVCVAGAGFFLAFLVLHSRFCGSPVLRNFTFAVSWRTEKILYRLDVGWPKHPEYFTGTTFCVAVDSLNGLVYIGQRGDNIPKILVFTEDGYFLRAWNYTVDTPHGIFAASTLYEQSVWITDVGSGFFGHTVKKYSSFGDLVQVLGTPGKKGTSLNPLQFDNPAELYVEDTGDIYIVDGDGGLNNRLIKLSQDFMILWLHGENGTGPAKFNIPHSVTLDSAGRVWVADRGNKRIQVFDKDTGEWLGAWNNCFTEEGPSSVRFTPDGKYLIVAQLNLSRLSVVAAPPVGSIGECSVISTIQLADQVLPHLLEVDRKTGAVYVAEIGAKQVQKYVPLNSYVPSFGS

SEQ ID NO: 184 - protein, 347 aa, Mus musculus
MARAWVCLAGAAFFLSCLVLHSRFCGSLVSRTFSFHVSWRMEDPLFRLDLGWPKNSEYFTGATFCVAVDSLNGLVYVAQRGDNIPKVLVFSEDGYFLRAWNYTVDTPHGMFVSGTPFEQSVWITDVGSGPYGHTVKKYNSLGDLVQVLGTPGKKGTGLNPLQFDNPAELYVDDTGEMYIVDGDGGLNNRLVKLSQDFMILWLRGENGTGPAKFNIPHSVTLDAVGRVWVADRGNKRLQVFDKDTGEWLGAWDNCFTEEGPSAVRFTPDGKYLIVAQLNLSRLSVLLAPPSGSIGDCSVVSTIQLADQVLPHLLEVDRKTGAVYVAEIGAKQIQKYIPWHSRTPAFGA

SEQ ID NO: 185 - DNA, 1172 bp, Homo sapiens
  source 1..1172: mol_type = unassigned DNA
gccccagccgttactggtccgcgcagtcagggcatcctccgcatcctccacatccttccatggctctgaagaataaattcagttgtttatggatcttgggtctgtgtttggtagccactacatcttccaaaatcccatccatcactgacccacactttatagacaactgcatagaagcccacaacgaatggcgtggcaaagtcaaccctcccgcggccgacatgaaatacatgatttgggataaaggtttagcaaagatggctaaagcatgggcaaaccagtgcaaatttgaacataatgactgtttggataaatcatataaatgctatgcagcttttgaatatgttggagaaaatatctggttaggtggaataaagtcattcacaccaagacatgccattacggcttggtataatgaaacccaattttatgattttgatagtctatcatgctccagagtctgtggccattatacacagttagtttgggccaattcattttatgtcggttgtgcagttgcaatgtgtcctaaccttgggggagcttcaactgcaatatttgtatgcaactacggacctgcaggaaattttgcaaatatgcctccttacgtaagaggagaatcttgctctctctgctcaaaagaagagaaatgtgtaaagaacctctgcaggactccacaacttattatacctaaccaaaatccatttctgaagccaacggggagagcacctcagcagacagcctttaatccattcagcttaggttttcttcttctgagaatcttttaatgtcatttatatacaaaagaaattctcaaatgttaaaataaaggaatagtttattgcttaatataacttatcatcactttgcttctttactgaatcttctacactcttgcctgatacctaaatttaatgtttgtttttaactcaaaaaatgtactgtagtatggaaaatggatagcagtagaataaagtcttaagattattttttaattacaaatccatatgtgtatcaaaagtgttccactctttaattttctaatgagaatggattatcattttagttatagtacatttagagaactaaagacttttcacatatcaaatagtttccttagataccaatagtttcctttatgaataccatcaacatcattcagaaacaaaatg

SEQ ID NO: 186 - DNA, 899 bp, Mus musculus
  source 1..899: mol_type = unassigned DNA
agtctccatggcaacactggctgccttgaacacattccccaggctaatgggaagacagagccagtttaaagtgtaatctctgttcttgcacattcactaggggtatccacgacatccttccatggccctgaagaagaaacttaattttttatggacattagttctgtatttgatagccagcagactgcccaaggcattcggcaaagatcttcctagggtgccaactatcacggatccgaaatttatagatgcattcctcaacatccacaatgaactgcgacgcaaggtccagcccccggcagctgatatgaatcaactgttttgggaccagcagctggcaaaactagcgaaagcatggactagagagtgcaaacttgcccataatccctgtatcaaacaacgatacgaatgtcttgaggactatgactttattggagaaaatatttatttgggtagaattgaaacccaaccagaagatgttgttattaattggtataatgaaagtaaatattttaattttgattttaatacctgttctgaaatgtgtggccattatacacaggtagtttgggccaaaacggttaaaattggttgtgcagtttcaaattgtcctaatctcaagggcttttcagctgggctatttgtatgtaattattcgccagcaggaaactttataggcttcagaccttacacgagaggagactcttgctctatgtgtggacaaaagacgtgtgagaatagtctctgccgtcccatgaatcggaagacacctcaccacaaagcggcatgccatgtacttgtgcttggttttattcttcagagcctactttaactgttatttctattcagaagaaaaattttgaatgctaaaaataaaagacttatttttctgactaaaa

SEQ ID NO: 187 - protein, 242 aa, Homo sapiens
MALKNKFSCLWILGLCLVATTSSKIPSITDPHFIDNCIEAHNEWRGKVNPPAADMKYMIWDKGLAKMAKAWANQCKFEHNDCLDKSYKCYAAFEYVGENIWLGGIKSFTPRHAITAWYNETQFYDFDSLSCSRVCGHYTQLVWANSFYVGCAVAMCPNLGGASTAIFVCNYGPAGNFANMPPYVRGESCSLCSKEEKCVKNLCRTPQLIIPNQNPFLKPTGRAPQQTAFNPFSLGFLLLRIF

SEQ ID NO: 188 - protein, 236 aa, Mus musculus
MALKKKLNFLWTLVLYLIASRLPKAFGKDLPRVPTITDPKFIDAFLNIHNELRRKVQPPAADMNQLFWDQQLAKLAKAWTRECKLAHNPCIKQRYECLEDYDFIGENIYLGRIETQPEDVVINWYNESKYFNFDFNTCSEMCGHYTQVVWAKTVKIGCAVSNCPNLKGFSAGLFVCNYSPAGNFIGFRPYTRGDSCSMCGQKTCENSLCRPMNRKTPHHKAACHVLVLGFILQSLL

SEQ ID NO: 189 - DNA, 1166 bp, Homo sapiens
  source 1..1166: mol_type = unassigned DNA
tagaaggaacagggagcggccaggatgctgaatctgctgctgctggcgctgcccgtcctggcgagccgcgcctacgcggcccctgccccaggccaggccctgcagcgagtgggcatcgtcgggggtcaggaggcccccaggagcaagtggccctggcaggtgagcctgagagtccacggcccatactggatgcacttctgcgggggctccctcatccacccccagtgggtgctgaccgcagcgcactgcgtgggaccggacgtcaaggatctggccgccctcagggtgcaactgcgggagcagcacctctactaccaggaccagctgctgccggtcagcaggatcatcgtgcacccacagttctacaccgcccagatcggagcggacatcgccctgctggagctggaggagccggtgaacgtctccagccacgtccacacggtcaccctgccccctgcctcagagaccttccccccggggatgccgtgctgggtcactggctggggcgatgtggacaatgatgagcgcctcccaccgccatttcctctgaagcaggtgaaggtccccataatggaaaaccacatttgtgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccctatcctgagcccccttccctgtcctaagcctgacgcctgcaccgggccctccagccctcccctgcccagatagctggtggtgggcgctaatcctcctgagtgctggacctcattaaagtgcatggaaatca

SEQ ID NO: 190 - DNA, 1191 bp, Mus musculus
  source 1..1191: mol_type = unassigned DNA
agagaagattgaagactgctaaagtgatctctcctggaccctgaagcagagtggccaagccattagagacctcgggctgttggaatgaacctaccttcctgctcccaggttcctggcttgtgcgccccacaacctgttgggcctagactagccctcacctccaactgggcccgcactactcctcactgtgtccaaatgctaaagctgctgctgctcacgctgcccctcctgtccagcctggtgcatgcagcccccggtccagctatgacacgagaaggcattgtggggggacaggaggcacatgggaacaagtggccctggcaggtgagcctgcgtgccaatgacacctactggatgcatttctgcggtggctccctcatccacccacagtgggtgctcactgcggcacactgtgtgggaccggatgttgctgaccccaacaaggtcagagtacagctccgtaagcagtacctctattaccatgaccacctgatgactgtgagccagatcatcacacaccccgacttctacatcgtccaggatggggcagacattgccctgctgaaactcacaaaccctgtgaacatttctgactatgtccaccctgtccccctacctcctgcctcagagaccttcccctcaggaacgttgtgctgggtgacaggctggggtaacatcgacaatggtgtaaacctgccgccaccatttcctttgaaggaggtgcaagttcccattatagaaaaccacctttgtgacttgaagtatcacaaaggtctcatcacaggtgacaatgtccacattgtccgagatgacatgctgtgtgctgggaatgaaggacatgactcctgccagggcgactccggaggacctctggtctgcaaggtagaagacacctggctgcaggcaggcgtggtcagctggggtgagggctgtgcacagcccaacaggcctggcatctacacccgggtcacctattacttggactggatccaccactatgtccccaaggacttctgagtcacatccaggatgacctccgttcctcccagcatgctgcttcctgcccgggtggcatccctgccttcctctcctgctccccatcctgagtcccaattcttctgccttccactcaagtagctacactgagcaggcgccgctctctgctatgcctcaataaaatgcgttaaagca

SEQ ID NO: 191 - protein, 275 aa, Homo sapiens
MLNLLLLALPVLASRAYAAPAPGQALQRVGIVGGQEAPRSKWPWQVSLRVHGPYWMHFCGGSLIHPQWVLTAAHCVGPDVKDLAALRVQLREQHLYYQDQLLPVSRIIVHPQFYTAQIGADIALLELEEPVNVSSHVHTVTLPPASETFPPGMPCWVTGWGDVDNDERLPPPFPLKQVKVPIMENHICDAKYHLGAYTGDDVRIVRDDMLCAGNTRRDSCQGDSGGPLVCKVNGTWLQAGVVSWGEGCAQPNRPGIYTRVTYYLDWIHHYVPKKP

SEQ ID NO: 192 - protein, 273 aa, Mus musculus
MLKLLLLTLPLLSSLVHAAPGPAMTREGIVGGQEAHGNKWPWQVSLRANDTYWMHFCGGSLIHPQWVLTAAHCVGPDVADPNKVRVQLRKQYLYYHDHLMTVSQIITHPDFYIVQDGADIALLKLTNPVNISDYVHPVPLPPASETFPSGTLCWVTGWGNIDNGVNLPPPFPLKEVQVPIIENHLCDLKYHKGLITGDNVHIVRDDMLCAGNEGHDSCQGDSGGPLVCKVEDTWLQAGVVSWGEGCAQPNRPGIYTRVTYYLDWIHHYVPKDF

SEQ ID NO: 193 - DNA, 1444 bp, Homo sapiens
  source 1..1444: mol_type = unassigned DNA
atttcgctttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaattaaagtga

SEQ ID NO: 194 - DNA, 1306 bp, Mus musculus
  source 1..1306: mol_type = unassigned DNA
tgccacctactcccttggatctgagctggacccttgcatctggcgtctacactgctgctgaaatcttctcaccgtgcacatccaaggatatctctatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgaaaggctcaaggccctctgccacagcgccctcctcacacagataggaaacaaagaaagattcataagagtcaggtggtcttggcctggtgggccttaagctccttcaggaatctgttctcccatcacatctcatctccccaaaggtggcacagctacctcagcatggtcccctccatcgcttctctcatattcactatacaagttgtttgtaagttttcatcaaaatattgttaaggggcgaagacgtcctcccctcaatgtgttagcagaagagcaagaactgataagctattgtttttgtgccaaagtgtttatgaaaacactcagtcaccccttatttaaaaatatttattgctatattttatactcatgaaagtacatgagcctatttatatttatttattttctatttattataatatttcttatcagatgaatttgaaacattttgaaacataccttattttgtggttctaataaagtaatgttatcacttcaca

SEQ ID NO: 195 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 196 - protein, 236 aa, Mus musculus
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 197 - DNA, 1762 bp, Homo sapiens
  source 1..1762: mol_type = unassigned DNA
acgaccagcgtgctgcgtcacggcggtgcgccggaagtggctgcggatttcgccggaaatcccggaagtgacagctttgggggtttgctgctggctctgactcccgtcctgcgatgggttgcgacgggggaacaatccccaagaggcatgaactggtgaaggggccgaagaaggttgagaagatttggcatttgaagaatgggaaagagtggtgcagtctacacagtgaaggaaaaaacaagaacgggaaggagtgggacatggaatatgatgtcgacaaagatgctgaattagtggcccaatggaactattgtactctaagtcaggaaatattaagacgaccaatagttgcctgtgaacttggcagactttataacaaagatgccgtcattgaatttctcttggacaaatctgcagaaaaggctcttgggaaggcagcatctcacattaaaagcattaagaatgtgacagagctgaagctttctgataatcctgcctgggaaggggataaaggaaacactaaaggtgacaagcacgatgacctccagcgggcgcgtttcatctgccccgttgtgggcctggagatgaacggccgacacaggttctgcttccttcggtgctgcggctgtgtgttttctgagcgagccttgaaagagataaaagcggaagtttgccacacgtgtggggctgccttccaggaggatgatgtcatcatgctcaatggcaccaaggaggatgtggacgtgctgaagacaaggatggaggagagaaggctgagagcgaagctggaaaagaaaacaaagaaacccaaggcagcagagtctgtttcaaaaccagatgtcagtgaagaagccccagggccatcaaaagttaagacagggaagcctgaagaagccagccttgattctagagagaagaaaaccaacttggctcccaaaagcacagcaatgaatgagagctcttctggaaaagctgggaagcctccgtgtggagccacaaagaggtccatcgctgacagtgaagaatcggaggcctacaagtccctctttaccactcacagctccgccaagcgctccaaggaggagtctgcccactgggtcacccacacgtcctactgcttctgaagcccgcactgccaccgctcctgccccagaaggttgtttagtttccacgtaggcaggtcgctttgtgcctctgagtgcgctgctgtgtgttctctctatagttctgtgtcataaagctgtcctggccagccttcaagctggtgtggccactcttgatgtgaggcgtgtcggttccaggggggacatgggaggggctgcacagtggcccgaggtcatgcttgcttccacctgcaggtgcatttggtcctttccatggccaggaagccctgtgggctgcactttttatgcttgcagtaacaagagactccagagtcctcaccggtgcagagttggcacatattaattaactaaaattctaatgatcttgctaccagcaataaatcaagtaggccaagtgaaactgggctttaaaaaggatggatttcaaatacactgtgcccactagaagcttcgaagggcctcgtccctctgctacagccctgggaggagccaggatccttgttggtctagctaaatactgttaggggagtgtgccccatctcatcatttcgaagatagcagagtcatagttgggcacccagtgattgggttcaaaaataaagctggtctgcctcttctctcaaaaaaaaaaaaaaaaa

SEQ ID NO: 198 - DNA, 2142 bp, Mus musculus
  source 1..2142: mol_type = unassigned DNA
aggtagggtttcgccagagaccccggaagcgacggcttctcgtctttgctaaggcttctggtttttgctacggtttctgatttcggctttgggatgggttgcgacggaggcaccatccccaagaggcacgagctggtgaaggggccgaagaaggtcgagaaggttgacaaagacgctgaactggtagcccaatggaactactgtaccctaagtcaggaaatcctgaggcgcccaatagttgcctgtgagcttggcagactgtataacaaagacgcggtcatcgagtttctgttggacaaatctgctgagaaggccctggggaaggcggcatcgcacatcagaagcatcaagaatgtgacagagctcaggctttctgacaaccccgcctgggaaggagataaagggaacaccaagggcgacaagcatgatgacctccagagagcgcggttcatctgccctgtggtgggcctggagatgaatggccggcacaggttttgcttcctccgatgctgcggttgtgtgttttctgaacgcgccttgaaggagataaaagctgaagtgtgtcacacgtgtggggctgccttccaggaggaggacatcattgtgctcaatggcaccaaggaggatgtggagatgctgaagaagaggatggaggagaggaggctgagggccaagctggaaaagaaaacaaagaaaccgaagacagcgaccgagtgtgcgtcaaagccgggtaccacacaagattccgcagggccatcaaaagtgaagtctggcaagcctgaggaggcggaccctgatcctagagagaagaaaagcaccccggctcccaggggcgcagcaactaatgggagtgcgtctgggaaagttggcaagcccccatgtggggccctgaagaggtccattgcagacagcgaggagtctgaaacctacaagtctatcttcaccagccacagctctgccaagcgctccaaggaggagtctgcgcactgggtcacccacacatcctactgcttctgaagcgggaccgggccttcaccctgggtttcccgtggccttctgtgccacacttgcttggtgtggcactgtgctataaagctgtcctctgcagtcctcagccagcgtggtcacttgctgtgagatcttgagggccctgggagtgagaagtggtgtgtgacctccctagtagcaggtccacaggccgtacctctcacctagcctggtgactgctgcttctattaggtaacaaaagactccagctctcggtgtacactcagctcataatggtcaaggagggagtggccccgagcttagggacagacttccaacagtcttagaccttgctcctcacagggttcttcttgcctgatcaaggactgcagagaggacgcatgcctgactgcctggtgcacactaacatcctttctctgtcttccttctctctccctccgctatcttggtaagagaatggcagccatccaggtgccggtggcaggcaggaatgactccttttccatccccacagttaaaattgggacctctgtgacacttcatgtggtgtgatagatctgcctctgggctgggcagatggctcactcagtggataaagcactcgccatggacacttgtggaccagagtttagatccccagaacctttatcaaagcccggtgagtgcagtagctgcctgtgatcccagcccctgtaaggcagggttccctcagcaaggcactgagttcagggttcgaggagaacctgcctcagacactagctggagtgcagttgagagattcccagtgtcagcctctggcctccacacttaaagtccctgttcccaaaccaaacatggtggcatgtctaatccaagcagtcagaagactgaggcaggaggatcgccgagatgtcagagctggcaaggttaaatagcaaaccctcacctcaggaaacaaccccagaaaagcccctgttcctgctgccctctggtttgctgcttgcaggcgatgaggcttggcctcaaatacacttgcatttattctgggacagtgctgggagcatggtaggagtgtgcagcagggtgggagtgagcatggtccaagggaccctgggcgcatgacctttgtacttagaaaattgaaataaaggttgt

SEQ ID NO: 199 - protein, 336 aa, Homo sapiens
MGCDGGTIPKRHELVKGPKKVEKIWHLKNGKEWCSLHSEGKNKNGKEWDMEYDVDKDAELVAQWNYCTLSQEILRRPIVACELGRLYNKDAVIEFLLDKSAEKALGKAASHIKSIKNVTELKLSDNPAWEGDKGNTKGDKHDDLQRARFICPVVGLEMNGRHRFCFLRCCGCVFSERALKEIKAEVCHTCGAAFQEDDVIMLNGTKEDVDVLKTRMEERRLRAKLEKKTKKPKAAESVSKPDVSEEAPGPSKVKTGKPEEASLDSREKKTNLAPKSTAMNESSSGKAGKPPCGATKRSIADSEESEAYKSLFTTHSSAKRSKEESAHWVTHTSYCF

SEQ ID NO: 200 - protein, 307 aa, Mus musculus
MGCDGGTIPKRHELVKGPKKVEKVDKDAELVAQWNYCTLSQEILRRPIVACELGRLYNKDAVIEFLLDKSAEKALGKAASHIRSIKNVTELRLSDNPAWEGDKGNTKGDKHDDLQRARFICPVVGLEMNGRHRFCFLRCCGCVFSERALKEIKAEVCHTCGAAFQEEDIIVLNGTKEDVEMLKKRMEERRLRAKLEKKTKKPKTATECASKPGTTQDSAGPSKVKSGKPEEADPDPREKKSTPAPRGAATNGSASGKVGKPPCGALKRSIADSEESETYKSIFTSHSSAKRSKEESAHWVTHTSYCF

SEQ ID NO: 201 - DNA, 2406 bp, Homo sapiens
  source 1..2406: mol_type = unassigned DNA
agttcccagcgccgggcggagcgccggacagagccccgcagcgccccgcggccgcgatggggccgaagcgcccgaagccccggagcccacaaactgccgggcccgcctcgccgccgggacccgggtgcctgggctcggcttgaagcggcggcggcgcaccggcacagccgcgggagcatgggcaggaggatgcggggcgccgccgccaccgcggggctctggctgctggcgctgggctcgctgctggcgctgtggggagggctcctgccgccgcggaccgagctgcccgcctcccggccgcccgaagaccgactcccacggcgcccggcccggagcggcggccccgcgcccgcgcctcgcttccctctgcccccgcccctggcgtgggacgcccgcggcggctccctgaaaactttccgggcgctgctcaccctggcggccggcgcggacggcccgccccggcagtcccggagcgagcccaggtggcacgtgtcagccaggcagccccggccggaggagagcgccgcggtgcacgggggcgtcttctggagccgcggcctggaggagcaggtgcccccgggcttttcggaggcccaggcggcggcgtggctggaggcggctcgcggcgcccggatggtggccctggagcgcgggggttgcgggcgcagctccaaccgactggcccgttttgccgacggcacccgcgcctgcgtgcgctacggcatcaacccggagcagattcagggcgaggccctgtcttactatctggcgcgcctgctgggcctccagcgccacgtgccgccgctggcactggctcgggtggaggctcggggcgcgcagtgggcgcaggtgcaggaggagctgcgcgctgcgcactggaccgagggcagcgtggtgagcctgacacgctggctgcccaacctcacggacgtggtggtgcccgcgccctggcgctcggaggacggccgtctgcgccccctccgggatgccgggggtgagctggccaacctcagccaggcggagctggtggacctagtacaatggaccgacttaatccttttcgactacctgacggccaacttcgaccggctcgtaagcaacctcttcagcctgcagtgggacccgcgcgtcatgcagcgtgccaccagcaacctgcaccgcggtccgggcggggcgctggtctttctggacaatgaggcgggcttggtgcacggctaccgggtagcaggcatgtgggacaagtataacgagccgctgttgcagtcagtgtgcgtgttccgcgagcggaccgcgcggcgcgtcctggagctgcaccgcggacaggacgccgcggcccggctgctgcgcctctaccggcgccacgagcctcgcttccccgagctggccgcccttgcagacccccacgctcagctgctacagcgccgcctcgacttcctcgccaagcacattttgcactgtaaggccaagtacggccgccggtctgggacttagtgtcaccgggaggaaaagagagagatctggggctggggtatggatgatggggggaagggcggtcgcctctgccactgtcagggaccagccggccaacgcccacccgcaaaggtgtctaaaaacttcagcttttcacccacctgcccctttctttcaatcccacgctgtttcctttcaaagttctgggaggacgaactcaccgaggcgagaagtgtaacattctctccacccagcttataaaaggattctttactgtgccagcacggggattggatccgaagaaactggctactggggtttggcccccgagtggccgtccctgtgggagatgcaccccattcttgggccccccctcattccctttccgaaaaaggaaaacttgcgtttgagccgttgagctaattctgcaattttctaccaaacagagcgctggtggccccggagcagggctgtgacattggctggtggagcccccttcctgtgttctccctttgttccagcgccgcgatggtgagatcactgttccaagcagggggacggctcgcgataggacaaagagagcaggacctccagactctggggagccctgcagaccttgacaatttgcctgactcattcctgacctcttgtcattttggcctgaaggctacaaattcagggtcagctgtatgcactaagtcaaataatgaatttcttcctccctctcgcaaccgaccaaaattttgacaacgatgatgttcaccagaaggaaaaaaaaaaatcagttttatgcactttattttgttttgattttcattttttattaagaaaaaattttattttacagaatttaccttctctgtatatatgtgcataaagtgtggtgtaaatatactaaacaaacttatatttcaataaaagggagtttaaaattta

SEQ ID NO: 202 - DNA, 2446 bp, Mus musculus
  source 1..2446: mol_type = unassigned DNA
gctgctgggaagccacggggaggcgagttcccagcgccggggcagagcgctggccagagccccgcagcgctccgcggccgcgatggggctccggcacccgaagccccggagccagctagcagccgggcccgcctcgccgcccgcacccgggcgcccgggcttggcttgaagcggcggcggtggcaccggcgcgccggcaggagcatggggaggaagatgcggggcgccgccgccgccgcggggctctggctgctggctttgagctcgctgctgacgctgtggggaggactcctgccaccgcggaccgagctgccagcctcccggccgcccgaagatcgactccctccgcatccgatccagagtggcggccccgcgcccgagccgcgattccctctgcccccgcccctagtatgggacgcccgcggcggctccctgaaaactttccgggcgctgctcaccctggcggccggcgcggataacccgcctaggaggcaccaggacgaccgcgggcggcacgagccctccgggctgtcctggccagaggagcgcagggcggtgcacgggggcgtcttctggagccgcggcctggaggagcaggttccccggggcttttccgaagcccaagcagcagcgtggctggaggtggcacggggtgctcgggtggtggctctggatcgcgggggctgcggacgcagttccaaccgcctagcccgctttgccgacggcacccgtgcctgtgtacgctacggcatcaacccagagcagatacagggcgaggccctgtcctactaccttgcgcgcctgctgggcctccagcgccacgtgccgccgctggcactggctcgggtggaggctcggggcgcgcagtgggtgcaggtgcaggaggagctgcgcaccgcgcactggaccgagggcagcgtggtgagcctgacgcgctggctgcctaacctcaccgacgtggtggtgcccgagccctggcgatcagaggacggccgtctgcggcccctgcgcgacgccgggggcgagctgaccaacctcagccaggcggagctggtggacttggtacaatggaccgatctgatcctcttcgattacctgacggccaactttgaccggcttgtgagcaacctcttcagcttacagtgggacccacgcgttatgcaccgcgctacgagcaacctgcaccgaggaccaggaggggcgttggtctttctggacaatgaggcgggcttggtgcacggctaccgggtagccggcatgtgggacaagtataacgaaccgctgctacagtcggtgtgtgtattccgagagcggactgctaggcgcgtcttggagctgcaccggggtcaggacgcggcggcccggttgctgcgcctctacagtcgccacgaaccgcgtttcccagagctggcggagctctcagaaccccacgctcagctgctacagcgccgccttgacttcctcgccaaacacattttgcactgcaaggccaagtacggccgccggcccggggacttaataacactccgaggaagagagggactggggtatgaatgatgggggttagggctgtcttctctgccacgggcaacgaccaaccagccaacgcccacccaatggtctgatcgccaagctgtctaaaaaaaaaaattgtgtgttacccacttgcccctttcttttcatgccaggctgtttcctttccaagttctggaagggcaaactcaccgaggcgagaagtgtaacattccctccacctagcttataaaaagagttctgtgcttgtgtggagattggatccgaagaaactggctgctggggtttggcccgggaatggcagtctctttgggagatgcagtccatttttgtcgccccctccaccccttcccaaaaaggaaaacttccgtttgagctaattcctctatttcctactaaacacagcgctggtggccccagagcaggctgtgacaagggctggtgaaacccccttcctgtgttcgccctttgttccggcgcgcgatggtgagataactgttccaagctgagggacagctctggataggacaaagagagcaagacttccccgcccctgggagccctccagaccctgacagtttgtctcacccattcctgaactcctatcattttggcctgaaggctatgaattcaggactagttataagcagagtcaataatgaatttcttcctgcgtctcccaattgaccaaaattgacaatgatgatgttcaccagagggggaaaaaaatcagtttcatgcactttactttttttaattttaattttttagtaagaaaacttttttatttgacagaatttgccttctgtgtgtatatatgtgcataaattgtgctgtaaatagactaaacaaacttatatttcaataaaagggagtttaaaatttagaacttaaaaaaaaaaaaaaaaaaaaa

SEQ ID NO: 203 - protein, 437 aa, Homo sapiens
MGRRMRGAAATAGLWLLALGSLLALWGGLLPPRTELPASRPPEDRLPRRPARSGGPAPAPRFPLPPPLAWDARGGSLKTFRALLTLAAGADGPPRQSRSEPRWHVSARQPRPEESAAVHGGVFWSRGLEEQVPPGFSEAQAAAWLEAARGARMVALERGGCGRSSNRLARFADGTRACVRYGINPEQIQGEALSYYLARLLGLQRHVPPLALARVEARGAQWAQVQEELRAAHWTEGSVVSLTRWLPNLTDVVVPAPWRSEDGRLRPLRDAGGELANLSQAELVDLVQWTDLILFDYLTANFDRLVSNLFSLQWDPRVMQRATSNLHRGPGGALVFLDNEAGLVHGYRVAGMWDKYNEPLLQSVCVFRERTARRVLELHRGQDAAARLLRLYRRHEPRFPELAALADPHAQLLQRRLDFLAKHILHCKAKYGRRSGT

SEQ ID NO: 204 - protein, 450 aa, Mus musculus
MGRKMRGAAAAAGLWLLALSSLLTLWGGLLPPRTELPASRPPEDRLPPHPIQSGGPAPEPRFPLPPPLVWDARGGSLKTFRALLTLAAGADNPPRRHQDDRGRHEPSGLSWPEERRAVHGGVFWSRGLEEQVPRGFSEAQAAAWLEVARGARVVALDRGGCGRSSNRLARFADGTRACVRYGINPEQIQGEALSYYLARLLGLQRHVPPLALARVEARGAQWVQVQEELRTAHWTEGSVVSLTRWLPNLTDVVVPEPWRSEDGRLRPLRDAGGELTNLSQAELVDLVQWTDLILFDYLTANFDRLVSNLFSLQWDPRVMHRATSNLHRGPGGALVFLDNEAGLVHGYRVAGMWDKYNEPLLQSVCVFRERTARRVLELHRGQDAAARLLRLYSRHEPRFPELAELSEPHAQLLQRRLDFLAKHILHCKAKYGRRPGDLITLRGREGLGYE

SEQ ID NO: 205 - DNA, 5223 bp, Homo sapiens
  source 1..5223: mol_type = unassigned DNA
gccattctcccgcctcagcctcccgagtagctgggactacaggcgccgccaccacgcctggctaatttttgtatttttagtagagacggggtttcaccgtgttagccaggatggtctcgatctcctgacctcgtgatccgcccgcctcggcctcccaaagtgctgggatgacaggcgtgagccaccgtgcccggcctggctctgtttttattagaaaaattctcaatcgtcgtcttttaagaatcattgacatagagtaactccacagcatgtgtcttcaagagcttccctaaaagattaaaggttatacaaaacttaaaagaagcagcaattctattcgcttgttattggacttgaaactccctttgacctcggaaactgaagatgaggttgccatgggaactgctggtactgcaatcattcattttgtgccttgcagatgattccacactgcatggcccgatttttattcaagaaccaagtcctgtaatgttccctttggattctgaggagaaaaaagtgaagctcaattgtgaagttaaaggaaatccaaaacctcatatcaggtggaagttaaatggaacagatgttgacactggtatggatttccgctacagtgttgttgaagggagcttgttgatcaataaccccaataaaacccaagatgctggaacgtaccagtgcacagcgacaaactcgtttggaacaattgttagcagagaagcaaagcttcagtttgcttatcttgacaactttaaaacaagaacaagaagcactgtgtctgtccgtcgaggtcaaggaatggtgctactgtgtggcccgccaccccattctggagagctgagttatgcctggatcttcaatgaatacccttcctatcaggataatcgccgctttgtttctcaagagactgggaatctgtatattgccaaagtagaaaaatcagatgttgggaattatacctgtgtggttaccaataccgtgacaaaccacaaggtcctggggccacctacaccactaatattgagaaatgatggagtgatgggtgaatatgagcccaaaatagaagtgcagttcccagaaacagttccgactgcaaaaggagcaacggtgaagctggaatgctttgctttaggaaatccagtaccaactattatctggcgaagagctgatggaaagccaatagcaaggaaagccagaagacacaagtcaaatggaattcttgagatccctaattttcagcaggaggatgctggtttatatgaatgtgtagctgaaaattccagagggaaaaatgtagcaaggggacagctaactttctatgctcaacctaattggattcaaaaaataaatgatattcacgtggccatggaagaaaatgtcttttgggaatgtaaagcaaatggaaggcctaagcctacatacaagtggctaaaaaatggcgaacctctgctaactcgggatagaattcaaattgagcaaggaacactcaacataacaatagtgaacctctcagatgctggcatgtatcagtgtttggcagagaataaacatggagttatcttttccaacgcagagcttagtgttatagctgtaggtccagatttttcaagaacactcttgaaaagagtaactcttgtcaaagtgggaggtgaagttgtcattgagtgtaagccaaaagcgtctccaaaacctgtttacacctggaagaaaggaagggatatattaaaagaaaatgaaagaattaccatttctgaagatggaaacctcagaatcatcaacgttactaaatcagacgctgggagttatacctgtatagccactaaccattttggaactgctagcagtactggaaacttggtagtgaaagatccaacaagggtaatggtacccccttccagtatggatgtcactgttggagagagtattgttttaccgtgccaggtaacgcatgatcactcgctagacatcgtgtttacttggtcatttaatggacacctgatagactttgacagagatggggaccactttgaaagagttggagggcaggattcagctggtgatttgatgatccgaaacatccaactgaagcatgctgggaaatatgtctgcatggtccaaacaagtgtggacaggctatctgctgctgcagacctgattgtaagaggtcctccaggtcccccagaggctgtgacaatagacgaaatcacagataccactgctcagctctcctggagacccgggcctgacaaccacagccccatcaccatgtatgtcattcaagccaggactccattctccgtgggctggcaagcagtcagtacagtcccagaactcattgatgggaagacattcacagcgaccgtggtgggtttgaacccttgggttgaatatgaattccgcacagttgcagccaacgtgattgggattggggagcccagccgcccctcagagaaacggagaacagaagaagctctccccgaagtcacaccagcgaatgtcagtggtggcggaggcagcaaatctgaactggttataacctgggagacggtccctgaggaattacagaatggtcgaggctttggttatgtggtggccttccggccctacggtaaaatgatctggatgctgacagtgctggcctcagctgatgcctctagatacgtgttcaggaatgagagcgtgcaccccttctctccctttgaggttaaagtaggtgtcttcaacaacaaaggagaaggccctttcagtcccaccacggtggtgtattctgcagaagaagaacccaccaaaccaccagccagtatctttgccagaagtctttctgccacagatattgaagttttctgggcctccccactggagaagaatagaggacgaatacaaggttatgaggttaaatattggagacatgaagacaaagaagaaaatgctagaaaaatacgaacagttggaaatcagacatcaacaaaaatcacgaacttaaaaggcagtgtgctgtatcacttagctgtcaaggcatataattctgctgggacaggcccctctagtgcaacagtcaatgtgacaacccgaaagccaccaccaagtcaaccccccggaaacatcatatggaattcatcagactccaaaattatcctgaattgggatcaagtgaaggccctggataatgagtcggaagtaaaaggatacaaagtcttgtacagatggaacagacaaagcagcacatctgtcattgaaacaaataaaacatcggtggagctttctttgcctttcgatgaagattatataatagaaattaagccattcagcgacggaggagatggcagcagcagtgaacaaattcgaattccaaagatatcaaatgcctacgcgagaggatctggggcttccacttcgaatgcatgtacgctgtcagccatcagtacaataatgatttccctcacagctaggtccagtttatgacaaaagttatctgaaggacttgctgtttataatataagcaacatttagctagttgttttgaagacacccagtactaagtaatattgttgttcaagtacatcttattactggaataaaaatgttttttgcttctttaggaatggcattatacagtacttcctcaaagcaaatctagctttgtctgaagtttctttggaaactctgcaatgcactgaagacatctgtaatatgatgttaccaaagcagtttacatatgtccttatatgcatattttttattatatatttagtgttttatagaattttttaaagttaacatataatgtagatattaattttttcctcggctgtaaaatgctatgggcaacacaatcatgcaattataatttgaaaatatttcctttaaagacagagtttgaaactcatcttggtaaataaattgcaaattactcgtacagttttacaaggatctcatggcagaacagcggaagacttggtccgtaactcaaggctgttgtatgcaaactactcttctagtggttaatgcacttgacatgtacagtatgtttcccatgccgttgtgattttgacatgtacagtatgttttcatgccgttgtgaattttgttgttcaacccaacttgagatggtttcaggaatggctgcaatctcaaaagctcaatgtactgcccaagaggcaccttgtgcaatattcccatccctgaatttagcattgtacaggaagattttcttttcactcaactgagttagcattgtctttgtggtagcattatcttagacattaaatttgaagtaacatatatcctgtagtaacatagacccaaagttactatagtcaaccaagtctttcaaaggataaaagatcattttattatctttaactgtatctattttgaatgtaaatttaaaaaaagtaattctctgtcaatggattcgtaatttctttacaaaatttcttatatataatatgtgtatctctgtaataatagagcccttcttttgaatcaaaattacatatggactttggaagattgctcctatttcaacaaatagttgctgcaagaatttttaatatgactctataaaagctctttagtacaattgtatggtttcttgatgattctgttttgcaataggtagcctagttgctttatatgctttaccttctaggtcttaaaatcacacattggaaaatgacaatatcaacaaaactgtattcttatgaaaagaactatttgttacaatgagaaaggttttgtgagtaaaccctactggacagtgacataaaaggcaatggaattttctataaacattttctcatgtaaacatgctgccaccttttcttctttctcagagaactcaaatttgctatagtttgtcatttttgcttacaaataatataacttttcaaccttctagttatatttatccaataaagtcataatcgggattccatttgtgtaaaaactagggtggtaaccggaaaaaaatattgtcagtatttcaagctgtgttcctttcttagtttgatatggttacttctatgttgaaataaaatttaaaatcacatgcaaaaaaaaaatcagcaaaataataaaatgaacgaaaaaaaatgacacccagggagttcccaaccccagtgtaaatgatatttaccagtgatctagcatatgtaatcttttttaaaggtattgttaataaatattctgtcatttgtaaagaaaaaaaaaaaaaaaaaa

SEQ ID NO: 206 - DNA, 5262 bp, Mus musculus
  source 1..5262: mol_type = unassigned DNA
acagggagggagaggaacgcgcggcagccacagagcacaaggttcataaccaggattggtcgcaagtagggagcttttgccactccgccgggaccttcggacgttggcggtggagcaagcccagttctcctaggcgctgtgcttggattcatattgcattgttgacaattctcacaggtgaaattcggccagagctcacttggaaatttctgcagattggtgtaagagaatcagattcttcatgctcagctaaaggaaacaatcctcgtggttcttattgaaaagaagagggattattgacacatactaattcaccagcatgtggctctcagaactgccctaagaggttatcaagaaatccacatctttctgcatctgactgtggacgctctgctgttttgaagattaaaggttgcacaaagcttaaaaggaacagaagcagcagttctattcacttgttattggacttgaaactcctttgacctgggaaactgaagatgaggttgccatgggaactgctagtgctgcaatcattcatgttgtgccttgcagatgactacacactgcatggcccagtttttgttcaagaaccaagtcatgtcatgtttcctttggattctgaggagaaaaaagtgaaactcagttgtgaagttaaagggaatccaaaacctcacatcaggtggaagataaatggaacagatgttgacattggtatggatttccgctacagtgttgttgacggcagcttgttgatcaataaccccaataaaacccaagatgctggaacgtaccagtgcatagcaacaaactcatttggaacaattgtaagcagagaagcaaagcttcagtttgcttatcttgaaaactttaagacaagaacaagaagcactgtgtcagtccgtcgaggtcagggaatggtccttctgtgcggcccgccaccccattctggagagctgagctatgcctggatcttcaatgaatacccttcctatcaggataaccgccgcttcgtttctcaagagactgggaatctgtatattgccaaagtggaaaaatctgatgttggtaattacacctgtgtagttaccaacactgtgaccaaccacaaggtcctgggaccgcccacaccactaatcctgagaaatgatggggtgatgggtgaatatgagcccaaaatagaagtacagtttccagaaaccgtcccggctgagaaaggaacaacagtcaaactggaatgctttgccttaggaaatccagtcccaactattctatggcgaagagctgatggcaagccaatagcaaggaaagccagaagacataagtcgaatggaattctggaaattcccaattttcagcaggaagatgccggttcatatgaatgtgtggctgaaaactccagaggcaaaaacgtagccaagggacagttgactttttatgctcaacctaactgggttcaaataataaatgatatccacgtggccatggaagagagtgtcttctgggaatgtaaagcaaatgggaggcctaaacctacctacagatggctaaaaaatggtgacccactgttaacacgggatagaatacaaatcgagcaaggaacactcaacataacaatagtgaatctctcagatgctggcatgtaccagtgtgtggcagaaaataaacatggagttatcttttccagcgctgagcttagtgtaatagctgaaagtccagatttttcccgaacactcttaaaaagagtaacacttgtcaaagtgggaggtgaagttgtcattgagtgcaagccaaaagcatccccaagacctgtctacacctggagaaagggaagagaaatattaagagaaaatgaaagaattaccatttctgaagatgggaacctcagaatcatcaatgtgactaaatctgatgctggaagttacacctgcatagccacgaaccattttggaactgccagcagtactgggaatgtgattgtgaaagacccaacaaaggtgatggttcctccttccagcatggatgtcactgtaggagaaagcattgttcttccatgccaggtaacacatgatcactccctggacattgtgtttacgtggacatttaatggacacttgatagactttgacaaggatggagaccactttgaaagagttggagggcaggattcagctggtgatttgatgatccgaaacatccaactgaagcatgctgggaagtatgtctgcatggtccaaacaagtgtagacaaactctctgttgctgcggacctgattgtaagaggtcctccaggtcctcctgaggctgtgaccatagatgaaatcacagacaccactgctcagctttcttggagaccaggacctgacaaccacagccccatcaccatgtatgttatccaagctaggactccattctccgtgggctggcaagcagtcaatacagttccagatctcgtcgacgggaagacattcacagcaactgtggtgggtttgaatccttgggttgaatatgaattccgcactgttgcagctaatgtgattggcattggggaacccagccggccctcagagaaacggaggacagaagaggctctccctgaagtcaccccggctaacgtcagtggtggtggaggcagcaaatctgaactggttataacctgggagacagtccctgaggaattacagaatggcagaggctttggttacgtggttgccttccggccacatggcaaaatgatctggatgctgaccgtgctggcttcagctgatgcctccagatacgtgttcaggaatgagagtgtgcgtccattctctccctttgaggttaaagtgggtgtcttcaataacaaaggggaaggccctttcagtcccaccacgctggtgtattctgctgaagaagaacccaccaagccaccagccagtatctttgccaggagtctttctgccacggatatcgaagttttctgggcctcccctattggaaagaacagaggacggattcaaggttatgaggtaaaatattggagacatgatgacaaggaagaaaatgctaaaaagatacgaacagttggaaatcagacatccacaaaaatcaccaacttgaaaggaagtgctctgtatcacttatctgtcaaggcgtataactcagctgggacaggaccctccagtgcaacagtcaatgtcacgaccagaaagccaccaccaagccaaccccctggaaacatcatatggaattcatcggactccaaaattatcctgaactgggatcaagtgaaggccctggataatgagtcagaagtaaaaggatacaaagtcttgtacagatggaacaggcaaagcagcacatctgtcattgaaaccaacaagacatcagtggagctttcattacctttcgatgaagattacatcatagagatcaagccattcagtgatggaggagatggcagcagcagtgagcaaatccgaatcccaaagatatcaaattcctatgcaagaggatcgggggcttctacctccaatgcgtgtacgctgtcagccatcagtacaataatgatttctctcacagccaggtctagtttatgacaaaagttatctgaaggacttgctatttataatataagcaacatttagctagttgttttgaagacacccagtactaagtaatattgttgttcaagtacatcttattactggaaaaaaatgttttttgcttcgttaggaatggcattatacagtacttcctcaaagcaaatctagctttgtctgaagtttctttggaaactctgcaatgcactgaagacatctgtaatatgatgttaccaaagcagtttacatatgtccttatatgcatattttttattatatatttagtgttttatagaattttttaaagttaacatataatatagatattgtttttttctttcttcctctgctataaaatgctctgggcaacacaaaccacacaattatgatttgagccgatttcctttaagggcagagtttgaaactcatcacagcaaatgaattacaaatgacttgtacagttttacaaggatccagtgtcaaaacagctggagactgggtctgtaactcaaggctgctgtatataaattatcccttgaatggttaatgcattgaatcgagtccccttgacatatgtgatatgttttcccaccctgttgtgaattttcttgttgaacacaatttgagatggtttcaggaactacacatcccaaaagctaaacttcccagcccaagaggtaccttgtgcaattctcccatccctgaacttagcatcatctggtaagactttatttgagcatcgtctttgtagtagcattatcttaggcattgaatttgaagttacatatcctgtagtaacatagatgaaaagttactatagtcaaacaagtctttcatggcataaaaggtaattttactctttagacatatctgttttgaatgtaaattttttaataattctgtcaatgcatttataatttcaaaatttcttatagataacatgtgtaactgtaatgatagagcccttcttttgaatgacaataacatatgtaatttgaaatttttttcctattttagcaaatagctgctgcaaagaattttacatataactctataactatccgtagtacagatgtattgtatcttgatgtctctcccgttttgcagtagctagtctggttgctttataagctttgcattctacatcttaaaatgatacaatgaaagatggcaatattgacaaaccttattcttacgaaaagaactatttattacgatgggggatgttttgtaagctgacaagggtggaacataaaaggcaatgagattttctataaagcgttgcacatgaaaacacacagtcacctttttcttctttcttggagagctcacgattgcctgtaatttgtcatttttgcttacacataacatagcctttcaacttgctaattatattcatccaataaaggcatcattgattccatttgtgtaaaaattaggatggtaacagggaaaatattgtcagtatttcagctgtgttcctttcttactttgatatggcatacttctatgttaaaatatgatttaaaatcacatgcaaaaaaatcaacaaagtaataaaatgaatgaaaaaaactgataccccaggtagttcccaaccccagtgtaaatgatatctaccagtgatctagcatatgtaatcttttttaaaggtattgttaataaatattctgtcatttgt

SEQ ID NO: 207 - protein, 1026 aa, Homo sapiens
MRLPWELLVLQSFILCLADDSTLHGPIFIQEPSPVMFPLDSEEKKVKLNCEVKGNPKPHIRWKLNGTDVDTGMDFRYSVVEGSLLINNPNKTQDAGTYQCTATNSFGTIVSREAKLQFAYLDNFKTRTRSTVSVRRGQGMVLLCGPPPHSGELSYAWIFNEYPSYQDNRRFVSQETGNLYIAKVEKSDVGNYTCVVTNTVTNHKVLGPPTPLILRNDGVMGEYEPKIEVQFPETVPTAKGATVKLECFALGNPVPTIIWRRADGKPIARKARRHKSNGILEIPNFQQEDAGLYECVAENSRGKNVARGQLTFYAQPNWIQKINDIHVAMEENVFWECKANGRPKPTYKWLKNGEPLLTRDRIQIEQGTLNITIVNLSDAGMYQCLAENKHGVIFSNAELSVIAVGPDFSRTLLKRVTLVKVGGEVVIECKPKASPKPVYTWKKGRDILKENERITISEDGNLRIINVTKSDAGSYTCIATNHFGTASSTGNLVVKDPTRVMVPPSSMDVTVGESIVLPCQVTHDHSLDIVFTWSFNGHLIDFDRDGDHFERVGGQDSAGDLMIRNIQLKHAGKYVCMVQTSVDRLSAAADLIVRGPPGPPEAVTIDEITDTTAQLSWRPGPDNHSPITMYVIQARTPFSVGWQAVSTVPELIDGKTFTATVVGLNPWVEYEFRTVAANVIGIGEPSRPSEKRRTEEALPEVTPANVSGGGGSKSELVITWETVPEELQNGRGFGYVVAFRPYGKMIWMLTVLASADASRYVFRNESVHPFSPFEVKVGVFNNKGEGPFSPTTVVYSAEEEPTKPPASIFARSLSATDIEVFWASPLEKNRGRIQGYEVKYWRHEDKEENARKIRTVGNQTSTKITNLKGSVLYHLAVKAYNSAGTGPSSATVNVTTRKPPPSQPPGNIIWNSSDSKIILNWDQVKALDNESEVKGYKVLYRWNRQSSTSVIETNKTSVELSLPFDEDYIIEIKPFSDGGDGSSSEQIRIPKISNAYARGSGASTSNACTLSAISTIMISLTARSSL

SEQ ID NO: 208 - protein, 1026 aa, Mus musculus
MRLPWELLVLQSFMLCLADDYTLHGPVFVQEPSHVMFPLDSEEKKVKLSCEVKGNPKPHIRWKINGTDVDIGMDFRYSVVDGSLLINNPNKTQDAGTYQCIATNSFGTIVSREAKLQFAYLENFKTRTRSTVSVRRGQGMVLLCGPPPHSGELSYAWIFNEYPSYQDNRRFVSQETGNLYIAKVEKSDVGNYTCVVTNTVTNHKVLGPPTPLILRNDGVMGEYEPKIEVQFPETVPAEKGTTVKLECFALGNPVPTILWRRADGKPIARKARRHKSNGILEIPNFQQEDAGSYECVAENSRGKNVAKGQLTFYAQPNWVQIINDIHVAMEESVFWECKANGRPKPTYRWLKNGDPLLTRDRIQIEQGTLNITIVNLSDAGMYQCVAENKHGVIFSSAELSVIAESPDFSRTLLKRVTLVKVGGEVVIECKPKASPRPVYTWRKGREILRENERITISEDGNLRIINVTKSDAGSYTCIATNHFGTASSTGNVIVKDPTKVMVPPSSMDVTVGESIVLPCQVTHDHSLDIVFTWTFNGHLIDFDKDGDHFERVGGQDSAGDLMIRNIQLKHAGKYVCMVQTSVDKLSVAADLIVRGPPGPPEAVTIDEITDTTAQLSWRPGPDNHSPITMYVIQARTPFSVGWQAVNTVPDLVDGKTFTATVVGLNPWVEYEFRTVAANVIGIGEPSRPSEKRRTEEALPEVTPANVSGGGGSKSELVITWETVPEELQNGRGFGYVVAFRPHGKMIWMLTVLASADASRYVFRNESVRPFSPFEVKVGVFNNKGEGPFSPTTLVYSAEEEPTKPPASIFARSLSATDIEVFWASPIGKNRGRIQGYEVKYWRHDDKEENAKKIRTVGNQTSTKITNLKGSALYHLSVKAYNSAGTGPSSATVNVTTRKPPPSQPPGNIIWNSSDSKIILNWDQVKALDNESEVKGYKVLYRWNRQSSTSVIETNKTSVELSLPFDEDYIIEIKPFSDGGDGSSSEQIRIPKISNSYARGSGASTSNACTLSAISTIMISLTARSSL

SEQ ID NO: 209 - DNA, 1610 bp, Homo sapiens
  source 1..1610: mol_type = unassigned DNA
acaagccagccagtccctgcagctccacaaactgacccatcctgggccttgttctccacagaatgggtctgctccttcccctggcactctgcatcctagtcctgtgctgcggagcaatgtctccaccccagctggccctcaacccctcggctctgctctcccggggctgcaatgactcagatgtgctggcagttgcaggctttgccctgcgggatattaacaaagacagaaaggatggctatgtgctgagactcaaccgagtgaacgacgcccaggaatacagacgggtttatggtcaatgcaaagcaatattttatatgaacaacccaagtagagttctctatttagctgcttataactgtactcttcgcccagtttcaaaaaaaaagatttacatgacgtgccctgactgcccaagctccatacccactgactcttccaatcaccaagtgctggaggctgccaccgagtctcttgcgaaatacaacaatgagaacacatccaagcagtattctctcttcaaagtcaccagggcttctagccagtgggtggtcggcccttcttactttgtggaatacttaattaaagaatcaccatgtactaaatcccaggccagcagctgttcacttcagtcctccgactctgtgcctgttggtctttgcaaaggttctctgactcgaacacactgggaaaagtttgtctctgtgacttgtgacttctttgaatcacaggctccagccactggaagtgaaaactctgctgttaaccagaaacctacaaaccttcccaaggtggaagaatcccagcagaaaaacacccccccaacagactccccctccaaagctgggccaagaggatctgtccaatatcttcctgacttggatgataaaaattcccaggaaaagggccctcaggaggcctttcctgtgcatctggacctaaccacgaatccccagggagaaaccctggatatttccttcctcttcctggagcctatggaggagaagctggtggtcctgcctttccccaaagaaaaagcacgcactgctgagtgcccagggccagcccagaatgccagccctcttgtccttccgccatgagaatcacacagagtcttctgtaggggtatggtgcgccgcatgacatgggaggcgatggggacgatggacagagacagagcgtgcacacgtagagtggctagtgaaggacgcctttttgactcttcttggtctcagcatgttgactgggattggaaataatgagactgagccctcggcttgggctgcactctaccctgtacactgccttgtaccctgagctgcatcacctcctaaactgagcagtctcataccatggagagatgcctctcttatgtcttcagccactcacttataaagatacttatcttttcagcagtatatatgtgctgaaatctcagcatgaaagcattgcatgagtaaagatactttccctaacaccgggaggcgtgagcaactaatgcccagcacagtttcagtccctaaattccctttaattccaggacaggtattagctgcgtagggcccataccatttccaaccctctacctcttttcatctgtctggaagtct

SEQ ID NO: 210 - DNA, 1365 bp, Mus musculus
  source 1..1365: mol_type = unassigned DNA
ttaatctctgtaaaggcgccacctctccacaggtttcataagcgttcgttttctgagctttgggaacgtgttcaaagcacaccagacttgcaaagctcttgattccggacagcttaccttgattcctgacattgtgcagtggctcagccagaacctgtgagtcacaggtagctgctgtgttacacaacatctaacaacgtctagccttctgcgatttctggtgcagggtcatgattagtgcatttgacagatttggcagagtggcttacagtgggaagacatgggatctctgttctacctcacattggatgtcttagagactgactgccacgtgctcagcaggaaggcacagaaggactgcaaaccgaggatgttctatgagtcggtttatggccagtgcaaagcaatgtttcacattaacaagccaagaagagttctctacttacctgcttataactgtacacttcgcccagtttccaaaagaaagactcatacaacgtgccctgactgccctagccccattgacttgtcaaaccccagtgctctggaagctgccacggagtcgcttgcaaagttcaacagtaagagcccctcaaaaaagtatgaactcgtcaaagtcaccaaggctatgaaccagtgggtgtctggccctgcttactatgtggaatatttgatcaaagaggcaccatgtaccaaatcccaggccagctgttcgctccagcactctgactctgagcccgttggtatttgccaaggttctacggtccaaagttctctgaggcacgttcctctgatccaacccgtagaaaagtctgtcactgtgacttgtgagttcttcgaatctcaggctcaggtccctggagatgagaaccctgctgttacccagggccctcagaaactccctcagaaaaacacggcccctaccagctccccctccgtaactgcaccaagaggatccatccagcacctccccgaactggatgatgagaagcccgaggagtccaagggagggagccctgaggaagcctttcctgtgcagctggatctaaccaccaatccccagggggacacactagatgtctccttcctgtacctggagcctggggacaagaagctggtggtgctgcctttccctggaaaggaacagcgctccgccgagtgcccagggcccgagaaggagaacaaccctctggttctcccaccctgagactccctagcagggtttcatagggctatggtccccagcactaaatgggaggtggtggggattgggaaggacacagacaatgaaatgtagacaggctaataaagtgtgtccttttgatgcttcttggcttcaaaaaaaaaaaaaaaaaaaaaaaaaaa

SEQ ID NO: 211 - protein, 345 aa, Homo sapiens
MGLLLPLALCILVLCCGAMSPPQLALNPSALLSRGCNDSDVLAVAGFALRDINKDRKDGYVLRLNRVNDAQEYRRVYGQCKAIFYMNNPSRVLYLAAYNCTLRPVSKKKIYMTCPDCPSSIPTDSSNHQVLEAATESLAKYNNENTSKQYSLFKVTRASSQWVVGPSYFVEYLIKESPCTKSQASSCSLQSSDSVPVGLCKGSLTRTHWEKFVSVTCDFFESQAPATGSENSAVNQKPTNLPKVEESQQKNTPPTDSPSKAGPRGSVQYLPDLDDKNSQEKGPQEAFPVHLDLTTNPQGETLDISFLFLEPMEEKLVVLPFPKEKARTAECPGPAQNASPLVLPP

SEQ ID NO: 212 - protein, 308 aa, Mus musculus
MGSLFYLTLDVLETDCHVLSRKAQKDCKPRMFYESVYGQCKAMFHINKPRRVLYLPAYNCTLRPVSKRKTHTTCPDCPSPIDLSNPSALEAATESLAKFNSKSPSKKYELVKVTKAMNQWVSGPAYYVEYLIKEAPCTKSQASCSLQHSDSEPVGICQGSTVQSSLRHVPLIQPVEKSVTVTCEFFESQAQVPGDENPAVTQGPQKLPQKNTAPTSSPSVTAPRGSIQHLPELDDEKPEESKGGSPEEAFPVQLDLTTNPQGDTLDVSFLYLEPGDKKLVVLPFPGKEQRSAECPGPEKENNPLVLPP

SEQ ID NO: 213 - skipped

SEQ ID NO: 214 - DNA, 1436 bp, Mus musculus
  source 1..1436: mol_type = unassigned DNA
ttgtgtattatgtagccagggacagcattctgaactctaccctggcacttcctgaacctcctagcttataaataccagggcaggccagagaatcagggaggagtgagcaaaaattctcagaccagctgggcctgtcctccaggaggatgagaaactccaagactgccatttcattctttatccttgtggctgtccttgggtcccaggctggcctcatacaagaaatggaaaaagaagatcgtcgctataaccctccaataattcatcaaggctttcaagacacttcttcagactgctgcttctcttatgccacacagatcccatgtaaaagatttatatattatttccccaccagtggtgggtgcatcaagccgggcatcatctttatcagcaggaggggaacccaggtctgtgccgaccccagcgatcggagagttcagaggtgcctaagcaccctgaagcaaggcccaagatctgggaacaaggtcattgcttgagaaggagggcaggcattgtcacccactttcttctgtcttccccagtgaccgcctgcctaggagaccttgtttttatagatatttaaagcatttatccttctgttcaggtttagagcagtcaacagtattcatgtggactccgcctgacacggttagagccatctggagttgtaaacatcaagattgtctttgagtaattgttgggttttttttcggtttctcagcagattataaatggatacattattagggtagtctttggggctttggaatgtgtctggttctgatacaagcttaagccgggtaatatctagctgagatgaaatcaattttgccctaggccatacatatgtccagctttgtgggttcccagttgttcgccctgccacaatagagcaatgagtgcccccaataaagtccactccatgtagccacaggactgtctctttcttcagattcatagaactacggggccagtctgaaactgggctcttggggtgaaattatctcactcactcaccttgagtacaggagggagatgggaccatataactttgaatattctaaaccaaaagacatcatggtataatttttaaaaattaagaacacggtttattcctctgagcttggtgcaaaacagaggaataccttttccagcagggcgtcttcttctctgcctgacttttatctgcggaacctgaccttcacaccctctacctgaggaagttcaggtagttgttggcaaatctacaagtaagaacctgcacaccaagtatctgggattcctggaatgcctctccatgcaaatgaggcattcccagtactttaaacttcatctagtaatttacatttaccctgaaaactccttcctatactccaaggttcatgtatagccctggtttaccccaataaagtgtatgcacacaagctgtttcactg

SEQ ID NO: 215 - skipped

SEQ ID NO: 216 - protein, 116 aa, Mus musculus
MRNSKTAISFFILVAVLGSQAGLIQEMEKEDRRYNPPIIHQGFQDTSSDCCFSYATQIPCKRFIYYFPTSGGCIKPGIIFISRRGTQVCADPSDRRVQRCLSTLKQGPRSGNKVIA

SEQ ID NO: 217 - DNA, 5898 bp, Homo sapiens
  source 1..5898: mol_type = unassigned DNA
aaaagtgagtccctgccttccctctctccgtctggctcctcccaggcctgtctggcaggggccggggtgcaggaggaggagacggcatccagtacagaggggctggacttggacccctgcagcagccctgcacaggagaagcggcatataaagccgcgctgcccgggagccgctcggccacgtccaccggagcatcctgcactgcagggccggtctctcgctccagcagagcctgcgcctttctgactcggtccggaacactgaaaccagtcatcactgcatctttttggcaaaccaggagctcagctgcaggaggcaggatggtctggaggctggtcctgctggctctgtgggtgtggcccagcacgcaagctggtcaccaggacaaagacacgaccttcgaccttttcagtatcagcaacatcaaccgcaagaccattggcgccaagcagttccgcgggcccgaccccggcgtgccggcttaccgcttcgtgcgctttgactacatcccaccggtgaacgcagatgacctcagcaagatcaccaagatcatgcggcagaaggagggcttcttcctcacggcccagctcaagcaggacggcaagtccaggggcacgctgttggctctggagggccccggtctctcccagaggcagttcgagatcgtctccaacggccccgcggacacgctggatctcacctactggattgacggcacccggcatgtggtctccctggaggacgtcggcctggctgactcgcagtggaagaacgtcaccgtgcaggtggctggcgagacctacagcttgcacgtgggctgcgacctcatagacagcttcgctctggacgagcccttctacgagcacctgcaggcggaaaagagccggatgtacgtggccaaaggctctgccagagagagtcacttcaggggtttgcttcagaacgtccacctagtgtttgaaaactctgtggaagatattctaagcaagaagggttgccagcaaggccagggagctgagatcaacgccatcagtgagaacacagagacgctgcgcctgggtccgcatgtcaccaccgagtacgtgggccccagctcggagaggaggcccgaggtgtgcgaacgctcgtgcgaggagctgggaaacatggtccaggagctctcggggctccacgtcctcgtgaaccagctcagcgagaacctcaagagagtgtcgaatgataaccagtttctctgggagctcattggtggccctcctaagacaaggaacatgtcagcttgctggcaggatggccggttctttgcggaaaatgaaacgtgggtggtggacagctgcaccacgtgtacctgcaagaaatttaaaaccatttgccaccaaatcacctgcccgcctgcaacctgcgccagtccatcctttgtggaaggcgaatgctgcccttcctgcctccactcggtggacggtgaggagggctggtctccgtgggcagagtggacccagtgctccgtgacgtgtggctctgggacccagcagagaggccggtcctgtgacgtcaccagcaacacctgcttggggccctccatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtggatggctgtttatccaacccctgcttcccgggagcccagtgcagcagcttccccgatgggtcctggtcatgcggctcctgccctgtgggcttcttgggcaatggcacccactgtgaggacctggacgagtgtgccctggtccccgacatctgcttctccaccagcaaggtgcctcgctgtgtcaacactcagcctggcttccactgcctgccctgcccgccccgatacagagggaaccagcccgtcggggtcggcctggaagcagccaagacggaaaagcaagtgtgtgagcccgaaaacccatgcaaggacaagacacacaactgccacaagcacgcggagtgcatctacctgggccacttcagcgaccccatgtacaagtgcgagtgccagacaggctacgcgggcgacgggctcatctgcggggaggactcggacctggacggctggcccaacctcaatctggtctgcgccaccaacgccacctaccactgcatcaaggataactgcccccatctgccaaattctgggcaggaagactttgacaaggacgggattggcgatgcctgtgatgatgacgatgacaatgacggtgtgaccgatgagaaggacaactgccagctcctcttcaatccccgccaggctgactatgacaaggatgaggttggggaccgctgtgacaactgcccttacgtgcacaaccctgcccagatcgacacagacaacaatggagagggtgacgcctgctccgtggacattgatggggacgatgtcttcaatgaacgagacaattgtccctacgtctacaacactgaccagagggacacggatggtgacggtgtgggggatcactgtgacaactgccccctggtgcacaaccctgaccagaccgacgtggacaatgaccttgttggggaccagtgtgacaacaacgaggacatagatgacgacggccaccagaacaaccaggacaactgcccctacatctccaacgccaaccaggctgaccatgacagagacggccagggcgacgcctgtgaccctgatgatgacaacgatggcgtccccgatgacagggacaactgccggcttgtgttcaacccagaccaggaggacttggacggtgatggacggggtgatatttgtaaagatgattttgacaatgacaacatcccagatattgatgatgtgtgtcctgaaaacaatgccatcagtgagacagacttcaggaacttccagatggtccccttggatcccaaagggaccacccaaattgatcccaactgggtcattcgccatcaaggcaaggagctggttcagacagccaactcggaccccggcatcgctgtaggttttgacgagtttgggtctgtggacttcagtggcacattctacgtaaacactgaccgggacgacgactatgccggcttcgtctttggttaccagtcaagcagccgcttctatgtggtgatgtggaagcaggtgacgcagacctactgggaggaccagcccacgcgggcctatggctactccggcgtgtccctcaaggtggtgaactccaccacggggacgggcgagcacctgaggaacgcgctgtggcacacggggaacacgccggggcaggtgcgaaccttatggcacgaccccaggaacattggctggaaggactacacggcctataggtggcacctgactcacaggcccaagactggctacatcagagtcttagtgcatgaaggaaaacaggtcatggcagactcaggacctatctatgaccaaacctacgctggcgggcggctgggtctatttgtcttctctcaagaaatggtctatttctcagacctcaagtacgaatgcagagatatttaaacaagatttgctgcatttccggcaatgccctgtgcatgccatggtccctagacacctcagttcattgtggtccttgtggcttctctctctagcagcacctcctgtcccttgaccttaactctgatggttcttcacctcctgccagcaaccccaaacccaagtgccttcagaggataaatatcaatggaactcagagatgaacatctaacccactagaggaaaccagtttggtgatatatgagactttatgtggagtgaaaattgggcatgccattacattgctttttcttgtttgtttaaaaagaatgacgtttacatataaaatgtaattacttattgtatttatgtgtatatggagttgaagggaatactgtgcataagccattatgataaattaagcatgaaaaatattgctgaactacttttggtgcttaaagttgtcactattcttgaattagagttgctctacaatgacacacaaatcccattaaataaattataaacaagggtcaattcaaatttgaagtaatgttttagtaaggagagattagaagacaacaggcatagcaaatgacataagctaccgattaactaatcggaacatgtaaaacagttacaaaaataaacgaactctcctcttgtcctacaatgaaagccctcatgtgcagtagagatgcagtttcatcaaagaacaaacatccttgcaaatgggtgtgacgcggttccagatgtggatttggcaaaacctcatttaagtaaaaggttagcagagcaaagtgcggtgctttagctgctgcttgtgccgctgtggcgtcggggaggctcctgcctgagcttccttccccagctttgctgcctgagaggaaccagagcagacgcacaggccggaaaaggcgcatctaacgcgtatctaggctttggtaactgcggacaagttgcttttacctgatttgatgatacatttcattaaggttccagttataaatattttgttaatatttattaagtgactatagaatgcaactccatttaccagtaacttattttaaatatgcctagtaacacatatgtagtataatttctagaaacaaacatctaataagtatataatcctgtgaaaatatgaggcttgataatattaggttgtcacgatgaagcatgctagaagctgtaacagaatacatagagaataatgaggagtttatgatggaaccttaaatatataatgttgccagcgattttagttcaatatttgttactgttatctatctgctgtatatggaattcttttaattcaaacgctgaaaagaatcagcatttagtcttgccaggcacacccaataatcagtcatgtgtaatatgcacaagtttgtttttgtttttgttttttttgttggttggtttgtttttttgctttaagttgcatgatctttctgcaggaaatagtcactcatcccactccacataaggggtttagtaagagaagtctgtctgtctgatgatggatagggggcaaatctttttcccctttctgttaatagtcatcacatttctatgccaaacaggaacaatccataactttagtcttaatgtacacattgcattttgataaaattaattttgttgtttcctttgaggttgatcgttgtgttgttgttttgctgcactttttacttttttgcgtgtggagctgtattcccgagaccaacgaagcgttgggatacttcattaaatgtagcgactgtcaacagcgtgcaggttttctgtttctgtgttgtggggtcaaccgtacaatggtgtgggagtgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacctcatttttaaagattaacaacaggaaataaattgtaaaaaaggttttctatacatgaaaaaaaaaaaaaaaaaa

SEQ ID NO: 218 - DNA, 5922 bp, Mus musculus
  source 1..5922: mol_type = unassigned DNA
gaggggctggatttggactgggtaaccaggtactctgagcaaatggcgagaccagaggacgaattatgacactccttcgaaagaatgaattaaacgtgcctccgcatgtgtctgattgacaggcctcctggggaaagggcgtataaaggagccggtcactgccgggaggcagagagagccagtccgatgtctgcagcctccctggccaggcctctcctctcctgccgcagctagtccccctcaggacagacagagtactggcgtcggtcaccattcacttgcaaacacaccaggtcacgtgaagaaacttcctggtgacactcaggctgtagctgtgcactcttcaaccacgaggttggttttctcctaagtgtcacaggtggagacaagatgctctgggcactggccctgctggctctgggcatagggccaagagcttctgctggtgaccacgtcaaggacacttcatttgaccttttcagcatcagcaacattaaccggaagaccatcggtgccaagcagttccgagggcctgaccccggggtgcccgcctaccgttttgtacggtttgactacatccccccagtgaacacagatgatctcaacaggattgtcaagcttgcaaggagaaaggagggcttcttcctcacagcccaactgaagcaggaccgcaagtctcggggaacgctcctggtgttggaaggccccggcacctcccagaggcagtttgagattgtgtccaatggcccaggggacactttggacctcaactactgggtagaaggcaatcagcataccaacttcctggaggatgtgggcctggctgactcccagtggaagaatgtgactgtgcaggtggccagtgacacctatagcctgtatgtgggctgcgatcttatcgacagtgtcaccctggaagaaccattctatgagcagctagaagtagacaggagcaggatgtacgtggccaaaggtgcatctcgagagagtcacttcaggggcttgctgcagaatgtccatctcgtgtttgcagattctgtggaagatatcttaagcaagaaaggctgtcaacacagccagggagctgaagtcaacaccatcagtgaacatacagagactctccatctgagccctcacatcaccacagatctcgtggtccagggtgtggagaaggcacaggaggtgtgtacgcactcctgcgaggagttgagcaacatgatgaatgagctctctggactgcacgtcatggtgaaccagctgagcaagaacctggagagagtgtctagtgataaccagttccttttggagctcattgggggccctctgaagacaagaaacatgtcagcctgtgtgcaggagggccgaatctttgcagaaaatgaaacctgggttgtggatagttgtaccacatgcacctgcaagaaatttaaaacagtctgccatcagatcacctgctcacctgcaacttgtgccaacccatcttttgtggaaggcgagtgctgtccatcctgttcacactctgcagacagtgatgagggctggtctccgtgggcagagtggaccgagtgttctgtcacctgtggctctgggacccagcagagaggccggtcttgtgatgtcaccagcaacacctgcctgggcccctccattcagacaaggacatgcagcctgggcaaatgtgatacgagaatccgtcagaatggaggctggagtcactggtcaccctggtcttcatgctccgtgacttgtggagttggcaatgtcacccgcatacgtctctgcaactcaccagtgccccagatgggtggcaagaactgcaagggcagcggccgggaaaccaaaccctgtcagcgtgatccgtgcccaattgatggccgctggagcccctggtccccttggtcagcctgcacagttacctgtgctggagggatccgtgagcgctcacgtgtttgcaacagccctgagccccagtatggagggaaggactgtgtcggggatgtgacagaacaccaaatgtgcaacaagagaagctgccctattgatgggtgcttatccaacccgtgttttcctggagccaagtgcaacagcttccctgatgggtcctggtcctgtggctcctgcccagtgggctttctgggcaatggtacccactgtgaggacctggatgagtgtgctgtggtcacagatatttgcttctcaactaacaaagctccccgctgtgtcaacaccaacccgggcttccactgcctgccttgtccaccacgctacaaggggaaccaacccttcggtgttggcctggaggatgctaggacagaaaaacaagtgtgtgagccagagaatccatgtaaggacaagactcacagctgccacaagaatgcagagtgcatctacctgggccactttagtgaccccatgtacaagtgtgagtgccagattggctacgcaggtgatgggctcatctgcggggaggactcagacctggatggctggcccaacaacaacctggtgtgtgctactaatgccacctaccactgcatcaaggacaactgccccaaactgccaaattccgggcaggaggattttgataaggatggaatcggagatgcttgtgacgaggacgatgacaatgacggtgtgagcgatgagaaggacaattgccagcttctcttcaatccccgtcaattagactatgacaaggatgaggttggagaccgctgtgacaactgcccctatgtgcacaacccagcacagatcgacacagacaacaatggcgagggggatgcctgctctgtggacattgacggagacgatgttttcaatgagcgagacaattgtccatatgtctacaacactgaccagagagatacggatggtgatggcgtgggtgaccactgtgacaattgtcctctgatgcacaacccagatcagatcgatcaggacaatgatctcgttggagaccagtgtgacaacaatgaggacatagatgatgacggccaccagaacaaccaagacaactgcccatacatctccaactccaaccaggctgaccatgacaacgacggcaagggcgatgcctgcgactctgatgatgacaatgatggtgttccagatgacagggacaactgtcggcttgtgttcaacccagaccaggaagactcggacggtgacggccgaggtgacatttgtaaagatgactttgacaatgataatgtcccagatattgatgatgtgtgccctgagaacaatgccatcactgagacagacttcagaaacttccagatggtccctctggatcccaaggggaccacacaaattgatcccaactgggtaattcgtcaccaaggcaaagagctggtgcagacagcaaactcagaccctggcatcgctgtaggtttcgacgagtttgggtctgtggacttcagtggcactttctatgtcaacactgaccgggatgatgactacgctggctttgtctttggctatcagtcaagcagccgcttctatgtggtgatgtggaagcaggtgacccagacctactgggaagacaagcccagtcgggcttacggctactctggtgtgtcactcaaagtggtaaactccacgactggtactggcgagcacctgaggaatgccctgtggcacacgggaaacacagaaggccaggtccggactctatggcatgaccccaaaaacattggctggaaagactacactgcctacaggtggcacctgattcacaggcctaagacaggctacatgagagtcttagtgcatgaaggaaagcaagtcatggctgactcaggaccaatttatgaccaaacctacgctggtggacggctgggcctgtttgtcttctcccaagagatggtctatttctcggacctcaagtatgagtgcagagatgcctagagagcagggctccagctccagcaatgtgctgcaaacaccccttcttagacacatcagtccatcttggcacttgtggcttttctgtcatttggcatttcctgtttcttgaccttaactgagtggatctacacctccttcatcagcaccaagtccaagtgtcttcaaaggagaaacatcaattgcactccaagagcttccagcctgctgctggaaaacatttggatgagatatgaggctcaccgtggagcgaagaccgagcattccgctgtgttgccttttcttgtttgtttaaaaagaatgacgtttacatgtaaatgtaattacttgcagtatttatgtgtatatggagtcgaagggagtattgtgtttaagtcattctcataaattaagcatgaaaaatatggctcacttaccctcagtgcttaatgttgtcactgtttttggattcgtgctgttctataatgacacacaaatcctgtgaggctgaacaggaagaaagatcaggtgaactcggctgcagtaaggaagaggccagaagacagtcatgtgagactgtggccccttgtccagtgatttgaaacattaactaattagaacataggaagtgatcgaaaagttagaagaaacgcagttcttacttggccacagttaaagctgactcgacagagacaaagaagctgcatcctttgaaaggggaatatttcagggttccagctgtaggcttctcagagctttcagaagaaaacctcagcagaggcaatgtccatttcagctgcctgcctgggttgggcagtgtggaaagcatacctggctggacgtcatcaggctctgcatactctgcatactgactgctaggatctttgtctgggcatcctttcctgtccctgctgctaggaaacagactggcaaataagttggcagatgcactttggtaccctgttcctactctaggctttagaatgatgcaaaagctgcttttgccaaatttggtaatgcatttccttaaggttccaggtacggctcttttgttaatatttattaaatgatttggaatatagcttcactcatcaataatttattttaaacatacaaagtaacaagtatgtagtatataatttctagaaataagcatatgacaaggatataatgatataaagacacacttacaaatatctgctgtttgaaatacaaggatttatgtcatgatagaatggaaccttaatatgcatccttaccaataattttaattcagttctttttactattatctctttgttgtatataggttctttttattttaatgcttaaggaaacatcatttgttttttccagacacacccattaatcagtcacatcattacagacatatgcttgatttattttattttgtttttgttttgctttaagctgcatgatctcccagcaggaagtactgcctctctcacttcacacgagctcagtggaagaatattttgtccctctggtggggtagttcacagtcccctttctagtaatggcaactgcatttctaagttagacagaaagaatccacgattttgatctgagtttccacaatttgtctagatggagttatttgtgttgaaatctctgagggtgagtttccttgtttcttgttatcctgcactttttagatttttgtggaactgtattcccaaggccagcacaggtcaggagacttctggccatgagggtgtgaagaacatttaggctctcacttgggcatttggttcaatggtacaaggtggtgggagcgatgtgaatacattcaatgtaccatattatttttgtaaattatttatgtagttttttttaaaaacagaagcttctcatatacgttaatgaaacattgtttttttttttttgccaaagattgtaaatatttatttatttgttcattgggttaaatttcaccactaaaacccctcatttagctagagcttcatttttataccaaagattatcaacagcaaataaattataaaaaggtttctg

SEQ ID NO: 219 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 220 - protein, 1172 aa, Mus musculus
MLWALALLALGIGPRASAGDHVKDTSFDLFSISNINRKTIGAKQFRGPDPGVPAYRFVRFDYIPPVNTDDLNRIVKLARRKEGFFLTAQLKQDRKSRGTLLVLEGPGTSQRQFEIVSNGPGDTLDLNYWVEGNQHTNFLEDVGLADSQWKNVTVQVASDTYSLYVGCDLIDSVTLEEPFYEQLEVDRSRMYVAKGASRESHFRGLLQNVHLVFADSVEDILSKKGCQHSQGAEVNTISEHTETLHLSPHITTDLVVQGVEKAQEVCTHSCEELSNMMNELSGLHVMVNQLSKNLERVSSDNQFLLELIGGPLKTRNMSACVQEGRIFAENETWVVDSCTTCTCKKFKTVCHQITCSPATCANPSFVEGECCPSCSHSADSDEGWSPWAEWTECSVTCGSGTQQRGRSCDVTSNTCLGPSIQTRTCSLGKCDTRIRQNGGWSHWSPWSSCSVTCGVGNVTRIRLCNSPVPQMGGKNCKGSGRETKPCQRDPCPIDGRWSPWSPWSACTVTCAGGIRERSRVCNSPEPQYGGKDCVGDVTEHQMCNKRSCPIDGCLSNPCFPGAKCNSFPDGSWSCGSCPVGFLGNGTHCEDLDECAVVTDICFSTNKAPRCVNTNPGFHCLPCPPRYKGNQPFGVGLEDARTEKQVCEPENPCKDKTHSCHKNAECIYLGHFSDPMYKCECQIGYAGDGLICGEDSDLDGWPNNNLVCATNATYHCIKDNCPKLPNSGQEDFDKDGIGDACDEDDDNDGVSDEKDNCQLLFNPRQLDYDKDEVGDRCDNCPYVHNPAQIDTDNNGEGDACSVDIDGDDVFNERDNCPYVYNTDQRDTDGDGVGDHCDNCPLMHNPDQIDQDNDLVGDQCDNNEDIDDDGHQNNQDNCPYISNSNQADHDNDGKGDACDSDDDNDGVPDDRDNCRLVFNPDQEDSDGDGRGDICKDDFDNDNVPDIDDVCPENNAITETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDKPSRAYGYSGVSLKVVNSTTGTGEHLRNALWHTGNTEGQVRTLWHDPKNIGWKDYTAYRWHLIHRPKTGYMRVLVHEGKQVMADSGPIYDQTYAGGRLGLFVFSQEMVYFSDLKYECRDA

SEQ ID NO: 221 - DNA, 4306 bp, Homo sapiens
  source 1..4306: mol_type = unassigned DNA
gcacacagccaggggccgcccgggctctcgacgcgccgacggccgggcggacggacggacggacgccgagcgcagtgccccgcgggtcttacaggagaggggaccgtcctgggctggcctggaccatggtgctgctctgggagcctgcaggagcctggcttgctctgggcctggccctggccctgggccccagcgtggccgcagctgcccctcggcaggactgcacgggcgtggagtgcccgccgctggagaactgcattgaggaggcgctggagccgggtgcctgctgtgccacgtgtgtgcagcagggctgcgcctgcgagggctaccagtactatgactgcctacagggtggcttcgtgcgcggccgcgtgcccgccggtcagtcctattttgtggacttcgggagcactgagtgctcctgcccaccaggcggcggcaagatcagctgccagttcatgctgtgcccggagctgccgcccaactgcatcgaggctgtagtggtggctgacagctgcccacagtgcggccaggtgggctgcgtccacgcgggccacaagtacgccgctggccacactgttcacctgccgccctgccgggcctgccactgccctgacgccggtggagagctcatctgctaccagctccccggttgccacgggaacttctcagatgccgaggagggtgaccccgagcgacactacgaagacccctacagctatgaccaggaggtggccgaggtggaagcagcaacagccctggggggtgaggtccaggcgggtgcagtccaggcaggcgcagggggccccccagctgctctgggaggtgggagtcagccactgtccaccatccaggcacccccctggccagctgtcctccccaggcccacagcggctgctgccctgggtcccccagccccagtgcaggccaaagctaggagagtgaccgaggacagtgaggaggaagaagaggaggaggaggagagagaggaaatggctgtcactgagcagctggcagcaggtggccacagggggctggatgggctgcccactacagccccagctggacccagtcttcctatccaggaggagagggcagaagctggggcaagggcagaagctggggcaaggcctgaagagaacctcatcctggatgcccaagccacgtcccgcagcactgggccggagggcgtgacgcatgcaccgagcctgggcaaggctgctctcgtcccaactcaggccgtgcctggctctcccagggacccagtcaagcccagcccccacaacatcctgtccacatcactgcctgatgcagcctggatcccacccacccgagaagtgcccaggaagccgcaagttctgccccattcccacgtggaggaggacacagaccccaactctgtccattctatccccagaagtagccctgaaggctccaccaaggacctgatcgagacttgctgcgcagccggacagcagtgggccattgacaatgacgagtgcctggagatccctgagagtggcactgaggacaacgtctgcaggacagcccagaggcactgctgtgtctcctacttgcaggagaagagctgcatggccggcgtcctgggagccaaggagggtgagacctgtggggctgaggacaacgacagctgcggcatctccctgtacaagcaatgctgtgactgctgtggcctgggcctccgcgtgcgggccgagggccagtcgtgtgagtccaatcctaacctgggctatccctgcaatcatgtcatgctctcctgctgtgagggtgaagagcctctcatagtacctgaggttcgccgacctccagagcccgcagctgcaccacggagagtttcagaggcagagatggcgggccgagaggccctgtcactgggcacagaggccgagctgccgaacagcctgccgggcgatgaccaggatgagtgccttctcctcccgggagagctgtgccagcacctttgcatcaatactgtgggttcttaccactgtgcctgctttcctggcttctcactgcaggacgatggccgcacttgccgcccagagggtcaccctccacagccggaagccccacaggagcctgcactgaagtcagaattttcccaggtggcctctaacaccatcccgctgccactgccgcagcccaatacctgcaaagacaatggaccctgcaagcaggtgtgcagcactgttgggggctcagccatatgctcctgttttcccggctatgccatcatggcggatggcgtgtcctgtgaagaccaagacgagtgcctgatgggtgctcacgattgtagccggcgacagttctgtgtgaacaccctgggatccttctactgtgtcaaccacacagtgctctgtgccgatggctatatcctcaatgcgcacaggaagtgcgtggacatcaacgagtgtgtgacggacctgcacacgtgcagccggggcgagcactgtgtgaacacactgggctccttccactgctacaaggcactcacctgtgagccaggctatgccctcaaggatggcgagtgcgaagacgtggatgagtgtgcgatgggcacgcacacctgccagccgggcttcttgtgccagaacaccaagggctccttctactgccaggccaggcagcgctgcatggatggcttcctgcaggatcctgaaggcaactgtgtggacatcaacgagtgcacgtcactgtccgagccatgtcggccaggcttcagctgcatcaacacggtgggctcctacacatgccagaggaacccgctgatctgcgcgcgcggctaccacgccagcgatgatgggaccaagtgtgtggacgtgaatgagtgtgagacaggtgtgcaccgctgcggtgagggccaagtgtgccacaacctccctggctcctaccgctgtgactgcaaagccggctttcagcgggatgcctttggccggggctgcatcgacgtgaatgagtgctgggcctcgccaggccgcctgtgccagcacacgtgtgagaacacactcggctcctaccgctgttcctgcgcctccgggttcctgctagcagcggacggcaagcgctgtgaagacgtgaatgagtgtgaggcccagcgctgcagccaggagtgtgccaacatctatggctcctaccagtgctactgccgccagggctaccagctggctgaggatgggcacacctgcacagacatcgacgagtgtgctcaaggcgccggcatcctctgcaccttccgctgtctcaacgtgccagggagctaccagtgtgcatgccctgagcagggctacaccatgacggccaacgggaggtcctgcaaggacgtggatgagtgtgcactgggtacccacaactgttccgaggctgagacctgccacaacatccagggtagcttccgctgcctgcgcttcgagtgtcctcccaactatgtccaagtctccaaaacgaagtgcgagcgcaccacgtgccatgacttcctggagtgccagaactcgccagcgcgcatcacgcactaccagctcaacttccagacgggcctcctggtgcctgcgcatatcttccgcattggccccgcgccagccttcacgggggacaccatcgccctgaacatcatcaagggcaatgaggagggctactttggcacgcgcaggctcaatgcctacacgggtgtggtctacctgcagcgggccgtgctggagccccgggactttgccctggacgtggagatgaagctctggaggcagggctccgtcaccaccttcctggccaagatgcacatcttcttcaccacctttgccctgtgaggtgccagcacgggccacctgcgggtgtggcgcagcccagggctcacactgcgtgggagggactgggtcactattgtggtttttactataactttgtaaattaacttaattttgctgacttgactcctgtggcttctggacccctcctctgccccgcaggaggaagttccacggcaggtggtgcgttcccacgcaggcaccaagtggaagcttgcacggtgggccacggccgtggcgggtgccctgtgggtgaggctgggtgatgacctgaggaccagagacacgcgaccatgttggggctcttggactcctctggatgacccgtccccaaagttgacattccatttcatgttccactgtgattaattcttttcttttttaaaaaatcattttaaagttttttgtttaactataaagtagtacatgtacattatataaaaaaaagttcaactagtatgaaagggttataaagtaacagaggaaaa

SEQ ID NO: 222 - DNA, 4336 bp, Mus musculus
  source 1..4336: mol_type = unassigned DNA
cctgtggggcggggtctcaaggcctggctagtcccgctcttctccaaccccgagccgcgcgccacccggactctggattcaccgacggcgggtgcgcggacagactgacggcggcgcagctcaacacagagcaccggggactcctgagacatgctgctccaggaatctgcaggagtgtggctggccttggctctggtcacagctctgactccgagcccaagcatggccgtcccgtggcaggattgcacaggtgctgagtgtcccctgctggagaactgcatcgaggaggcgttggagccaggagcctgctgtgctacctgtgtgcagcagggctgtgcctgcgaaggctaccagtactacgactgcgtacaaggagggtttgtggatggccgtgtaccagccggccagtcctactttgtggactttggcagcaccgagtgctcctgcccacccggtggtggcaagatcagctgccagttcatgctgtgccctgagctaccacccaactgcattgaagctgtagtagtagccgacagctgcccgcagtgtggccaggtgggctgtgtccacagtggccgtaagtatgctgctggccataccgtccacctctcatcctgccgggcttgccactgtcctgacgctggtggtgagctcatttgctaccagcttcctggttgccatgggaacttctcggatgctgaggagggtgactctgagaggcagtatgaagacccctacagctatgaccaggaggtggcagaggcagaagctactacggccattgtgaacgaggtccaggcaggtgcagagggccccccagctgctctgggaggagggaatctgccaccgtcctccatcagagtaacaccctggccagttgcccttcccagacccacagcagctgctgccctgggtcccccagcacctgtgcaggccaaagccaggagagtgacattggacactgaagaggatgaggaagaagaagaggaggagacactagtcactgagccaccaacagcaggcagtcctggaaggctggacagcctgcccacaagatccccagccagacctggtttccctgtccaggagaaggaggctgaagctaaggcagggcctgaggaaaaccttattcctgatgcccaggtcactcctcgaagtgtcatgcaggagggtgctgcacctctgccaaggtcaggcctggctgctctcagtccatctctggccactgacagctcttcagaggacccagtaaagcccagcgatcaccccactctatctacactgccacctgaccgagcccaggtctccccctccccagagacacctgaagaaataccccagcatccacagttgctacctcgattccgggcagaagaagacatagaccccaactcagtacattctgtccccagaggtgatcttgatggctccaccaaggacctaatcgagacctgttgtgcagcgggccagcagtgggccatcgacaatgatgagtgccaggagatccccgagaatggtgcccagagtgacatctgtaggatagcccagaggcaatgctgcatctcctacttgaaagagaagagctgtgtggctggggtcatgggagccaaggagggtgagacctgtggagcagaggacaatgatacctgcggcgtctcactctacaagcaatgctgtgactgctgtggcctgggactccgagtgcgggctgagggccagtcctgtgagtccaaccccaacctgggctatccctgcaaccatgtcatgctctcctgctgtgaaggtgaagagcccctcatagtgcctgaggtccgccggccaccagagcccgaggctgctcctcgcagagtttcagagatggagatggcaagccgggaggccctgtcactgggcacagaggctgaactgcccaatagcctgccgggagatgaccaggatgagtgcctgatgctccctggggagctttgccagcatctttgcatcaacaccgtcggctcctaccgctgtgcctgcttccctggattcgagctgcagggtgatggccgtacctgccgcccagatagaggtgccccacagttggacactgcacgagagtctgcaccgaggtctgagtctgcccaggtatctcccaacaccatcccactgccggtgccacagcccaacacctgcaaagacaatgggccctgccgtcaggtgtgtcgtgttgttggggacacagctatgtgctcctgcttccctggctacgccatcatggcagatggtgtgtcctgtgaagacatcaatgagtgtgtgacagacctgcacacgtgcacccgggccgagcactgtgtgaacacgccaggttccttccaatgctacaaggcgctcacctgtgagccaggctatgtcctcacagatggtgaatgcacagacgtggacgaatgtgtgacgggcacacacaactgccaggcaggcttctcatgccagaacaccaagggctccttctactgccaggcccggcaacgctgcatggatggcttcttgcaggaccccgagggcaactgtgtggacatcaacgaatgcacatcgttactggagccttgccgctcagggttcagctgcatcaacacagtgggctcctacacctgtcagaggaacccactggtctgcggtcgcggttaccatgctaacgaggagggctctgaatgtgtggatgtgaatgagtgtgagacaggtgtgcatcgctgtggcgagggccaactgtgctataacctccctggatcctaccgctgtgactgcaagcccggcttccagagggatgcattcggcaggacttgcattgatgtgaacgaatgctgggtctcgccgggccgcctgtgccagcacacatgtgagaacacaccgggctcctaccgctgctcctgcgctgctggcttccttttggccgcagatggcaaacattgtgaagatgtgaacgagtgcgagactcggcgctgcagccaggaatgtgccaacatctatggctcctatcagtgctactgccgtcagggctaccagctggcagaggatgggcatacctgcacagacatcgatgagtgtgcacagggcgcgggcattctctgtaccttccgctgtgtcaacgtgcctgggagctaccagtgtgcatgcccagagcaagggtatacaatgatggccaacgggaggtcctgcaaggacctggatgagtgtgcactgggcacccacaactgctctgaggctgagacctgccacaatatccaggggagtttccgctgcctgcgctttgattgtccacccaactatgtccgtgtctcagaaacgaagtgcgagcgcaccacatgccaggatatcacggaatgtcaaacctcaccagctcgcatcacgcactaccagctcaatttccagacaggcctactggtacctgcacatatcttccgcatcggccctgctcccgcctttgctggggacaccatctccctgaccatcacgaagggcaatgaggagggctacttcgtcacacgcagactcaatgcctacactggtgtggtatccctgcagcggtctgttctggagccgcgggactttgccctagatgtggagatgaagctttggcgccagggctctgtcactaccttcctggccaagatgtacatcttcttcaccacttttgccccatgaggtgacatgtcaggcaatccctccaggtgatgcctgggcggtgggcagctgcgccactcctaagtggctttttgctgtgactctgtaacttaacttaatcatgctgagctggttggtcttgagtctctaccctagagggagggagatgcaccccagcaggcactgagtacaggccagggtcacccgaggctagatggtgacctgcaaactggaaacagccatagggggcttctgaactccactcctcaactatggctacagctgacattccattccttcatccactgtgttcctcaattaaaaaaaaaaatcagctgtgcatggtagcacagacctttaatcctagcactggggaggcagaggtaggtagatctctgagttccaggccagcctggtctacactgggagttctaaccagccagagctacatagagagaccctatctcaacaaggaaaaaacgaaagaaatctctgtgagttccaggccagcctggtctacgctgggagttctaaccagccagagctacatagagagatcctatctcaacaaggaaaaatgaaagaaatcattttaaaaggtttttttttttgctgttgttgtttaatgataagagtagcacatatacattattaaaaatgatcaaatagcacagaaaggtta

SEQ ID NO: 223 - protein, 1231 aa, Homo sapiens
MVLLWEPAGAWLALGLALALGPSVAAAAPRQDCTGVECPPLENCIEEALEPGACCATCVQQGCACEGYQYYDCLQGGFVRGRVPAGQSYFVDFGSTECSCPPGGGKISCQFMLCPELPPNCIEAVVVADSCPQCGQVGCVHAGHKYAAGHTVHLPPCRACHCPDAGGELICYQLPGCHGNFSDAEEGDPERHYEDPYSYDQEVAEVEAATALGGEVQAGAVQAGAGGPPAALGGGSQPLSTIQAPPWPAVLPRPTAAAALGPPAPVQAKARRVTEDSEEEEEEEEEREEMAVTEQLAAGGHRGLDGLPTTAPAGPSLPIQEERAEAGARAEAGARPEENLILDAQATSRSTGPEGVTHAPSLGKAALVPTQAVPGSPRDPVKPSPHNILSTSLPDAAWIPPTREVPRKPQVLPHSHVEEDTDPNSVHSIPRSSPEGSTKDLIETCCAAGQQWAIDNDECLEIPESGTEDNVCRTAQRHCCVSYLQEKSCMAGVLGAKEGETCGAEDNDSCGISLYKQCCDCCGLGLRVRAEGQSCESNPNLGYPCNHVMLSCCEGEEPLIVPEVRRPPEPAAAPRRVSEAEMAGREALSLGTEAELPNSLPGDDQDECLLLPGELCQHLCINTVGSYHCACFPGFSLQDDGRTCRPEGHPPQPEAPQEPALKSEFSQVASNTIPLPLPQPNTCKDNGPCKQVCSTVGGSAICSCFPGYAIMADGVSCEDQDECLMGAHDCSRRQFCVNTLGSFYCVNHTVLCADGYILNAHRKCVDINECVTDLHTCSRGEHCVNTLGSFHCYKALTCEPGYALKDGECEDVDECAMGTHTCQPGFLCQNTKGSFYCQARQRCMDGFLQDPEGNCVDINECTSLSEPCRPGFSCINTVGSYTCQRNPLICARGYHASDDGTKCVDVNECETGVHRCGEGQVCHNLPGSYRCDCKAGFQRDAFGRGCIDVNECWASPGRLCQHTCENTLGSYRCSCASGFLLAADGKRCEDVNECEAQRCSQECANIYGSYQCYCRQGYQLAEDGHTCTDIDECAQGAGILCTFRCLNVPGSYQCACPEQGYTMTANGRSCKDVDECALGTHNCSEAETCHNIQGSFRCLRFECPPNYVQVSKTKCERTTCHDFLECQNSPARITHYQLNFQTGLLVPAHIFRIGPAPAFTGDTIALNIIKGNEEGYFGTRRLNAYTGVVYLQRAVLEPRDFALDVEMKLWRQGSVTTFLAKMHIFFTTFAL

SEQ ID NO: 224 - protein, 1174 aa, Mus musculus
MLLQESAGVWLALALVTALTPSPSMAVPWQDCTGAECPLLENCIEEALEPGACCATCVQQGCACEGYQYYDCVQGGFVDGRVPAGQSYFVDFGSTECSCPPGGGKISCQFMLCPELPPNCIEAVVVADSCPQCGQVGCVHSGRKYAAGHTVHLSSCRACHCPDAGGELICYQLPGCHGNFSDAEEGDSERQYEDPYSYDQEVAEAEATTAIVNEVQAGAEGPPAALGGGNLPPSSIRVTPWPVALPRPTAAAALGPPAPVQAKARRVTLDTEEDEEEEEEETLVTEPPTAGSPGRLDSLPTRSPARPGFPVQEKEAEAKAGPEENLIPDAQVTPRSVMQEGAAPLPRSGLAALSPSLATDSSSEDPVKPSDHPTLSTLPPDRAQVSPSPETPEEIPQHPQLLPRFRAEEDIDPNSVHSVPRGDLDGSTKDLIETCCAAGQQWAIDNDECQEIPENGAQSDICRIAQRQCCISYLKEKSCVAGVMGAKEGETCGAEDNDTCGVSLYKQCCDCCGLGLRVRAEGQSCESNPNLGYPCNHVMLSCCEGEEPLIVPEVRRPPEPEAAPRRVSEMEMASREALSLGTEAELPNSLPGDDQDECLMLPGELCQHLCINTVGSYRCACFPGFELQGDGRTCRPDRGAPQLDTARESAPRSESAQVSPNTIPLPVPQPNTCKDNGPCRQVCRVVGDTAMCSCFPGYAIMADGVSCEDINECVTDLHTCTRAEHCVNTPGSFQCYKALTCEPGYVLTDGECTDVDECVTGTHNCQAGFSCQNTKGSFYCQARQRCMDGFLQDPEGNCVDINECTSLLEPCRSGFSCINTVGSYTCQRNPLVCGRGYHANEEGSECVDVNECETGVHRCGEGQLCYNLPGSYRCDCKPGFQRDAFGRTCIDVNECWVSPGRLCQHTCENTPGSYRCSCAAGFLLAADGKHCEDVNECETRRCSQECANIYGSYQCYCRQGYQLAEDGHTCTDIDECAQGAGILCTFRCVNVPGSYQCACPEQGYTMMANGRSCKDLDECALGTHNCSEAETCHNIQGSFRCLRFDCPPNYVRVSETKCERTTCQDITECQTSPARITHYQLNFQTGLLVPAHIFRIGPAPAFAGDTISLTITKGNEEGYFVTRRLNAYTGVVSLQRSVLEPRDFALDVEMKLWRQGSVTTFLAKMYIFFTTFAP

SEQ ID NO: 225 - DNA, 2091 bp, Homo sapiens
  source 1..2091: mol_type = unassigned DNA
actcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattg

SEQ ID NO: 226 - DNA, 2051 bp, Mus musculus
  source 1..2051: mol_type = unassigned DNA
aagaccccgacacaccgtcccgagcgcggtttttcggctgtggtccagcccgtggccgtcggagtcgtcatgcagtccctgcgtaccacgctcctgtctttgctactgctgctgctagcggctccttccttggcgttgccgtcggggaccggccgctcggccccagctgccaccgtctgtcccgagcactgcgatcccacccgctgcgccccgccgcccacggactgcgagggtggccgcgtccgcgacgcgtgcggctgctgcgaggtgtgcggcgcgctcgagggtgcagcgtgcggcctgcaggagggtccctgcggcgaggggctgcaatgcgtagtgcccttcggggtgccggcctcggccacagtacgacggcgcgcacaggccggcctgtgcgtgtgtgccagcagcgagccggtgtgtggtagcgacgccaagacctacaccaacctgtgccagctgcgcgccgccagccgccgctccgagaagcttcgccagccgccggtcatcgtcctgcagcgcggcgcctgcggccaagggcaggaagatcccaacagtttgcgtcataagtacaactttattgctgatgtggtggagaagatcgccccggctgtggttcacattgaactatatcgcaagcttcctttctcgaagagggaggtgccagtggccagtgggtcaggattcatcgtatcggaggatggactgattgtgacaaatgctcacgtggtcaccaacaaaaaccgggtcaaggttgagctgaagaatggagctacctatgaagccaaaatcaaggatgtggatgaaaaggcggacattgcgcttatcaagattgaccaccagggaaagctgccagtcctgctgctcggccgctcctcagagctgagacctggagaatttgtagttgccattggaagccccttttctcttcaaaacacagtcaccactgggatcgtcagcaccacccagcgaggcggcaaagagctgggacttcggaactccgatatggactacattcagacagacgctatcatcaattatggaaattccggaggcccgttagtaaacctggatggcgaggtgattgggattaacaccttgaaggtgacggcgggcatctccttcgcaattccatccgataagataaaaaagttcttgacagagtcccacgatcgacaggccaaagggaaagctgtcaccaagaagaagtatattgggatccgaatgatgtcgctcacatctagcaaagccaaagagctgaaggaccgtcaccgagacttcccggatgtgctctctggggcatatatcattgaagtcattcctgacaccccggcagaagccggagggctcaaggaaaatgacgtcatcatcagcatcaacggacagtctgtggtcactgccaatgacgtcagcgatgtcatcaaaaaggagaacaccctgaacatggttgtccgcaggggcaatgaagacattgtgattaccgtgattcctgaagaaatcgacccctaggcagaggcaggagccagacttcatgtttccctcaaagactccaggggatggcgcatgagaactgagctggtgacgcaggacacccgggacctttgtccaacattttgcttgttcagggaacacccctgccgacagaatccttcttgatagtttgcgggcaaaacaaatgtaatgttgcaggtgcacaggcagaagtcctgccttctgtatgctatgtatgcagcgtgcttttccttacaagcttgggctgttcctgcttacacagtcaacatttgtcccttcccttagcctaagttgtctgactaatgcagttgacggatgcgtaggcaagaggaaggtcctcgcagccatggtcttgggcgtgtttacacttttcctccgagtcggcactcagaggaatttgatgccccgagaccacaggtgggtgaccctggcttctgagatggccaaaaatgcctcttaggaatcctggaacccggagtacagtgactttgagtttcagctattaaaatacttcttcatacgtaaaaaaaaaaaaa

SEQ ID NO: 227 - protein, 480 aa, Homo sapiens
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 228 - protein, 480 aa, Mus musculus
MQSLRTTLLSLLLLLLAAPSLALPSGTGRSAPAATVCPEHCDPTRCAPPPTDCEGGRVRDACGCCEVCGALEGAACGLQEGPCGEGLQCVVPFGVPASATVRRRAQAGLCVCASSEPVCGSDAKTYTNLCQLRAASRRSEKLRQPPVIVLQRGACGQGQEDPNSLRHKYNFIADVVEKIAPAVVHIELYRKLPFSKREVPVASGSGFIVSEDGLIVTNAHVVTNKNRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAVTKKKYIGIRMMSLTSSKAKELKDRHRDFPDVLSGAYIIEVIPDTPAEAGGLKENDVIISINGQSVVTANDVSDVIKKENTLNMVVRRGNEDIVITVIPEEIDP

SEQ ID NO: 229 - DNA, 4464 bp, Homo sapiens
  source 1..4464: mol_type = unassigned DNA
gcgcactccagccctgcagcctccggagtcagtgccgcgcgcccgccgccccgcgccttcctgctcgccgcacctccgggagccggggcgcacccagcccgcagcgccgcctccccgcccgcgccgcctccgaccgcaggccgagggccgccactggccggggggaccgggcagcagcttgcggccgcggagccgggcaacgctggggactgcgccttttgtccccggaggtccctggaagtttgcggcaggacgcgcgcggggaggcggcggaggcagccccgacgtcgcggagaacagggcgcagagccggcatgggcatcgggcgcagcgaggggggccgccgcggggcagccctgggcgtgctgctggcgctgggcgcggcgcttctggccgtgggctcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttccccgagggggacgtctgcatcgccatgacgccgcccaatgccaccgaagcctccaagccccaaggcacaacggtgtgtcctccctgtgacaacgagttgaaatctgaggccatcattgaacatctctgtgccagcgagtttgcactgaggatgaaaataaaagaagtgaaaaaagaaaatggcgacaagaagattgtccccaagaagaagaagcccctgaagttggggcccatcaagaagaaggacctgaagaagcttgtgctgtacctgaagaatggggctgactgtccctgccaccagctggacaacctcagccaccacttcctcatcatgggccgcaaggtgaagagccagtacttgctgacggccatccacaagtgggacaagaaaaacaaggagttcaaaaacttcatgaagaaaatgaaaaaccatgagtgccccacctttcagtccgtgtttaagtgattctcccgggggcagggtggggagggagcctcgggtggggtgggagcgggggggacagtgccccgggaacccggtgggtcacacacacgcactgcgcctgtcagtagtggacattgtaatccagtcggcttgttcttgcagcattcccgctcccttccctccatagccacgctccaaaccccagggtagccatggccgggtaaagcaagggccatttagattaggaaggtttttaagatccgcaatgtggagcagcagccactgcacaggaggaggtgacaaaccatttccaacagcaacacagccactaaaacacaaaaagggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatgtaatagtgacagcaaagggaccaggggagaggcattgccttctctgcccacagtctttccgtgtgattgtctttgaatctgaatcagccagtctcagatgccccaaagtttcggttcctatgagcccggggcatgatctgatccccaagacatgtggaggggcagcctgtgcctgcctttgtgtcagaaaaaggaaaccacagtgagcctgagagagacggcgattttcgggctgagaaggcagtagttttcaaaacacatagttaaaaaagaaacaaatgaaaaaaattttagaacagtccagcaaattgctagtcagggtgaattgtgaaattgggtgaagagcttaggattctaatctcatgttttttccttttcacatttttaaaagaacaatgacaaacacccacttatttttcaaggttttaaaacagtctacattgagcatttgaaaggtgtgctagaacaaggtctcctgatccgtccgaggctgcttcccagaggagcagctctccccaggcatttgccaagggaggcggatttccctggtagtgtagctgtgtggctttccttcctgaagagtccgtggttgccctagaacctaacaccccctagcaaaactcacagagctttccgtttttttctttcctgtaaagaaacatttcctttgaacttgattgcctatggatcaaagaaattcagaacagcctgcctgtccccccgcactttttacatatatttgtttcatttctgcagatggaaagttgacatgggtggggtgtccccatccagcgagagagtttcaaaagcaaaacatctctgcagtttttcccaagtaccctgagatacttcccaaagcccttatgtttaatcagcgatgtatataagccagttcacttagacaactttacccttcttgtccaatgtacaggaagtagttctaaaaaaaatgcatattaatttcttcccccaaagccggattcttaattctctgcaacactttgaggacatttatgattgtccctctgggccaatgcttatacccagtgaggatgctgcagtgaggctgtaaagtggccccctgcggccctagcctgacccggaggaaaggatggtagattctgttaactcttgaagactccagtatgaaaatcagcatgcccgcctagttacctaccggagagttatcctgataaattaacctctcacagttagtgatcctgtccttttaacaccttttttgtggggttctctctgacctttcatcgtaaagtgctggggaccttaagtgatttgcctgtaattttggatgattaaaaaatgtgtatatatattagctaattagaaatattctacttctctgttgtcaaactgaaattcagagcaagttcctgagtgcgtggatctgggtcttagttctggttgattcactcaagagttcagtgctcatacgtatctgctcattttgacaaagtgcctcatgcaaccgggccctctctctgcggcagagtccttagtggaggggtttacctggaacattagtagttaccacagaatacggaagagcaggtgactgtgctgtgcagctctctaaatgggaattctcaggtaggaagcaacagcttcagaaagagctcaaaataaattggaaatgtgaatcgcagctgtgggttttaccaccgtctgtctcagagtcccaggaccttgagtgtcattagttactttattgaaggttttagacccatagcagctttgtctctgtcacatcagcaatttcagaaccaaaagggaggctctctgtaggcacagagctgcactatcacgagcctttgtttttctccacaaagtatctaacaaaaccaatgtgcagactgattggcctggtcattggtctccgagagaggaggtttgcctgtgatttcctaattatcgctagggccaaggtgggatttgtaaagctttacaataatcattctggatagagtcctgggaggtccttggcagaactcagttaaatctttgaagaatatttgtagttatcttagaagatagcatgggaggtgaggattccaaaaacattttatttttaaaatatcctgtgtaacacttggctcttggtacctgtgggttagcatcaagttctccccagggtagaattcaatcagagctccagtttgcatttggatgtgtaaattacagtaatcccatttcccaaacctaaaatctgtttttctcatcagactctgagtaactggttgctgtgtcataacttcatagatgcaggaggctcaggtgatctgtttgaggagagcaccctaggcagcctgcagggaataacatactggccgttctgacctgttgccagcagatacacaggacatggatgaaattcccgtttcctctagtttcttcctgtagtactcctcttttagatcctaagtctcttacaaaagctttgaatactgtgaaaatgttttacattccatttcatttgtgttgtttttttaactgcattttaccagatgttttgatgttatcgcttatgttaatagtaattcccgtacgtgttcattttattttcatgctttttcagccatgtatcaatattcacttgactaaaatcactcaattaatcaa

SEQ ID NO: 230 - DNA, 4389 bp, Mus musculus
  source 1..4389: mol_type = unassigned DNA
attcgagctgcagcgtcgcaagtaggtgcagcagcccgcagcccgccgcgcctctgcgagccgggacagcactcggccccgcgcgctccccgccccgcgccagccccgccgcggcgacctgctgcagcggaggaccccatcgatcggagacccggggagcagcgcgcagccgccgagccggacggggcccggcactgcgcctttgtccccggaggctccgggaagtttgcagcgggacgcgcgcgtgaaggcagcgtgggcagccccgacgtcgccgagcaacatgggcgtcgggcgcagcgcgcggggtcgcggcggggccgcctcgggagtgctgctggcgttggccgccgctctgctggccgcgggttcggccagcgagtacgactacgtgagcttccagtccgacatcggctcgtatcagagcgggcgcttctacaccaagcccccgcagtgcgtggacatcccggtggacctgaggctgtgccacaacgtgggctacaagaagatggtgctgcccaacctgctggagcacgagaccatggcagaggtgaagcagcaggccagcagctgggtgccgctgctcaacaagaactgccacatgggcacccaggtcttcctctgttcgctcttcgcgcccgtctgtctggaccggcccatctacccgtgtcgctggctctgcgaggccgtgcgcgactcgtgcgagccggtcatgcagttcttcggcttctactggcccgagatgctcaaatgtgacaagttccccgagggcgacgtctgcatcgccatgaccccgcccaataccacggaagcctctaagccccaaggtacaaccgtgtgtcctccatgcgacaacgagttgaagtcagaggccatcattgaacatctctgtgcaagcgagtttgcactgaggatgaaaatcaaagaagtgaagaaggaaaacggtgacaagaagattgtccccaagaagaagaaacccttgaagctggggcccatcaagaagaaggagctgaagcggcttgtgctgttcctgaagaacggtgccgactgtccctgccaccagctggacaacctcagccacaactttctcatcatgggccgcaaggtgaagagccagtacctgctgacagccattcacaagtgggacaagaaaaacaaggagttcaaaaacttcatgaagagaatgaaaaaccacgagtgtcccaccttccagtctgtttttaagtgatactggggcggactggggaaggggagtgtggcttggggtgagggtgggggcgcgtggatgaccctggctcttgggggctcacatattgctctcacccattacagttgtggcttttgcattgcacctggctctgttcctacagcgaaccctctcccttccctccatagccacatccagctaaggccacggccctttagattaggaaggcttttttttttttaagggctgcagcagggccagcaggcacggtgcaaaaggagaggcagaatcctttcactgagcctcgggcacaaaaaacagaaaatgttggccggtttggaaaaacaaaacaaaacggattgtaaagaactgcagacggacagctgctcagctcaacgttgttcgggacatcattaccaattgcttgtggagtctaagcctctacaggtagaagagtctgatcattgccaagccaggctgctttcagtttattattaatccccctctttctgccttagatagaccatcgccaccttcaaaacacacacacacacacacacacacacacacacacacacacacacacacacacacacttctgaaagtagccagggtatcccagtatagaacgggatagctaagggtttgggtgggaggccactgcctactctaccttcagcttttgaactggccacctttgataggaaactgtaggtctcagatggacacttctaccagtccatcgggatacaaggatgccaggcaagggtctgcttttgtatgaaggaggtacgtgggcatgaagagacatgaggcatttcaggctgagaagccaacagctactagttttcaacaatagagtggaagaaatgagcaaaggtagaaatgtcaagcaggtcacaagtcagggtgattgggggaatcctgtgccaacagcctcactttgtaattccatctgtcacatttcaaaagaacagcagcataagacagggataaaagcccacataccctccaaggcttgagtaaaagtccacactcagcatttcaaagactaacgtcgttgactgcccaaggctgccctcttaatacaccgcctatgcatgtgctgtggaaggcaactctgtgcatgtgctgtggaggagatgggcctcatggctgtgcctggctgcccaggaatcagtatagctgtggaaggagacagtatccatagactctgcttttctgcaaggaaagcccttttccttatacatgattgcctataattcagacaaatttaaaatcgctgcctgcctgagccctccacctttacttttgcattcctccggtcatattcttttgaggctaaagtgccctatccgaggagatggtttcaaaggctaactaatctgcagctttcccaagtgcccagaggtatttctcaaagttggtatgcttaataagtgatgtaaatattccagttctcttaggcagccttactcctgttgtccctgtagttctgaaaaaaaaaaaaagttaatttcttccaccaaagtctgataattccccaccgcatccatggggctacagtgactgtccctttggcatactcaatccagaggtaggaatgattttgaagaatcatctagtggccttggattgacccagaaacaggaaaactctggttacttctgaaagactccagcctgaaaactcaccatgccaacttagttactgacctgagtggggtagtgacctaattaacttcatgtttagtagtcctattatcttaacccttttaagaatacttccaaccttaatcacaatgttatgaggaaccttaggtgatttgcctatcattgtggatgatttaaaaatatgtatgtgcattagtaattagaaatagtctaacctcattgtccataactgaaatgcaaagtctcctgagtgtgtagatctgggccttgagccactactagttcagtgcttggtgaggggtctgctcattttgggaaagtgtatcatgtggcagggccctctccgagtggcgggagactgcctacatgatttttttttttccctggggatgttagcagtttcacagcataaaagggaggacatgttctggggggctccagattgggaaccctcaggaagtaacttcctcaggaaggattcataaccaattgatcgatcaattgatcatgtgaactgcagcagctgtctgtccccttacttcagagtccccagcttcagagttttgttgcggacctgtgaggatttcagacacacacacacacacacacagaccagttttgtctatactacatcagcagttccagacaaaaagggaggctatctaggggcccgaaagctgcatgatcagcagcctttgtccttttcaacaaaaggtgtaacaaaccaatgtgcagactcattagctttgtccctggcttccaatggaggtggtccatgtgcgctctgaccagcgttgccaatgaaaaggtcagatttggaaagctttaaaatagtcattccggagagtcagggaaagctaaacccaccaaacttttgggaactatcaatagctatcttagaaaatagacttgagaggcaagaattgtaagaatatgtttgctttttaaaacattctgtatcctcagagctcatggttggtgggttagcactaggtcctccctggggctgagtctaagcccagttccaggcttcctaagaatgtgtaaattacagaaatcacatttcccaaagctagaacctgttttatcttgttagcctccctgtaaccagatgctctgttggaccttcatagccggacgtggttcaagatgtgctccagaagcagacccggggtcacatctccaggatggcatgttggctgctctgacctggcgctgtgggtcctaagcgcagagacggaattcctgttggccctggttcccccctcctcgcaccccctgaggactccactttatagcctaagccttttatacaaaagctttgaatactgtgaagatgttttacattccttctcattcctgttgtttcttaactacatttgaccagatgttttgatgttatcacgtatgttaatagtgattcccaggtgtgttttgttttcatgctttccctgccaatgtattcacttcactaaaatcactcgatcaataaaaaaaaaaaaaaaaaaa

SEQ ID NO: 231 - protein, 314 aa, Homo sapiens
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 232 - protein, 314 aa, Mus musculus
MGVGRSARGRGGAASGVLLALAAALLAAGSASEYDYVSFQSDIGSYQSGRFYTKPPQCVDIPVDLRLCHNVGYKKMVLPNLLEHETMAEVKQQASSWVPLLNKNCHMGTQVFLCSLFAPVCLDRPIYPCRWLCEAVRDSCEPVMQFFGFYWPEMLKCDKFPEGDVCIAMTPPNTTEASKPQGTTVCPPCDNELKSEAIIEHLCASEFALRMKIKEVKKENGDKKIVPKKKKPLKLGPIKKKELKRLVLFLKNGADCPCHQLDNLSHNFLIMGRKVKSQYLLTAIHKWDKKNKEFKNFMKRMKNHECPTFQSVFK

SEQ ID NO: 233 - DNA, 1719 bp, Homo sapiens
  source 1..1719: mol_type = unassigned DNA
cttctcccgagcgagctccgcaggagacacaggcgctggctgccccgtccgctctccgcctccgccgcgccctcctcgcccgggatgggcccccccgccgccgccggatccctcgcctcccggccgccgccgttgcgctcgccgcgctcgcactgaagcccgggccctcgcgcgccgcggttcgccccgcagcctcgccccctgcccacccgggcggccgtagggcggtcacgatgctgccgcccttaccctcccgcctcgggctgctgctgctgctgctcctgtgcccggcgcacgtcggcggactgtggtgggctgtgggcagccccttggttatggaccctaccagcatctgcaggaaggcacggcggctggccgggcggcaggccgagttgtgccaggctgagccggaagtggtggcagagctagctcggggcgcccggctcggggtgcgagagtgccagttccagttccgcttccgccgctggaattgctccagccacagcaaggcctttggacgcatcctgcaacaggacattcgggagacggccttcgtgttcgccatcactgcggccggcgccagccacgccgtcacgcaggcctgttctatgggcgagctgctgcagtgcggctgccaggcgccccgcgggcgggcccctccccggccctccggcctgcccggcacccccggaccccctggccccgcgggctccccggaaggcagcgccgcctgggagtggggaggctgcggcgacgacgtggacttcggggacgagaagtcgaggctctttatggacgcgcggcacaagcggggacgcggagacatccgcgcgttggtgcaactgcacaacaacgaggcgggcaggctggccgtgcggagccacacgcgcaccgagtgcaaatgccacgggctgtcgggatcatgcgcgctgcgcacctgctggcagaagctgcctccatttcgcgaggtgggcgcgcggctgctggagcgcttccacggcgcctcacgcgtcatgggcaccaacgacggcaaggccctgctgcccgccgtccgcacgctcaagccgccgggccgagcggacctcctctacgccgccgattcgcccgacttctgcgcccccaaccgacgcaccggctcccccggcacgcgcggtcgcgcctgcaatagcagcgccccggacctcagcggctgcgacctgctgtgctgcggccgcgggcaccgccaggagagcgtgcagctcgaagagaactgcctgtgccgcttccactggtgctgcgtagtacagtgccaccgctgccgtgtgcgcaaggagctcagcctctgcctgtgacccgccgcccggccgctagactgacttcgcgcagcggtggctcgcacctgtgggacctcagggcaccggcaccgggcgcctctcgccgctcgagcccagcctctccctgccaaagcccaactcccagggctctggaaatggtgaggcgaggggcttgagaggaacgcccacccacgaaggcccagggcgccagacggccccgaaaaggcgctcggggagcgtttaaaggacactgtacaggccctccctccccttggcctctaggaggaaacagttttttagactggaaaaaagccagtctaaaggcctctggatactgggctccccagaactgctggccacaggatggtgggtgaggttagtatcaataaagatatttaaaccacca

SEQ ID NO: 234 - DNA, 2068 bp, Mus musculus
  source 1..2068: mol_type = unassigned DNA
gatttcttctctccagcgagctccgcaggagacacaggcgctggctgctgggtccgctctccgcctccgccgcgccctcctcgcccgggatgggcccccccgccgccaccgccgccggagccctagtctccgggccgccgcctcggtcgccgcgtttgccctgaagcccggtgcccgcgcgccccggctcaccccgcagcttcactccccacccccagccgcctccccggccagactgcggtagagctctcaggatgctgccgccggtgccctcccgcctcggactgctgctgctgctcttgtgccccgcgcacgtcgatggactgtggtgggccgtgggcagccccttggtcatggatcctaccagcatctgcaggaaggccaggcggctggcaggaagacaggccgagctgtgccaggcggagccggaagtagtggcagagcttgcccgaggcgcaagactgggggttcgagaatgtcagttccagttccgtttccgacgctggaactgctccagccacagcaaggcctttgggcgcgtcctgcagcaggacatccgagagacagctttcgtgtttgcaatcaccgcagctggtgccagccacgcggtcactcaagcctgttccatgggagagctcctacagtgtggttgtcaggcaccccgcgggcgggcaccgcctaggccctccggccttctgggcactcctggacctccaggaccaactggctctccagatgctagcgcagcctgggagtggggaggctgcggagacgatgtggacttcggggatgagaagtcaagactctttatggatgcgcagcacaagcggggccgtggagatatccgtgcattggtgcaactgcacaacaacgaggcgggcaggctggcggtgcggagtcacacgcgcaccgagtgtaagtgccatgggctttcgggttcctgcgctctgcgcacctgctggcagaagctgcctccgttccgcgaggtgggcgcacggctgctggagcgcttccacggcgcctcgcgcgtcatgggcaccaacgacggcaaagctctgctgcctgcggtccgcacactcaagcctcccggacgagcggatctcctctacgcagccgattcacccgacttctgcgcccctaaccggcgcacgggttcgccgggcacgcgcggacgcgcctgcaacagcagtgccccggacctcagcggctgcgacctgttgtgctgcggtcgcgggcaccgccaggagagcgtacagctcgaggagaactgtctgtgccgcttccactggtgctgcgtggtgcaatgccaccgctgccgggtgcgcaaggaactcagcctgtgcctctgacccgtcgcctgcctcggaactgctggcagccacctctgggccatctacaggactattagattccagcagggggcgctgtctgagtccagcagctccctaggaaaagtacctatccaggccttgggaaattacaggggccagccaggaacttggggtttacaccagcccacgaaagcccgggggaacattcccctgaaaggccctttgctagttcctgcaggagatcactccccttggccccccagatggaaataaaaaagccagactctgccctctggaaataatattcctcagaattactgggatggatgggtgagtttagtatcaataaagacatttaaatccacagtttgagaggaggtagtcctagagggccaggatatggaagaaagttggtcctggtagagttagcatttcagtctgagtctatcattccacctgttaccaaggcatgacggagtgagggaggacttatggaaaggcagaaaagagggtgagggagggctcctggaaacctttagcacaccttctgttctttcttggctctcgagaacttgtggtcccatgttaaaggaacacaaagaggccagaagcttctagtagtgggcttagttgtcaggcgtctgtgtgaacttttatggcattctgtctttctgtgactcctgatgaaattaacggtcatgagattaaaagctcgtcatgaactgtaaaatgcaacg

SEQ ID NO: 235 - protein, 365 aa, Homo sapiens
MLPPLPSRLGLLLLLLLCPAHVGGLWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 236 - protein, 364 aa, Mus musculus
MLPPVPSRLGLLLLLLCPAHVDGLWWAVGSPLVMDPTSICRKARRLAGRQAELCQAEPEVVAELARGARLGVRECQFQFRFRRWNCSSHSKAFGRVLQQDIRETAFVFAITAAGASHAVTQACSMGELLQCGCQAPRGRAPPRPSGLLGTPGPPGPTGSPDASAAWEWGGCGDDVDFGDEKSRLFMDAQHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 237 - DNA, 8352 bp, Homo sapiens
  source 1..8352: mol_type = unassigned DNA
agaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcctctcctcattcctttggttttggcccaaatgattatgtttcctctttttgggaagatttctctgggtattttgatatttgtcctgatagaaggagactttccatcattaacagcacaaacctacttatctatagaggagatccaagaacccaagagtgcagtttcttttctcctgcctgaagaatcaacagacctttctctagctaccaaaaagaaacagcctctggaccgcagagaaactgaaagacagtggttaatcagaaggcggagatctattctgtttcctaatggagtgaaaatctgcccagatgaaagtgttgcagaggctgtggcaaatcatgtgaagtattttaaagtccgagtgtgtcaggaagctgtctgggaagccttcaggactttttgggatcgacttcctgggcgtgaggaatatcattactggatgaatttgtgtgaggatggagtcacaagtatatttgaaatgggcacaaattttagtgaatctgtggaacatagaagcttaatcatgaagaaactgacttatgcaaaggaaactgtaagcagctctgaactgtcttctccagttcctgttggtgatacttcaacattgggagacactactctcagtgttccacatccagaggtggacgcctatgaaggtgcctcagagagcagcttggaaaggccagaggagagtattagcaatgaaattgagaatgtgatagaagaagccacaaaaccagcaggtgaacagattgcagaattcagtatccaccttttggggaagcagtacagggaagaactacaggattcctccagctttcaccaccagcaccttgaagaagaatttatttcagaggttgaaaatgcatttactgggttaccaggctacaaggaaattcgtgtacttgaatttaggtcccccaaggaaaatgacagtggcgtagatgtttactatgcagttaccttcaatggtgaggccatcagcaataccacctgggacctcattagccttcactccaacaaggtggaaaaccatggccttgtggaactggatgataaacccactgttgtttatacaatcagtaacttcagagattatattgctgagacattgcagcagaattttttgctggggaactcttccttgaatccagatcctgattccctgcagcttatcaatgtgagaggagttttgcgtcaccaaactgaagatctagtttggaacacccaaagttcaagtcttcaggcaacgccgtcatctattctggataatacctttcaagctgcatggccctcagcagatgaatccatcaccagcagtattccaccacttgatttcagctctggtcctccctcagccactggcagggaactctggtcagaaagtcctttgggtgatttagtgtctacacacaaattagcctttccctcgaagatgggcctcagctcttccccagaggttttagaggttagcagcttgactcttcattctgtcaccccggcagtgcttcagactggcttgcctgtggcttctgaggaaaggacttctggatctcacttggtagaagatggattagccaatgttgaagagtcagaagattttctttctattgattcattgccttcaagttcattcactcaacctgtgccaaaagaaacaataccatccatggaagactctgatgtgtccttaacatcttcaccatatctgacctcttctataccttttggcttggactccttgacctccaaagtcaaagaccaattaaaagtgagccctttcctgccagatgcatccatggaaaaagagttaatatttgacggtggtttaggttcagggtctgggcaaaaggtagatctgattacttggccatggagtgagacttcatcagagaagagcgctgaaccactgtccaagccgtggcttgaagatgatgattcacttttgccagctgagattgaagacaagaaactagttttagttgacaaaatggattccacagaccaaattagtaagcactcaaaatatgaacatgatgacagatccacacactttccagaggaagagcctcttagtgggcctgctgtgcccatcttcgcagatactgcagctgaatctgcgtctctaaccctccccaagcacatatcagaagtacctggtgttgatgattactcagttaccaaagcacctcttatactgacatctgtagcaatctctgcctctactgataaatcagatcaggcagatgccatcctaagggaggatatggaacaaattactgagtcatccaactatgaatggtttgacagtgaggtttcaatggtaaagccagatatgcaaactttgtggactatattgccagaatcagagagagtttggacaagaacttcttccctagagaaattgtccagagacatattggcaagtacaccacagagtgctgacaggctctggttatctgtgacacagtctaccaaattgcctccaaccacaatctccaccctgctagaggatgaagtaattatgggtgtacaggatatttcgttagaactggaccggataggcacagattactatcagcctgagcaagtccaagagcaaaatggcaaggttggtagttatgtggaaatgtcaacaagtgttcactccacagagatggttagtgtggcttggcccacagaaggaggagatgacttgagttatacccagacttcaggagctttggtggttttcttcagcctccgagtgactaacatgatgttttcagaagatctgtttaataaaaactccttggagtataaagccctggagcaaagattcttagaattgctggttccctatctccagtcaaatctcacggggttccagaacttagaaatcctcaacttcagaaatggcagcattgtggtgaacagtcgaatgaagtttgccaattctgtccctcctaacgtcaacaatgcggtgtacatgattctggaagacttttgtaccactgcctacaataccatgaacttggctattgataaatactctcttgatgtggaatcaggtgatgaagccaacccttgcaagtttcaggcctgtaatgaattttcagagtgtctggtcaacccctggagtggagaagcaaagtgcagatgcttccctggatacctgagtgtggaagaacggccctgtcagagtctctgtgacctacagcctgacttctgcttgaatgatggaaagtgtgacattatgcctgggcacggggccatttgtaggtgccgggtgggtgagaactggtggtaccgaggcaagcactgtgaggaatttgtgtctgagcccgtgatcataggcatcactattgcctccgtggttggacttcttgtcatcttttctgctatcatctacttcttcatcaggactctccaagcacaccatgacaggagtgaaagagagagtcccttcagtggctccagcaggcagcctgacagcctctcatctattgagaatgctgtgaagtacaaccccgtgtatgaaagtcacagggctggatgtgagaagtatgagggaccctatcctcagcatcccttctacagctctgctagcggagacgtgattggtgggctgagcagagaagaaatcagacagatgtatgagagcagtgagctttccagagaggaaattcaagagagaatgagagttttggaactgtatgccaatgatcctgagtttgcagcttttgtgagagagcaacaagtggaagaggtttaaccaaaactcctgttctgaaactgattagaagcctggagaagatggagattacttgttacttatgtcatataattaacctggattttaaacactgttggaagaagagttttctatgaaaaaattaaatatagggcacactgtttttttttcagcttaagttttcagaatgtagtaagagatgttaccatttttatttctataaagactgaatgctgtgtttaaataaattgaaaactacgtaaactctggaaagtattctataagaaaaagctggacctaggatttagggctgcagttgctgtctgcttctgaatcactgggggtgtctcctaagaactgtgcctcgggtttttcaataggaaaataaggtgagactcttccatggagaagtctggcttagtaagggaatattttggagcatatttgttattgttgggtagaaggggaaacctttgaaatagtcttaatggatagtttataatcaggcattcatctaaataaaattctttcttttttttgatcaaactgctaaagaaaaagcaaacttttttgctgcaggatagaattcgggttgtatttttatgcttctatcagtgtcttgcatttcctgagcatattaatcaagaccatgttattgttatgttaactatgttaatatcctttttttcttagttttcaggatgaatgcatatatatatatatatatatatatatatatatatatatatatatatatatatataaagtttagaagattcccattaatctaaagacaaatttaatcacatgggcagggagggagtgagtgtcgggtcaagaccccagtgctgagaatatattttgtttcttgattgctggactgaggttccctgattgcttctgaaatctaaagatgtcttgggcaaatcctgtctctcctctagttcctggtgtattaatttggaaaacagcacttaactacctcacataatatagatgagctcattttcattgcttctcctttgtgtaatacctcctgtctaaattaactcctgcttatacaaaacagggcaaagcttttattacaattaaaatattttaaaaactgaacagataggaagtccataccttatataaattttgacagaaaatggaaattttcctctttccccctagtgttttttttgaatgggttgctttggagggggagaggaggaggccaaatcatcatcatcatgatattaaattgcatatgcaataggttattattaatgcagatactttatgctagatgtcttcatgcttgccaaaaccaattctgaacattttgatttatttctgaccagatataaataaccagtataaactgaccataagataaaatacttggttcacttttttttttctcaacttgatgactgccaattctgctcattatgaggacaccggaaactaaatatctgggctcagtgcatgccacagtaaagctagtgaaagccttgaagatggccaggtattttgcaaagtatgtatggataacaggttgtgtgtgtgtatgtgtgaatgtgtgtgtgcgtgtatgtgtgtgtgcgtgtgcgcgctaatgcgcatgtgcgcccgcgccaaggtggctcccttatcctagcatttcctttcattccctactcacataatggatcctctaacagttcagtataggtacactttccactattttattgacatgttctagtcttaatattggataattagacactttccactgggcccatttgaaaagtaattgataattacctttaagacattttaagtgttaatgatatttgtcactactaagtgcttagttttatttataaaactttcaaagttttctgtttaagactgtaaactgttttcctatactacgtgttagatgaaagtgtattttatcacaaattaaccttagaatgcaactctttgaatgtgtgcttgctggaagaattccagatggacacaaatccctctagggcaggtcaccatgcagaacacttaggagaagcacgaactgagccagaggagggttgttgttttccacaaactaagcagtgaagtgggaatttgggaattctgacacacgtcaccatctctgatttccttttgcgtgaccttttcatgcaagagcaaaacacttagctcttcatgaatgtctgctccacaccaatgaaatggaagagtaaaggagcccctcaactatcagggaaagaacaggaggcctcagttgaggaatacaacttttgtcctgcctttgctatagctgaaccactgttgaaaggttgaagaacacagtttgcccagcaaagatccatccactcatgtacaaagcaaagcactaaaggagctattagtttgatggaaaaaactacccctaaagagcttctttctaaaagaccttcctcgaaggtgatttaatggcatgtcatcatttaaaagtcttaggctgaactgtcatttacactgcacaattgaggaatgaaattttctgagtattagaacgttcttcttaacctagcaatataatattcaacaagactatcaccttatgtgaaaatactaaaatgcagttagcaataaaatgcatggtaaataaccattttaaaaatgttattataaaaggttgtaagatactgtggtgttttacagatgtagcctttataattgaattatatacactatccttttttatgggttatggataaaggggaatttatttgataaatagggctccattttcaccattgcaatggtttaagggtgtttgttcacgtttatgtggcctgaagtcccatttttatcactattattatttgagatggagtctcgctctgtcacccaggctggagtgcaatggggcgatctcagctcctggcaacctctgcctcctgggttcaagcgattctcgtgcttcagcttcctgagtagctggggttacagacacccaccaccatgcccagctaatttttatatttttagtagagatggggtttcaccgtcaccatgttggccagcctggtctcaaactcctgacctcaggtgatccacctgccttggcctcccaaagtgctgggattacaggcatgagccaccgcacctggccccatttaatttatataataaaacaagtgcaggctttacctgattttattctcaccttctgttttcttcttctgcattatctgtcaccatgggttggctcctccccactttctcttttttattcttttctctcttttttgccacactaaattgtaatgtgtttgaacaactctgacttaatattacctctaacaaaaagctggaaactgcacaaatattctttgtaggctctaaaagtaggttggctgaagtatgatacctgaactaattattcatttgtatgcttgttttataagtttgacttccatttcatgatttttttggtagctttatgttatttctgttttcagcaatgggtactttgacatctttctgtctctgaattgaattttcattttgcaacggaagagaagacaacatatttattccataatgaaagagctaatgaagtagaataactattaaattgagatgttttagcaaaaaaacaaaaacacaaaccccaaacgggttgttttgttttatttttttctaaaacatttctaacaattcccagtaggaatgggtggggccagccttcctcatttacaatccagctgcagtgagtcaagatcctagcttattctcccaattgtgtaataaatgcagaaaaatgatgtgacggtcactattctcaaatccattgcattgtattccacaaaggatatcctggcggaaggtggaaatatgggagggtaccatgatgtaaagaattgcctggaaatttccttgagtcatgcattttgtaaaaccttacattactgctatgagttaataagagacaggttaaaccactcccctcaggatagtaagactggttaaaaacactgctctcagcccacacagttatagctacagcctaggttaaatgattcctcatcctggtgtttttattctcatgtttaattacttctttaaagattaagattatatatagaaaaaataattgtggtatcagagaatacaggtataatttttcatatatcaggactatgtgatgatacttaagcaatagtgtacatacagtaatatatgtttaatatcgagtatggataatgaaggacttttctttttcaccttgtttataatgacattttatccagggtttgaatcaaataaattcatgttaactgtactattttattgaaatgttctagtcttattattggataattagaaataaacattttaaagtctttatgaataaataaggatgttttcctatgtatacaactgtactattttcattagtaataaacatcactttcaagaa

SEQ ID NO: 238 - DNA, 6992 bp, Mus musculus
  source 1..6992: mol_type = unassigned DNA
atagtgtccctattaagtcagaaaagaaaccagagcttctcggaaaggatgacattttatttacgctaactgacaatggttttagagtggcttcccactcccaggcataaacttttatctgcagggactttttttgggaaaagtttgttagggagcattttagaactccgaatgctcccttgaggattttgtcatctgttttctttggttttggcccagatgattatgtttctccctgtggggaggatgtctctgggcattttgatacttttcctgacaggaggaaaccttgtatcagcctcagaagagagacaagaacccatgcatgccgtctctgtcctctcacctgagaagtcaacagacctttccctgcctaccaggaagaggcagcttttggatgccacggagactgggaggaggtggctgcttagaaggcggcgatctattctgttcccaaatggagtaaaaatttgctccagtgaaaccgttgcagaggctgtggcaaaccatgtgaagtattttaaagcccgagtgtgccaggaagccatctgggaagccttcaggacgttttgggatcgacttcctgggcgtgatgaatatcgtcactggatgaatttatgtgaggatggagtcacaagtgtatttgaaatgggcgcccattttagtcagtctgtggaacatagaaacctaatcatgaagaaactggcttacacaagggaagctgagagcagctcctgcaaggatcagtcctgtgggcctgagttgtcctttccagttcctattggtgagacctcaacactgacaggtgctgtctccagtgcttcctatccagggttggcttcggagagcagcgcagcgtcaccgcaggagagtatcagcaatgaaattgagaatgtgacagaggagcccacacaaccagctgctgaacagattgcggaattcagcatccaacttctggggaagcgatacagtgaagaactgcgggatccctccagcgccctctaccggctcctcgtggaagagtttatttcagaggttgaaaaagcattcacagggttacctggctacaagggcatccgtgttctggaattcagggccccggaggaaaatgacagtgggatagatgttcactatgcagttaccttcaatggcgaagccatcagcaataccacctgggacctcataagccttcactccaacaaggtagaaaaccatggccttgtagagatggatgataaacccactgctgtctatacaattagtaacttcagagattatatcgctgagacgctgcaccagaactttttgatgggaaattcctctttgaatccagatcccaagtctctccagctcatcaatgtgagaggagttttgctcccccaaacagaagacatagtttggaacacccaaagttcaagtcttcaggtgacaacatcctctattttggataatacccttcaagctgaatggctctcagcagataccaccaccaccaccaccaccaccatttcaccatttggtttcagctccagttctccctcagccactggcagggaactccagtcacaaagtgctttgcgtgacgtagtgtctacctccaagttagcttctcccacgaaggtggtcctcagttctctcccagagattttagggggtagcagcttgactcttcattctgttaccccagcagtgcttcagcctgacctgcctgtggctcctgagggaaggacttctggatcgttcatattagaagatgggttagccagcactgaagaattagaagatacttctattgatggattgccttcaagcccattaattcaacctgtgccaaaagaaacagtaccacctatggaagactctgacacggctctcttgtccacaccacatctgacctcttctgctatagaagaccttactaaagacatagggacaccttctggcttggagtccttggcttccaacatctcagaccagttggaagtgatcccatggtttccagacacctctgtggaaaaagacttcatttttgaaagtggcttgggttctgggtctgggaaagatgtagatgtgattgattggccatggagtgagacttcattagagaagaccactaaaccactgtcaaagtcatggtctgaagaacaggatgcactattaccaactgagggtagagaaaaattacatatagatggcagagtagattccacagaacaaattattgaatcatcagaacatagatatggagataggcccatacattttatagaggaagaatcccatgttagatctactatacccatctttgtagagtccgcaactccacctacatctccaatcttttcaaaacacacttcagatgtaccagacattgattcttactcacttaccaaaccacccttcttaccggtaactatagcaatccctgcttccactaagaaaacagatgaggtactcaaggaagatatggtacatacagaatcatccagtcacaaagaacttgacagtgaggttccagtgtcaaggccagatatgcagcctgtgtggaccatgttgccagaatcagatacagtttggacaagaacttcttccttagggaaattgtccagagacacattggcaagtacaccagagagcactgacagactctggttgaaagcttccatgacacagtccactgaattgccttcaaccacccactccacccagctagaggaggaagtaataatggcggtccaggatatttcattagaactagatcaggtaggcacagattattatcagtccgagctaactgaagaacaacatggcaaggctgacagctatgtggaaatgtctaccagtgttcactacacagagatgcctattgtggctctgcccacaaaaggaggtgtcttgagtcacacccagactgcaggagcattggtggttttcttcagcctccgcgtgacaaacatgttgttttcagaagacttgtttaacaaaaactctttggaatataaagccctggaacaaagattcttagaactgctggttccctatctccagtcaaatctgtcagggttccagaacctagaaatcctgagtttcagaaacggcagcattgtggtgaacagccgagtgaggttcgccgagtctgcccctcctaatgtcaacaaggccatgtataggattctggaagacttttgtaccactgcctaccaaaccatgaacttggatatcgataagtactccctggacgtggaatcaggtgatgaggccaacccttgcaagtttcaggcctgtaatgaattttctgagtgtttggtaaatccatggagtggagaagcaaagtgcaaatgctaccctgggtacctgagtgtggatgaactgccttgtcaaagtctctgtgatctacagcctgacttctgcttgaacgatggaaagtgtgacattatgcctgggcatggagccatttgtagatgccgggttggttcaaactggtggtatcgaggccaacactgtgaggagtttgtgtctgagccctttgtcataggcatcactatagcctctgtggttagctttctccttgttgcttctgctgtcgtcttcttccttgtgaagatgcttcaagctcagaatgtcaggagagaaaggcagaggcccaccagctccagcaggcaccctgacagtctgtcatctgttgagaatgctatgaagtataaccctgcatatgagagccacttggctggatgtgaactgtatgagaaatcctatagccaacatcccttctatagctctgctagtgaagaggtgattggtggtctgagcagagaagaaatcagacagatgtatgaaagtagcgacctttccaaagaggaaattcaagagagaatgaggattttggaactctatgctaatgatcctgagtttgcagcttttgtgagagagcatcaaatggaggagctttaacttaaatgcctgattcttgacaccaatcagaagcttggaggagatggagaaggcttgttctctctgctgtttaactaatccagaagaagagtttgtattgaagaataaataaggaaacatgggacgcacttctcattccaacactgcagcttaattttttggaatggagcaaaaaaaaaataagtgatgtattttatttctacattaagagatgtgtcaaaagaaaattaaagtggtgtgaactctgattttgtaacatattctaaaagcaaacaaataaaacagaaccaaaccaaaagcttaaagccagaccttggagttggggctgcagtgcctctgactctgactttttgagagcatctctaagaactatggcccaggctttctagtaagaacataaagtgagactaatgagtaaagcttagaatgcgactgttttgtgacatacttgttaaagtcgaatgagatagaggaagctttgaagtaattttaatatagtttaaactcaaacactcatctaaataaaaattaggcttttggaacagattgctgagtcaggcaatctttaggtgcagtatattttgtttatgtttgatgcttgcttctatctgttcttgagcttcttgagcccatagatcaagactacaatgctcttaaattagttatgtcaatatttgccacagtttggtcctcaattaggcacccttaagaggaagcaaattgaggaattattttcatcagcttggcttgtggacataccagtgggcctttttcttgattattaattgatgtagaaaggcccagctcactatgggtggtactatccttaggcaggggtttggggagttaagttgcaaaagaaaggtaaagccagctacaagaagccagccaataagcactttcctttgtggtttcttcttcaaactcctgtcttggcttctctctatggtagactataacctataagccaaataaactctttctttctgtcctagttagggtttccattgttgtgaccaaggcaactcttttaaggacaacatttaatcgaggctggcttgtagattgaggttcagtccattatcatcaaagctggaagcatggcagcatccaggcagtcaaggcactggaggagctgagagttcttcatcttcatctaaaggaagccaggagcagactagctttcaggcagctaggatgagggtcttaaaacacatgcccacagtgacacacctactctaacaaggccacacctactccaacaaggccatactttcaaatagtgccactccctgggccaaacatatttacaccaccataccttctcaaaggtgcttttgcttagtgataacagagaaagcaaatgattataataccctattctttaagctttcaggaatagtacccatgcgtgaagttttcatggtttctatttttcttatgcaaaagtaattatggggctgtaagggtctgagtgttaggtaaataccaggacagaccctctggtttttagatgggctgacaggcaaagttattacttagatatgttccctgactgcttctgaggtaggacagtgtagtggacaattctaggacctactgtagttcctggtgtaggaattttgaaaacagcacatacctacctcacataatgtcagtgatatcatttgttgcttctttgtctaaagcttgttgtctaggtgaaatcccacctaccataacaggacatttttaaaattgtgaatctatactttataaatagaagttaaaaatggaagtttttctctttttacctatggttattttgaatggataatttgggtgataggctgtaaaggcgaattaatcatgatgaatagtttaaagatacataggcactagatgattgttaattcaagctatttctgaggacaccttcatgtttgcaacaaatctcttgaagttcattagttatttcagaccaagtataaaatggcagcaggataaaatacctggttcatttatttttcttaaatcagggactgtaagtttcatagttatgaggtcagcaaaaactggagttggaagaatagacaacttacttagaagaaaacagtgatttaggagactaacttacatttattaaattgggaagttttagccaagaaaaatgtgggtcagtttccccccaccctctcaaatgtttccaatgattccaagtaaagataggtggggccagccttcattagtaacccagctgcagtgaatcaggattctcgatcattctattccctagtgtgtgatcaatgaagaaaaatgatgtaaaggttacagtccttaaactgagagcattgtgtacgcaaagtaccctgcccagaggtggggagatgggaggatattataacatagttggtgatgtaaaaaaaaaattacctggaaatttctttgattcatgcattttgtaatgtgtagactgttctaataagtcaacaagagacaagtgaagccactccccactggatactaagccactgactaaaacacgtctctcaactacatagttactattacagctcaggccaaatgattagtaatcctgattttttctcttgtatttaattaattaagggcttaattgggggaaaaatcatggtataagagactacatgtaaaaaaaaagagactacatgtataaatatatgtctggactacttgatgattcctaagcagtaatgtatatatcctaatatatacttaatatggagtatggttaatggcatgctattcttttttatttaatttatggcagtttatccaatgtggggtcaaacaaagtcaatttactatactactctcttgaacatttctagtcataatattagttgattagaaatacaaatttaagtctttatgtatgtataaggatccttcgattcacatgcatactgtgatgtatccattagtaataaacatgtctttcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 239 - protein, 1241 aa, Homo sapiens
MIMFPLFGKISLGILIFVLIEGDFPSLTAQTYLSIEEIQEPKSAVSFLLPEESTDLSLATKKKQPLDRRETERQWLIRRRRSILFPNGVKICPDESVAEAVANHVKYFKVRVCQEAVWEAFRTFWDRLPGREEYHYWMNLCEDGVTSIFEMGTNFSESVEHRSLIMKKLTYAKETVSSSELSSPVPVGDTSTLGDTTLSVPHPEVDAYEGASESSLERPEESISNEIENVIEEATKPAGEQIAEFSIHLLGKQYREELQDSSSFHHQHLEEEFISEVENAFTGLPGYKEIRVLEFRSPKENDSGVDVYYAVTFNGEAISNTTWDLISLHSNKVENHGLVELDDKPTVVYTISNFRDYIAETLQQNFLLGNSSLNPDPDSLQLINVRGVLRHQTEDLVWNTQSSSLQATPSSILDNTFQAAWPSADESITSSIPPLDFSSGPPSATGRELWSESPLGDLVSTHKLAFPSKMGLSSSPEVLEVSSLTLHSVTPAVLQTGLPVASEERTSGSHLVEDGLANVEESEDFLSIDSLPSSSFTQPVPKETIPSMEDSDVSLTSSPYLTSSIPFGLDSLTSKVKDQLKVSPFLPDASMEKELIFDGGLGSGSGQKVDLITWPWSETSSEKSAEPLSKPWLEDDDSLLPAEIEDKKLVLVDKMDSTDQISKHSKYEHDDRSTHFPEEEPLSGPAVPIFADTAAESASLTLPKHISEVPGVDDYSVTKAPLILTSVAISASTDKSDQADAILREDMEQITESSNYEWFDSEVSMVKPDMQTLWTILPESERVWTRTSSLEKLSRDILASTPQSADRLWLSVTQSTKLPPTTISTLLEDEVIMGVQDISLELDRIGTDYYQPEQVQEQNGKVGSYVEMSTSVHSTEMVSVAWPTEGGDDLSYTQTSGALVVFFSLRVTNMMFSEDLFNKNSLEYKALEQRFLELLVPYLQSNLTGFQNLEILNFRNGSIVVNSRMKFANSVPPNVNNAVYMILEDFCTTAYNTMNLAIDKYSLDVESGDEANPCKFQACNEFSECLVNPWSGEAKCRCFPGYLSVEERPCQSLCDLQPDFCLNDGKCDIMPGHGAICRCRVGENWWYRGKHCEEFVSEPVIIGITIASVVGLLVIFSAIIYFFIRTLQAHHDRSERESPFSGSSRQPDSLSSIENAVKYNPVYESHRAGCEKYEGPYPQHPFYSSASGDVIGGLSREEIRQMYESSELSREEIQERMRVLELYANDPEFAAFVREQQVEEV

SEQ ID NO: 240 - protein, 1243 aa, Mus musculus
MIMFLPVGRMSLGILILFLTGGNLVSASEERQEPMHAVSVLSPEKSTDLSLPTRKRQLLDATETGRRWLLRRRRSILFPNGVKICSSETVAEAVANHVKYFKARVCQEAIWEAFRTFWDRLPGRDEYRHWMNLCEDGVTSVFEMGAHFSQSVEHRNLIMKKLAYTREAESSSCKDQSCGPELSFPVPIGETSTLTGAVSSASYPGLASESSAASPQESISNEIENVTEEPTQPAAEQIAEFSIQLLGKRYSEELRDPSSALYRLLVEEFISEVEKAFTGLPGYKGIRVLEFRAPEENDSGIDVHYAVTFNGEAISNTTWDLISLHSNKVENHGLVEMDDKPTAVYTISNFRDYIAETLHQNFLMGNSSLNPDPKSLQLINVRGVLLPQTEDIVWNTQSSSLQVTTSSILDNTLQAEWLSADTTTTTTTTISPFGFSSSSPSATGRELQSQSALRDVVSTSKLASPTKVVLSSLPEILGGSSLTLHSVTPAVLQPDLPVAPEGRTSGSFILEDGLASTEELEDTSIDGLPSSPLIQPVPKETVPPMEDSDTALLSTPHLTSSAIEDLTKDIGTPSGLESLASNISDQLEVIPWFPDTSVEKDFIFESGLGSGSGKDVDVIDWPWSETSLEKTTKPLSKSWSEEQDALLPTEGREKLHIDGRVDSTEQIIESSEHRYGDRPIHFIEEESHVRSTIPIFVESATPPTSPIFSKHTSDVPDIDSYSLTKPPFLPVTIAIPASTKKTDEVLKEDMVHTESSSHKELDSEVPVSRPDMQPVWTMLPESDTVWTRTSSLGKLSRDTLASTPESTDRLWLKASMTQSTELPSTTHSTQLEEEVIMAVQDISLELDQVGTDYYQSELTEEQHGKADSYVEMSTSVHYTEMPIVALPTKGGVLSHTQTAGALVVFFSLRVTNMLFSEDLFNKNSLEYKALEQRFLELLVPYLQSNLSGFQNLEILSFRNGSIVVNSRVRFAESAPPNVNKAMYRILEDFCTTAYQTMNLDIDKYSLDVESGDEANPCKFQACNEFSECLVNPWSGEAKCKCYPGYLSVDELPCQSLCDLQPDFCLNDGKCDIMPGHGAICRCRVGSNWWYRGQHCEEFVSEPFVIGITIASVVSFLLVASAVVFFLVKMLQAQNVRRERQRPTSSSRHPDSLSSVENAMKYNPAYESHLAGCELYEKSYSQHPFYSSASEEVIGGLSREEIRQMYESSDLSKEEIQERMRILELYANDPEFAAFVREHQMEEL

(19) United States
(12) Patent Application Publication
(10) Pub. No.: US 2025/0082689 A1
(43) Pub. Date: Mar. 13, 2025

(54) COMPOSITIONS AND METHODS FOR ENHANCING CARDIOMYOCYTE TRANSPLANT ENGRAFTMENT

(71) Applicants: UNIVERSITY OF WASHINGTON, Seattle, WA (US); INTERNATIONAL CENTRE FOR GENETIC ENGINEERING AND BIOTECHNOLOGY (ICGEB), Trieste (IT)

(72) Inventors: Charles E. MURRY, Seattle, WA (US); Mauro GIACCA, Trieste (IT); Francesca BORTOLOTTI, Trieste (IT); Hiroshi TSUCHIDA, Seattle, WA (US)

(73) Assignees: UNIVERSITY OF WASHINGTON, Seattle, WA (US); INTERNATIONAL CENTRE FOR GENETIC ENGINEERING AND BIOTECHNOLOGY (ICGEB), Trieste (IT)

(21) Appl. No.: 18/290,609

(22) PCT Filed: Jul. 20, 2022
(86) PCT No.: PCT/US2022/073937

§ 371 Date: Jan. 19, 2024

Related U.S. Application Data
(60) Provisional application No. 63/224,136, filed on Jul. 21, 2021.

(51) Int. Cl. A61K 35/34; A61K 38/17; A61K 38/19; A61K 38/20; A61K 38/21; A61K 38/46; A61K 38/48; A61K 38/55; C12N 5/077; C12N 15/86

(52) U.S. Cl. CPC A61K 35/34; A61K 38/1709; A61K 38/195; A61K 38/20; A61K 38/21; A61K 38/465; A61K 38/482; A61K 38/55; C12N 5/0657; C12N 15/86; C12Y 301/01004; C12Y 301/03013; C12Y 304/21108; C12N 2750/14143

(57) ABSTRACT

Described herein are compositions and methods related to enhancing cardiomyocyte transplant engraftment and methods of administering a transplant composition.

### CROSS-REFERENCE TO RELATED APPLICATIONS

This application claims benefit under 35 U.S.C. § 119 (e) of U.S. Provisional Application No. 63/224,136 filed Jul. 21, 2021, the contents of which are incorporated herein by reference in their entirety.

### SEQUENCE LISTING

The instant application contains a Sequence Listing which has been submitted in XML format via EFS-Web and is hereby incorporated by reference in its entirety. Said XML copy, created on Jul. 15, 2022, is named 034186-190460WOPT_SL.xml and is 517,630 bytes in size.

### TECHNICAL FIELD

The technology described herein relates to compositions and methods for enhancing cardiomyocyte transplant engraftment and uses thereof.

### BACKGROUND

Enhancing the engraftment of cardiomyocytes (e.g., in vitro-differentiated cardiomyocytes) is necessary for their use in regenerative therapies and the treatment of cardiac diseases. Many cardiomyocyte cell-based therapies have shown a lack of clinical efficacy due, at least in part, to the inability to produce mature or viable differentiated cardiomyocytes, inability to form mature cell-cell interactions with native cardiomyocytes, and a lack of viability and contractile function post-engraftment. Thus, new cellular targets and compositions that enhance cardiomyocyte transplant engraftment into mammalian subjects are needed to overcome these challenges.

### SUMMARY

The technology described herein relates to the discovery of compositions that enhance cardiomyocyte transplant engraftment.

In one aspect, described herein is a composition comprising cardiomyocytes in admixture with an isolated polypeptide selected from the group consisting of: the polypeptides listed in Table 1.

In another aspect, described herein is a composition comprising cardiomyocytes in admixture with two or more isolated polypeptides selected from the group consisting of: the polypeptides listed in Table 1.

In another embodiment of any of the aspects, the cardiomyocytes are differentiated in vitro from embryonic stem cells or from induced pluripotent stem cells.

In another aspect, described herein is a transplant composition comprising cardiomyocytes in admixture with an isolated polypeptide selected from the group consisting of: the polypeptides listed in Table 1.

In another aspect, described herein is a transplant composition comprising cardiomyocytes in admixture with two or more isolated polypeptides selected from the group consisting of: the polypeptides listed in Table 1.

In another aspect, described herein is a composition comprising cardiomyocytes that have been contacted with an isolated polypeptide selected from the group consisting of: the polypeptides listed in Table 1.

In another aspect, described herein is a composition comprising cardiomyocytes that have been contacted with two or more isolated polypeptides selected from the group consisting of: the polypeptides listed in Table 1.

In another aspect, described herein is a transplant composition comprising cardiomyocytes that have been contacted with an isolated polypeptide selected from the group consisting of: the polypeptides listed in Table 1.

In another aspect, described herein is a transplant composition comprising cardiomyocytes that have been contacted with two or more isolated polypeptides selected from the group consisting of: the polypeptides listed in Table 1.

In another aspect, described herein is a composition comprising cardiomyocytes and a nucleic acid construct encoding a polypeptide selected from the group consisting of: the polypeptides listed in Table 1.

In another aspect, described herein is a composition comprising cardiomyocytes and one or more nucleic acid constructs encoding two or more polypeptides selected from the group consisting of: the polypeptides listed in Table 1.

In another aspect, described herein is a composition comprising cardiomyocytes in admixture with an isolated metabolic factor polypeptide that promotes transplant engraftment of the cardiomyocytes, or with a nucleic acid construct that encodes such a factor.

In another aspect, described herein is a composition comprising cardiomyocytes in admixture with an isolated vascular remodeling, extracellular matrix, proteoglycan or cell adhesion polypeptide that promotes transplant engraftment of the cardiomyocytes, or with a nucleic acid construct that encodes such a factor.

In another aspect, described herein is a composition comprising cardiomyocytes in admixture with an isolated canonical Wnt pathway polypeptide.

In another aspect, described herein is a composition comprising cardiomyocytes that have been contacted with an isolated canonical Wnt pathway polypeptide.

In another aspect, described herein is a composition comprising cardiomyocytes in admixture with an isolated serine protease polypeptide.

In another aspect, described herein is a composition comprising cardiomyocytes that have been contacted with an isolated serine protease polypeptide.

In another aspect, described herein is a composition comprising cardiomyocytes in admixture with an isolated serine protease inhibitor polypeptide.

In another aspect, described herein is a composition comprising cardiomyocytes that have been contacted with an isolated serine protease inhibitor polypeptide.

In another aspect, described herein is a composition comprising cardiomyocytes in admixture with an isolated signaling polypeptide of the interleukin family, interferon signaling family, or chemokine family.

In another aspect, described herein is a composition comprising cardiomyocytes that have been contacted with an isolated signaling polypeptide of the interleukin family, interferon signaling family, or chemokine family.

In another aspect, described herein is a composition comprising cardiomyocytes in admixture with an isolated TLR binding polypeptide, a lipocalin polypeptide or a secretaglobin polypeptide.

In another aspect, described herein is a composition comprising cardiomyocytes that have been contacted with an isolated TLR binding polypeptide, a lipocalin polypeptide or a secretaglobin polypeptide.

In another aspect, described herein is a cardiac delivery device comprising a composition or transplant composition described herein.

In one embodiment of any of the aspects, the cardiomyocytes are human cardiomyocytes.

In another embodiment of any of the aspects, the cardiomyocytes are differentiated in vitro from embryonic stem cells or from induced pluripotent stem cells.

In another embodiment of any of the aspects, the construct is in a vector.

In another embodiment of any of the aspects, the construct or constructs is/are in admixture with a transfection reagent.

In another embodiment of any of the aspects, the cardiomyocytes are human cardiomyocytes.

In another embodiment of any of the aspects, the cardiomyocytes are differentiated in vitro from embryonic stem cells or induced pluripotent stem cells.

In another embodiment of any of the aspects, the isolated metabolic factor is selected from the group consisting of: a polypeptide that promotes lipid hydrolysis and a polypeptide that modulates insulin or IGF signaling.

In another embodiment of any of the aspects, the polypeptide that promotes lipid hydrolysis is selected from LIPM, PSAP and PLA2G2C, and the polypeptide that modulates insulin or IGF signaling is selected from SERPINA12, HTRA1 and FETUB.

In another embodiment of any of the aspects, the composition comprises two or more polypeptide factors selected from the groups consisting of vascular remodeling, extracellular matrix, proteoglycan and cell adhesion polypeptides.

In another embodiment of any of the aspects, the vascular remodeling polypeptide is selected from the group consisting of Table 6, the extracellular matrix polypeptide is selected from the group consisting of the polypeptides listed in Table 7, the proteoglycan polypeptide is selected from Table 8, and the cell adhesion polypeptide is selected from the polypeptides listed in Table 9.

In another embodiment of any of the aspects, the canonical Wnt pathway polypeptide is selected from the group consisting of the polypeptides listed in Table 2.

In another embodiment of any of the aspects, the serine protease polypeptide is selected from the group consisting of the polypeptides listed in Table 10.

In another embodiment of any of the aspects, the isolated serine protease inhibitor polypeptide is selected from the group consisting of the polypeptides listed in Table 11.

In another embodiment of any of the aspects, the signaling polypeptide of the interleukin family is selected from the group consisting of the polypeptides listed in Table 12, the signaling polypeptide of the interferon signaling family is selected from the polypeptides listed in Table 13, and the signaling polypeptide of the chemokine family is selected from the group consisting of the polypeptides listed in Table 14.

In another embodiment of any of the aspects, the TLR binding polypeptide is selected from the polypeptides listed in Table 15, the lipocalin polypeptide is selected from the polypeptides listed in Table 16, and the secretaglobin polypeptide is selected from the polypeptides listed in Table 17.

In another aspect, described herein is a method of transplanting cardiomyocytes, the method comprising administering a composition described herein to cardiac tissue.

In another aspect, described herein is a method of enhancing cardiomyocyte transplant engraftment, the method comprising contacting a cardiomyocyte population with a polypeptide selected from the group consisting of the polypeptides listed in Table 1, and transplanting the cardiomyocyte population to cardiac tissue.

In another aspect, described herein is a method of enhancing cardiomyocyte transplant engraftment, the method comprising contacting a cardiomyocyte population with a nucleic acid that encodes a polypeptide selected from the group consisting of the polypeptides listed in Table 1, and transplanting the cardiomyocyte population to cardiac tissue.

In another aspect, described herein is a method of enhancing cardiomyocyte transplant engraftment, the method comprising contacting a cardiomyocyte population with a vector that encodes a polypeptide selected from the group consisting of the polypeptides listed in Table 1, and transplanting the cardiomyocyte population to cardiac tissue.

In one embodiment of any of the aspects, the engraftment of the administered cardiomyocytes is increased relative to engraftment of cardiomyocytes that were not in admixture with or had not been contacted with the polypeptide or polypeptides.

In another embodiment of any of the aspects, the vector comprises an AAV vector.

In another embodiment of any of the aspects, the cardiomyocyte population is a human cardiomyocyte population.

In another embodiment of any of the aspects, the cardiomyocyte population is differentiated in vitro from embryonic stem cells or induced pluripotent stem cells.

In another embodiment of any of the aspects, the induced pluripotent stem cells are differentiated from induced pluripotent stem cells derived from the transplant recipient.

### BRIEF DESCRIPTION OF THE DRAWINGS

FIG. 1 demonstrates the process for functional selection of secreted factors for the enhancement of cardiac engraftment. (1) Secreted factors from the mouse are cloned into an adeno-associated viral (AAV) vector; (2) AAV vectors coding for secreted factors are selected and produced (3) embryonic stem cell-derived cardiomyocyte (ES-CMs) are contacted with AAV vectors coding for the secreted factors ex vivo; (4) ES-CMs are transduce following a myocardial infarction (MI) in a mouse model via intracardiac administration; (5) viable ES-CMs containing pro-survival factors are extracted; (6) DNA was isolated from the viable cells and primers were generated for the known secreted factors; (7) Next generation sequencing was performed on the extracted DNA to identify and validate the pro-survival/pro-engraftment factors.

FIG. 2 demonstrates a workflow of an individual round of testing of the secreted factors.

FIG. 3 shows in vivo factor enrichment and selection of pro-survival factors based on Z score (post transcription counts/pre-transcription counts).

FIG. 4 demonstrates the final AAV pool that was enriched 7 days following a myocardial infarction.

### DETAILED DESCRIPTION

The compositions and methods described herein are related, in part, to the discovery that secreted factors can enhance cardiac engraftment following myocardial infarction. The combination of some secreted factors with cardiomyocytes improves the engraftment of the cardiomyocytes in vivo. Next-generation sequencing of the engrafted cardiomyocytes confirmed and identified the secreted factors that are beneficial for cardiomyocyte viability and function post-engraftment. Accordingly, also provided herein are compositions for the treatment and prevention of heart diseases and myocardial infarction in which cardiomyocyte engraftment is promoted using factors as described herein.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “cardiomyocyte” refers to a cell that is from cardiac muscle or a heart or a cell that comprises phenotypic and/or structural features associated with cardiac muscle (e.g., contraction, electrical phenotypes, sarcomeres, actin and myosin expression, etc.). The cardiomyocyte can be a native cardiomyocyte isolated from an organism or a cardiomyocyte that is differentiated from a stem cell or cardiac precursor (e.g., in-vitro differentiated cardiomyocytes).

As used herein the term “human stem cell” refers to a human cell that can self-renew and differentiate to at least one cell type. The term “human stem cell” encompasses human stem cell lines, human-derived induced pluripotent stem (iPS) cells, human embryonic stem cells, human pluripotent cells, human multipotent stem cells, amniotic stem cells, placental stem cells, or human adult stem cells.

As used herein, “in vitro-differentiated cardiomyocytes” refers to cardiomyocytes that are generated in culture, typically via step-wise differentiation from a precursor cell such as a human embryonic stem cell, an induced pluripotent stem cell, an early mesoderm cell, a lateral plate mesoderm cell or a cardiac progenitor cell. Thus, while cardiomyocytes in vivo are ultimately derived from a stem cell, i.e., during development of a tissue or organism, a stem cell-derived cardiomyocyte as described herein has been created by in vitro differentiation from a stem cell. As used herein, a cell differentiated in vitro from a stem cell, e.g., an induced pluripotent stem (iPS) cell or embryonic stem cell (“ES cell” or “ESC”), is a “stem-cell derived cardiomyocyte” or “in vitro-differentiated cardiomyocyte” if it has, at a minimum, spontaneous beating or contraction, and expression of cardiac troponin T (cTnT). Methods for differentiating stem cells in vitro to cardiomyocytes are known in the art and described elsewhere herein.

As used herein the term “admixture” refers to a composition comprising two or more elements. For example, an admixture as used herein can refer to a composition comprising cardiomyocytes and an isolated polypeptide as described herein.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of cardiomyocytes, refers to a population of cells that contain fewer than about 20%, more preferably fewer than about 15%, 10%, 8%, 7%, most preferably fewer than about 5%, 4%, 3%, 2%, 1%, or less than 1%, of cells that are not cardiomyocytes, respectively.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues provided herein are preferred to be in the “L” isomeric form. However, residues in the “D” isomeric form may be substituted for any L-amino acid residue provided the desired properties of the polypeptide are retained.

As used herein the term “isolated polypeptide” refers to a polypeptide removed from an organism or cell in which it was originally found, or a descendent of such a polypeptide. Optionally an isolated polypeptide has been encoded by a nucleic acid or a vector; expressed in a heterologous cell; and purified by methods known in the art.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snoRNA), small nuclear RNA (snRNA), and small temporal RNA (stRNA), and the like, and any combination thereof.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+ (% conservative changes). It should be understood that a sequence that has a specified percent similarity to a reference sequence necessarily encompasses a sequence with the same specified percent identity to that reference sequence. The skilled person will be aware of various computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, plasmids, mini-chromosomes, phage, naked DNA, a cloning vector, an expression vector, a plasmid, phage, transposon, cosmid, artificial chromosome, virus, virion, etc. See, for example, U.S. Pat. Nos. 4,980,285; 5,631,150; 5,707,828; 5,759,828; 5,888,783, and 5,919,670, and Sambrook et al, Molecular Cloning: A Laboratory Manual, 2nd Ed., Cold Spring Harbor Press (1989).

Vectors can include, for example, an adenovirus associated virus (AAV) vectors, such as a recombinant AAV vector (rAAV). Gene therapy vectors using AAV can infect both dividing and quiescent cells and persist in an extrachromosomal state without integrating into the genome of the host cell. Integration of virally carried genes into the host genome can also occur.

The recombinant AAV vector can be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene can comprise one or more regions that encode one or more isolated polypeptides as described herein (see e.g., Table 1). The transgene can comprise a region encoding, for example, an isolated polypeptide as described herein and/or an expression control sequence (e.g., a poly-A tail).

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed herein is a “cis-acting” plasmid containing the transgene, in which the selected transgene sequence and associated regulatory elements are flanked by the 5′ and 3′ AAV ITR sequences. The AAV ITR sequences can be obtained from any known AAV, including presently identified mammalian AAV types. In some embodiments, the rAAV vector comprises at least one ITR having a serotype selected from AAV1, AAV2, AAV5, AAV6, AAV6.2, AAV7, AAV8, AAV9, AAV10, AAV11, AAVrh10, and variants thereof. In some embodiments, the vector further comprises AAV ITRs. In some embodiments, the ITR is an AAV1, AAV2, AAV3b, AAV4, AAV5, AAV6, AAV7, AAV8, AAV9, AAV10, AAV11, AAV12, AAV13, or AAVrh10 ITR.

In some embodiments, the rAAV further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAV11, AAVrh10, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16 (10): 1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and can be utilized.

In some embodiments, the rAAV has tissue-specific targeting capabilities, such that a transgene of the rAAV can be delivered specifically to one or more predetermined tissue(s). The AAV capsid is one element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV2, AAV3, AAV4, AAV5, AAV6, AAV8, AAVrh8, AAV9, AAV10, and AAVrh10. In some embodiments, an AAV capsid protein is of a serotype derived from a non-human primate, for example AAVrh8 or AAVrh10 serotype. In some embodiments, an AAV capsid protein is of an AAV9 serotype. In some embodiments, an AAV capsid protein is of an AAVrh10 serotype. In some embodiments, the capsid protein is an AAV1, AAV2, AAV3b, AAV4, AAV5, AAV6, AAV7, AAV8, AAV9, AAV10, AAV11, AAV12, AAV13, or AAVrh10 capsid protein or any chimera thereof. In some embodiments, recombinant AAV (rAAV) is a haploid rAAV. In some embodiments, the haploid rAAV comprises chimeric capsid proteins.

In one embodiment, the viral capsid is modified. In one embodiment, the modified viral capsid is a chimeric capsid. A “chimeric’ capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the

### LITERATURE

In one embodiment, the modified viral capsid is a haploid capsid. As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric is present. See e.g., US Patent Application 2019/0002841, or U.S. Pat. No. 8,906,675 or International Application WO2021127455A1; the contents of each of which are incorporated herein by reference in their entireties.

As used herein, the term “expression” or “expressed” or “positive for” refers to a cell (e.g., a cardiomyocytes) that has a detectable level of a given nucleic acid, vector or polypeptide. The nucleic acid, vector, or polypeptide can be detected by any method available to one of skill in the art. For example, a polypeptide as described herein can be expressed by the cardiomyocytes following contact with a vector or an agent that induces expression of that polypeptide. The expression can be transient or stable expression by the cardiomyocytes.

As used herein, the term “expression vector” refers to a vector that directs expression of an RNA or polypeptide described herein from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the terms “positive for” or “expresses a marker” refer to expression of mRNA encoding a marker or factor described herein (including, but not limited to a given secreted factor) is detectable above background levels using RT-PCR. The expression level of a marker or factor can be compared to the expression level obtained from a negative control (i.e., cells known to lack the marker) or by isotype controls (i.e., a control antibody that has no relevant specificity and only binds non-specifically to cell proteins, lipids or carbohydrates). Thus, a cell that “expresses” a marker (or is “positive for a marker”) has an expression level detectable above the expression level determined for the negative control for that marker.

The term “marker” as used herein is used to describe a characteristic and/or phenotype of a cell. Markers can be used for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

The term “differentiate”, or “differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (e.g., a human cardiac progenitor cell or mid-primitive streak cardiogenic mesoderm progenitor cell), which in turn can differentiate into other types of precursor cells further down the pathway (such as a tissue specific precursor, such as a cardiomyocyte precursor), and then to an end-stage differentiated cell, which plays a characteristic role in a certain tissue type, and may or may not retain the capacity to proliferate further. Methods for in vitro differentiation of stem cells to cardiomyocytes are known in the art and/or described herein below.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

The term “reprogramming” as used herein refers to a process that alters or reverses the differentiation state of a differentiated cell (e.g. a somatic cell). Stated another way, reprogramming refers to a process of driving the differentiation of a cell backwards to a more undifferentiated or more primitive type of cell. The cell to be reprogrammed can be either partially or terminally differentiated prior to reprogramming. In some embodiments, reprogramming encompasses complete reversion of the differentiation state of a differentiated cell (e.g., a somatic cell) to a pluripotent state. In some embodiments, reprogramming also encompasses partial reversion of the differentiation state of a differentiated cell (e.g., a somatic cell) to a multipotent state. In some embodiments, reprogramming encompasses complete or partial reversion of the differentiation state of a differentiated cell (e.g., a somatic cell) to an undifferentiated cell. Reprogramming also encompasses partial reversion of the differentiation state of a somatic cell to a state that renders the cell more susceptible to complete reprogramming to a pluripotent state when subjected to additional manipulations.

Reprogramming involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

As used herein, the terms “induced pluripotent stem cell,” “iPSC,” “hPSC,” and “human pluripotent stem cell” are used interchangeably herein and refer to a pluripotent cell artificially derived from a differentiated somatic cell. iPSCs are capable of self-renewal and differentiation into cell fate-committed stem cells, including cells of the cardiac lineages, as well as various types of mature cells.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell. As used herein, “iPSC-CMs” or “induced pluripotent stem cell-derived cardiomyocytes” are used interchangeably to refer to cardiomyocytes derived from an induced pluripotent stem cell.

As used herein, the terms, “maturation” or “mature phenotype” or “mature cardiomyocytes” when applied to cardiomyocytes refers to the phenotype of a cell that comprises a phenotype similar to adult cardiomyocytes and does not comprise at least one feature of a fetal cardiomyocyte. In some embodiments, markers which indicate increased maturity of an in vitro-differentiated cell include, but are not limited to, electrical maturity, metabolic maturity, genetic marker maturity, and contractile maturity.

As used herein, “treating” or “administering” are used interchangeably in the context of the placement of a composition as described herein, into a subject, by a method or route which results in at least partial localization of the compositions described herein at a desired site, such as the heart or a region thereof, such that a desired effect(s) is produced. The agent described herein can be administered by any appropriate route which results in delivery to a desired location in the subject. The half-life of the agent after administration to a subject can be as short as a few minutes, hours, or days, e.g., twenty-four hours, to a few days, to as long as several years, i.e., long-term. In some embodiments of any of the aspects, the term “treatment” refers to the administration of the compositions described herein comprising cardiomyocytes in admixture with an isolated polypeptide, cardiomyocytes that have been previously contacted with an isolated polypeptide or a nucleic acid encoding such isolated polypeptide. The administering can be done by contacting the cardiomyocytes by direct injection (e.g., directly administered to a target cell or tissue) or intracardiac injection to the subject in need thereof. Administering can be transient, local, or systemic.

As described herein, a “genetically modified cell” is a cell which either carries a heterologous genetic material or construct, or which comprises a genome that has been manipulated, e.g., by mutation, including but not limited to site-directed mutation. The introduction of a heterologous genetic material generally results in a change in gene or protein expression relative to an un-modified cell. Introduction of RNA can transiently promote expression of a foreign or heterologous product, as can the introduction of a vector that does not integrate or replicate within the cell. Introduction of a construct that integrates into a cell's genome or replicates with the cell's nucleic acid will be more stable through successive cell divisions. In one embodiment, genetic modification is in addition to or separate from the introduction of a construct or constructs that reprogram a somatic cell to a stem cell phenotype, such as an iPS cell phenotype. Genetic modifications are known to those of skill in the art and can include, but are not limited to, the introduction of genetic material via viral vector or modification using CRISPR/Cas or similar system for site specific recombination.

As used herein, the term “contacting” when used in reference to a cell, encompasses both introducing an agent, surface, hormone, etc. to the cell in a manner that permits physical contact of the cell with the agent, surface, hormone etc., and introducing an element, such as a genetic construct or vector, that permits the expression of an agent, such as a miRNA, polypeptide, or other expression product in the cell. It should be understood that a cell genetically modified to express an agent, is “contacted” with the agent, as are the cell's progeny that express the agent.

As used herein, the terms “disease” or “disorder” refers to a disease, syndrome, or disorder, partially or completely, directly or indirectly, caused by one or more abnormalities in the genome, physiology, or behavior, or health of a subject. The disease or disorder can be a cardiac disease or disorder.

As used herein, the term, “cardiac disease” refers to a disease that affects the circulatory system of a subject. Non-limiting examples of cardiac diseases include cardiomyopathy, cardiac arrhythmias, myocardial infarction, heart failure, cardiac hypertrophy, long QT syndrome, arrhythmogenic right ventricular dysplasia (ARVD), catecholaminergic polymorphic ventricular tachycardia (CPVT), Barth syndrome, and Duchenne muscular dystrophy.

“Treatment” of a cardiac disorder, a cardiac disease, or a cardiac injury (e.g., myocardial infarction) as referred to herein refers to therapeutic intervention that enhances cardiac function and/or enhances cardiomyocyte engraftment and/or enhances cardiomyocyte transplant or graft vascularization in a treated area, thus improving the function of e.g., the heart. That is, cardiac “treatment” is oriented to the function of the heart (e.g., enhanced function within an infarcted area), and/or other site treated with the compositions described herein. A therapeutic approach that improves the function of the heart, for example as assessed by measuring left-ventricular end-systolic dimension (LVESD)) or cardiac output by at least 10%, and preferably by at least 20%, 30%, 40%, 50%, 75%, 90%, 100% or more, e.g., 2-fold, 5-fold, 10-fold or more, up to and including full function, relative to such function prior to such therapy is considered effective treatment. Effective treatment need not cure or directly impact the underlying cause of the heart disease or disorder to be considered effective treatment.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease.

As used herein, the term “transplanting” or “engraftment” is used in the context of the placement of cells, e.g. stem cells, cardiomyocytes, as described herein into a subject, by a method or route which results in at least partial localization of the introduced cells at a desired site, such as a site of injury or repair, such that a desired effect(s) is produced. The cells e.g. cardiomyocytes, or their differentiated progeny (e.g. cardiac fibroblasts etc.) and cardiomyocytes can be implanted directly to the heart or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of the cardiomyocytes is desired as cardiomyocytes generally do not proliferate to an extent that the heart can heal from an acute injury comprising cell death. In other embodiments, the cells can be administered via an indirect systemic route of administration, such as an intraperitoneal or intravenous route.

As used herein, the term “scaffold” refers to a structure, comprising a biocompatible material that provides a surface suitable for adherence and proliferation of cells. A scaffold can further provide mechanical stability and support. A scaffold can be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used herein, a “substrate” refers to a structure, comprising a biocompatible material that provides a surface suitable for adherence and proliferation of cells. A nanopatterned or micropatterned substrate can further provide mechanical stability and support. A substrate can be in a particular shape or form so as to influence or delimit a three-dimensional shape or form assumed by a population of proliferating cells. Such shapes or forms include, but are not limited to, films (e.g., a form with two-dimensions substantially greater than the third dimension), ribbons, cords, sheets, flat discs, cylinders, spheres, 3-dimensional amorphous shapes, etc. The substrate can be nanopatterned or micropatterned to permit the formation of engineered tissues on the substrate.

As used herein, the term “implantable in a subject” refers to any non-living (e.g., acellular) implantable structure that upon implantation does not generate an appreciable immune response in the host organism. Thus, an implantable structure should not for example, be or contain an irritant, or contain LPS etc.

The term “agent” or “activator” as used herein means any compound or substance such as, but not limited to, a small molecule, nucleic acid, polypeptide, peptide, drug, ion, etc. An “agent” can be any chemical, entity or moiety, including without limitation, synthetic and naturally-occurring proteinaceous and non-proteinaceous entities. In some embodiments of any of the aspects, an agent is nucleic acid, nucleic acid analogues, proteins, antibodies, peptides, aptamers, oligomer of nucleic acids, amino acids, or carbohydrates including g without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, the term “modulates” refers to an effect including increasing or decreasing a given parameter as those terms are defined herein.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a disease, or a biological sample that has not been contacted with a composition, polypeptide, or nucleic acid encoding such polypeptide as disclosed herein).

As used herein, an “appropriate control” refers to an untreated, otherwise identical cell or population (e.g., a biological sample that was not contacted by an agent or composition described herein, or not contacted in the same manner, e.g., for a different duration, as compared to a non-control cell).

As used herein, the term “phenotypic characteristic,” as applied to in vitro differentiated cells (e.g., cardiomyocytes), or culture of in vitro-differentiated cells, refers to any of the parameters described herein as measures of cell function. A “change in a phenotypic characteristic” as described herein is indicated by a statistically significant increase or decrease in a functional property with respect to a reference level or appropriate control.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

### Cell Preparations

The compositions and methods described herein use cardiomyocytes in an admixture with an isolated polypeptide, or cardiomyocytes that have been contacted with an isolated polypeptide or a nucleic acid encoding such polypeptide from Table 1. Such cardiomyocytes can be introduced or engrafted into a subject for the treatment of heart disease (e.g., myocardial infarction or heart failure). The cardiomyocytes described herein can be isolated from a human subject or differentiated from stem cells or a cardiac precursor.

In some embodiments of any of the aspects described herein, the cardiomyocytes are human cardiomyocytes.

In some embodiments, the cardiomyocytes are differentiated in vitro from embryonic stem cells or from induced pluripotent stem cells.

The following describes various stem cells that can be used to prepare cardiomyocytes.

Embryonic Stem Cells: Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of pluripotent stem cells can include amnion-derived or placental-derived stem cells. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Cardiomyocytes useful in the compositions and methods described herein can be differentiated from both embryonic stem cells and induced pluripotent stem cells, among others. In one embodiment, the compositions and methods provided herein use human cardiomyocytes differentiated from embryonic stem cells. Alternatively, in some embodiments, the compositions and methods provided herein do not encompass generation or use of human cardiogenic cells made from cells taken from a viable human embryo.

Embryonic stem cells and methods for their retrieval are well known in the art and are described, for example, in Trounson A O Reprod Fertil Dev (2001) 13:523, Roach M L Methods Mol Biol (2002) 185:1, and Smith A G Annu Rev Cell Dev Biol (2001) 17:435. The term “embryonic stem cell” is used to refer to the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. The single blastomere cell is co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the human cardiomyocytes described herein are not derived from embryonic stem cells or any other cells of embryonic origin.

Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns.

Induced Pluripotent Stem Cells (iPSCs)

In some embodiments, the compositions and methods described herein utilize cardiomyocytes that are differentiated in vitro from induced pluripotent stem cells. An advantage of using iPSCs to generate cardiomyocyte for the compositions described herein is that the cells can be derived from the same subject to which the desired human cardiomyocytes are to be administered. That is, a somatic cell can be obtained from a subject, reprogrammed to an induced pluripotent stem cell, and then re-differentiated into a human cardiomyocyte to be administered to the subject (e.g., autologous cells). Since the cardiomyocytes (or their differentiated progeny) are essentially derived from an autologous source, the risk of engraftment rejection or allergic responses is reduced compared to the use of cells from another subject or group of subjects. In some embodiments, the cardiomyocytes useful for the compositions described herein are derived from non-autologous sources. In addition, the use of iPSCs negates the need for cells obtained from an embryonic source. Thus, in one embodiment, the stem cells used to generate cardiomyocytes for use in the compositions and methods described herein are not embryonic stem cells.

Although differentiation is generally irreversible under physiological contexts, several methods have been developed in recent years to reprogram somatic cells to induced pluripotent stem cells. Exemplary methods are known to those of skill in the art and are described briefly herein below.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell. It should be noted that placing many primary cells in culture can lead to some loss of fully differentiated characteristics. However, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state with the capacity for self-renewal and differentiation to cells of all three germ cell lineages. The resulting cells are referred to as “reprogrammed cells,” or “induced pluripotent stem cells (iPSCs or iPS cells).”

The specific approach or method used to generate pluripotent stem cells from somatic cells (e.g., any cell of the body with the exclusion of a germ line cell; fibroblasts etc.) is not critical to the claimed invention. Thus, any method that re-programs a somatic cell to the pluripotent phenotype would be appropriate for use in the methods described herein.

iPS cells can be generated or derived from terminally differentiated somatic cells, as well as from adult stem cells, or somatic stem cells. That is, a non-pluripotent progenitor cell can be rendered pluripotent or multipotent by reprogramming.

The efficiency of reprogramming (i.e., the number of reprogrammed cells) of cells derived from a population of starting cells can be enhanced by the addition of various small molecules as shown by Shi, Y., et al (2008) Cell-Stem Cell 2:525-528, Huangfu, D., et al (2008) Nature Biotechnology 26 (7): 795-797, and Marson, A., et al (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

When reprogrammed cells are used for generation of human cardiomyocytes to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells from which they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; B-III-tubulin; a-smooth muscle actin (a-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; B-catenin, and Bmi1. Such cells can also be characterized by the down-regulation of markers characteristic of the somatic cell from which the induced pluripotent stem cell is derived.

### In Vitro Differentiation

The methods and compositions described herein can use in vitro differentiated cardiomyocytes. Methods for the differentiation of either cell type from ESCs or IPSCs are known in the art. See, e.g., LaFlamme et al., Nature Biotech 25:1015-1024 (2007), which describes the differentiation of cardiomyocytes which is incorporated herein by reference in its entirety. These approaches use various factors and conditions to activate and guide differentiation programs leading to their respective cell types. Pathways and certain of the factors involved in them are discussed in the following.

In certain embodiments, the step-wise differentiation of ESCs or iPSCs to cardiomyocytes proceeds in the following order: ESC or iPSC >cardiogenic mesoderm >cardiac progenitor cells >cardiomyocytes (see e.g., US 2017/024086, the contents of which are incorporated herein by reference in its entirety).

As will be appreciated by those of skill in the art, in vitro-differentiation of cardiomyocytes produces an end-result of a cell having the phenotypic and morphological features of the desired cell type but that the differentiation steps of in vitro-differentiation need not be the same as the differentiation that occurs naturally in the embryo. That is, during differentiation to a cardiomyocyte, it is specifically contemplated herein that the step-wise differentiation approach utilized to produce such cells need not proceed through every progenitor cell type that has been identified during embryogenesis and can essentially “skip” over certain stages of development that occur during embryogenesis.

### Monitoring Differentiation of Cardiomyocytes and Functional Characterization

As will be appreciated by one of skill in the art, an in vitro-differentiated cardiomyocyte described herein will lack markers of hematopoietic or hemogenic cells, vascular endothelial cells, embryonic stem cells or induced pluripotent stem cells. In one embodiment of the methods described herein, one or more cell surface markers are used to determine the degree of differentiation along the spectrum of embryonic stem cells or iPSCs to e.g., fully differentiated cardiomyocytes.

In some embodiments, antibodies or similar agents specific for a given marker, or set of markers, can be used to separate and isolate the desired cells using fluorescent activated cell sorting (FACS), panning methods, magnetic particle selection, particle sorter selection and other methods known to persons skilled in the art, including density separation (Xu et al. (2002) Circ. Res. 91:501; U.S. Ser. No. 20/030,022367) and separation based on other physical properties (Doevendans et al. (2000) J. Mol. Cell. Cardiol. 32:839-851). Negative selection can be performed, including selecting and removing cells with undesired markers or characteristics, for example fibroblast markers, epithelial cell markers etc.

Undifferentiated ES cells express genes that can be used as markers to detect the presence of undifferentiated cells. Exemplary ES cell markers include stage-specific embryonic antigen (SSEA)-3, SSEA-4, TRA-I-60, TRA-1-81, alkaline phosphatase or those described in e.g., U.S.S.N. 2003/0224411; or Bhattacharya (2004) Blood 103 (8): 2956-64, each herein incorporated by reference in their entirety. Exemplary markers expressed on cardiac progenitor cells include, but are not limited to, TMEM88, GATA4, ISL1, MYL4, and NKX2-5.

Exemplary markers expressed on cardiomyocytes include, but are not limited to, NKX2-5, MYH6, MYL7, TBX5, ATP2a2, RYR2, and cTnT.

In some embodiments, the desired cells (e.g., in vitro-differentiated cardiomyocytes) are an enriched population of cells; that is, the percentage of in vitro-differentiated cardiomyocytes (e.g., percent of cells) in a population of cells is at least 10% of the total number of cells in the population. For example, an enriched population comprises at least 15% definitive cardiomyocytes, at least 20%, at least 30%, at least 40%, at least 50%, at least 60%, at least 70%, at least 80%, at least 90%, at least 95%, at least 99% or even 100% of the population comprises human in vitro-differentiated cardiomyocytes. In some embodiments, a population of cells comprises at least 100 cells, at least 500 cells, at least 1000 cells, at least 1×10⁴ cells, at least 1×10¹¹ cells, at least 1×10⁶ cells, at least 1×10⁷ cells, at least 1×10⁸ cells, at least 1×10⁹ cells, at least 1×10¹⁰ cells, at least 1×10¹¹ cells, at least 1×10¹² cells, at least 1×10¹³ cells, at least 1×10¹⁴ cells, at least 1×10¹⁵ cells, or more.

Matured cardiomyocytes, including stem-cell derived cardiomyocytes, permit evaluation of the response of mature cardiomyocytes to various treatments or stimuli. In various embodiments, quantifiable parameters of stem cell-derived cardiomyocytes can include contractile force, contractility, altered contraction, frequency of contraction, contraction duration, contraction stamina, cardiomyocyte size, sarcomere organization, length, circumference, structure, multinucleate status, metabolic respiratory capacity, oxygen consumption, electrophysiological and biophysical parameters. In some embodiments, quantifiable parameters include survival and/or division or regeneration of the e.g., stem cell-derived cardiomyocytes.

While most parameters will provide a quantitative readout, in some instances a semi-quantitative or qualitative result will be acceptable. Characteristically a range of parameter readout values will be obtained for each parameter from a multiplicity of the same assays. Variability is expected and a range of values for each of the set of test parameters will be obtained using standard statistical methods to provide useful values.

Confirmation of cardiomyocyte differentiation and maturation can be assessed by assaying sarcomere morphology and structural characterization of actin and myosin. The structure of cardiac sarcomeres is highly ordered, thus one with ordinary skill in the art can recognize these proteins (actin, myosin, alpha-actinin, titin) and their arrangement in tissues or collections of cultured cells can be used as markers to identify mature muscle cells and tissues. Developing cardiac cells undergo “sarcomerogenesis,” which creates new sarcomere units within the cell. The degree of sarcomere organization provides a measure of cardiomyocyte maturity.

Immunofluorescence assays and electron microscopy for a-actinin, B-myosin, actin, cTnT, tropomyosin, and collagen, among others can be used to identify and measure sarcomere structures. Immunofluorescent images can be quantified for sarcomere alignment, pattern strength, and sarcomere length. This can be accomplished by staining the protein within the sarcomeres (e.g., a-actinin) and qualitatively or quantitatively determining if the sarcomeres are aligned. For a quantitative measurement of sarcomere alignment, several methods can be employed such as using a scanning gradient and Fourier transform script to determine the position of the proteins within the sarcomeres. This is done by using each image taken by a microscope and camera for individual analysis. Using a directional derivative, the image gradient for each segment can be calculated to determine the local alignment of sarcomeres. The pattern strength can be determined by calculating the maximum peaks of one-dimensional Fourier transforms in the direction of the gradient. The lengths of sarcomeres can be calculated by measuring the intensity profiles of the sarcomeres along this same gradient direction.

Cellular morphology can be used to identify structurally mature stem cell-derived cardiomyocytes. Non-limiting examples of morphological and structural parameters include, but are not limited to, sarcomere length, Z-band width, binucleation percentages, nuclear eccentricity, cell area, and cell aspect ratio.

The cell activity and maturation can be determined by a number of parameters such as electrical maturity, metabolic maturity, or contractile maturity of a cardiomyocyte.

Mature cardiomyocytes have functional ion channels that permit the synchronization of cardiac muscle contraction. The electrical function of cardiomyocytes can be measured by a variety of methods. Non-limiting examples of such methods include whole cell patch clamp (manual or automated), multielectrode arrays, field potential stimulation, calcium imaging and optical mapping, among others. Cardiomyocytes can be electrically stimulated during whole cell current clamp or field potential recordings to produce an electrical and/or contractile response. Furthermore, cardiomyocytes can be genetically modified, for example, to express a channel rhodopsin that allows for optical stimulation of the cells. It is also contemplated herein that the methods described herein can be used to stimulate and/or detect electrical impulses and cardiac electrical coupling of mature cardiomyocytes.

Measurement of field potentials and biopotentials of cardiomyocytes can be used to determine the differentiation stage and cell maturity. Without limitations, the following parameters can be used to determine electrophysiological function of e.g., cardiomyocytes: change in field potential duration (FPD), quantification of FPD, beat frequency, beats per minute, upstroke velocity, resting membrane potential, amplitude of action potential, maximum diastolic potential, time constant of relaxation, action potential duration (APD) of 90% repolarization, interspike interval, change in beat interval, current density, activation and inactivation kinetics, among others.

Electrical maturity is determined by one or more of the following markers as compared to a reference level: increased gene expression of an ion channel gene, increased sodium current density, increased inwardly-rectifying potassium channel current density, decreased action potential frequency, decreased calcium wave frequency, and decreased field potential frequency.

Adult cardiomyocytes have been shown to have enhanced oxidative cellular metabolism compared with fetal cardiomyocytes marked by increased mitochondrial function and spare respiratory capacity. Metabolic assays can be used to determine the differentiation stage and cell maturity of the stem cell-derived cardiomyocytes as described herein. Non-limiting examples of metabolic assays include cellular bioenergetics assays (e.g., Seahorse Bioscience XF Extracellular Flux Analyzer), and oxygen consumption tests.

Specifically, cellular metabolism can be quantified by oxygen consumption rate (OCR), OCR trace during a fatty acid stress test, maximum change in OCR, maximum change in OCR after FCCP addition, and maximum respiratory capacity among other parameters.

Furthermore, a metabolic challenge or lactate enrichment assay can provide a measure of stem cell-derived cardiomyocyte maturity or a measure of the effects of various treatments of such cells. Most mammalian cells generally use glucose as their main energy source. However, cardiomyocytes are capable of energy production from different sources such as lactate or fatty acids. In some embodiments, lactate-supplemented and glucose-depleted culture medium, or the ability of cells to use lactate or fatty acids as an energy source is useful to identify mature cardiomyocytes and variations in their function.

As one of skill in the art will recognize, metabolic assays can be used as a functional endpoint in a screening assay or toxicity assay to determine the effects of a given agent on cardiac function using the cardiomyocytes, described herein. For example, hypertrophic cardiomyopathy can be associated with a more fetal metabolic phenotype, thus an agent that shifts the metabolic phenotype of the mature cardiomyocytes to a more fetal metabolic phenotype can be indicative of cardiotoxicity of the given agent.

Metabolic maturity of in vitro-differentiated cardiomyocytes is determined by one or more of the following markers as compared to a reference level: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

Contractility of cardiomyocytes can be measured by optical tracking methods such as video analysis. In addition to optical tracking, impedimetric measurements can also be performed. For example, the cardiomyocytes described herein can have contractility or beat rate measurements determined by xCelligence™ real time cell analysis (ACEA BIOSCIENCES, Inc., San Diego, CA).

A useful parameter to determine cardiomyocyte function is beat rate. The frequency of the contraction, beat rate, change in beat interval (ABI), or beat period, can be used to determine stem cell differentiation stage, stem cell-derived cardiomyocyte maturity, and the effects of a given treatment on such rate. Beat rate can be measured by optical tracking. The beat rate is typically elevated in fetal cardiomyocytes and is reduced as cardiomyocytes develop. Without limitations, contractile parameters can also include contractile force, contraction velocity, relaxation velocity, contraction angle distribution, or contraction anisotropic ratio.

Contractile maturity is determined by one or more of the following markers as compared to a reference level: increased beat frequency, increased contractile force, increased level or activity of a-myosin heavy chain (a-MHC), increased level or activity of sarcomeres, decreased circularity index, increased level or activity of troponin, increased level or activity of titin N2b, increased cell area, and increased aspect ratio.

### Scaffold Compositions

In one aspect, the cardiomyocytes described herein can be admixed with or cultured on a preparation that provides a scaffold or patterned substrate to support the cells. Such a scaffold or patterned substrate can provide a physical advantage in securing the cells in a given location, e.g., after implantation, as well as a biochemical advantage in providing, for example, extracellular cues for the further maturation or, e.g., maintenance of phenotype until the cells are established.

Biocompatible synthetic, natural, as well as semi-synthetic polymers, can be used for synthesizing polymeric particles that can be used as a scaffold material. In general, for the practice of the methods described herein, it is preferable that a scaffold biodegrades such that the cardiomyocytes can be isolated from the polymer prior to implantation or such that the scaffold degrades over time in a subject and does not require removal. Thus, in one embodiment, the scaffold provides a temporary structure for growth and/or delivery of cardiomyocytes to a subject in need thereof. In some embodiments, the scaffold permits human cells to be grown in a shape suitable for transplantation or administration into a subject in need thereof, thereby permitting removal of the scaffold prior to implantation and reducing the risk of rejection or allergic response initiated by the scaffold itself.

Examples of polymers which can be used include natural and synthetic polymers, although synthetic polymers are preferred for reproducibility and controlled release kinetics. Synthetic polymers that can be used include biodegradable polymers such as poly(lactide) (PLA), poly (glycolic acid) (PGA), poly (lactide-co-glycolide) (PLGA), and other polyhydroxyacids, poly (caprolactone), polycarbonates, polyamides, polyanhydrides, polyphosphazene, polyamino acids, polyortho esters, polyacetals, polycyanoacrylates and biodegradable polyurethanes; non-biodegradable polymers such as polyacrylates, ethylene-vinyl acetate polymers and other acyl-substituted cellulose acetates and derivatives thereof; polyurethanes, polystyrenes, polyvinyl chloride, polyvinyl fluoride, poly (vinyl imidazole), chlorosulphonated polyolefins, and polyethylene oxide. Examples of biodegradable natural polymers include proteins such as albumin, collagen, fibrin, silk, synthetic polyamino acids and prolamines; polysaccharides such as alginate, heparin; and other naturally occurring biodegradable polymers of sugar units. Alternately, combinations of the aforementioned polymers can be used. In one aspect, a natural polymer that is not generally found in the extracellular matrix can be used.

PLA, PGA and PLA/PGA copolymers are particularly useful for forming biodegradable scaffolds. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature. The following U.S. Patents, the teachings of which are hereby incorporated by reference, describe in detail suitable polylactides, their properties and their preparation: U.S. Pat. No. 1,995,970 to Dorough; U.S. Pat. No. 2,703,316 to Schneider; U.S. Pat. No. 2,758,987 to Salzberg; U.S. Pat. No. 2,951,828 to Zeile; U.S. Pat. No. 2,676,945 to Higgins; and U.S. Pat. Nos. 2,683,136; 3,531,561 to Trehu.

PGA is a homopolymer of glycolic acid (hydroxyacetic acid). In the conversion of glycolic acid to poly (glycolic acid), glycolic acid is initially reacted with itself to form the cyclic ester glycolide, which in the presence of heat and a catalyst is converted to a high molecular weight linear-chain polymer. PGA polymers and their properties are described in more detail in “Cyanamid Research Develops World's First Synthetic Absorbable Suture”, Chemistry and Industry, 905 (1970).

Fibers can be formed by melt-spinning, extrusion, casting, or other techniques well known in the polymer processing area. Preferred solvents, if used to remove a scaffold prior to implantation, are those which are completely removed by the processing or which are biocompatible in the amounts remaining after processing.

Polymers for use in the matrix should meet the mechanical and biochemical parameters necessary to provide adequate support for the cells with subsequent growth and proliferation. The polymers can be characterized with respect to mechanical properties such as tensile strength using an INSTRON tester, for polymer molecular weight by gel permeation chromatography (GPC), glass transition temperature by differential scanning calorimetry (DSC) and bond structure by infrared (IR) spectroscopy.

The substrate or scaffold can be nanopatterned or micropatterned with grooves and ridges that permit growth of cardiac tissues on the scaffold. Scaffolds can be of any desired shape and can comprise a wide range of geometries that are useful for the methods described herein. A non-limiting list of shapes includes, for example, patches, hollow particles, tubes, sheets, cylinders, spheres, and fibers, among others. The shape or size of the scaffold should not substantially impede cell growth, cell differentiation, cell proliferation or any other cellular process, nor should the scaffold induce cell death via e.g., apoptosis or necrosis. In addition, care should be taken to ensure that the scaffold shape permits appropriate surface area for delivery of nutrients from the surrounding medium to cells in the population, such that cell viability is not impaired. The scaffold porosity can also be varied as desired by one of skill in the art.

In some embodiments, attachment of the cells to a polymer is enhanced by coating the polymers with compounds such as basement membrane components, fibronectin, agar, agarose, gelatin, gum arabic, collagens types I, II, III, IV, and V, laminin, glycosaminoglycans, polyvinyl alcohol, mixtures thereof, and other hydrophilic and peptide attachment materials known to those skilled in the art of cell culture or tissue engineering. Examples of a material for coating a polymeric scaffold include polyvinyl alcohol and collagen. As will be appreciated by one of skill in the art, Matrigel™ is not suitable for administration to a human subject, thus the compositions described herein do not include Matrigel™.

In some embodiments it can be desirable to add bioactive molecules/factors to the scaffold. A variety of bioactive molecules can be delivered using the matrices described herein.

In one embodiment, the bioactive factors include growth factors. Examples of growth factors include platelet derived growth factor (PDGF), transforming growth factor alpha or beta (TGFB), bone morphogenic protein 4 (BMP4), fibroblastic growth factor 7 (FGF7), fibroblast growth factor 10 (FGF10), epidermal growth factor (EGF/TGFα), vascular endothelium growth factor (VEGF), some of which are also angiogenic factors.

These factors are known to those skilled in the art and are available commercially or described in the literature. Bioactive molecules can be incorporated into the matrix and released over time by diffusion and/or degradation of the matrix, or they can be suspended with the cell suspension.

Secreted Factors that Enhance Engraftment of Transplanted Cardiomyocytes

In one aspect, described herein is a composition comprising cardiomyocytes in admixture with an isolated polypeptide or a nucleic acid encoding said polypeptide selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fstl1; Spp1; Ifne; Prl8a6; Srpx2; Dhrs11; Apom; Fam3c; Apbh/Scgb1b2; Timp3; Vwa; Tff3; Vstm2a; Grem1; Tac4; Cxc15; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2.

In another aspect, described herein is a composition comprising cardiomyocytes in admixture with two or more isolated polypeptides or nucleic acids encoding said polypeptides selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fst11; Spp1; Ifne; Prl8a6; Srpx2; Dhrs11; Apom; Fam3c; Apbh/Scgb1b2; Timp3; Vwa; Tff3; Vstm2a; Grem1; Tac4; Cxc15; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2.

In another aspect, described herein is a composition comprising cardiomyocytes in admixture with three or more, four or more, five or more, six or more, seven or more, eight or more, nine or more, or ten or more isolated polypeptides or nucleic acids encoding said polypeptides selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fstl1; Spp1; Ifne; Prl8a6; Srpx2; Dhrs11; Apom; Fam3c; Apbh/Scgb1b2; Timp3; Vwa; Tff3; Vstm2a; Grem1; Tac4; Cxc15; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2.

In another aspect, described herein is a composition comprising cardiomyocytes that have been contacted with an isolated polypeptide or nucleic acid encoding said polypeptide selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fstl1; Spp1; Ifne; Prl8a6; Srpx2; Dhrs11; Apom; Fam3c; Apbh/Scgb1b2; Timp3; Vwa; Tff3; Vstm2a; Grem1; Tac4; Cxc15; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2.

In another aspect, described herein is a composition comprising cardiomyocytes that have been contacted with two or more isolated polypeptides or nucleic acids encoding said polypeptides selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fst11; Spp1; Ifne; Prl8a6; Srpx2; Dhrs11; Apom; Fam3c; Apbh/Scgb1b2; Timp3; Vwa; Tff3; Vstm2a; Grem1; Tac4; Cxcl5; Serpine1; Gsn; Oxt; Ctfl; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2.

In another aspect, described herein is a composition comprising cardiomyocytes that have been contacted with three or more, four or more, five or more, six or more, seven or more, eight or more, nine or more, or ten or more isolated polypeptides or nucleic acids encoding said polypeptides selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fstl1; Spp1; Ifne; Prl8a6; Srpx2; Dhrs11; Apom; Fam3c; Apbh/Scgb1b2; Timp3; Vwa; Tff3; Vstm2a; Grem1; Tac4; Cxc15; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2.

TABLE 1
(below) is an exemplary list of the isolated polypeptides as described herein and their
associated gene, mRNA, and amino acid sequences for both human and mouse.

| | | | SEQ ID NOS: | | | |
|---|---|---|---|---|---|---|
| | | | mRNA | | protein | |
| Gene | Polypeptide | | sequences | | sequences | |
| Name | Name | Known Function | human | mouse | human | mouse |
| Rai2 | retinoic acid | vertebrate anteroposterior axis | 1 | 2 | 3 | 4 |
| | induced 2 | formation and cellular | | | | |
| | | differentiation; gene expression | | | | |
| Hmgb1 | high mobility | modulates transcription; DNA | 5 | 6 | 7 | 8 |
| | group box 1 | organization; inflammation; | | | | |
| | | cellular differentiation; tumor cell | | | | |
| | | migration | | | | |
| Furin | furin, paired | subtilisin-like proprotein | 9 | 10 | 11 | 12 |
| | basic amino acid | convertase family, which includes | | | | |
| | cleaving enzyme | proteases that process protein and | | | | |
| | | peptide precursors trafficking | | | | |
| | | through modulated or constitutive | | | | |
| | | branches of the secretory pathway | | | | |
| Cnpy4 | canopy FGF | regulation of the subcellular | 13 | 14 | 15 | 16 |
| | signaling | distribution of tlr4 | | | | |
| | regulator 4 | | | | | |
| C8a | complement | late-acting complement protein; | 17 | 18 | 19 | 20 |
| | component 8, | immunomodulatory function | | | | |
| | alpha subunit | | | | | |
| Il7 | interleukin 7 | T cell development and | 21 | 22 | 23 | 24 |
| | | homeostasis; immunomodulatory | | | | |
| | | function | | | | |
| Wnt3a | wingless-type | development; oncogenesis; | 25 | 26 | 27 | 28 |
| | MMTV | regulation of cell fate | | | | |
| | integration site | | | | | |
| | family, member | | | | | |
| | 3a | | | | | |
| Mmp21 | matrix | matrix metalloproteinase | 29 | 30 | 31 | 32 |
| | metallo- | superfamily that is known to | | | | |
| | proteinase 21 | hydrolyze extracellular matrix | | | | |
| | | components | | | | |
| Tinagl1 | tubulointerstitial | extracellular matrix protein | 33 | 34 | 35 | 36 |
| | nephritis | | | | | |
| | antigen-like | | | | | |
| | protein 1 | | | | | |
| Lipm | lipase family, | epidermal barrier function | 37 | 38 | 39 | 40 |
| | member m | | | | | |
| Klk8 | kallikrein 8 | serine protease; diverse | 41 | 42 | 43 | 44 |
| | | physiologic functions in many | | | | |
| | | tissues | | | | |
| Sepina12 | serpin peptidase | members of the serpin class of | 45 | 46 | 47 | 48 |
| | inhibitor, clade a, | protease inhibitors, like | | | | |
| | member 12: | SERPINA12, function as suicide | | | | |
| | serpin A12 | inhibitors by presenting a cleavable | | | | |
| | | bait sequence in their reactive | | | | |
| | | center loop that upon cleavage | | | | |
| | | forms an acyl-enzyme complex | | | | |
| | | and becomes distorted, preventing | | | | |
| | | release of the protease | | | | |
| Serpind1 | heparin cofactor | heparin cofactor II is a serine | 49 | 50 | 51 | 52 |
| | ii; hcf2 | protease inhibitor in plasma that | | | | |
| | | rapidly inhibits thrombin in the | | | | |
| | | presence of dermatan sulfate or | | | | |
| | | heparin | | | | |
| Gpc1 | glypican 1; gpc1 | antiangiogenic protein; modulates | 53 | 54 | 55 | 56 |
| | | growth, metastasis, and | | | | |
| | | angiogenesis | | | | |
| Psap | prosaposin | modulates lysosomal hydrolysis of | 57 | 58 | 59 | 60 |
| | | sphingolipids | | | | |
| Prss29 | serine protease | epithelial differentiation | 61 | 62 | 63 | 64 |
| | 29 | | | | | |
| Ambp | alpha-1 | regulation of the inflammatory | 65 | 66 | 67 | 68 |
| | microglobulin/ | process | | | | |
| | bikunin precursor | | | | | |
| Cpa6 | carboxypeptidase | carboxypeptidases have functions | 69 | 70 | 71 | 72 |
| | a6 | ranging from digestion of food to | | | | |
| | | selective biosynthesis of | | | | |
| | | neuroendocrine peptides | | | | |
| Scgb3a2 | secretoglobin, | controls the transcription of genes, | 73 | 74 | 75 | 76 |
| | family 3a, | such as surfactant proteins | | | | |
| | member 2 | | | | | |
| Reg3d | regenerating | immunomodulatory function | 77 | 78 | 79 | 80 |
| | islet-derived 3- | | | | | |
| | gamma | | | | | |
| Abpz/ | secretoglobin, | immunomodulatory function | 81 | 82 | 83 | 84 |
| Scgb2b24 | family 2B, | | | | | |
| | member 24; | | | | | |
| | Scgb2b3 | | | | | |
| Npff | neuropeptide FF- | neural activity | 85 | 86 | 87 | 88 |
| | amide peptide | | | | | |
| Fam3b | family with | modulates insulin secretion | 89 | 90 | 91 | 92 |
| | sequence | | | | | |
| | similarity 3, | | | | | |
| | member b | | | | | |
| Fstl1 | follistatin-related | modulates cell migration and | 93 | 94 | 95 | 96 |
| | protein | wound repair | | | | |
| Spp1 | secreted | bone mineralization | 97 | 98 | 99 | 100 |
| | phosphoprotein | | | | | |
| | 1; Osteopontin | | | | | |
| Ifne | interferon, | cell proliferation and activation of | 101 | 102 | 103 | 104 |
| | epsilon; IFN-ε | the immune system | | | | |
| Prl8a6 | prolactin-8A6 | Lactation hormone | 105 | 106 | 107 | 108 |
| Srpx2 | sushi repeat- | immunomodulatory function; | 109 | 110 | 111 | 112 |
| | containing | regulation of apoptosis; | | | | |
| | protein, x-linked, | development; synaptogenesis | | | | |
| | 2 | | | | | |
| Dhrs11 | short-chain | metabolism of steroid hormones, | 113 | 114 | 115 | 116 |
| | dehydrogenase/ | prostaglandins, retinoids, lipids, | | | | |
| | reductase family, | and xenobiotics | | | | |
| | member 11 | | | | | |
| Apom | apolipoprotein m | immunomodulatory function; | 117 | 118 | 119 | 120 |
| | | lymphocyte trafficking; regulation | | | | |
| | | of cholesterol (HDL) | | | | |
| Fam3c | family with | immunomodulatory function; | 121 | 122 | 123 | 124 |
| | sequence | neural function | | | | |
| | similarity 3, | | | | | |
| | member c | | | | | |
| Apbh/ | Secretoglobin, | immunomodulatory function; | 125 | 126 | 127 | 128 |
| Scgb1b2 | family 1B, | development | | | | |
| | member 2; | | | | | |
| | androgen- | | | | | |
| | binding protein | | | | | |
| | eta precursor | | | | | |
| Timp3 | tissue inhibitor | modulates angiogenesis; modulates | 129 | 130 | 131 | 132 |
| | of | extracellular matrix protein | | | | |
| | metallo- | function; modulates cell migration | | | | |
| | proteinase 3 | | | | | |
| Vwa | von Willebrand | extracellular matrix protein | 133 | 134 | 135 | 136 |
| | Factor A-like | | | | | |
| | domain; vwa2 | | | | | |
| Tff3 | trefoil factor 3 | transcriptional regulator, | 137 | 138 | 139 | 140 |
| | | immunomodulatory function, | | | | |
| | | hyperplasia | | | | |
| Vstm2a | V-set and | regulation of the early stage of | 141 | 142 | 143 | 144 |
| | transmembrane | white and brown preadipocyte cell | | | | |
| | domain- | differentiation | | | | |
| | containing | | | | | |
| | protein 2A | | | | | |
| Grem1 | gremlin 1 | development, cell proliferation | 145 | 146 | 147 | 148 |
| | homolog, | | | | | |
| | cysteine knot | | | | | |
| | superfamily | | | | | |
| Tac4 | tachykinin 4 | modulates B-cell development | 149 | 150 | 151 | 152 |
| Cxcl5 | chemokine, cxc | immunomodulatory function | 153 | 154 | 155 | 156 |
| | motif, ligand 5 | | | | | |
| Serpine1 | serpin peptidase | Serine protease; modulates blood | 157 | 158 | 159 | 160 |
| | inhibitor, clade e | clotting | | | | |
| | (nexin, | | | | | |
| | plasminogen | | | | | |
| | activator | | | | | |
| | inhibitor type 1 | | | | | |
| Gsn | gelsolin | Modulates amyloidosis; modulates | 161 | 162 | 163 | 164 |
| | | actin release into the extracellular | | | | |
| | | space; modulates apoptosis | | | | |
| Oxt | oxytocin | hormone; neural activity; | 165 | 166 | 167 | 168 |
| | | modulates development | | | | |
| Ctf1 | cardiotrophin 1 | modulates cardiac hypertrophy; | 169 | 170 | 171 | 172 |
| | | modulates cardiac development; | | | | |
| | | modulates neural activity | | | | |
| Mdk | MIDKINE | retinoic acid-responsive, heparin- | 173 | 174 | 175 | 176 |
| | | binding growth factor expressed in | | | | |
| | | various cell types during | | | | |
| | | embryogenesis; angiogenesis; | | | | |
| | | tumorigenesis | | | | |
| Pla2g2c | phospholipase | modulates phospholipid signaling | 177 | 178 | 179 | 180 |
| | a2, group v | | | | | |
| Nhlrc3 | MKIAA4083 | development | 181 | 182 | 183 | 184 |
| | protein | | | | | |
| Glipr1l1 | glipr1-like | Cell proliferation and cell growth | 185 | 186 | 187 | 188 |
| | protein 1 | | | | | |
| Mcpt7 | mouse mast cell | unknown | 189 | 190 | 191 | 192 |
| | protease 7; | | | | | |
| | TPSAB1 | | | | | |
| Il12a | interleukin 12a | immunomodulatory function | 193 | 194 | 195 | 196 |
| 2410001C21Rik | 2410001C21Rik | modulates DNA replication | 197 | 198 | 199 | 200 |
| | protein; Rtf2 | | | | | |
| Fjx1 | four-jointed box | modulates cell growth and gene | 201 | 202 | 203 | 204 |
| | 1 | expression | | | | |
| Cntn4 | contactin 4 | modulates formation, maintenance, | 205 | 206 | 207 | 208 |
| | | and plasticity of functional | | | | |
| | | neuronal networks | | | | |
| Fetub | fetuin b | immunomodulatory function; | 209 | 210 | 211 | 212 |
| | | osteogenesis and bone resorption, | | | | |
| | | regulation of the insulin and | | | | |
| | | hepatocyte growth factor receptors | | | | |
| Ccl6 | chemokine (C-C | differentiation; immunomodulatory | 213 | 214 | 215 | 216 |
| | motif) ligand 6 | function | | | | |
| Thbs2 | thrombospondin | neural activity; synaptogenesis; | 217 | 218 | 219 | 220 |
| | II | cell modulates cell adhesion and | | | | |
| | | migration | | | | |
| Fbln2 | fibulin 2 | development; differentiation | 221 | 222 | 223 | 224 |
| Htra1 | HTRA serine | modulates extracellular matrix | 225 | 226 | 227 | 228 |
| | peptidase 1 | components; development; | | | | |
| | | oncogenesis | | | | |
| Sfrp1 | secreted frizzled- | anti-apoptosis; cell growth | 229 | 230 | 231 | 232 |
| | related protein 1 | | | | | |
| Wnt6 | wingless-type | cell growth; differentiation; | 233 | 234 | 235 | 236 |
| | MMTV | development | | | | |
| | integration site | | | | | |
| | family, member | | | | | |
| | 6 | | | | | |
| Impg2 | Interphoto- | Photoreception; | 237 | 238 | 239 | 240 |
| | receptor matrix | | | | | |
| | proteoglycan 2 | | | | | |

In some embodiments of any of the aspects, a composition or method described herein can comprise an isolated polypeptide or nucleic acid encoding said polypeptide selected from the group consisting of: at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or 60 of the polypeptides or nucleic acids listed in Table 1.

In some embodiments of any of the aspects, a composition or method described herein can comprise an isolated polypeptide or nucleic acid encoding said polypeptide selected from the group consisting of: at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, or at most 60 of the polypeptides or nucleic acids listed in Table 1.

In some embodiments of any of the aspects, a composition or method described herein can comprise isolated murine polypeptides or murine nucleic acids encoding said murine polypeptides selected from the group consisting of: Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fst11; Spp1; Ifne; Prl8a6; Srpx2; Dhrs11; Apom; Fam3c; Apbh/Scgb1b2; Timp3; Vwa; Tff3; Vstm2a; Grem1; Tac4; Cxc15; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2. In some embodiments, a human homolog of a polypeptide can be used in place of the mouse homolog of the polypeptide.

In some embodiments of any of the aspects, a composition or method described herein can comprise isolated human polypeptides or human nucleic acids encoding said human polypeptides selected from the group consisting of: Rai2; Hmgb1; Furin; Cnpy4; C8a; Il7; Wnt3a; Mmp21; Tinagl1; Lipm; Klk8; Sepina12; Serpind1; Gpc1; Psap; Ambp; Cpa6; Scgb3a2; Npff; Fam3b; Fstl1; Spp1; Ifne; Srpx2; Dhrs11; Apom; Fam3c; Timp3; Vwa; Tff3; Vstm2a; Grem1; Tac4; Cxc15; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Thbs2; Fbln2; Htra1; Sfrp1; Wnt6; and Impg2. In some embodiments, a mouse homolog of a polypeptide can be used in place of the human homolog of the polypeptide. In some embodiments, a mouse homolog, or another mammalian homolog (e.g., chimp, rat, etc.), of a polypeptide can be used when there is no human homolog known for the polypeptide (e.g., for Prss29, Reg3d; Abpz/Scgb2b24; Prl8a6; Apbh/Scgb1b2; and/or Ccl6).

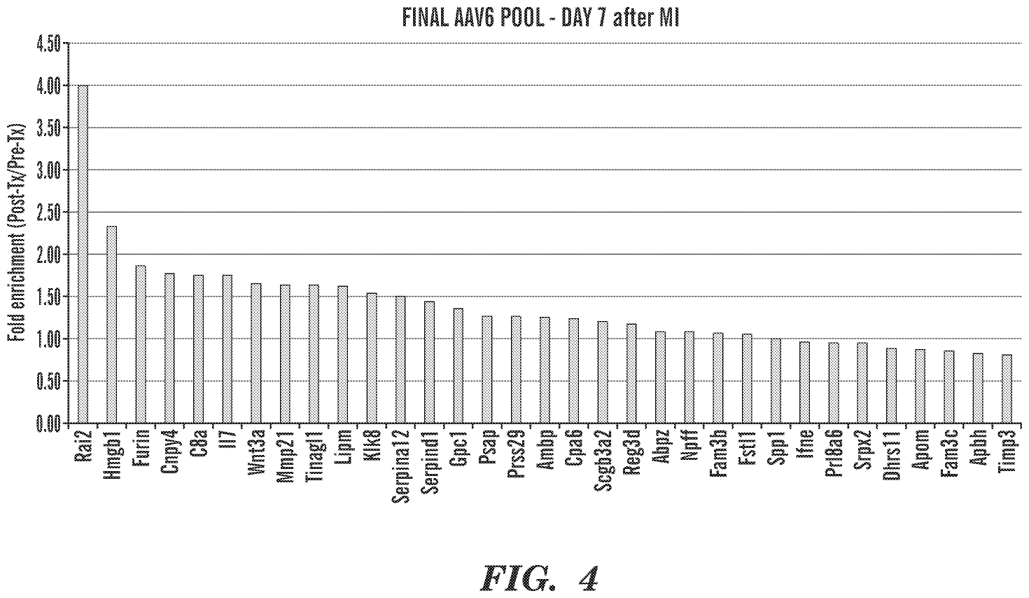

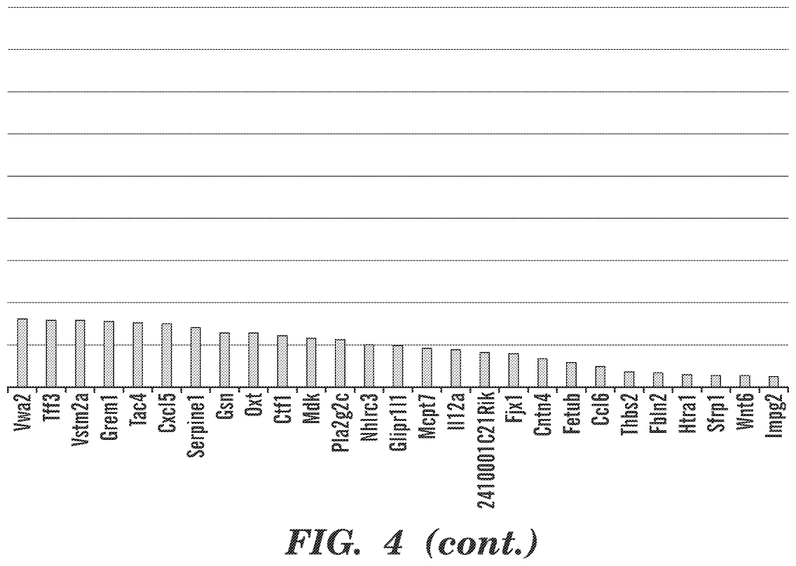

The genes and proteins in Table 1 are listed in order of decreasing fold enrichment, e.g., with Rai2 having the highest fold enrichment (see e.g., FIG. 4). In some embodiments of any of the aspects, a composition or method described herein can comprise an isolated polypeptide or nucleic acid encoding said polypeptide selected from the group consisting of: the first 1, the first 2, the first 3, the first 4, the first 5, the first 6, the first 7, the first 8, the first 9, the first 10, the first 11, the first 12, the first 13, the first 14, the first 15, the first 16, the first 17, the first 18, the first 19, the first 20, the first 21, the first 22, the first 23, the first 24, the first 25, the first 26, the first 27, the first 28, the first 29, the first 30, the first 31, the first 32, the first 33, the first 34, the first 35, the first 36, the first 37, the first 38, the first 39, the first 40, the first 41, the first 42, the first 43, the first 44, the first 45, the first 46, the first 47, the first 48, the first 49, the first 50, the first 51, the first 52, the first 53, the first 54, the first 55, the first 56, the first 57, the first 58, the first 59, or 60 of the polypeptides or nucleic acids listed in Table 1.

The sequences provided herein can be modified, comprise conservative amino acid substitutions, or have additional amino acids that can improve targeting or efficacy of the composition described herein. It is contemplated that other mammalian sequences can be used (e.g., rat, rabbit, guinea pig, sheep, cow, dog, horse, etc.) that are homologous to the polypeptides or nucleic acids encoding said polypeptides as described herein.

In particular, a human homologue of a murine protein, mRNA, or cDNA is expressly contemplated as being useful in the methods and compositions described herein.

The term “homology” as used herein refers to sequence similarity between two peptides or between two nucleic acid molecules. Homology can be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology refers to a function of the number of identical or similar amino acids at positions shared by the compared sequences. A sequence which is “unrelated” or “non-homologous” shares less than 40% identity. Determination of homologs of the genes or peptides described herein may be easily ascertained by the skilled artisan.

The sequences provided here can be modified, comprise conservative amino acid substitutions, or have additional amino acids that can improve targeting or efficacy of the composition described herein.

The term “conservative substitution,” when describing a polypeptide, refers to a change in the amino acid composition of the polypeptide that does not substantially alter the polypeptide's activity, for example, a conservative substitution refers to substituting an amino acid residue for a different amino acid residue that has similar chemical properties. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity. Conservative substitution tables providing functionally similar amino acids are well known in the art. For example, the following six groups each contain amino acids that are conservative substitutions for one another: 1) Alanine (A), Serine(S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.” Insertions or deletions are typically in the range of about 1 to 5 amino acids.

### Cellular Signaling and Functional Activity of Secreted Proteins

In one aspect, described herein is a composition comprising cardiomyocytes in admixture with an isolated metabolic factor polypeptide that promotes transplant engraftment of the cardiomyocytes, or with a nucleic acid construct that encodes such a factor.

Tables 2-17 provide the functional activity and/or signaling pathways related to the genes and polypeptides described herein that enhance engraftment of cardiomyocytes as shown in Table 1.

In another aspect, described herein is a composition comprising cardiomyocytes in admixture with an isolated canonical Wnt pathway polypeptide.

In another aspect, described herein is a composition comprising cardiomyocytes that have been contacted with an isolated canonical Wnt pathway polypeptide.

In some embodiments, the canonical Wnt pathway polypeptide is selected from the group consisting of the isolated polypeptides or nucleic acids encoding said polypeptides in Table 2. In some embodiments of any of the aspects, a composition or method described herein can comprise an isolated polypeptide or nucleic acid encoding said polypeptide selected from the group consisting of: at least 1, at least 2, at least 3, at least 4, or 5 of the polypeptides or nucleic acids listed in Table 2. In some embodiments of any of the aspects, a composition or method described herein can comprise an isolated polypeptide or nucleic acid encoding said polypeptide selected from the group consisting of: at most 1, at most 2, at most 3, at most 4, or at most 5 of the polypeptides or nucleic acids listed in Table 2.

Wnt signaling is involved in cellular growth, development, differentiation, cell fate determination, and cell survival. The Wnt polypeptides are secreted glycoproteins that bind to the Frizzled (Fz) receptor and activate a complex signaling cascade. The canonical and non-canonical Wnt signalling pathways are known in the art. See for example, Komiya et al. Organogenesis. 2008 April-Jun; 4 (2): 68-75, which is incorporated herein by reference in its entirety. Table 2 shows the secreted proteins identified to improve cardiomyocyte engraftment that are associated with canonical Wnt signaling.

TABLE 2
Canonical Wnt Pathway

| Canonical Wnt Pathway | Fold Enrichment |
|---|---|
| WNT3A | 1.65 |
| TINAGL1 | 1.63 |
| SFRP1 | 0.14 |
| WNT6 | 0.13 |
| GPC1 (antagonist) | 1.37 |

In some embodiments of the aspects described herein, the isolated metabolic factor is selected from the group consisting of a polypeptide that promotes lipid hydrolysis and a polypeptide that modulates insulin or IGF signaling.

In other embodiments, the polypeptide that promotes lipid hydrolysis is selected from LIPM, PSAP and PLA2G2C, and the polypeptide that modulates insulin or IGF signaling is selected from SERPINA12, HTRA1 and FETUB. In some embodiments, the two or more polypeptides or nucleic acids encoding said polypeptides described herein are selected from Tables 3-5.

In some embodiments of any of the aspects, a composition or method described herein can comprise an isolated polypeptide or nucleic acid encoding said polypeptide selected from the group consisting of: at least 1, at least 2, or 3 of the polypeptides or nucleic acids listed in Table 3. In some embodiments of any of the aspects, a composition or method described herein can comprise an isolated polypeptide or nucleic acid encoding said polypeptide selected from the group consisting of: at most 1, at most 2, or at most 3 of the polypeptides or nucleic acids listed in Table 3.

In some embodiments of any of the aspects, a composition or method described herein can comprise an isolated polypeptide or nucleic acid encoding said polypeptide selected from the group consisting of: at least 1 or 2 of the polypeptides or nucleic acids listed in Table 4. In some embodiments of any of the aspects, a composition or method described herein can comprise an isolated polypeptide or nucleic acid encoding said polypeptide selected from the group consisting of: at most 1 or at most 2 of the polypeptides or nucleic acids listed in Table 4.

In some embodiments of any of the aspects, a composition or method described herein can comprise an isolated polypeptide or nucleic acid encoding said polypeptide selected from the group consisting of: at least 1 or 2 of the polypeptides or nucleic acids listed in Table 5. In some embodiments of any of the aspects, a composition or method described herein can comprise an isolated polypeptide or nucleic acid encoding said polypeptide selected from the group consisting of: at most 1 or at most 2 of the polypeptides or nucleic acids listed in Table 5.

Lipid synthesis and degradation of lipids are important in cellular metabolism and the ability for cells to produce and maintain cellular membranes. Lipids are degraded and hydrolyzed by lipases that allow for further lipid metabolism and the production of cellular adenosine triphosphates (ATP). For example, LIPM is a lipase which is associated with improving epidermal barrier function. See for example, Toulza et al. Genome Biol (2007), which is incorporated herein by reference in its entirety.

TABLE 3
Lipid Hydrolysis

| Lipid Hydrolysis | Fold Enrichment |
|---|---|
| LIPM | 1.63 |
| PSAP | 1.27 |
| PLA2G2C | 0.57 |

Secreted proteins that improve cardiac engraftment were also identified to modulate or inhibit insulin and insulin-like growth factor (IGF) signaling. IGF can stimulate cell growth in many different cell types.

TABLE 4
Modulates insulin/IGF Signaling

| Augments Insulin/IGF | |
|---|---|
| Signaling | Fold Enrichment |
| SERPINA12 | 1.48 |
| HTRA1 | 0.15 |

TABLE 5
Inhibits Insulin/IGF Signaling

| Inhibit Insulin/IGF | |
|---|---|
| Signaling | Fold Enrichment |
| FETUB | 0.30 |
| HTRA1 | 0.15 |

In another aspect, described herein is a composition comprising cardiomyocytes in admixture with an isolated vascular remodeling, extracellular matrix, proteoglycan or cell adhesion polypeptide that promotes transplant engraftment of the cardiomyocytes, or with a nucleic acid construct that encodes such a factor.

In some embodiments, the composition comprises two or more polypeptide factors selected from the groups consisting of vascular remodeling, extracellular matrix, proteoglycan and cell adhesion polypeptides. In some embodiments, the composition comprises three or more, four or more, five or more polypeptide factors selected from Tables 6-9.

In some embodiments, the vascular remodeling polypeptide is selected from Table 6, the extracellular matrix polypeptide is selected from Table 7, the proteoglycan polypeptide is selected from Table 8 and the cell adhesion polypeptide is selected from Table 9.

In some embodiments of any of the aspects, a composition or method described herein can comprise an isolated polypeptide or nucleic acid encoding said polypeptide selected from the group consisting of: at least 1, at least 2, at least 3, at least 4, at least 5, at least 6, at least 7, at least 8, at least 9, at least 10, at least 11, at least 12, at least 13, at least 14, at least 15, or 16 of the polypeptides or nucleic acids listed in Table 6. In some embodiments of any of the aspects, a composition or method described herein can comprise an isolated polypeptide or nucleic acid encoding said polypeptide selected from the group consisting of: at most 1, at most 2, at most 3, at most 4, at most 5, at most 6, at most 7, at most 8, at most 9, at most 10, at most 11, at most 12, at most 13, at most 14, at most 15, or at most 16 of the polypeptides or nucleic acids listed in Table 6.

In some embodiments of any of the aspects, a composition or method described herein can comprise an isolated polypeptide or nucleic acid encoding said polypeptide selected from the group consisting of: at least 1, at least 2, at least 3, at least 4, or 5 of the polypeptides or nucleic acids listed in Table 7. In some embodiments of any of the aspects, a composition or method described herein can comprise an isolated polypeptide or nucleic acid encoding said polypeptide selected from the group consisting of: at most 1, at most 2, at most 3, at most 4, or at most 5 of the polypeptides or nucleic acids listed in Table 7.

In some embodiments of any of the aspects, a composition or method described herein can comprise an isolated polypeptide or nucleic acid encoding said polypeptide selected from the group consisting of: at least 1 or 2 of the polypeptides or nucleic acids listed in Table 8. In some embodiments of any of the aspects, a composition or method described herein can comprise an isolated polypeptide or nucleic acid encoding said polypeptide selected from the group consisting of: at most 1 or at most 2 of the polypeptides or nucleic acids listed in Table 8.

In some embodiments of any of the aspects, a composition or method described herein can comprise an isolated polypeptide or nucleic acid encoding said polypeptide selected from the group consisting of: at least 1 or 2 of the polypeptides or nucleic acids listed in Table 9. In some embodiments of any of the aspects, a composition or method described herein can comprise an isolated polypeptide or nucleic acid encoding said polypeptide selected from the group consisting of: at most 1 or at most 2 of the polypeptides or nucleic acids listed in Table 9.

The secreted proteins that enhance cardiomyocyte engraftment can also improve vascular remodeling and cellular adhesion. Vascular remodeling is the process in which blood vessels under go structural alterations that changes cell growth, cell death, cell migration, and production or degradation of extracellular matrix proteins. The structural alteration can include changes in vessel or cell wall mass, modulation of cell populations, or modulation of external vessel or cellular diameter.

TABLE 6
Vascular Remodeling

| Vascular Remodeling | Fold Enrichment |
|---|---|
| HMGB1 | 2.32 |
| FURIN | 1.87 |
| WNT3A | 1.65 |
| TINAGL1 | 1.63 |
| SERPIND1 | 1.43 |
| GPC1 | 1.37 |
| FSTL1 | 1.05 |
| SPP1 | 0.99 |
| SRPX2 | 0.94 |
| TFF3 | 0.80 |
| GREM1 | 0.78 |
| CXCL5 | 0.75 |
| OXT | 0.64 |
| CTF1 | 0.61 |
| MDK | 0.58 |
| SFRP1 | 0.14 |

TABLE 7
Extracellular Matrix (ECM)

| Extracellular | |
|---|---|
| Matrix | Fold Enrichment |
| TINAGL1 | 1.63 |
| GPC1 | 1.37 |
| VWA2 | 0.81 |
| FBLN2 | 0.17 |
| IMPG2 | 0.13 |

TABLE 8
Proteoglycan

| Proteoglycan | Fold Enrichment |
|---|---|
| GPC1 | 1.37 |
| IMPG2 | 0.13 |

TABLE 9
Cell Adhesion

| Cell Adhesion | Fold Enrichment |
|---|---|
| CNTN4 | 0.34 |
| THBS2 | 0.18 |

In another aspect, described herein is a composition comprising cardiomyocytes in admixture with an isolated serine protease polypeptide.

In another aspect, described herein is a composition comprising cardiomyocytes that have been contacted with an isolated serine protease polypeptide.

In some embodiments, the serine protease polypeptide is selected from Table 10. In some embodiments of any of the aspects, a composition or method described herein can comprise an isolated polypeptide or nucleic acid encoding said polypeptide selected from the group consisting of: at least 1, at least 2, at least 3, at least 4, or 5 of the polypeptides or nucleic acids listed in Table 10. In some embodiments of any of the aspects, a composition or method described herein can comprise an isolated polypeptide or nucleic acid encoding said polypeptide selected from the group consisting of: at most 1, at most 2, at most 3, at most 4, or at most 5 of the polypeptides or nucleic acids listed in Table 10.

TABLE 10
Serine Proteases

| Serine Protease | Fold Enrichment |
|---|---|
| FURIN | 1.87 |
| KLK8 | 1.55 |
| PRSS29P | 1.27 |
| TPSAB1/MCPT7 | 0.47 |
| HTRA1 | 0.15 |

In another aspect, described herein is a composition comprising cardiomyocytes in admixture with an isolated serine protease inhibitor polypeptide.

In another aspect, described herein is a composition comprising cardiomyocytes that have been contacted with an isolated serine protease inhibitor polypeptide.

In some embodiments, the isolated serine protease inhibitor polypeptide is selected from Table 11. In some embodiments of any of the aspects, a composition or method described herein can comprise an isolated polypeptide or nucleic acid encoding said polypeptide selected from the group consisting of: at least 1, at least 2, at least 3, at least 4, or 5 of the polypeptides or nucleic acids listed in Table 11. In some embodiments of any of the aspects, a composition or method described herein can comprise an isolated polypeptide or nucleic acid encoding said polypeptide selected from the group consisting of: at most 1, at most 2, at most 3, at most 4, or at most 5 of the polypeptides or nucleic acids listed in Table 11.

The secreted polypeptide described herein can enhance cardiomyocyte engraftment by modulating serine protease function. Serine proteases are enzymes that cleave other proteins and polypeptides and are responsible for the regulation of various physiological functions (e.g., immune response, cell growth and proliferation, coagulation, differentiation, etc.).

TABLE 11
Serine Protease Inhibitors

| Serine Protease | Fold Enrichment |
|---|---|
| FURIN | 1.87 |
| KLK8 | 1.55 |
| PRSS29P | 1.27 |
| TPSAB1/MCPT7 | 0.47 |
| HTRA1 | 0.15 |

In another aspect, described herein is a composition comprising cardiomyocytes in admixture with an isolated signaling polypeptide of the interleukin family, interferon signaling family, or chemokine family.

In another aspect, described herein is a composition comprising cardiomyocytes that have been contacted with an isolated signaling polypeptide of the interleukin family, interferon signaling family, or chemokine family.

In some embodiments, the signaling polypeptide of the interleukin family is selected from Table 12, the signaling polypeptide of the interferon signaling family is selected from Table 13, and the signaling polypeptide of the chemokine family is selected from Table 14. In some embodiments of any of the aspects, a composition or method described herein can comprise an isolated polypeptide or nucleic acid encoding said polypeptide selected from the group consisting of: at least 1, at least 2, or 3 of the polypeptides or nucleic acids listed in Table 12. In some embodiments of any of the aspects, a composition or method described herein can comprise an isolated polypeptide or nucleic acid encoding said polypeptide selected from the group consisting of: at most 1, at most 2, or at most 3 of the polypeptides or nucleic acids listed in Table 12.

In some embodiments of any of the aspects, a composition or method described herein can comprise an isolated polypeptide or nucleic acid encoding said polypeptide selected from the group consisting of: at least 1 or 2 of the polypeptides or nucleic acids listed in Table 13. In some embodiments of any of the aspects, a composition or method described herein can comprise an isolated polypeptide or nucleic acid encoding said polypeptide selected from the group consisting of: at most 1 or at most 2 of the polypeptides or nucleic acids listed in Table 13.

In some embodiments of any of the aspects, a composition or method described herein can comprise an isolated polypeptide or nucleic acid encoding said polypeptide selected from the group consisting of: at least 1 or 2 of the polypeptides or nucleic acids listed in Table 14. In some embodiments of any of the aspects, a composition or method described herein can comprise an isolated polypeptide or nucleic acid encoding said polypeptide selected from the group consisting of: at most 1 or at most 2 of the polypeptides or nucleic acids listed in Table 14.

The secreted polypeptide(s) described herein can be immunomodulatory polypeptides such as interleukins, interferons, or chemokines.

As used herein, the term “immunomodulatory” refers to an agent, polypeptide, gene, or nucleic acid as described herein that modulates (increases, decreases, suppresses or potentiates) one or more activities or functions or cells of the immune system. An immunomodulator can modulate the activity of the innate immune system, the adaptive immune system, or both.

An “immunomodulatory polypeptide” is a polypeptide that has immunomodulatory activity. Non-limiting examples include cytokines, immune checkpoint molecules (checkpoint receptors and ligands therefor), or other polypeptides that modulate activities or functions or cells of the immune system.

TABLE 12
Interleukin family

| Interleukin Family | Fold Enrichment |
|---|---|
| IL7 | 1.76 |
| CTF1 | 0.61 |
| IL12A | 0.44 |

TABLE 13
Interferon Signaling

| Interferon Signaling | Fold Enrichment |
|---|---|
| IFNE | 0.97 |
| IL12A | 0.44 |

TABLE 14
Chemokine Family

| Chemokine Family | Fold Enrichment |
|---|---|
| CXCL5 | 0.75 |
| CCL6 | 0.25 |

In another aspect, described herein is a composition comprising cardiomyocytes in admixture with an isolated TLR binding polypeptide, a lipocalin polypeptide, or a secretoglobin polypeptide.

In another aspect, described herein is a composition comprising cardiomyocytes that have been contacted with an isolated TLR binding polypeptide, a lipocalin polypeptide, or a secretoglobin polypeptide.

In some embodiments, the TLR binding polypeptide is selected from Table 15, the lipocalin polypeptide is selected from Table 16 and the secretoglobin polypeptide is selected from Table 17. In some embodiments of any of the aspects, a composition or method described herein can comprise an isolated polypeptide or nucleic acid encoding said polypeptide selected from the group consisting of: at least 1 or 2 of the polypeptides or nucleic acids listed in Table 15. In some embodiments of any of the aspects, a composition or method described herein can comprise an isolated polypeptide or nucleic acid encoding said polypeptide selected from the group consisting of: at most 1 or at most 2 of the polypeptides or nucleic acids listed in Table 15.

In some embodiments of any of the aspects, a composition or method described herein can comprise an isolated polypeptide or nucleic acid encoding said polypeptide selected from the group consisting of: at least 1 or 2 of the polypeptides or nucleic acids listed in Table 16. In some embodiments of any of the aspects, a composition or method described herein can comprise an isolated polypeptide or nucleic acid encoding said polypeptide selected from the group consisting of: at most 1 or at most 2 of the polypeptides or nucleic acids listed in Table 16.

In some embodiments of any of the aspects, a composition or method described herein can comprise an isolated polypeptide or nucleic acid encoding said polypeptide selected from the group consisting of: at least 1, at least 2, or 3 of the polypeptides or nucleic acids listed in Table 17. In some embodiments of any of the aspects, a composition or method described herein can comprise an isolated polypeptide or nucleic acid encoding said polypeptide selected from the group consisting of: at most 1, at most 2, or at most 3 of the polypeptides or nucleic acids listed in Table 17.

Additional immunomodulatory polypeptides can include toll-like receptors (TLRs) or polypeptides that modulate TLR signaling, lipocalins, or secretoglobins. Secretoglobins can also regulate thyroid-specific expression of genes that are responsible for cellular differentiation and cell growth during development.

TABLE 15
Toll Like Receptor Binding

| TLR Binding | Fold Enrichment |
|---|---|
| HMGB1 (↑ TLR) | 2.32 |
| CNPY4 (↓ TLR) | 1.77 |

TABLE 16
Lipocalins

| Lipocalins | Fold Enrichment |
|---|---|
| AMBP | 1.25 |
| APOM | 0.88 |

TABLE 17
Secretoglobins

| Secretoglobins | Fold Enrichment |
|---|---|
| SCGB3A2 | 1.21 |
| SCGB2B3/ABPZ | 1.08 |
| SCGB1B2/APBH | 0.83 |

In another aspect of any of the embodiments described herein, the isolated polypeptide(s) or a nucleic acid(s) encoding said polypeptide(s) can be selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fstl1; Spp1; Ifne; Prl8a6; Srpx2; Dhrs11; Apom; Fam3c; Apbh/Scgb1b2; Timp3; Vwa; Tff3; Vstm2a; Grem1; Tac4; Cxcl5; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2, in any combination. Multiple isolated polypeptides and/or nucleic acids encoding the polypeptides in Tables 1-19 can be used in the compositions and admixtures described herein. Tables 18-19 provide examples of exemplary combinations of isolated polypeptide(s) or a nucleic acid(s) encoding said polypeptide(s) that can be used and/or comprised by the compositions or methods described herein. Table 18 shows exemplary combinations of nucleic acids (or their encoded polypeptides) with the first nucleic acid (or its encoded polypeptide) indicated in the first row, the second nucleic acid (or its encoded polypeptide) indicated in the first column, and “x” indicating an exemplary combination. Table 19 shows exemplary combinations of at least one nucleic acid (or their encoded polypeptide) selected from the indicated table(s), with “x” indicating that at least one nucleic acid (or their encoded polypeptide) is selected from the indicated table(s) for the exemplary combination.

TABLE 18
Exemplary combinations of genes and polypeptides as described herein

| | Rai2 | Hmgb1 | Furin | Cnpy4 | C8a | Il7 | Wnt3a | Mmp21 |
|---|---|---|---|---|---|---|---|---|
| Rai2 | | x | x | x | x | x | x | x |
| Hmgb1 | x | | x | x | x | x | x | x |
| Furin | x | x | | x | x | x | x | x |
| Cnpy4 | x | x | x | | x | x | x | x |
| C8a | x | x | x | x | | x | x | x |
| Il7 | x | x | x | x | x | | x | x |
| Wnt3a | x | x | x | x | x | x | | x |
| Mmp21 | x | x | x | x | x | x | x | |
| Tinagl1 | x | x | x | x | x | x | x | x |
| Lipm | x | x | x | x | x | x | x | x |
| Klk8 | x | x | x | x | x | x | x | x |
| Sepina12 | x | x | x | x | x | x | x | x |
| Serpind1 | x | x | x | x | x | x | x | x |
| Gpc1 | x | x | x | x | x | x | x | x |
| Psap | x | x | x | x | x | x | x | x |
| Prss29 | x | x | x | x | x | x | x | x |
| Ambp | x | x | x | x | x | x | x | x |
| Cpa6 | x | x | x | x | x | x | x | x |
| Scgb3a2 | x | x | x | x | x | x | x | x |
| Reg3d | x | x | x | x | x | x | x | x |
| Abpz; Scgb2b24 | x | x | x | x | x | x | x | x |
| Npff | x | x | x | x | x | x | x | x |
| Fam3b | x | x | x | x | x | x | x | x |
| Fstl1 | x | x | x | x | x | x | x | x |
| Spp1 | x | x | x | x | x | x | x | x |
| Ifne | x | x | x | x | x | x | x | x |
| Prl8a6 | x | x | x | x | x | x | x | x |
| Srpx2 | x | x | x | x | x | x | x | x |
| Dhrs11 | x | x | x | x | x | x | x | x |
| Apom | x | x | x | x | x | x | x | x |
| Fam3c | x | x | x | x | x | x | x | x |
| Apbh; Scgb1b2 | x | x | x | x | x | x | x | x |
| Timp3 | x | x | x | x | x | x | x | x |
| Vwa | x | x | x | x | x | x | x | x |
| Tff3 | x | x | x | x | x | x | x | x |
| Vstm2a | x | x | x | x | x | x | x | x |
| Grem1 | x | x | x | x | x | x | x | x |
| Tac4 | x | x | x | x | x | x | x | x |
| Cxcl5 | x | x | x | x | x | x | x | x |
| Serpine1 | x | x | x | x | x | x | x | x |
| Gsn | x | x | x | x | x | x | x | x |
| Oxt | x | x | x | x | x | x | x | x |
| Ctf1 | x | x | x | x | x | x | x | x |
| Mdk | x | x | x | x | x | x | x | x |
| Pla2g2c | x | x | x | x | x | x | x | x |
| Nhlrc3 | x | x | x | x | x | x | x | x |
| Glipr1l1 | x | x | x | x | x | x | x | x |
| Mcpt7 | x | x | x | x | x | x | x | x |
| Il12a | x | x | x | x | x | x | x | x |
| 2410001C21Rik | x | x | x | x | x | x | x | x |
| Fjx1 | x | x | x | x | x | x | x | x |
| Cntn4 | x | x | x | x | x | x | x | x |
| Fetub | x | x | x | x | x | x | x | x |
| Ccl6 | x | x | x | x | x | x | x | x |
| Thbs2 | x | x | x | x | x | x | x | x |
| Fbln2 | x | x | x | x | x | x | x | x |
| Htra1 | x | x | x | x | x | x | x | x |
| Sfrp1 | x | x | x | x | x | x | x | x |
| Wnt6 | x | x | x | x | x | x | x | x |
| Impg2 | x | x | x | x | x | x | x | x |
| | Tinagl1 | Lipm | Klk8 | Sepina12 | Serpind1 | Gpc1 | Psap | Prss29 |
| Rai2 | x | x | x | x | x | x | x | x |
| Hmgb1 | x | x | x | x | x | x | x | x |
| Furin | x | x | x | x | x | x | x | x |
| Cnpy4 | x | x | x | x | x | x | x | x |
| C8a | x | x | x | x | x | x | x | x |
| Il7 | x | x | x | x | x | x | x | x |
| Wnt3a | x | x | x | x | x | x | x | x |
| Mmp21 | x | x | x | x | x | x | x | x |
| Tinagl1 | | x | x | x | x | x | x | x |
| Lipm | x | | x | x | x | x | x | x |
| Klk8 | x | x | | x | x | x | x | x |
| Sepina12 | x | x | x | | x | x | x | x |
| Serpind1 | x | x | x | x | | x | x | x |
| Gpc1 | x | x | x | x | x | | x | x |
| Psap | x | x | x | x | x | x | | x |
| Prss29 | x | x | x | x | x | x | x | |
| Ambp | x | x | x | x | x | x | x | x |
| Cpa6 | x | x | x | x | x | x | x | x |
| Scgb3a2 | x | x | x | x | x | x | x | x |
| Reg3d | x | x | x | x | x | x | x | x |
| Abpz; | x | x | x | x | x | x | x | x |
| Scgb2b24 | | | | | | | | |
| Npff | x | x | x | x | x | x | x | x |
| Fam3b | x | x | x | x | x | x | x | x |
| Fstl1 | x | x | x | x | x | x | x | x |
| Spp1 | x | x | x | x | x | x | x | x |
| Ifne | x | x | x | x | x | x | x | x |
| Prl8a6 | x | x | x | x | x | x | x | x |
| Srpx2 | x | x | x | x | x | x | x | x |
| Dhrs11 | x | x | x | x | x | x | x | x |
| Apom | x | x | x | x | x | x | x | x |
| Fam3c | x | x | x | x | x | x | x | x |
| Apbh; Scgb1b2 | x | x | x | x | x | x | x | x |
| Timp3 | x | x | x | x | x | x | x | x |
| Vwa | x | x | x | x | x | x | x | x |
| Tff3 | x | x | x | x | x | x | x | x |
| Vstm2a | x | x | x | x | x | x | x | x |
| Grem1 | x | x | x | x | x | x | x | x |
| Tac4 | x | x | x | x | x | x | x | x |
| Cxcl5 | x | x | x | x | x | x | x | x |
| Serpine1 | x | x | x | x | x | x | x | x |
| Gsn | x | x | x | x | x | x | x | x |
| Oxt | x | x | x | x | x | x | x | x |
| Ctf1 | x | x | x | x | x | x | x | x |
| Mdk | x | x | x | x | x | x | x | x |
| Pla2g2c | x | x | x | x | x | x | x | x |
| Nhlrc3 | x | x | x | x | x | x | x | x |
| GliprIll | x | x | x | x | x | x | x | x |
| Mcpt7 | x | x | x | x | x | x | x | x |
| Il12a | x | x | x | x | x | x | x | x |
| 2410001C21Rik | x | x | x | x | x | x | x | x |
| Fjx1 | x | x | x | x | x | x | x | x |
| Cntn4 | x | x | x | x | x | x | x | x |
| Fetub | x | x | x | x | x | x | x | x |
| Ccl6 | x | x | x | x | x | x | x | x |
| Thbs2 | x | x | x | x | x | x | x | x |
| Fbln2 | x | x | x | x | x | x | x | x |
| Htral | x | x | x | x | x | x | x | x |
| Sfrp1 | x | x | x | x | x | x | x | x |
| Wnt6 | x | x | x | x | x | x | x | x |
| Impg2 | x | x | x | x | x | x | x | x |
| | | | | | Abpz; | | | |
| | Ambp | Cpa6 | Scgb3a2 | Reg3d | Scgb2b24 | Npff | Fam3b | |
| Rai2 | x | x | x | x | x | x | x | |
| Hmgb1 | x | x | x | x | x | x | x | |
| Furin | x | x | x | x | x | x | x | |
| Cnpy4 | x | x | x | x | x | x | x | |
| C8a | x | x | x | x | x | x | x | |
| Il7 | x | x | x | x | x | x | x | |
| Wnt3a | x | x | x | x | x | x | x | |
| Mmp21 | x | x | x | x | x | x | x | |
| Tinagl1 | x | x | x | x | x | x | x | |
| Lipm | x | x | x | x | x | x | x | |
| Klk8 | x | x | x | x | x | x | x | |
| Sepina12 | x | x | x | x | x | x | x | |
| Serpind1 | x | x | x | x | x | x | x | |
| Gpc1 | x | x | x | x | x | x | x | |
| Psap | x | x | x | x | x | x | x | |
| Prss29 | x | x | x | x | x | x | x | |
| Ambp | | x | x | x | x | x | x | |
| Cpa6 | x | | x | x | x | x | x | |
| Scgb3a2 | x | x | | x | x | x | x | |
| Reg3d | x | x | x | | x | x | x | |
| Abpz; Scgb2b24 | x | x | x | x | | x | x | |
| Npff | x | x | x | x | x | | x | |
| Fam3b | x | x | x | x | x | x | | |
| Fstl1 | x | x | x | x | x | x | x | |
| Spp1 | x | x | x | x | x | x | x | |
| Ifne | x | x | x | x | x | x | x | |
| Prl8a6 | x | x | x | x | x | x | x | |
| Srpx2 | x | x | x | x | x | x | x | |
| Dhrs11 | x | x | x | x | x | x | x | |
| Apom | x | x | x | x | x | x | x | |
| Fam3c | x | x | x | x | x | x | x | |
| Apbh; Scgb1b2 | x | x | x | x | x | x | x | |
| Timp3 | x | x | x | x | x | x | x | |
| Vwa | x | x | x | x | x | x | x | |
| Tff3 | x | x | x | x | x | x | x | |
| Vstm2a | x | x | x | x | x | x | x | |
| Grem1 | x | x | x | x | x | x | x | |
| Tac4 | x | x | x | x | x | x | x | |
| Cxcl5 | x | x | x | x | x | x | x | |
| Serpine1 | x | x | x | x | x | x | x | |
| Gsn | x | x | x | x | x | x | x | |
| Oxt | x | x | x | x | x | x | x | |
| Ctf1 | x | x | x | x | x | x | x | |
| Mdk | x | x | x | x | x | x | x | |
| Pla2g2c | x | x | x | x | x | x | x | |
| Nhlrc3 | x | x | x | x | x | x | x | |
| Glipr1l1 | x | x | x | x | x | x | x | |
| Mcpt7 | x | x | x | x | x | x | x | |
| Il12a | x | x | x | x | x | x | x | |
| 2410001C21Rik | x | x | x | x | x | x | x | |
| Fix1 | x | x | x | x | x | x | x | |
| Cntn4 | x | x | x | x | x | x | x | |
| Fetub | x | x | x | x | x | x | x | |
| Ccl6 | x | x | x | x | x | x | x | |
| Thbs2 | x | x | x | x | x | x | x | |
| Fbln2 | x | x | x | x | x | x | x | |
| Htra1 | x | x | x | x | x | x | x | |
| Sfrp1 | x | x | x | x | x | x | x | |
| Wnt6 | x | x | x | x | x | x | x | |
| Impg2 | x | x | x | x | x | x | x | |
| | Fstl1 | Spp1 | Ifne | Prl8a6 | Srpx2 | Dhrs11 | Apom | Fam3c |
| Rai2 | x | x | x | x | x | x | x | x |
| Hmgb1 | x | x | x | x | x | x | x | x |
| Furin | x | x | x | x | x | x | x | x |
| Cnpy4 | x | x | x | x | x | x | x | x |
| C8a | x | x | x | x | x | x | x | x |
| Il7 | x | x | x | x | x | x | x | x |
| Wnt3a | x | x | x | x | x | x | x | x |
| Mmp21 | x | x | x | x | x | x | x | x |
| Tinagl1 | x | x | x | x | x | x | x | x |
| Lipm | x | x | x | x | x | x | x | x |
| Klk8 | x | x | x | x | x | x | x | x |
| Sepina12 | x | x | x | x | x | x | x | x |
| Serpind1 | x | x | x | x | x | x | x | x |
| Gpc1 | x | x | x | x | x | x | x | x |
| Psap | x | x | x | x | x | x | x | x |
| Prss29 | x | x | x | x | x | x | x | x |
| Ambp | x | x | x | x | x | x | x | x |
| Cpa6 | x | x | x | x | x | x | x | x |
| Scgb3a2 | x | x | x | x | x | x | x | x |
| Reg3d | x | x | x | x | x | x | x | x |
| Abpz; Scgb2b24 | x | x | x | x | x | x | x | x |
| Npff | x | x | x | x | x | x | x | x |
| Fam3b | x | x | x | x | x | x | x | x |
| Fstl1 | | x | x | x | x | x | x | x |
| Spp1 | x | | x | x | x | x | x | x |
| Ifne | x | x | | x | x | x | x | x |
| Prl8a6 | x | x | x | | x | x | x | x |
| Srpx2 | x | x | x | x | | x | x | x |
| Dhrs11 | x | x | x | x | x | | x | x |
| Apom | x | x | x | x | x | x | | x |
| Fam3c | x | x | x | x | x | x | x | |
| Apbh; Scgb1b2 | x | x | x | x | x | x | x | x |
| Timp3 | x | x | x | x | x | x | x | x |
| Vwa | x | x | x | x | x | x | x | x |
| Tff3 | x | x | x | x | x | x | x | x |
| Vstm2a | x | x | x | x | x | x | x | x |
| Grem1 | x | x | x | x | x | x | x | x |
| Tac4 | x | x | x | x | x | x | x | x |
| Cxcl5 | x | x | x | x | x | x | x | x |
| Serpine1 | x | x | x | x | x | x | x | x |
| Gsn | x | x | x | x | x | x | x | x |
| Oxt | x | x | x | x | x | x | x | x |
| Ctf1 | x | x | x | x | x | x | x | x |
| Mdk | x | x | x | x | x | x | x | x |
| Pla2g2c | x | x | x | x | x | x | x | x |
| Nhlrc3 | x | x | x | x | x | x | x | x |
| Glipr1l1 | x | x | x | x | x | x | x | x |
| Mcpt7 | x | x | x | x | x | x | x | x |
| Il12a | x | x | x | x | x | x | x | x |
| 2410001C21Rik | x | x | x | x | x | x | x | x |
| Fjx1 | x | x | x | x | x | x | x | x |
| Cntn4 | x | x | x | x | x | x | x | x |
| Fetub | x | x | x | x | x | x | x | x |
| Ccl6 | x | x | x | x | x | x | x | x |
| Thbs2 | x | x | x | x | x | x | x | x |
| Fbln2 | x | x | x | x | x | x | x | x |
| Htral | x | x | x | x | x | x | x | x |
| Sfrp1 | x | x | x | x | x | x | x | x |
| Wnt6 | x | x | x | x | x | x | x | x |
| Impg2 | x | x | x | x | x | x | x | x |
| | Apbh; | | | | | | | |
| | Scgb1b2 | Timp3 | Vwa | Tff3 | Vstm2a | Greml | Tac4 | |
| Rai2 | x | x | x | x | x | x | x | |
| Hmgb1 | x | x | x | x | x | x | x | |
| Furin | x | x | x | x | x | x | x | |
| Cnpy4 | x | x | x | x | x | x | x | |
| C8a | x | x | x | x | x | x | x | |
| Il7 | x | x | x | x | x | x | x | |
| Wnt3a | x | x | x | x | x | x | x | |
| Mmp21 | x | x | x | x | x | x | x | |
| Tinagl1 | x | x | x | x | x | x | x | |
| Lipm | x | x | x | x | x | x | x | |
| Klk8 | x | x | x | x | x | x | x | |
| Sepina12 | x | x | x | x | x | x | x | |
| Serpind1 | x | x | x | x | x | x | x | |
| Gpc1 | x | x | x | x | x | x | x | |
| Psap | x | x | x | x | x | x | x | |
| Prss29 | x | x | x | x | x | x | x | |
| Ambp | x | x | x | x | x | x | x | |
| Cpa6 | x | x | x | x | x | x | x | |
| Scgb3a2 | x | x | x | x | x | x | x | |
| Reg3d | x | x | x | x | x | x | x | |
| Abpz; Scgb2b24 | x | x | x | x | x | x | x | |
| Npff | x | x | x | x | x | x | x | |
| Fam3b | x | x | x | x | x | x | x | |
| Fstl1 | x | x | x | x | x | x | x | |
| Spp1 | x | x | x | x | x | x | x | |
| Ifne | x | x | x | x | x | x | x | |
| Prl8a6 | x | x | x | x | x | x | x | |
| Srpx2 | x | x | x | x | x | x | x | |
| Dhrs11 | x | x | x | x | x | x | x | |
| Apom | x | x | x | x | x | x | x | |
| Fam3c | x | x | x | x | x | x | x | |
| Apbh; Scgb1b2 | | x | x | x | x | x | x | |
| Timp3 | x | | x | x | x | x | x | |
| Vwa | x | x | | x | x | x | x | |
| Tff3 | x | x | x | | x | x | x | |
| Vstm2a | x | x | x | x | | x | x | |
| Grem1 | x | x | x | x | x | | x | |
| Tac4 | x | x | x | x | x | x | | |
| Cxcl5 | x | x | x | x | x | x | x | |
| Serpine1 | x | x | x | x | x | x | x | |
| Gsn | x | x | x | x | x | x | x | |
| Oxt | x | x | x | x | x | x | x | |
| Ctf1 | x | x | x | x | x | x | x | |
| Mdk | x | x | x | x | x | x | x | |
| Pla2g2c | x | x | x | x | x | x | x | |
| Nhlrc3 | x | x | x | x | x | x | x | |
| Glipr1l1 | x | x | x | x | x | x | x | |
| Mcpt7 | x | x | x | x | x | x | x | |
| Il12a | x | x | x | x | x | x | x | |
| 2410001C21Rik | x | x | x | x | x | x | x | |
| Fjx1 | x | x | x | x | x | x | x | |
| Cntn4 | x | x | x | x | x | x | x | |
| Fetub | x | x | x | x | x | x | x | |
| Ccl6 | x | x | x | x | x | x | x | |
| Thbs2 | x | x | x | x | x | x | x | |
| Fbln2 | x | x | x | x | x | x | x | |
| Htra1 | x | x | x | x | x | x | x | |
| Sfrp1 | x | x | x | x | x | x | x | |
| Wnt6 | x | x | x | x | x | x | x | |
| Impg2 | x | x | x | x | x | x | x | |
| | Cxcl5 | Serpine1 | Gsn | Oxt | Ctf1 | Mdk | Pla2g2c | Nhlrc3 |
| Rai2 | x | x | x | x | x | x | x | x |
| Hmgb1 | x | x | x | x | x | x | x | x |
| Furin | x | x | x | x | x | x | x | x |
| Cnpy4 | x | x | x | x | x | x | x | x |
| C8a | x | x | x | x | x | x | x | x |
| Il7 | x | x | x | x | x | x | x | x |
| Wnt3a | x | x | x | x | x | x | x | x |
| Mmp21 | x | x | x | x | x | x | x | x |
| Tinagl1 | x | x | x | x | x | x | x | x |
| Lipm | x | x | x | x | x | x | x | x |
| Klk8 | x | x | x | x | x | x | x | x |
| Sepina12 | x | x | x | x | x | x | x | x |
| Serpind1 | x | x | x | x | x | x | x | x |
| Gpc1 | x | x | x | x | x | x | x | x |
| Psap | x | x | x | x | x | x | x | x |
| Prss29 | x | x | x | x | x | x | x | x |
| Ambp | x | x | x | x | x | x | x | x |
| Cpa6 | x | x | x | x | x | x | x | x |
| Scgb3a2 | x | x | x | x | x | x | x | x |
| Reg3d | x | x | x | x | x | x | x | x |
| Abpz; Scgb2b24 | x | x | x | x | x | x | x | x |
| Npff | x | x | x | x | x | x | x | x |
| Fam3b | x | x | x | x | x | x | x | x |
| Fstl1 | x | x | x | x | x | x | x | x |
| Spp1 | x | x | x | x | x | x | x | x |
| Ifne | x | x | x | x | x | x | x | x |
| Prl8a6 | x | x | x | x | x | x | x | x |
| Srpx2 | x | x | x | x | x | x | x | x |
| Dhrs11 | x | x | x | x | x | x | x | x |
| Apom | x | x | x | x | x | x | x | x |
| Fam3c | x | x | x | x | x | x | x | x |
| Apbh; Scgb1b2 | x | x | x | x | x | x | x | x |
| Timp3 | x | x | x | x | x | x | x | x |
| Vwa | x | x | x | x | x | x | x | x |
| Tff3 | x | x | x | x | x | x | x | x |
| Vstm2a | x | x | x | x | x | x | x | x |
| Grem1 | x | x | x | x | x | x | x | x |
| Tac4 | x | x | x | x | x | x | x | x |
| Cxcl5 | | x | x | x | x | x | x | x |
| Serpine1 | x | | x | x | x | x | x | x |
| Gsn | x | x | | x | x | x | x | x |
| Oxt | x | x | x | | x | x | x | x |
| Ctf1 | x | x | x | x | | x | x | x |
| Mdk | x | x | x | x | x | | x | x |
| Pla2g2c | x | x | x | x | x | x | | x |
| Nhlrc3 | x | x | x | x | x | x | x | |
| Glipr1l1 | x | x | x | x | x | x | x | x |
| Mcpt7 | x | x | x | x | x | x | x | x |
| Il12a | x | x | x | x | x | x | x | x |
| 2410001C21Rik | x | x | x | x | x | x | x | x |
| Fjx1 | x | x | x | x | x | x | x | x |
| Cntn4 | x | x | x | x | x | x | x | x |
| Fetub | x | x | x | x | x | x | x | x |
| Ccl6 | x | x | x | x | x | x | x | x |
| Thbs2 | x | x | x | x | x | x | x | x |
| Fbln2 | x | x | x | x | x | x | x | x |
| Htra1 | x | x | x | x | x | x | x | x |
| Sfrp1 | x | x | x | x | x | x | x | x |
| Wnt6 | x | x | x | x | x | x | x | x |
| Impg2 | x | x | x | x | x | x | x | x |
| | Glipr1l1 | Mcpt7 | Il12a | 2410001C21Rik | Fjx1 | Cntn4 | Fetub | Ccl6 |
| Rai2 | x | x | x | x | x | x | x | x |
| Hmgb1 | x | x | x | x | x | x | x | x |
| Furin | x | x | x | x | x | x | x | x |
| Cnpy4 | x | x | x | x | x | x | x | x |
| C8a | x | x | x | x | x | x | x | x |
| Il7 | x | x | x | x | x | x | x | x |
| Wnt3a | x | x | x | x | x | x | x | x |
| Mmp21 | x | x | x | x | x | x | x | x |
| Tinagl1 | x | x | x | x | x | x | x | x |
| Lipm | x | x | x | x | x | x | x | x |
| Klk8 | x | x | x | x | x | x | x | x |
| Sepina12 | x | x | x | x | x | x | x | x |
| Serpind1 | x | x | x | x | x | x | x | x |
| Gpc1 | x | x | x | x | x | x | x | x |
| Psap | x | x | x | x | x | x | x | x |
| Prss29 | x | x | x | x | x | x | x | x |
| Ambp | x | x | x | x | x | x | x | x |
| Cpa6 | x | x | x | x | x | x | x | x |
| Scgb3a2 | x | x | x | x | x | x | x | x |
| Reg3d | x | x | x | x | x | x | x | x |
| Abpz; Scgb2b24 | x | x | x | x | x | x | x | x |
| Npff | x | x | x | x | x | x | x | x |
| Fam3b | x | x | x | x | x | x | x | x |
| Fstl1 | x | x | x | x | x | x | x | x |
| Spp1 | x | x | x | x | x | x | x | x |
| Ifne | x | x | x | x | x | x | x | x |
| Prl8a6 | x | x | x | x | x | x | x | x |
| Srpx2 | x | x | x | x | x | x | x | x |
| Dhrs11 | x | x | x | x | x | x | x | x |
| Apom | x | x | x | x | x | x | x | x |
| Fam3c | x | x | x | x | x | x | x | x |
| Apbh; Scgb1b2 | x | x | x | x | x | x | x | x |
| Timp3 | x | x | x | x | x | x | x | x |
| Vwa | x | x | x | x | x | x | x | x |
| Tff3 | x | x | x | x | x | x | x | x |
| Vstm2a | x | x | x | x | x | x | x | x |
| Grem1 | x | x | x | x | x | x | x | x |
| Tac4 | x | x | x | x | x | x | x | x |
| Cxcl5 | x | x | x | x | x | x | x | x |
| Serpine1 | x | x | x | x | x | x | x | x |
| Gsn | x | x | x | x | x | x | x | x |
| Oxt | x | x | x | x | x | x | x | x |
| Ctf1 | x | x | x | x | x | x | x | x |
| Mdk | x | x | x | x | x | x | x | x |
| Pla2g2c | x | x | x | x | x | x | x | x |
| Nhlrc3 | x | x | x | x | x | x | x | x |
| Glipr1l1 | | x | x | x | x | x | x | x |
| Mcpt7 | x | | x | x | x | x | x | x |
| Il12a | x | x | | x | x | x | x | x |
| 2410001C21Rik | x | x | x | | x | x | x | x |
| Fjx1 | x | x | x | x | | x | x | x |
| Cntn4 | x | x | x | x | x | | x | x |
| Fetub | x | x | x | x | x | x | | x |
| Ccl6 | x | x | x | x | x | x | x | |
| Thbs2 | x | x | x | x | x | x | x | x |
| Fbln2 | x | x | x | x | x | x | x | x |
| Htra1 | x | x | x | x | x | x | x | x |
| Sfrp1 | x | x | x | x | x | x | x | x |
| Wnt6 | x | x | x | x | x | x | x | x |
| Impg2 | x | x | x | x | x | x | x | x |

| | Thbs2 | Fbln2 | Htra1 | Sfrp1 | Wnt6 | Impg2 |
|---|---|---|---|---|---|---|
| Rai2 | x | x | x | x | x | x |
| Hmgb1 | x | x | x | x | x | x |
| Furin | x | x | x | x | x | x |
| Cnpy4 | x | x | x | x | x | x |
| C8a | x | x | x | x | x | x |
| Il7 | x | x | x | x | x | x |
| Wnt3a | x | x | x | x | x | x |
| Mmp21 | x | x | x | x | x | x |
| Tinagl1 | x | x | x | x | x | x |
| Lipm | x | x | x | x | x | x |
| Klk8 | x | x | x | x | x | x |
| Sepina12 | x | x | x | x | x | x |
| Serpind1 | x | x | x | x | x | x |
| Gpc1 | x | x | x | x | x | x |
| Psap | x | x | x | x | x | x |
| Prss29 | x | x | x | x | x | x |
| Ambp | x | x | x | x | x | x |
| Cpa6 | x | x | x | x | x | x |
| Scgb3a2 | x | x | x | x | x | x |
| Reg3d | x | x | x | x | x | x |
| Abpz; | x | x | x | x | x | x |
| Scgb2b24 | | | | | | |
| Npff | x | x | x | x | x | x |
| Fam3b | x | x | x | x | x | x |
| Fstl1 | x | x | x | x | x | x |
| Spp1 | x | x | x | x | x | x |
| Ifne | x | x | x | x | x | x |
| Prl8a6 | x | x | x | x | x | x |
| Srpx2 | x | x | x | x | x | x |
| Dhrs11 | x | x | x | x | x | x |
| Apom | x | x | x | x | x | x |
| Fam3c | x | x | x | x | x | x |
| Apbh; | x | x | x | x | x | x |
| Scgb1b2 | | | | | | |
| Timp3 | x | x | x | x | x | x |
| Vwa | x | x | x | x | x | x |
| Tff3 | x | x | x | x | x | x |
| Vstm2a | x | x | x | x | x | x |
| Grem1 | x | x | x | x | x | x |
| Tac4 | x | x | x | x | x | x |
| Cxcl5 | x | x | x | x | x | x |
| Serpine1 | x | x | x | x | x | x |
| Gsn | x | x | x | x | x | x |
| Oxt | x | x | x | x | x | x |
| Ctf1 | x | x | x | x | x | x |
| Mdk | x | x | x | x | x | x |
| Pla2g2c | x | x | x | x | x | x |
| Nhlrc3 | x | x | x | x | x | x |
| Glipr1l1 | x | x | x | x | x | x |
| Mcpt7 | x | x | x | x | x | x |
| Il12a | x | x | x | x | x | x |
| 2410001C21Rik | x | x | x | x | x | x |
| Fjx1 | x | x | x | x | x | x |
| Cntn4 | x | x | x | x | x | x |
| Fetub | x | x | x | x | x | x |
| Ccl6 | x | x | x | x | x | x |
| Thbs2 | | x | x | x | x | x |
| Fbln2 | x | | x | x | x | x |
| Htra1 | x | x | | x | x | x |
| Sfrp1 | x | x | x | | x | x |
| Wnt6 | x | x | x | x | | x |
| Impg2 | x | x | x | x | x | |

Table 19: Exemplary table combinations of genes and polypeptides as described herein.

Table 2 include members of the canonical Wnt pathway. Tables 3-5 include proteins associated with lipid hydrolysis and insulin or IGF signaling. Tables 6-9 include proteins associated with vascular remodeling, extracellular matrix, proteoglycan and cell adhesion polypeptides. Table 10 includes serine proteases. Table 11 includes serine protease inhibitors. Tables 12-14 include members of the interleukin family, interferon signaling family, or chemokine family. Tables 15-17 include isolated TLR binding polypeptides, lipocalin polypeptides, or secretoglobin polypeptides.

| Table | Tables | Tables | Table | Table | Tables | Tables |
|---|---|---|---|---|---|---|
| 2 | 3-5 | 6-9 | 10 | 11 | 12-14 | 15-17 |
| X | | | | | | |
| | X | | | | | |
| X | X | | | | | |
| | | X | | | | |
| X | | X | | | | |
| | X | X | | | | |
| X | X | X | | | | |
| | | | X | | | |
| X | | | X | | | |
| | X | | X | | | |
| X | X | | X | | | |
| | | X | X | | | |
| X | | X | X | | | |
| | X | X | X | | | |
| X | X | X | X | | | |
| | | | | X | | |
| X | | | | X | | |
| | X | | | X | | |
| X | X | | | X | | |
| | | X | | X | | |
| X | | X | | X | | |
| | X | X | | X | | |
| X | X | X | | X | | |
| | | | X | X | | |
| X | | | X | X | | |
| | X | | X | X | | |
| X | X | | X | X | | |
| | | X | X | X | | |
| X | | X | X | X | | |
| | X | X | X | X | | |
| X | X | X | X | X | | |
| | | | | | X | |
| X | | | | | X | |
| | X | | | | X | |
| X | X | | | | X | |
| | | X | | | X | |
| X | | X | | | X | |
| | X | X | | | X | |
| X | X | X | | | X | |
| | | | X | | X | |
| X | | | X | | X | |
| | X | | X | | X | |
| X | X | | X | | X | |
| | | X | X | | X | |
| X | | X | X | | X | |
| | X | X | X | | X | |
| X | X | X | X | | X | |
| | | | | X | X | |
| X | | | | X | X | |
| | X | | | X | X | |
| X | X | | | X | X | |
| | | X | | X | X | |
| X | | X | | X | X | |
| | X | X | | X | X | |
| X | X | X | | X | X | |
| | | | X | X | X | |
| X | | | X | X | X | |
| | X | | X | X | X | |
| X | X | | X | X | X | |
| | | X | X | X | X | |
| X | | X | X | X | X | |
| | X | X | X | X | X | |
| X | X | X | X | X | X | |
| | | | | | | X |
| X | | | | | | X |
| | X | | | | | X |
| X | X | | | | | X |
| | | X | | | | X |
| X | | X | | | | X |
| | X | X | | | | X |
| X | X | X | | | | X |
| | | | X | | | X |
| X | | | X | | | X |
| | X | | X | | | X |
| X | X | | X | | | X |
| | | X | X | | | X |
| X | | X | X | | | X |
| | X | X | X | | | X |
| X | X | X | X | | | X |
| | | | | X | | X |
| X | | | | X | | X |
| | X | | | X | | X |
| X | X | | | X | | X |
| | | X | | X | | X |
| X | | X | | X | | X |
| | X | X | | X | | X |
| X | X | X | | X | | X |
| | | | X | X | | X |
| X | | | X | X | | X |
| | X | | X | X | | X |
| X | X | | X | X | | X |
| | | X | X | X | | X |
| X | | X | X | X | | X |
| | X | X | X | X | | X |
| X | X | X | X | X | | X |
| | | | | | X | X |
| X | | | | | X | X |
| | X | | | | X | X |
| X | X | | | | X | X |
| | | X | | | X | X |
| X | | X | | | X | X |
| | X | X | | | X | X |
| X | X | X | | | X | X |
| | | | X | | X | X |
| X | | | X | | X | X |
| | X | | X | | X | X |
| X | X | | X | | X | X |
| | | X | X | | X | X |
| X | | X | X | | X | X |
| | X | X | X | | X | X |
| X | X | X | X | | X | X |
| | | | | X | X | X |
| X | | | | X | X | X |
| | X | | | X | X | X |
| X | X | | | X | X | X |
| | | X | | X | X | X |
| X | | X | | X | X | X |
| | X | X | | X | X | X |
| X | X | X | | X | X | X |
| | | | X | X | X | X |
| X | | | X | X | X | X |
| | X | | X | X | X | X |
| X | X | | X | X | X | X |
| | | X | X | X | X | X |
| X | | X | X | X | X | X |
| | X | X | X | X | X | X |
| X | X | X | X | X | X | X |

### Treatment of Cardiac Disease

In some aspects, provided herein are methods for the treatment and prevention of a cardiac injury or a cardiac disease or disorder in a subject in need thereof. The methods described herein can be used to treat, ameliorate, prevent or slow the progression of a number of diseases or their symptoms, such as those resulting in pathological damage to the structure and/or function of the heart.

The terms “cardiac disease,” “cardiac disorder,” and “cardiac injury,” are used interchangeably herein and refer to a condition and/or disorder relating to the heart, including the functional engraftment and vascularization of cardiomyocytes into e.g., infarcted zones. Such cardiac diseases or cardiac-related disease include, but are not limited to, myocardial infarction, heart failure, cardiomyopathy, congenital heart defect (e.g., non-compaction cardiomyopathy), hypertrophic cardiomyopathy, dilated cardiomyopathy, myocarditis, heart failure, arrhythmogenic right ventricular dysplasia (ARVD), cardiac arrhythmia, cardiomyopathy, long QT syndrome, catecholaminergic polymorphic ventricular tachycardia (CPVT), Barth syndrome, and Duchenne muscular dystrophy-related cardiac disease, and cardiomegaly.

In one aspect, described herein is a transplant composition comprising any of the compositions described herein in any combination. The transplant composition can be administered to a subject in need of treatment of a cardiac disease.

As used herein, the terms “administering,” “introducing” and “transplanting” are used interchangeably in the context of the placement of cells, e.g. cardiomyocytes, as described herein into a subject, by a method or route which results in at least partial localization of the introduced cells at a desired site, such as a site of injury or repair, such that a desired effect(s) is produced. The cardiomyocytes can be implanted directly to the heart, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of the cardiomyocytes is desired as cardiomyocytes do not proliferate to an extent that the heart can heal from an acute injury comprising cardiomyocyte death.

In other embodiments, the cells can be administered via an indirect systemic route of administration, such as an intraperitoneal or intravenous route.

When provided prophylactically, the cardiomyocytes can be administered to a subject in advance of any symptom of a disorder, e.g., heart failure due to prior myocardial infarction or left ventricular insufficiency, congestive heart failure etc. Accordingly, the prophylactic administration of a population of cells serves to prevent a cardiac heart failure disorder or maladaptive cardiac remodeling.

In some embodiments of the aspects described herein, the population of cells being administered according to the methods described herein comprises allogeneic cells or their obtained from one or more donors. As used herein, “allogeneic” refers to a cardiomyocyte obtained from or derived from (e.g., differentiated from) one or more different donors of the same species, where the genes at one or more loci are not identical. For example, cardiomyocytes being administered to a subject can be derived from umbilical cord blood obtained from one more unrelated donor subjects, or from one or more non-identical siblings. In some embodiments, syngeneic cell populations can be used, such as those obtained from genetically identical animals, or from identical twins. In other embodiments of this aspect, the cardiomyocytes are autologous cells; that is, the cells are obtained or isolated from a subject (or derived from) and administered to the same subject, i.e., the donor and recipient are the same.

In another aspect, described herein is a cardiac delivery device comprising a composition described herein.

In another aspect, described herein is a method of transplanting cardiomyocytes, the method comprises administering a composition described herein to cardiac tissue, optionally using a cardiac delivery device as described herein.

In one embodiment, the engraftment of the administered cardiomyocytes is increased relative to engraftment of cardiomyocytes that were not in admixture with or had not been contacted with the polypeptide or polypeptides.

In another aspect, described herein is a method of enhancing cardiomyocyte transplant engraftment, the method comprising contacting a cardiomyocyte population with a polypeptide selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fst11; Spp1; Ifne; Prl8a6; Srpx2; Dhrs11; Apom; Fam3c; Apbh/Scgb1b2; Timp3; Vwa; Tff3; Vstm2a; Grem1; Tac4; Cxc15; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2, and transplanting the cardiomyocyte population to cardiac tissue.

In another aspect, described herein is a method of enhancing cardiomyocyte transplant engraftment, the method comprising contacting a cardiomyocyte population with a nucleic acid that encodes a polypeptide selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fstl1; Spp1; Ifne; Prl8a6; Srpx2; Dhrs11; Apom; Fam3c; Apbh/Scgb1b2; Timp3; Vwa; Tff3; Vstm2a; Grem1; Tac4; Cxc15; Serpine1; Gsn; Oxt; Ctfl; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2, and transplanting the cardiomyocyte population to cardiac tissue.

The polypeptide or nucleic acid encoding such polypeptide as described herein can be expressed transiently, conditionally, or constitutively by the cardiomyocyte population. Various techniques and methods are known in the art for delivering nucleic acids to cells.

Preferably, the polypeptide(s) or nucleic acid(s) encoding such polypeptide(s) described herein would be administered to the cardiomyocyte population to be used for engraftment, could be washed off prior to transplantation into a subject, and the polypeptide(s) would be transiently expressed during the period of engraftment (e.g., about 5 days). The contact time of the polypeptide(s) or nucleic acid(s) encoding such polypeptides described herein with the cardiomyocyte population can be about 5 minutes or more, 10 minutes or more, 1 hour or more, 1 day or more, 5 days or more in vitro followed by transplantation of the cardiomyocytes into a subject.

In some embodiments, the cardiomyocyte population is further contacted with a LIPOFECTAMINE for transient expression of the nucleic acid(s) encoding the polypeptide(s) described herein. Methods of transfecting cardiomyocytes are known in the art. See for example, Si-Tayeb et al. BMC Dev Biol. (2010; 10:81), which is incorporated herein by reference in its entirety.

Nucleic acids encoding the polypeptides described herein can be stably integrated in the genome of the cell and operably linked to a promoter active in the cell. Alternatively, nucleic acids encoding the polypeptides described herein can be operably linked to a promoter in an expression construct. Expression constructs include any nucleic acid constructs capable of directing expression of a gene or other nucleic acid sequence of interest (e.g., any of the sequences of Table 1) and can transfer such a nucleic acid sequence of interest to the cardiomyocytes for engraftment.

In yet another aspect, described herein is a method of enhancing cardiomyocyte transplant engraftment, the method comprising contacting a cardiomyocyte population with a vector that encodes a polypeptide selected from the group consisting of Rai2; Hmgb1; Furin; Cnpy4; C8a; Il7; Wnt3a; Mmp21; Tinagl1; Lipm; Klk8; Sepina12; Serpind1; Gpc1; Psap; Prss29; Ambp; Cpa6; Scgb3a2; Reg3d; Abpz/Scgb2b24; Npff; Fam3b; Fstl1; Spp1; Ifne; Prl8a6; Srpx2; Dhrs11; Apom; Fam3c; Apbh/Scgb1b2; Timp3; Vwa; Tff3; Vstm2a; Grem1; Tac4; Cxcl5; Serpine1; Gsn; Oxt; Ctf1; Mdk; Pla2g2c; Nhlrc3; Glipr111; Mcpt7; Il12a; 2410001C21Rik; Fjx1; Cntn4; Fetub; Ccl6; Thbs2; Fbln2; Htra1; Sfrp1; Wnt6; and Impg2, and transplanting the cardiomyocyte population to cardiac tissue.

In some embodiments, the vector comprises an adenovirus associated vector (AAV).

Another non-integrative viral vector that can be used is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Alternatively, a minicircle vector can be used. Minicircle vectors are circularized vectors in which the plasmid backbone has been released leaving only the eukaryotic promoter and cDNA(s) that are to be expressed. Additional vectors that can be used to contact the cardiomyocyte population as described herein with a nucleic acid include lentiviral vectors, such as Epstein Barr, Human immunodeficiency virus (HIV), and hepatitis B virus (HBV).

In some embodiments of any of the aspects, the cardiomyocyte population is a human cardiomyocyte population. In some embodiments, the cardiomyocyte population is differentiated in vitro from embryonic stem cells or induced pluripotent stem cells. In some embodiments, the induced pluripotent stem cells are differentiated from induced pluripotent stem cells derived from the transplant recipient.

### Pharmaceutically Acceptable Carriers

The methods of administering human cardiomyocytes to a subject as described herein involve the use of therapeutic compositions comprising such cells. Therapeutic compositions contain a physiologically tolerable carrier together with the cell composition and optionally at least one additional bioactive agent, polypeptide(s), nucleic acid(s) encoding said polypeptide, or factor(s) as described herein, dissolved or dispersed therein as an active ingredient.

In a preferred embodiment, the therapeutic composition is not substantially immunogenic when administered to a mammal or human patient for therapeutic purposes, unless so desired. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset, transplant rejection, allergic reaction, and the like. A pharmaceutically acceptable carrier will not promote the raising of an immune response to an agent with which it is admixed, unless so desired. The preparation of a composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared.

A transplant composition for humans may include one or more pharmaceutically acceptable carrier or materials as excipients. In contrast, a cell culture composition (not for human transplant) typically will use research reagents like cell culture media as an excipient. Cardiomyocytes could also be administered in an FDA-approved matrix/scaffold or in combination with FDA-approved drugs as described above.

In general, the compositions comprising cardiomyocytes described herein are administered as suspension formulations where the cells are admixed with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the human cardiac progenitor cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

### Administration and Efficacy

Provided herein are methods for treating a cardiac disease, a cardiac disorder, a cardiac injury, heart failure, or myocardial infarction comprising administering cardiomyocytes to a subject in need thereof. In some embodiments, methods and compositions are provided herein for the prevention of an anticipated disorder e.g., heart failure following myocardial injury.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions and formulations as disclosed herein will be decided by the attending physician within the scope of sound medical judgment. The exact amount required will vary depending on factors such as the type of disease being treated.

The term “effective amount” as used herein refers to the amount of a population of cardiomyocytes needed to alleviate at least one or more symptoms of a disease or disorder, including but not limited to an injury, disease, or disorder. An “effective amount” relates to a sufficient amount of a composition to provide the desired effect, e.g., treat a subject having an infarct zone following myocardial infarction, improve cardiomyocyte engraftment, prevent onset of heart failure following cardiac injury, enhance vascularization of a graft, etc. The term “therapeutically effective amount” therefore refers to an amount of human cardiomyocytes or a composition such cells that is sufficient to promote a particular effect when administered to a typical subject, such as one who has, or is at risk for, a cardiac disease or disorder. An effective amount as used herein would also include an amount sufficient to prevent or delay the development of a symptom of the disease, alter the course of a disease symptom (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting the myocardium prior to administering the cells according to the methods described herein. In some embodiments, the subject is first diagnosed as being at risk of developing a disease (e.g., heart failure following myocardial injury) or disorder prior to administering the cells.

For use in the various aspects described herein, an effective amount of human cardiomyocytes comprises at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2× 109, at least 3×10⁹, at least 4×10⁹, at least 5×10⁹ or more cardiomyocytes.

In some embodiments, a composition comprising cardiomyocytes treated with any one or more of the polypeptides or nucleic acids encoding such polypeptides described herein permits engraftment of the cells in the heart at an efficiency at least 20% greater than the engraftment when such cardiomyocytes are administered alone; in other embodiments, such efficiency is at least 30%, at least 40%, at least 50%, at least 60%, at least 70%, at least 80%, at least 90%, at least 1-fold, at least 2-fold, at least 5-fold, at least 10-fold, at least 100-fold or more than the efficiency of engraftment when cardiomyocytes are administered alone without the polypeptides or nucleic acids encoding such polypeptides described herein.

In some embodiments, an effective amount of cardiomyocytes are administered to a subject by intracardiac administration or delivery. As defined herein, “intracardiac” administration or delivery refers to all routes of administration whereby a population of cardiomyocytes is administered in a way that results in direct contact of these cells with the myocardium of a subject, including, but not limited to, direct cardiac injection, intra-myocardial injection(s), intra-infarct zone injection, injection during surgery (e.g., cardiac bypass surgery, during implantation of a cardiac mini-pump or a pacemaker) etc. In some such embodiments, the cells are injected into the myocardium (e.g., cardiomyocytes), or into the cavity of the atria and/or ventricles. In some embodiments, intracardiac delivery of cells includes administration methods whereby cells are administered, for example as a cell suspension, to a subject undergoing surgery via a single injection or multiple “mini” injections into the desired region of the heart.

In some embodiments, an effective amount of cardiomyocytes is administered to a subject by systemic administration, such as intravenous administration.

The phrases “systemic administration,” “administered systemically”, “peripheral administration” and “administered peripherally” are used herein refer to the administration of a population of cardiomyocytes other than directly into a target site, tissue, or organ, such as the heart, such that it enters, instead, the subject's circulatory system.

The choice of formulation will depend upon the specific composition used and the number of cardiomyocytes to be administered; such formulations can be adjusted by the skilled practitioner. However, as an example, where the composition is cardiomyocytes in a pharmaceutically acceptable carrier, the composition can be a suspension of the cells in an appropriate buffer (e.g., saline buffer) at an effective concentration of cells per mL of solution. The formulation can also include cell nutrients, a simple sugar (e.g., for osmotic pressure regulation) or other components to maintain the viability of the cells. Alternatively, the formulation can comprise a scaffold, such as a biodegradable scaffold.

In some embodiments, additional agents to aid in treatment of the subject can be administered before or following treatment with the cardiomyocytes as described. Such additional agents can be used to prepare the target tissue for administration of the progenitor cells. Alternatively, the additional agents can be administered after the cardiomyocytes to support the engraftment and growth of the administered cell into the heart, or other desired administration site. In some embodiments, the additional agent comprises growth factors, such as VEGF or PDGF. Other exemplary agents can be used to reduce the load on the heart while the cardiomyocytes are engrafting (e.g., beta blockers, medications to lower blood pressure etc.).

The efficacy of treatment can be determined by the skilled clinician. However, a treatment is considered “effective treatment,” as the term is used herein, if any one or all of the symptoms, or other clinically accepted symptoms or markers of disease, e.g., cardiac disease, heart failure, cardiac injury and/or a cardiac disorder are reduced, e.g., by at least 10% following treatment with a composition comprising human cardiomyocytes as described herein. Methods of measuring these indicators are known to those of skill in the art and/or described herein.

Indicators of a cardiac disease or cardiac disorder, or cardiac injury include functional indicators or parameters, e.g., stroke volume, heart rate, left ventricular ejection fraction, heart rate, heart rhythm, blood pressure, heart volume, regurgitation, etc. as well as biochemical indicators, such as a decrease in markers of cardiac injury, such as serum lactate dehydrogenase, or serum troponin, among others. As one example, myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heartbeat. The term ejection fraction applies to both the right and left ventricles. LVEF refers to the left ventricular ejection fraction (LVEF). Fractional shortening refers to the difference between end-diastolic and end-systolic dimensions divided by end-diastolic dimension.

Non-limiting examples of clinical tests that can be used to assess cardiac functional parameters include echocardiography (with or without Doppler flow imaging), electrocardiogram (EKG), exercise stress test, Holter monitoring, or measurement of β-natriuretic peptide.

Where necessary or desired, animal models of injury or disease can be used to gauge the effectiveness of a particular composition as described herein. For example, an isolated working rabbit or rat heart model, or a coronary ligation model in either canines or porcines can be used. Animal models of cardiac function are useful for monitoring infarct zones, coronary perfusion, electrical conduction, left ventricular end diastolic pressure, left ventricular ejection fraction, heart rate, blood pressure, degree of hypertrophy, diastolic relaxation function, cardiac output, heart rate variability, and ventricular wall thickness, etc.

In some embodiments, a composition comprising the cardiomyocytes as described herein is delivered at least 6 hours following the initiation of reperfusion, for example, following a myocardial infarction. During an ischemic insult and subsequent reperfusion, the microenvironment of the heart or that of the infarcted zone can be too “hostile” to permit engraftment of cardiomyocytes administered to the heart. Thus, in some embodiments it is preferable to administer such compositions at least 6 hours, at least 12 hours, at least 18 hours, at least 24 hours, at least 36 hours, at least 48 hours, at least 60 hours, at least 72 hours, at least 84 hours, at least 96 hours, at least 5 days, at least 6 days, at least 7 days, at least 8 days, at least 9 days, at least 10 days or more following the initiation of reperfusion. In some embodiments, the compositions comprising cardiomyocytes as described herein can be administered to an infarcted zone, peri-infarcted zone, ischemic zone, penumbra, or the border zone of the heart at any length of time after a myocardial infarction (e.g., at least 1 month, at least 6 months, at least one year, at least 2 years, at least 5 years, at least 10 years, at least 20 years, at least 30 years or more), however as will be appreciated by those of skill in the art, the success of engraftment following a lengthy interval of time after infarct will depend on a number of factors, including but not limited to, amount of scar tissue deposition, density of scar tissue, size of the infarcted zone, degree of vascularization surrounding the infarcted zone, etc. As such, earlier intervention by administration of compositions comprising cardiomyocytes may be more efficacious than administration after e.g., a month or more after infarct.

Compositions comprising cardiomyocytes as described herein can be administered to any desired region of the heart including, but not limited to, an infarcted zone, peri-infarcted zone, ischemic zone, penumbra, the border zone, areas of wall thinning, areas of non-compaction, or in area(s) at risk of maladaptive cardiac remodeling.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A composition comprising cardiomyocytes in admixture with an isolated polypeptide selected from the group consisting of: the polypeptides listed in Table 1.
2. A composition comprising cardiomyocytes in admixture with two or more isolated polypeptides selected from the group consisting of: the polypeptides listed in Table 1.
3. The composition of paragraph 1 or paragraph 2, wherein the cardiomyocytes are human cardiomyocytes.
4. The composition of any one of paragraphs 1-3, wherein the cardiomyocytes are differentiated in vitro from embryonic stem cells or from induced pluripotent stem cells.
5. A transplant composition comprising the composition of any one of paragraphs 1-4.
6. A composition comprising cardiomyocytes that have been contacted with an isolated polypeptide selected from the group consisting of: the polypeptides listed in Table 1.
7. A composition comprising cardiomyocytes that have been contacted with two or more isolated polypeptides selected from the group consisting of: the polypeptides listed in Table 1.
8. The composition of paragraph 6 or paragraph 1 or paragraph 2, wherein the cardiomyocytes are human cardiomyocytes.
9. The composition of any one of paragraphs 1-3, wherein the cardiomyocytes are differentiated in vitro from embryonic stem cells or from induced pluripotent stem cells.
10. A transplant composition comprising the composition of any one of paragraphs 6-9.
11. A composition comprising cardiomyocytes and a nucleic acid construct encoding a polypeptide selected from the group consisting of: the polypeptides listed in Table 1.
12. A composition comprising cardiomyocytes and one or more nucleic acid constructs encoding two or more polypeptides selected from the group consisting of: the polypeptides listed in Table 1.
13. The composition of paragraph 11 or 12, wherein the construct is in a vector.
14. The composition of paragraph 11 or 12, wherein the construct or constructs is/are in admixture with a transfection reagent.
15. The composition of any one of paragraphs 11-14, wherein the cardiomyocytes are human cardiomyocytes.
16. The composition of any one of paragraphs 11-15, wherein the cardiomyocytes are differentiated in vitro from embryonic stem cells or induced pluripotent stem cells.
17. A transplant composition comprising the composition of any one of paragraphs 11-16.
18. A composition comprising cardiomyocytes in admixture with an isolated metabolic factor polypeptide that promotes transplant engraftment of the cardiomyocytes, or with a nucleic acid construct that encodes such a factor.
19. The composition of paragraph 18, wherein the isolated metabolic factor is selected from the group consisting of a polypeptide that promotes lipid hydrolysis and a polypeptide that modulates insulin or IGF signaling.
20. The composition of paragraph 18, wherein the polypeptide that promotes lipid hydrolysis is selected from LIPM, PSAP and PLA2G2C, and the polypeptide that modulates insulin or IGF signaling is selected from SERPINA12, HTRA1 and FETUB.
21. A composition comprising cardiomyocytes in admixture with an isolated vascular remodeling, extracellular matrix, proteoglycan or cell adhesion polypeptide that promotes transplant engraftment of the cardiomyocytes, or with a nucleic acid construct that encodes such a factor.
22. The composition of paragraph 21, which comprises two or more polypeptide factors selected from the groups consisting of vascular remodeling, extracellular matrix, proteoglycan and cell adhesion polypeptides.
23. The composition of paragraph 21, wherein the vascular remodeling polypeptide is selected from the group consisting of the polypeptides listed in Table 6, the extracellular matrix polypeptide is selected from the group consisting of the polypeptides listed in Table 7, the proteoglycan polypeptide is selected from the polypeptides listed in Table 8, and the cell adhesion polypeptide is selected from the polypeptides listed in Table 9.
24. A composition comprising cardiomyocytes in admixture with an isolated canonical Wnt pathway polypeptide.
25. A composition comprising cardiomyocytes that have been contacted with an isolated canonical Wnt pathway polypeptide.
26. The composition of paragraph 24 or 25, wherein the canonical Wnt pathway polypeptide is selected from the group consisting of the polypeptides listed in Table 2.
27. A composition comprising cardiomyocytes in admixture with an isolated serine protease polypeptide.
28. A composition comprising cardiomyocytes that have been contacted with an isolated serine protease polypeptide.
29. The composition of paragraph 27 or 28, wherein the serine protease polypeptide is selected from the group consisting of the polypeptides listed in Table 10.
30. A composition comprising cardiomyocytes in admixture with an isolated serine protease inhibitor polypeptide.
31. A composition comprising cardiomyocytes that have been contacted with an isolated serine protease inhibitor polypeptide.
32. The composition of paragraph 30 or 31, wherein the isolated serine protease inhibitor polypeptide is selected from the group consisting of the polypeptides listed in Table 11.
33. A composition comprising cardiomyocytes in admixture with an isolated signaling polypeptide of the interleukin family, interferon signaling family, or chemokine family.
34. A composition comprising cardiomyocytes that have been contacted with an isolated signaling polypeptide of the interleukin family, interferon signaling family, or chemokine family.
35. The composition of paragraph 33 or 34, wherein the signaling polypeptide of the interleukin family is selected from the group consisting of the polypeptides listed in Table 12, the signaling polypeptide of the interferon signaling family is selected from the polypeptides listed in Table 13, and the signaling polypeptide of the chemokine family is selected from the group consisting of the polypeptides listed in Table 14.
36. A composition comprising cardiomyocytes in admixture with an isolated TLR binding polypeptide, a lipocalin polypeptide or a secretaglobin polypeptide.
37. A composition comprising cardiomyocytes that have been contacted with an isolated TLR binding polypeptide, a lipocalin polypeptide or a secretaglobin polypeptide.
38. The composition of paragraph 36 or 37, wherein the TLR binding polypeptide is selected from the polypeptides listed in Table 15, the lipocalin polypeptide is selected from the polypeptides listed in Table 16, and the secretaglobin polypeptide is selected from the polypeptides listed in Table 17.
39. A cardiac delivery device comprising a composition of any one of paragraphs 1-38.
40. A method of transplanting cardiomyocytes, the method comprising administering a composition of any one of paragraphs 1-38 to cardiac tissue, optionally using the cardiac delivery device of paragraph 39.
41. The method of paragraph 40, wherein the engraftment of the administered cardiomyocytes is increased relative to engraftment of cardiomyocytes that were not in admixture with or had not been contacted with the polypeptide or polypeptides.
42. A method of enhancing cardiomyocyte transplant engraftment, the method comprising contacting a cardiomyocyte population with a polypeptide selected from the group consisting of the polypeptides listed in Table 1, and transplanting the cardiomyocyte population to cardiac tissue.
43. A method of enhancing cardiomyocyte transplant engraftment, the method comprising contacting a cardiomyocyte population with a nucleic acid that encodes a polypeptide selected from the group consisting of the polypeptides listed in Table 1, and transplanting the cardiomyocyte population to cardiac tissue.
44. A method of enhancing cardiomyocyte transplant engraftment, the method comprising contacting a cardiomyocyte population with a vector that encodes a polypeptide selected from the group consisting of the polypeptides listed in Table 1, and transplanting the cardiomyocyte population to cardiac tissue.
45. The method of paragraph 44, wherein the vector comprises an AAV vector.
46. The method of any one of paragraphs 42-45, wherein the cardiomyocyte population is a human cardiomyocyte population.
47. The method of any one of paragraphs 42-46, wherein the cardiomyocyte population is differentiated in vitro from embryonic stem cells or induced pluripotent stem cells.
48. The method of paragraph 47, wherein the induced pluripotent stem cells are differentiated from induced pluripotent stem cells derived from the transplant recipient.

The technology described herein is further illustrated by the following examples which in no way should be construed as being further limiting.

### EXAMPLES

### Example 1: Improved Survival of Engrafted Stem Cell-Derived Cardiomyocytes by Expression of Secreted Factors

### Functional Selection Strategy

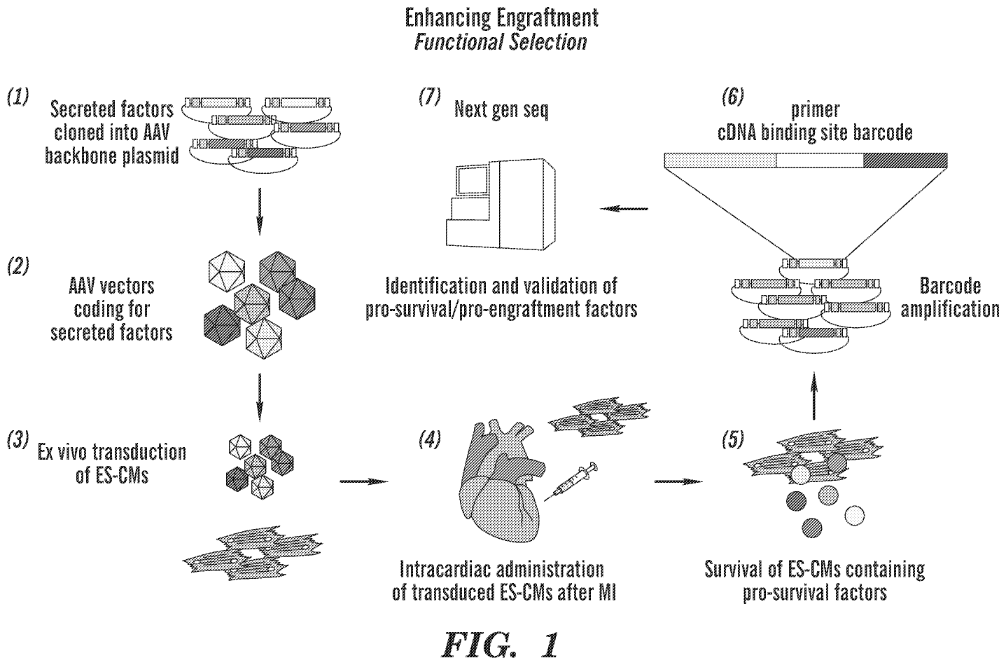

AAV plasmids, each encoding a different secreted factor and unique DNA barcode, were transduced as pools into stem cell-derived cardiomyocytes. The transduced cardiomyocytes were transplanted into the heart wall of mice at the time of myocardial infarction. Barcodes associated with factors that promote engraftment of the cardiomyocytes were enriched, whereas barcodes associated with factors that inhibit engraftment were depleted (FIG. 1).

### Execution of Functional Selection Strategy

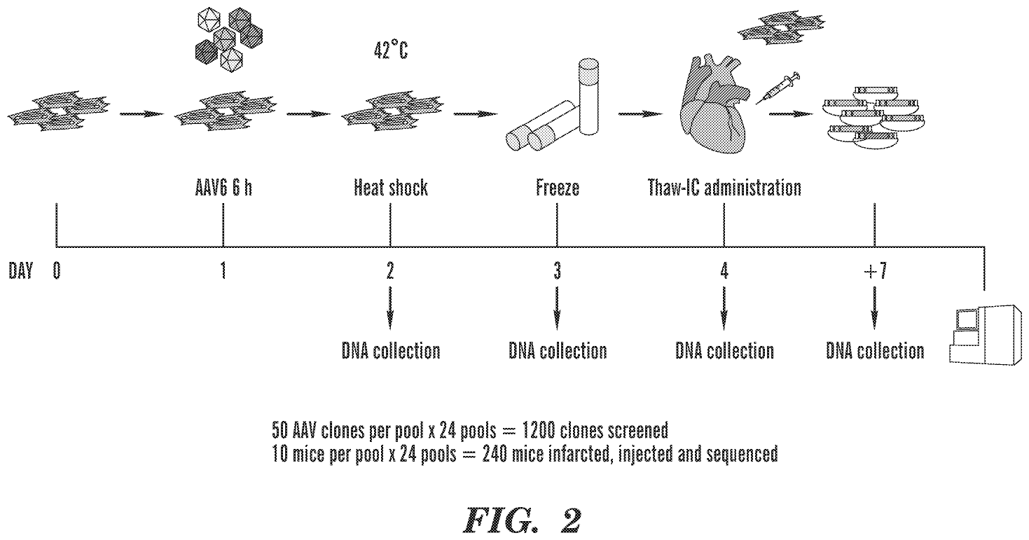

Stem cell-derived cardiomyocytes were transduced with a pool of 50 AAV clones and cryopreserved before transplant. For transplantation, NOD scid gamma (NSG) mice were subjected to cardiac infarction by permanent occlusion of the left anterior descending artery. Immediately after occlusion, the thawed transduced cardiomyocytes were injected into the left ventricular wall at the site of infarction. DNA sequencing of the thawed cardiomyocytes before transplant provided a baseline reference for subsequent enrichment or depletion. Three days post-injection, the mice were sacrificed, and the hearts were collected and snap frozen in liquid nitrogen for subsequent DNA sequencing. (FIG. 2).

### Results of Screen

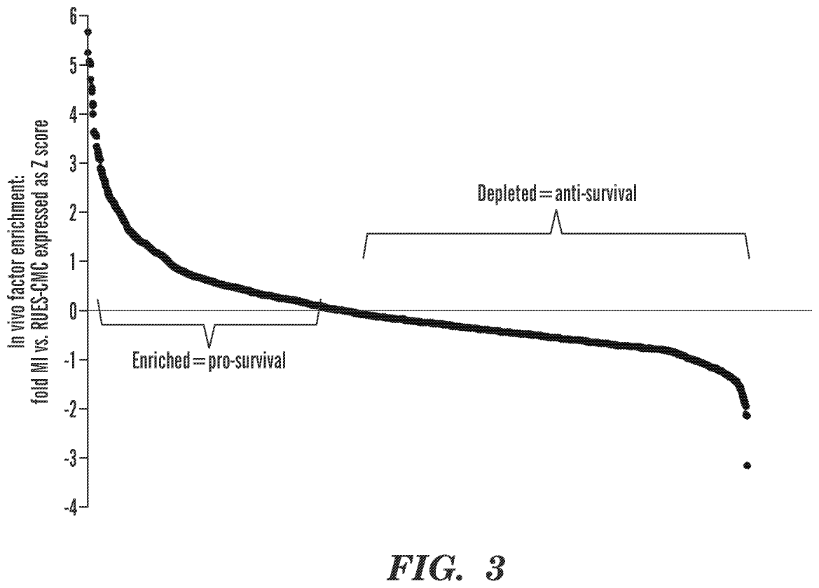

2400 clones were ranked according to Z score for the ratio of barcode counts before transplant to barcode counts after transplant. A positive ratio reflects enrichment of a barcode and indicates the encoded factor promoted cardiomyocyte engraftment. In contrast, a negative ratio indicates the encoded factor inhibited cardiomyocyte engraftment (FIG. 3).

### Top Hits Promoting Cardiomyocyte Engraftment

Screened clones with the highest Z score for enrichment were retested as a pool. The resultant data confirm that these factors promoted cardiomyocyte engraftment to varied extents. (FIG. 4). See e.g., Ruozi et al. (2015) Nat Commun 6:7388, the content of which is incorporated herein by reference in its entirety.

### Sequences

In some embodiments of any of the aspects, at least one human nucleic acid sequence is selected from the group consisting of: SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, at least 99% identical, or more to the sequence of at least one of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, or a protein coding region (CDS) thereof, e.g., that maintains the same functions when translated into a protein, or a codon-optimized version thereof.

In some embodiments of any of the aspects, at least one murine nucleic acid sequence is selected from the group consisting of: SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical, or more to the sequence of at least one of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, or a protein coding region (CDS) thereof, e.g., that maintains the same functions when translated into a protein, or a codon-optimized version thereof.

In some embodiments of any of the aspects, at least one human polypeptide sequence is selected from the group consisting of: SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, at least 99% identical, or more to the sequence of at least one of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239 that maintains the same functions.

In some embodiments of any of the aspects, at least one murine polypeptide sequence is selected from the group consisting of: SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, at least 99% identical, or more to the sequence of at least one of SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240 that maintains the same functions.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA. In some embodiments, RNA can be used in place of DNA as described herein. In some embodiments, DNA can be used in place of RNA as described herein.

Exemplary RAI2 sequences include:
Homo sapiens retinoic acid induced 2 (RAI2), RefSeqGene on chromosome X, NCBI Reference Sequence: NG_016739.1, NG_016739.1:5001-66289 Homo sapiens retinoic acid induced 2 (RAI2), RefSeqGene on chromosome X;
Rai2 retinoic acid induced 2 [Mus musculus (house mouse)], Mus musculus strain C57BL/6J chromosome X, GRCm38.p6 C57BL/6 J, NCBI Reference Sequence: NC_000086.7, >NC_000086.7:161717036-161779494 Mus musculus strain C57BL/6J chromosome X, GRCm38.p6 C57BL/6J;
SEQ ID NO: 1-Homo sapiens retinoic acid induced 2 (RAI2), transcript variant 2, mRNA, NCBI Reference Sequence: NM_021785.4;
SEQ ID NO: 2-Mus musculus retinoic acid induced 2 (Rai2), transcript variant 2, mRNA, NCBI Reference Sequence: NM_001103367.1;
SEQ ID NO: 3-Retinoic acid-induced protein 2 isoform 1 [Homo sapiens], NCBI Reference Sequence: NP_068557.3;
SEQ ID NO: 4-Retinoic acid-induced protein 2 [Mus musculus], NCBI Reference Sequence: NP_001096837.1.

Exemplary Hmgb1 sequences include:
HMGB1 high mobility group box 1 [Homo sapiens (human)], Homo sapiens chromosome 13, GRCh38.p13 Primary Assembly, NCBI Reference Sequence: NC_000013.11, >NC 000013.11: c30617597-30456704 Homo sapiens chromosome 13, GRCh38.p13 Primary Assembly;
Hmgb1 high mobility group box 1 [Mus musculus (house mouse)], Mus musculus strain C57BL/6J chromosome 5, GRCm38.p6 C57BL/6 J, NCBI Reference Sequence: NC_000071.6, >NC_000071.6: c149053057-149046702 Mus musculus strain C57BL/6J chromosome 5, GRCm38.p6 C57BL/6J;
SEQ ID NO: 5-Homo sapiens high mobility group box 1 (HMGB1), transcript variant 1, mRNA NCBI Reference Sequence: NM_001313893.1;
SEQ ID NO: 6-Mus musculus high mobility group box 1 (Hmgb1), transcript variant 2, mRNA NCBI Reference Sequence: NM_010439.4;
SEQ ID NO: 7-High mobility group protein B1 isoform 1 [Homo sapiens] NCBI Reference Sequence: NP_001300822.1;
SEQ ID NO: 8-High mobility group protein B1 [Mus musculus] NCBI Reference Sequence: NP_034569.1.

Exemplary FURIN sequences include:
FURIN furin, paired basic amino acid cleaving enzyme [Homo sapiens (human)], Homo sapiens chromosome 15, GRCh38.p13 Primary Assembly, NCBI Reference Sequence: NC_000015.10, >NC 000015.10:90868588-90883458 Homo sapiens chromosome 15, GRCh38.p13 Primary Assembly; Furin: furin (paired basic amino acid cleaving enzyme) [Mus musculus (house mouse)], Mus musculus strain C57BL/6J chromosome 7, GRCm38.p6 C57BL/6J, NCBI Reference Sequence: NC_000073.6, >NC 000073.6: c80405441-80389194 Mus musculus strain C57BL/6J chromosome 7, GRCm38.p6 C57BL/6J;
SEQ ID NO: 9-Homo sapiens furin, paired basic amino acid cleaving enzyme (FURIN), transcript variant 2, mRNA, NCBI Reference Sequence: NM_001289823.1;
SEQ ID NO: 10, Mus musculus furin (paired basic amino acid cleaving enzyme) (Furin), transcript variant 2, mRNA, NCBI Reference Sequence: NM_001081454.2;
SEQ ID NO: 11, furin preproprotein [Homo sapiens], NCBI Reference Sequence: NP_001276752.1;
SEQ ID NO: 12, furin preproprotein [Mus musculus], NCBI Reference Sequence: NP_001074923.1.

Exemplary Cnpy4 sequences include:
NPY4 canopy FGF signaling regulator 4 [Homo sapiens (human)], Homo sapiens chromosome 15, GRCh38.p13 Primary Assembly, NCBI Reference Sequence: NC_000007.14, >NC_000007.14 (100119634-100125508) Homo sapiens chromosome 15, GRCh38.p13 Primary Assembly;
Cnpy4 canopy FGF signaling regulator 4 [Mus musculus (house mouse)], Mus musculus strain C57BL/6J chromosome 5, GRCm38.p6 C57BL/6J, NCBI Reference Sequence: NC_000071.6, >NC_000071.6 (138187535-138193894) Mus musculus strain C57BL/6J chromosome 5, GRCm38.p6 C57BL/6J;
SEQ ID NO: 13, Homo sapiens canopy FGF signaling regulator 4 (CNPY4), mRNA, NCBI Reference Sequence: NM_152755.2;
SEQ ID NO: 14, Mus musculus canopy FGF signaling regulator 4 (Cnpy4), mRNA, NCBI Reference Sequence: NM_178612.4;
SEQ ID NO: 15, protein canopy homolog 4 precursor [Homo sapiens], NCBI Reference Sequence: NP_689968.1;
SEQ ID NO: 16, protein canopy homolog 4 precursor [Mus musculus], NCBI Reference Sequence: NP_848727.1.

Exemplary C8 sequences include:
Homo sapiens complement C8 alpha chain (C8A), Homo sapiens chromosome 1, GRCh38.p13 Primary Assembly, NCBI Reference Sequence: NC_000001.11, >NC_000001.11 (56854770-56918221) Homo sapiens chromosome 1, GRCh38.p13 Primary Assembly;
C8a complement component 8, alpha polypeptide [Mus musculus (house mouse)], Mus musculus strain C57BL/6J chromosome 4, GRCm38.p6 C57BL/6J, NCBI Reference Sequence: NC_000070.6, >NC_000070.6 (104815679-104876487, complement) Mus musculus strain C57BL/6J chromosome 4, GRCm38.p6 C57BL/6J;
SEQ ID NO: 17, Homo sapiens complement C8 alpha chain (C8A), mRNA, NCBI Reference Sequence: NM_000562.2;
SEQ ID NO: 18, PREDICTED: Mus musculus complement component 8, alpha polypeptide (C8a), transcript variant X1, mRNA, NCBI Reference Sequence: XM_006502941.3;
SEQ ID NO: 19, complement component C8 alpha chain preproprotein [Homo sapiens], NCBI Reference Sequence: NP_000553.1;
SEQ ID NO: 20, complement component C8 alpha chain isoform X1 [Mus musculus], NCBI Reference Sequence: XP_006503004.1.

Exemplary IL7 sequences include:
Homo sapiens interleukin 7 (IL7), Homo sapiens chromosome 8, GRCh38.p13 Primary Assembly, NCBI Reference Sequence: NC_000008.11, >NC_000008.11 (78675870-78806830, complement) Homo sapiens chromosome 8, GRCh38.p13 Primary Assembly;
Mus musculus interleukin 7 (117), Mus musculus strain C57BL/6J chromosome 3, GRCm38.p6 C57BL/6J, NCBI Reference Sequence: NC_000069.6, >NC_000069.6 (7572028-7613760, complement) Mus musculus strain C57BL/6J chromosome 3, GRCm38.p6 C57BL/6J;
SEQ ID NO: 21, Homo sapiens interleukin 7 (IL7), transcript variant 1, mRNA, NCBI Reference Sequence: NM_000880.4;
SEQ ID NO: 22, Mus musculus interleukin 7 (Il7), transcript variant 1, mRNA, NCBI Reference Sequence: NM_008371.5;
SEQ ID NO: 23, interleukin-7 isoform 1 precursor [Homo sapiens], NCBI Reference Sequence: NP_000871.1;
SEQ ID NO: 24, interleukin-7 isoform b [Mus musculus], NCBI Reference Sequence: NP_001300817.1. Exemplary WNT3A sequences include:

Homo sapiens Wnt family member 3A (WNT3A), Homo sapiens chromosome 1, GRCh38.p13 Primary Assembly, NCBI Reference Sequence: NC_000001.11, >NC_000001.11 (228006998-228067113) Homo sapiens chromosome 1, GRCh38.p13 Primary Assembly;
Mus musculus wingless-type MMTV integration site family, member 3A (Wnt3a), Mus musculus strain C57BL/6J chromosome 11, GRCm38.p6 C57BL/6J, NCBI Reference Sequence: NC_000077.6, >NC_000077.6 (59248036-59290751, complement) Mus musculus strain C57BL/6J chromosome 11, GRCm38.p6 C57BL/6J;
SEQ ID NO: 25, Homo sapiens Wnt family member 3A (WNT3A), mRNA, NCBI Reference Sequence: NM_033131.4;
SEQ ID NO: 26, Mus musculus wingless-type MMTV integration site family, member 3A (Wnt3a), mRNA, NCBI Reference Sequence: NM_009522.2;
SEQ ID NO: 27, protein Wnt-3a precursor [Homo sapiens], NCBI Reference Sequence: NP_149122.1;
SEQ ID NO: 28, protein Wnt-3a precursor [Mus musculus], NCBI Reference Sequence: NP_033548.1.

Exemplary MMP21 sequences include:
Homo sapiens matrix metallopeptidase 21 (MMP21), Homo sapiens chromosome 10, GRCh38.p13 Primary Assembly, NCBI Reference Sequence: NC_000010.11, >NC_000010.11 (125766453-125775821, complement) Homo sapiens chromosome 10, GRCh38.p13 Primary Assembly;
Mus musculus matrix metallopeptidase 21 (Mmp21), Mus musculus strain C57BL/6J chromosome 7, GRCm38.p6 C57BL/6J, NCBI Reference Sequence: NC_000073.6, >NC_000073.6 (133674270-133680061, complement) Mus musculus strain C57BL/6J chromosome 7, GRCm38.p6 C57BL/6J;
SEQ ID NO: 29, Homo sapiens matrix metallopeptidase 21 (MMP21), mRNA, NCBI Reference Sequence: NM_147191.1;
SEQ ID NO: 30, Mus musculus matrix metallopeptidase 21 (Mmp21), transcript variant 1, mRNA, Sequence ID: NM_152944.1;
SEQ ID NO: 31, matrix metalloproteinase-21 preproprotein [Homo sapiens], NCBI Reference Sequence: NP_671724.1;
SEQ ID NO: 32, matrix metalloproteinase-21 isoform 2 precursor [Mus musculus], NCBI Reference Sequence: NP_001307145.1.

Exemplary TINAGL1 sequences include:
Homo sapiens tubulointerstitial nephritis antigen like 1 (TINAGL1), Homo sapiens chromosome 1, GRCh38.p13 Primary Assembly, NCBI Reference Sequence: NC_000001.11, >NC_000001.11 (31576384-31587686) Homo sapiens chromosome 1, GRCh38.p13 Primary Assembly;
Mus musculus tubulointerstitial nephritis antigen-like 1 (Tinagl1), Mus musculus strain C57BL/6J chromosome 4, GRCm38.p6 C57BL/6J, NCBI Reference Sequence: NC_000070.6, >NC_000070.6 (130165600-130175122, complement) Mus musculus strain C57BL/6J chromosome 4, GRCm38.p6 C57BL/6J;
SEQ ID NO: 33, Homo sapiens tubulointerstitial nephritis antigen like 1 (TINAGL1), transcript variant 2, mRNA, NCBI Reference Sequence: NM_001204414.1;
SEQ ID NO: 34, Mus musculus tubulointerstitial nephritis antigen-like 1 (Tinagl1), transcript variant 2, mRNA, NCBI Reference Sequence: NM_001168333.1;
SEQ ID NO: 35, tubulointerstitial nephritis antigen-like isoform 2 precursor [Homo sapiens], NCBI Reference Sequence: NP_001191343.1;
SEQ ID NO: 36, tubulointerstitial nephritis antigen-like precursor [Mus musculus], NCBI Reference Sequence: NP_001161805.1.

Exemplary LIPM sequences include:
Homo sapiens lipase family member M (LIPM), Homo sapiens chromosome 10, GRCh38.p13 Primary Assembly, NCBI Reference Sequence: NC_000010.11, >NC_000010.11 (88802730-88822022) Homo sapiens chromosome 10, GRCh38.p13 Primary Assembly;
Mus musculus lipase, family member M (Lipm), Mus musculus strain C57BL/6J chromosome 19, GRCm38.p6 C57BL/6J, NCBI Reference Sequence: NC_000085.6, >NC_000085.6 (34100943-34122687) Mus musculus strain C57BL/6J chromosome 19, GRCm38.p6 C57BL/6J;
SEQ ID NO: 37, Homo sapiens lipase family member M (LIPM), mRNA, NCBI Reference Sequence: NM_001128215.1;
SEQ ID NO: 38, Mus musculus lipase, family member M (Lipm), mRNA, NCBI Reference Sequence: NM_023903.1;
SEQ ID NO: 39, lipase member M precursor [Homo sapiens], NCBI Reference Sequence: NP_001121687.1;
SEQ ID NO: 40, lipase member M precursor [Mus musculus], NCBI Reference Sequence: NP_076392.1.

Exemplary KLK8 sequences include:
KLK8 kallikrein related peptidase 8 [Homo sapiens (human)], Homo sapiens chromosome 19, GRCh38.p13 Primary Assembly, NCBI Reference Sequence: NC_000019.10, >NC_000019.10 (50996008-51001702, complement) Homo sapiens chromosome 19, GRCh38.p13 Primary Assembly;
Mus musculus kallikrein related-peptidase 8 (Klk8), Mus musculus strain C57BL/6J chromosome 7, GRCm38.p6 C57BL/6J, NCBI Reference Sequence: NC_000073.6, >NC_000073.6 (43797532-43803826) Mus musculus strain C57BL/6J chromosome 7, GRCm38.p6 C57BL/6J;
SEQ ID NO: 41, Homo sapiens kallikrein related peptidase 8 (KLK8), transcript variant 5, mRNA, NCBI Reference Sequence: NM_001281431.1;
SEQ ID NO: 42, Mus musculus kallikrein related-peptidase 8 (Klk8), transcript variant 2, mRNA, NCBI Reference Sequence: NM_001324398.1;
SEQ ID NO: 43, kallikrein-8 isoform 5 [Homo sapiens], NCBI Reference Sequence: NP_001268360.1;
SEQ ID NO: 44, kallikrein-8 preproprotein [Mus musculus], NCBI Reference Sequence: NP_001311327.1.

Exemplary SERPINA 12 sequences include:
SERPINA12 serpin family A member 12 [Homo sapiens (human)], Homo sapiens chromosome 14, GRCh38.p13 Primary Assembly, NCBI Reference Sequence: NC_000014.9, >NC_000014.9 (94481651-94517844, complement) Homo sapiens chromosome 14, GRCh38.p13 Primary Assembly;
Mus musculus serine (or cysteine) peptidase inhibitor, clade A (alpha-1 antiproteinase, antitrypsin), member 12 (Serpina12), Mus musculus strain C57BL/6J chromosome 12, GRCm38.p6 C57BL/6J, NCBI Reference Sequence: NC_000078.6, >NC_000078.6 (104028769-104044443, complement) Mus musculus strain C57BL/6J chromosome 12, GRCm38.p6 C57BL/6J;
SEQ ID NO: 45, PREDICTED: Homo sapiens serpin family A member 12 (SERPINA12), transcript variant X7, mRNA, NCBI Reference Sequence: XM_011536455.1;
SEQ ID NO: 46, Mus musculus serine (or cysteine) peptidase inhibitor, clade A (alpha-1 antiproteinase, antitrypsin), member 12 (Serpina12), mRNA, NCBI Reference Sequence: NM_026535.2;
SEQ ID NO: 47, serpin A12 isoform X2 [Homo sapiens], NCBI Reference Sequence: XP_011534757.1;
SEQ ID NO: 48, serpin A12 precursor [Mus musculus], NCBI Reference Sequence: NP_080811.1.

Exemplary SERPIND1 sequences include:
SERPIND1 serpin family D member 1 [Homo sapiens (human)] Homo sapiens chromosome 22, GRCh38.p13 Primary Assembly, NCBI Reference Sequence: NC_000022.11, >NC_000022.11 (20774113-20787720) Homo sapiens chromosome 22, GRCh38.p13 Primary Assembly;
Serpind1 serine (or cysteine) peptidase inhibitor, clade D, member 1 [Mus musculus (house mouse)], Mus musculus strain C57BL/6J chromosome 16, GRCm38.p6 C57BL/6J, NCBI Reference Sequence: NC_000082.6, >NC_000082.6 (17331371-17343574) Mus musculus strain C57BL/6J chromosome 16, GRCm38.p6 C57BL/6J;
SEQ ID NO: 49, Homo sapiens serpin family D member 1 (SERPIND1), mRNA, NCBI Reference Sequence: NM_000185.4;
SEQ ID NO: 50, Mus musculus serine (or cysteine) peptidase inhibitor, clade D, member 1 (Serpind1), transcript variant 2, mRNA, NCBI Reference Sequence: NM_001331047.1;
SEQ ID NO: 51, heparin cofactor 2 precursor [Homo sapiens], NCBI Reference Sequence: NP_000176.2;
SEQ ID NO: 52, heparin cofactor 2 precursor [Mus musculus], NCBI Reference Sequence: NP_001317976.1.

Exemplary GPC1 sequences include:
GPC1 glypican 1 [Homo sapiens (human)], Homo sapiens chromosome 2, GRCh38.p13 Primary Assembly NCBI Reference Sequence: NC_000002.12, >NC_000002.12 (240435663-240468076) Homo sapiens chromosome 2, GRCh38.p13 Primary Assembly;
Gpc1 glypican 1 [Mus musculus (house mouse)], Mus musculus strain C57BL/6J chromosome 1, GRCm38.p6 C57BL/6J, NCBI Reference Sequence: NC_000067.6,>NC_000067.6 (92831645-92860211) Mus musculus strain C57BL/6J chromosome 1, GRCm38.p6 C57BL/6J;
SEQ ID NO: 53, Homo sapiens glypican 1 (GPC1), mRNA, NCBI Reference Sequence: NM_002081.3;
SEQ ID NO: 54, Mus musculus glypican 1 (Gpc1), mRNA, NCBI Reference Sequence: NM_016696.5;
SEQ ID NO: 55, glypican-1 precursor [Homo sapiens], NCBI Reference Sequence: NP_002072.2;
SEQ ID NO: 56, glypican-1 precursor [Mus musculus], NCBI Reference Sequence: NP_057905.1.

Exemplary PSAP sequences include:
PSAP prosaposin [Homo sapiens (human)], Homo sapiens chromosome 10, GRCh38.p13 Primary Assembly, NCBI Reference Sequence: NC_000010.11, >NC_000010.11 (71816298-71851251, complement) Homo sapiens chromosome 10, GRCh38.p13 Primary Assembly;
Psap prosaposin [Mus musculus (house mouse)], Mus musculus strain C57BL/6J chromosome 10, GRCm38.p6 C57BL/6J, NCBI Reference Sequence: NC_000076.6, >NC_000076.6 (60277628-60302600) Mus musculus strain C57BL/6J chromosome 10, GRCm38.p6 C57BL/6J;
SEQ ID NO: 57, Homo sapiens prosaposin (PSAP), transcript variant 2, mRNA, NCBI Reference Sequence: NM_001042465.3;
SEQ ID NO: 58, Mus musculus prosaposin (Psap), transcript variant 1, mRNA, NCBI Reference Sequence: NM_001146120.1;
SEQ ID NO: 59, prosaposin isoform b preproprotein [Homo sapiens], NCBI Reference Sequence: NP_001035930.1;
SEQ ID NO: 60, prosaposin isoform A precursor [Mus musculus], NCBI Reference Sequence: NP_001139592.1.

Exemplary PRSS29P sequences include:
PRSS29P serine protease 29, pseudogene [Homo sapiens (human)], Homo sapiens chromosome 16, GRCh38.p13 Primary Assembly, NCBI Reference Sequence: NC_000016.10, >NC_000016.10 (1261003-1264004, complement) Homo sapiens chromosome 16, GRCh38.p13 Primary Assembly;
Mus musculus strain C57BL/6J chromosome 17, GRCm38.p6 C57BL/6J, NCBI Reference Sequence: NC_000083.6, >NC_000083.6 (25318654-25322684) Mus musculus strain C57BL/6J chromosome 17, GRCm38.p6 C57BL/6J;
SEQ ID NO: 61, NONE-PSEUDOGENE; see e.g., mouse homolog SEQ ID NO: 62;
SEQ ID NO: 62, Mus musculus protease, serine 29 (Prss29), mRNA, NCBI Reference Sequence: NM_053260.3;
SEQ ID NO: 63, NONE-PSEUDOGENE; see mouse homolog, SEQ ID NO: 64;
SEQ ID NO: 64, serine protease 29 precursor [Mus musculus], NCBI Reference Sequence: NP_444490.2.

Exemplary AMBP sequences include:
AMBP alpha-1-microglobulin/bikunin precursor [Homo sapiens (human)], Homo sapiens chromosome 9, GRCh38.p13 Primary Assembly, NCBI Reference Sequence: NC_000009.12, >NC_000009.12 (114060127-114078300, complement) Homo sapiens chromosome 9, GRCh38.p13 Primary Assembly;
Ambp alpha 1 microglobulin/bikunin precursor [Mus musculus (house mouse)], Mus musculus strain C57BL/6J chromosome 4, GRCm38.p6 C57BL/6J, NCBI Reference Sequence: NC_000070.6, >NC_000070.6 (63143275-63154172, complement) Mus musculus strain C57BL/6J chromosome 4, GRCm38.p6 C57BL/6J;
SEQ ID NO: 65, Homo sapiens alpha-1-microglobulin/bikunin precursor (AMBP), mRNA, NCBI Reference Sequence: NM_001633.4;
SEQ ID NO: 66, Mus musculus alpha 1 microglobulin/bikunin precursor (Ambp), mRNA, NCBI Reference Sequence: NM_007443.4;
SEQ ID NO: 67, protein AMBP preproprotein [Homo sapiens], NCBI Reference Sequence: NP_001624.1;
SEQ ID NO: 68, protein AMBP preproprotein [Mus musculus], NCBI Reference Sequence: NP_031469.1.

Exemplary CPA6 sequences include:
CPA6 carboxypeptidase A6 [Homo sapiens (human)], Homo sapiens chromosome 8, GRCh38.p13 Primary Assembly, NCBI Reference Sequence: NC_000008.11, >NC_000008.11 (67422038-67747114, complement) Homo sapiens chromosome 8, GRCh38.p13 Primary Assembly;
Cpa6 carboxypeptidase A6 [Mus musculus (house mouse)], Mus musculus strain C57BL/6J chromosome 1, GRCm38.p6 C57BL/6J, NCBI Reference Sequence: NC_000067.6, >NC_000067.6 (10295791-10720053, complement) Mus musculus strain C57BL/6J chromosome 1, GRCm38.p6 C57BL/6J;
SEQ ID NO: 69, Homo sapiens carboxypeptidase A6 (CPA6), mRNA, NCBI Reference Sequence: NM_020361.5;
SEQ ID NO: 70, Mus musculus carboxypeptidase A6 (Cpa6), transcript variant 2, mRNA, NCBI Reference Sequence: NM_001289497.1;
SEQ ID NO: 71, carboxypeptidase A6 preproprotein [Homo sapiens], NCBI Reference Sequence: NP_065094.3;
SEQ ID NO: 72, carboxypeptidase A6 isoform 2 [Mus musculus], NCBI Reference Sequence: NP_001276426.1.

Exemplary SCGB3A2 sequences include:
SCGB3A2 secretoglobin family 3A member 2 [Homo sapiens (human)], Homo sapiens chromosome 5, GRCh38.p13 Primary Assembly, NCBI Reference Sequence: NC_000005.10, >NC_000005.10 (147878711-147882191) Homo sapiens chromosome 5, GRCh38.p13 Primary Assembly;
Scgb3a2 secretoglobin, family 3A, member 2 [Mus musculus (house mouse)], Mus musculus strain C57BL/6J chromosome 18, GRCm38.p6 C57BL/6J, NCBI Reference Sequence: NC_000084.6, >NC_000084.6 (43764281-43767399) Mus musculus strain C57BL/6J chromosome 18, GRCm38.p6 C57BL/6J;
SEQ ID NO: 73, Homo sapiens secretoglobin family 3A member 2 (SCGB3A2), mRNA, NCBI Reference Sequence: NM_054023.5;
SEQ ID NO: 74, Mus musculus secretoglobin, family 3A, member 2 (Scgb3a2), transcript variant 1, mRNA, NCBI Reference Sequence: NM_001289643.1;
SEQ ID NO: 75, secretoglobin family 3A member 2 precursor [Homo sapiens], NCBI Reference Sequence: NP_473364.1;
SEQ ID NO: 76, secretoglobin family 3A member 2 isoform 1 [Mus musculus], NCBI Reference Sequence: NP_001276572.1.

Exemplary Reg3d sequences include:
Reg3d regenerating islet-derived 3 delta [Mus musculus (house mouse)], Mus musculus strain C57BL/6J chromosome 6, GRCm38.p6 C57BL/6J, NCBI Reference Sequence: NC_000072.6, >NC_000072.6 (78375874-78378865, complement) Mus musculus strain C57BL/6J chromosome 6, GRCm38.p6 C57BL/6J
SEQ ID NO: 77, see murine homolog, SEQ ID NO: 78;
SEQ ID NO: 78, Mus musculus regenerating islet-derived 3 delta (Reg3d), transcript variant 2, mRNA NCBI Reference Sequence: NM_001161741.1;
SEQ ID NO: 79, see murine homolog, SEQ ID NO: 80;
SEQ ID NO: 80, regenerating islet-derived 3 delta isoform 2 precursor [Mus musculus], NCBI Reference Sequence: NP_001155213.1.

Exemplary Scgb2b24 sequences include:
Scgb2b24 secretoglobin, family 2B, member 24 [Mus musculus (house mouse)], Mus musculus strain C57BL/6J chromosome 7, GRCm38.p6 C57BL/6J, NCBI Reference Sequence: NC_000073.6, >NC_000073.6 (33737193-33739295, complement) Mus musculus strain C57BL/6J chromosome 7, GRCm38.p6 C57BL/6J;
SEQ ID NO: 81, see murine homolog, SEQ ID NO: 82;
SEQ ID NO: 82, Mus musculus secretoglobin, family 2B, member 24 (Scgb2b24), mRNA, NCBI Reference Sequence: NM_177446.2;
SEQ ID NO: 83, see murine homolog, SEQ ID NO: 84;
SEQ ID NO: 84, secretoglobin family 2B member 24 precursor [Mus musculus], NCBI Reference Sequence: NP_803229.1.

Exemplary NPFF sequences include:
NPFF neuropeptide FF-amide peptide precursor [Homo sapiens (human)], Homo sapiens chromosome 12, GRCh38.p13 Primary Assembly, NCBI Reference Sequence: NC_000012.12, >NC_000012.12 (53506688-53507484, complement) Homo sapiens chromosome 12, GRCh38.p13 Primary Assembly;
Npff neuropeptide FF-amide peptide precursor [Mus musculus (house mouse)], Mus musculus strain C57BL/6J chromosome 15, GRCm38.p6 C57BL/6J, NCBI Reference Sequence: NC_000081.6, >NC_000081.6 (102523839-102524621, complement) Mus musculus strain C57BL/6J chromosome 15, GRCm38.p6 C57BL/6J;
SEQ ID NO: 85, Homo sapiens neuropeptide FF-amide peptide precursor (NPFF), transcript variant 2, mRNA, NCBI Reference Sequence: NM_001320296.2;
SEQ ID NO: 86, Mus musculus neuropeptide FF-amide peptide precursor (Npff), mRNA, NCBI Reference Sequence: NM_018787.1;
SEQ ID NO: 87, pro-FMRFamide-related neuropeptide FF isoform 2 [Homo sapiens], NCBI Reference Sequence: NP_001307225.1;
SEQ ID NO: 88, pro-FMRFamide-related neuropeptide FF preproprotein [Mus musculus], NCBI Reference Sequence: NP_061257.1.

Exemplary FAM3B sequences include:
FAM3B family with sequence similarity 3 member B [Homo sapiens (human)], Homo sapiens chromosome 21, GRCh38.p13 Primary Assembly, NCBI Reference Sequence: NC_000021.9, >NC_000021.9 (41304229-41357727) Homo sapiens chromosome 21, GRCh38.p13 Primary Assembly;
Fam3b family with sequence similarity 3, member B [Mus musculus (house mouse)], Mus musculus strain C57BL/6J chromosome 16, GRCm38.p6 C57BL/6J, NCBI Reference Sequence: NC_000082.6, >NC_000082.6 (97470965-97504936, complement) Mus musculus strain C57BL/6J chromosome 16, GRCm38.p6 C57BL/6J;
SEQ ID NO: 89, Homo sapiens family with sequence similarity 3 member B (FAM3B), transcript variant 1, mRNA, NCBI Reference Sequence: NM_058186.4;
SEQ ID NO: 90, Mus musculus family with sequence similarity 3, member B (Fam3b), mRNA, NCBI Reference Sequence: NM_020622.3;
SEQ ID NO: 91, protein FAM3B isoform a precursor [Homo sapiens], NCBI Reference Sequence: NP_478066.3;
SEQ ID NO: 92, protein FAM3B precursor [Mus musculus], NCBI Reference Sequence: NP_065647.1.

Exemplary FSTL1 sequences include:
FSTL1 follistatin like 1 [Homo sapiens (human)], Homo sapiens chromosome 3, GRCh38.p13 Primary Assembly, NCBI Reference Sequence: NC_000003.12, >NC_000003.12 (120392293-120450993, complement) Homo sapiens chromosome 3, GRCh38.p13 Primary Assembly;
Fstl1 follistatin-like 1 [Mus musculus (house mouse)], Mus musculus strain C57BL/6J chromosome 16, GRCm38.p6 C57BL/6J, NCBI Reference Sequence: NC_000082.6,>NC_000082.6 (37777055-37836516) Mus musculus strain C57BL/6J chromosome 16, GRCm38.p6 C57BL/6J;
SEQ ID NO: 93, Homo sapiens follistatin like 1 (FSTL1), mRNA, NCBI Reference Sequence: NM_007085.5;
SEQ ID NO: 94, Mus musculus follistatin-like 1 (Fstl1), mRNA, NCBI Reference Sequence: NM_008047.5;
SEQ ID NO: 95, follistatin-related protein 1 precursor [Homo sapiens], NCBI Reference Sequence: NP_009016.1;
SEQ ID NO: 96, follistatin-related protein 1 precursor [Mus musculus], NCBI Reference Sequence: NP_032073.2.

Exemplary SPP1 sequences include:
SPP1 secreted phosphoprotein 1 [Homo sapiens (human)], Homo sapiens chromosome 4, GRCh38.p13 Primary Assembly, NCBI Reference Sequence: NC_000004.12, >NC_000004.12 (87975650-87983411) Homo sapiens chromosome 4, GRCh38.p13 Primary Assembly;
Spp1 secreted phosphoprotein 1 [Mus musculus (house mouse)], Mus musculus strain C57BL/6J chromosome 5, GRCm38.p6 C57BL/6J, NCBI Reference Sequence: NC_000071.6, >NC_000071.6 (104435111-104441053) Mus musculus strain C57BL/6J chromosome 5, GRCm38.p6 C57BL/6J;
SEQ ID NO: 97, Homo sapiens secreted phosphoprotein 1 (SPP1), transcript variant 2, mRNA, NCBI Reference Sequence: NM_000582.2;
SEQ ID NO: 98, Mus musculus secreted phosphoprotein 1 (Spp1), transcript variant 1, mRNA, NCBI Reference Sequence: NM_001204201.1;
SEQ ID NO: 99, osteopontin isoform OPN-b precursor [Homo sapiens], NCBI Reference Sequence: NP_000573.1;
SEQ ID NO: 100, osteopontin isoform 1 [Mus musculus], NCBI Reference Sequence: NP_001191130.1.

Exemplary IFNE sequences include:
IFNE interferon epsilon [Homo sapiens (human)], Homo sapiens chromosome 9, GRCh38.p13 Primary Assembly, NCBI Reference Sequence: NC_000009.12, >NC_000009.12 (21480839-21482313, complement), Homo sapiens chromosome 9, GRCh38.p13 Primary Assembly;
Ifne interferon epsilon [Mus musculus (house mouse)], Mus musculus strain C57BL/6J chromosome 4, GRCm38.p6 C57BL/6J, NCBI Reference Sequence: NC_000070.6, >NC_000070.6 (88879538-88880201, complement) Mus musculus strain C57BL/6J chromosome 4, GRCm38.p6 C57BL/6J;
SEQ ID NO: 101, Homo sapiens interferon epsilon (IFNE), mRNA, NCBI Reference Sequence: NM_176891.4;
SEQ ID NO: 102, Mus musculus interferon epsilon (Ifne), mRNA, NCBI Reference Sequence: NM_177348.2;
SEQ ID NO: 103, interferon epsilon precursor [Homo sapiens], NCBI Reference Sequence: NP_795372.1;
SEQ ID NO: 104, interferon epsilon precursor [Mus musculus], NCBI Reference Sequence: NP_796322.1.

Exemplary Prl8a6 sequences include:
Prl8a6 prolactin family 8, subfamily a, member 6 [Mus musculus (house mouse)], Mus musculus strain C57BL/6J chromosome 13, GRCm38.p6 C57BL/6J, NCBI Reference Sequence: NC_000079.6, >NC_000079.6 (27432681-27438688, complement) Mus musculus strain C57BL/6J chromosome 13, GRCm38.p6 C57BL/6J;
SEQ ID NO: 105, see murine homolog, SEQ ID NO: 106;
SEQ ID NO: 106, Mus musculus prolactin family 8, subfamily a, member 6 (Prl8a6), transcript variant 1, mRNA, NCBI Reference Sequence: NM_001271378.1;
SEQ ID NO: 107, see murine homolog, SEQ ID NO: 108;
SEQ ID NO: 108, prolactin-8A6 isoform a precursor [Mus musculus], NCBI Reference Sequence: NP_001258307.1.

Exemplary SRPX2 sequences include:
SRPX2 sushi repeat containing protein X-linked 2 [Homo sapiens (human)], Homo sapiens chromosome X, GRCh38.p13 Primary Assembly, NCBI Reference Sequence: NC_000023.11, >NC_000023.11 (100644199-100675788) Homo sapiens chromosome X, GRCh38.p13 Primary Assembly;
Srpx2 sushi-repeat-containing protein, X-linked 2 [Mus musculus (house mouse)], Mus musculus strain C57BL/6J chromosome X, GRCm38.p6 C57BL/6J, NCBI Reference Sequence: NC_000086.7, >NC_000086.7 (133908416-133932448) Mus musculus strain C57BL/6J chromosome X, GRCm38.p6 C57BL/6J;
SEQ ID NO: 109, Homo sapiens sushi repeat containing protein X-linked 2 (SRPX2), mRNA, NCBI Reference Sequence: NM_014467.3;
SEQ ID NO: 110, Mus musculus sushi-repeat-containing protein, X-linked 2 (Srpx2), transcript variant 2, mRNA, NCBI Reference Sequence: NM_001083895.3;
SEQ ID NO: 111, sushi repeat-containing protein SRPX2 precursor [Homo sapiens], NCBI Reference Sequence: NP_055282.1;
SEQ ID NO: 112, sushi repeat-containing protein SRPX2 precursor [Mus musculus], NCBI Reference Sequence: NP_001077364.2.

Exemplary DHRS11 sequences include:
DHRS11 dehydrogenase/reductase 11 [Homo sapiens (human)], Homo sapiens chromosome 17, GRCh38.p13 Primary Assembly, NCBI Reference Sequence: NC_000017.11, >NC_000017.11 (36591846-36600804) Homo sapiens chromosome 17, GRCh38.p13 Primary Assembly;
Dhrs11 dehydrogenase/reductase (SDR family) member 11 [Mus musculus (house mouse)], Mus musculus strain C57BL/6J chromosome 11, GRCm38.p6 C57BL/6J, NCBI Reference Sequence: NC_000077.6, >NC_000077.6 (84820721-84829067, complement) Mus musculus strain C57BL/6J chromosome 11, GRCm38.p6 C57BL/6J;
SEQ ID NO: 113, Homo sapiens dehydrogenase/reductase 11 (DHRS11), mRNA, NCBI Reference Sequence: NM_024308.4;
SEQ ID NO: 114, Mus musculus dehydrogenase/reductase (SDR family) member 11 (Dhrs11), mRNA, NCBI Reference Sequence: NM_177564.5;
SEQ ID NO: 115, dehydrogenase/reductase SDR family member 11 precursor [Homo sapiens], NCBI Reference Sequence: NP_077284.2;
SEQ ID NO: 116, dehydrogenase/reductase SDR family member 11 precursor [Mus musculus], NCBI Reference Sequence: NP_808232.2.

Exemplary APOM sequences include:
APOM apolipoprotein M [Homo sapiens (human)], Homo sapiens chromosome 6, GRCh38.p13 Primary Assembly, NCBI Reference Sequence: NC_000006.12, >NC_000006.12 (31652404-31658210) Homo sapiens chromosome 6, GRCh38.p13 Primary Assembly;
Apom apolipoprotein M [Mus musculus (house mouse)], Mus musculus strain C57BL/6J chromosome 17, GRCm38.p6 C57BL/6J, NCBI Reference Sequence: NC_000083.6, >NC_000083.6 (35128997-35131801, complement) Mus musculus strain C57BL/6J chromosome 17, GRCm38.p6 C57BL/6J;
SEQ ID NO: 117. Homo sapiens apolipoprotein M (APOM), transcript variant 2, mRNA, NCBI Reference Sequence: NM_001256169.2;
SEQ ID NO: 118, Mus musculus apolipoprotein M (Apom), mRNA, NCBI Reference Sequence: NM_018816.2;
SEQ ID NO: 119, apolipoprotein M isoform 2 [Homo sapiens], NCBI Reference Sequence: NP_001243098.1;
SEQ ID NO: 120, apolipoprotein M precursor [Mus musculus], NCBI Reference Sequence: NP_061286.1.

Exemplary FAM3C sequences include:
FAM3C family with sequence similarity 3 member C [Homo sapiens (human)], Homo sapiens chromosome 7, GRCh38.p13 Primary Assembly, NCBI Reference Sequence: NC_000007.14, >NC 000007.14 (121348851-121396369, complement) Homo sapiens chromosome 7, GRCh38.p13 Primary Assembly;
Fam3c family with sequence similarity 3, member C [Mus musculus (house mouse)], Mus musculus strain C57BL/6J chromosome 6, GRCm38.p6 C57BL/6J, NCBI Reference Sequence: NC_000072.6, >NC_000072.6 (22306520-22356081, complement) Mus musculus strain C57BL/6J chromosome 6, GRCm38.p6 C57BL/6J;
SEQ ID NO: 121, Homo sapiens family with sequence similarity 3 member C (FAM3C), transcript variant 2, mRNA, NCBI Reference Sequence: NM_001040020.1;
SEQ ID NO: 122, Mus musculus family with sequence similarity 3, member C (Fam3c), mRNA, NCBI Reference Sequence: NM_138587.4;
SEQ ID NO: 123, protein FAM3C precursor [Homo sapiens], NCBI Reference Sequence: NP_001035109.1;
SEQ ID NO: 124, protein FAM3C precursor [Mus musculus], NCBI Reference Sequence: NP_613053.3.

Exemplary Scgb1b2 sequences include:
SCGB1B2P secretoglobin family 1B member 2, pseudogene [Homo sapiens (human)], Homo sapiens chromosome 19, GRCh38.p14 Primary Assembly, NCBI Reference Sequence: NC_000019.10, >NC_000019.10 (34576729-34677159 complement) Homo sapiens chromosome 19, GRCh38.p14 Primary Assembly;
Scgb1b2 secretoglobin, family 1B, member 2 [Mus musculus (house mouse)], Mus musculus strain C57BL/6J chromosome 7, GRCm38.p6 C57BL/6J, NCBI Reference Sequence: NC_000073.6, >NC_000073.6 (31290519-31291816, complement) Mus musculus strain C57BL/6J chromosome 7, GRCm38.p6 C57BL/6J;
SEQ ID NO: 125, Homo sapiens secretoglobin family 1B member 2, pseudogene (SCGB1B2P), transcript variant 1, non-coding RNA, NCBI Reference Sequence: NR_027620.4;
SEQ ID NO: 126, Mus musculus secretoglobin, family 1B, member 2 (Scgb1b2), mRNA, NCBI Reference Sequence: NM_020563.3;
SEQ ID NO: 127, see murine homolog, SEQ ID NO: 128;
SEQ ID NO: 128, androgen-binding protein eta precursor [Mus musculus], NCBI Reference Sequence: NP_065588.1.

Exemplary TIMP3 sequences include:
TIMP3 TIMP metallopeptidase inhibitor 3 [Homo sapiens (human)], Homo sapiens chromosome 22, GRCh38.p13 Primary Assembly, NCBI Reference Sequence: NC_000022.11, >NC_000022.11 (32800816-32863041) Homo sapiens chromosome 22, GRCh38.p13 Primary Assembly;
Timp3 tissue inhibitor of metalloproteinase 3 [Mus musculus (house mouse)], Mus musculus strain C57BL/6J chromosome 10, GRCm38.p6 C57BL/6J, NCBI Reference Sequence: NC_000076.6, >NC_000076.6 (86300412-86349505) Mus musculus strain C57BL/6J chromosome 10, GRCm38.p6 C57BL/6J;
SEQ ID NO: 129, Homo sapiens TIMP metallopeptidase inhibitor 3 (TIMP3), mRNA, NCBI Reference Sequence: NM_000362.4;
SEQ ID NO: 130, Mus musculus tissue inhibitor of metalloproteinase 3 (Timp3), mRNA, NCBI Reference Sequence: NM_011595.2;
SEQ ID NO: 131, metalloproteinase inhibitor 3 precursor [Homo sapiens], NCBI Reference Sequence: NP_000353.1;
SEQ ID NO: 132, metalloproteinase inhibitor 3 precursor [Mus musculus], NCBI Reference Sequence: NP_035725.1.

Exemplary VWA2 sequences include:
Homo sapiens von Willebrand factor A domain containing 2 (VWA2), Homo sapiens chromosome 10, GRCh38.p14 Primary Assembly, NCBI Reference Sequence: NC_000010.11, >NC_000010.11 (114239254-114294500) Homo sapiens chromosome 10, GRCh38.p14 Primary Assembly;
Mus musculus Willebrand factor A domain containing 2 (VWA2), Mus musculus strain C57BL/6J chromosome 19, GRCm38.p6 C57BL/6J, NCBI Reference Sequence: NC_000085.7, >NC_000085.7 (56862848-56900510) Mus musculus strain C57BL/6J chromosome 19, GRCm38.p6 C57BL/6J;
SEQ ID NO: 133, Homo sapiens von Willebrand factor A domain containing 2 (VWA2) mRNA, NCBI Reference Sequence: NM_001272046.2;
SEQ ID NO: 134. Mus musculus von Willebrand factor A domain containing 2 (Vwa2), mRNA, NCBI Reference Sequence: NM_172840.2;
SEQ ID NO: 135, von Willebrand factor A domain-containing protein 2 precursor [Homo sapiens], NCBI Reference Sequence: NP_001258975.1;
SEQ ID NO: 136, von Willebrand factor A domain-containing protein 2 precursor [Mus musculus], NCBI Reference Sequence: NP_766428.2.

Exemplary TFF3 sequences include:
TFF3 trefoil factor 3 [Homo sapiens (human)], Homo sapiens chromosome 21, GRCh38.p13 Primary Assembly, NCBI Reference Sequence: NC_000021.9, >NC_000021.9 (42311667-42315596, complement) Homo sapiens chromosome 21, GRCh38.p13 Primary Assembly;
Tff3 trefoil factor 3, intestinal [Mus musculus (house mouse)], Mus musculus strain C57BL/6J chromosome 17, GRCm38.p6 C57BL/6J, NCBI Reference Sequence: NC_000083.6, >NC_000083.6 (31125306-31129611, complement) Mus musculus strain C57BL/6J chromosome 17, GRCm38.p6 C57BL/6J;
SEQ ID NO: 137, Homo sapiens trefoil factor 3 (TFF3), mRNA, NCBI Reference Sequence: NM 003226.3;
SEQ ID NO: 138, Mus musculus trefoil factor 3, intestinal (Tff3), mRNA, NCBI Reference Sequence: NM_011575.2;
SEQ ID NO: 139, trefoil factor 3 precursor [Homo sapiens], NCBI Reference Sequence: NP_003217.3;
SEQ ID NO: 140, trefoil factor 3 precursor [Mus musculus], NCBI Reference Sequence: NP_035705.1.

Exemplary VSTM2A sequences include:
VSTM2A V-set and transmembrane domain containing 2A [Homo sapiens (human)], Homo sapiens chromosome 7, GRCh38.p13 Primary Assembly, NCBI Reference Sequence: NC_000007.14, >NC_000007.14 (54542064-54571080) Homo sapiens chromosome 7, GRCh38.p13 Primary Assembly;
Vstm2a V-set and transmembrane domain containing 2A [Mus musculus (house mouse)], Mus musculus strain C57BL/6J chromosome 11, GRCm38.p6 C57BL/6J, NCBI Reference Sequence: NC_000077.6, >NC_000077.6 (16257742-16284551) Mus musculus strain C57BL/6J chromosome 11, GRCm38.p6 C57BL/6J;
SEQ ID NO: 141, PREDICTED: Homo sapiens V-set and transmembrane domain containing 2A (VSTM2A), transcript variant X3, mRNA, NCBI Reference Sequence: XM_006715666.3;
SEQ ID NO: 142, Mus musculus V-set and transmembrane domain containing 2A (Vstm2a), transcript variant 1, mRNA, NCBI Reference Sequence: NM_001290539.2;
SEQ ID NO: 143, V-set and transmembrane domain-containing protein 2A isoform X3 [Homo sapiens], NCBI Reference Sequence: XP_006715729.1;
SEQ ID NO: 144, V-set and transmembrane domain-containing protein 2A isoform 1 precursor [Mus musculus], NCBI Reference Sequence: NP_001277468.1.

Exemplary GREM1 sequences include:
GREM1 gremlin 1, DAN family BMP antagonist [Homo sapiens (human)], Homo sapiens chromosome 15, GRCh38.p13 Primary Assembly, NCBI Reference Sequence: NC_000015.10, >NC_000015.10 (32718004-32745106) Homo sapiens chromosome 15, GRCh38.p13 Primary Assembly;
Grem1 gremlin 1, DAN family BMP antagonist [Mus musculus (house mouse)], Mus musculus strain C57BL/6J chromosome 2, GRCm38.p6 C57BL/6J, NCBI Reference Sequence: NC_000068.7, >NC_000068.7 (113748674-113759317, complement) Mus musculus strain C57BL/6J chromosome 2, GRCm38.p6 C57BL/6J;
SEQ ID NO: 145, Homo sapiens gremlin 1, DAN family BMP antagonist (GREM1), transcript variant 3, mRNA, NCBI Reference Sequence: NM_001191322.2;
SEQ ID NO: 146, Mus musculus gremlin 1, DAN family BMP antagonist (Grem1), mRNA, NCBI Reference Sequence: NM_011824.4;
SEQ ID NO: 147, gremlin-1 isoform 3 [Homo sapiens], NCBI Reference Sequence: NP_001178251.1;
SEQ ID NO: 148, gremlin-1 precursor [Mus musculus], NCBI Reference Sequence: NP_035954.1.

Exemplary TAC4 sequences include:
TAC4 tachykinin precursor 4 [Homo sapiens (human)], Homo sapiens chromosome 17, GRCh38.p13 Primary Assembly, NCBI Reference Sequence: NC_000017.11, >NC_000017.11 (49838309-49848017, complement) Homo sapiens chromosome 17, GRCh38.p13 Primary Assembly;
Tac4 tachykinin 4 [Mus musculus (house mouse)], Mus musculus strain C57BL/6J chromosome 11, GRCm38.p6 C57BL/6J, NCBI Reference Sequence: NC_000077.6, >NC_000077.6 (95261529-95269265) Mus musculus strain C57BL/6J chromosome 11, GRCm38.p6 C57BL/6J;
SEQ ID NO: 149, Homo sapiens tachykinin precursor 4 (TAC4), transcript variant beta, mRNA, NCBI Reference Sequence: NM_001077503.1;
SEQ ID NO: 150, Mus musculus tachykinin 4 (Tac4), mRNA, NCBI Reference Sequence: NM_053093.2;
SEQ ID NO: 151, tachykinin-4 isoform beta precursor [Homo sapiens], NCBI Reference Sequence: NP_001070971.1;
SEQ ID NO: 152, tachykinin-4 preproprotein [Mus musculus], NCBI Reference Sequence: NP_444323.1.

Exemplary CXCL5 sequences include:
CXCL5 C-X-C motif chemokine ligand 5 [Homo sapiens (human)], Homo sapiens chromosome 4, GRCh38.p13 Primary Assembly, NCBI Reference Sequence: NC_000004.12, >NC_000004.12 (73995642-73998677, complement) Homo sapiens chromosome 4, GRCh38.p13 Primary Assembly;
Cxcl5 chemokine (C-X-C motif) ligand 5 [Mus musculus (house mouse)], Mus musculus strain C57BL/6J chromosome 5, GRCm38.p6 C57BL/6J, NCBI Reference Sequence: NC_000071.6, >NC_000071.6 (90759298-90761625) Mus musculus strain C57BL/6J chromosome 5, GRCm38.p6 C57BL/6J;
SEQ ID NO: 153, Homo sapiens C-X-C motif chemokine ligand 5 (CXCL5), mRNA, NCBI Reference Sequence: NM_002994.5;
SEQ ID NO: 154, Mus musculus chemokine (C-X-C motif) ligand 5 (Cxcl5), mRNA, NCBI Reference Sequence: NM_009141.3;
SEQ ID NO: 155, C-X-C motif chemokine 5 precursor [Homo sapiens], NCBI Reference Sequence: NP_002985.1;
SEQ ID NO: 156, C-X-C motif chemokine 5 precursor [Mus musculus], NCBI Reference Sequence: NP_033167.2.

Exemplary SERPINE1 sequences include:
SERPINE1 serpin family E member 1 [Homo sapiens (human)], Homo sapiens chromosome 7, GRCh38.p13 Primary Assembly, NCBI Reference Sequence: NC_000007.14, >NC_000007.14 (101127089-101139266) Homo sapiens chromosome 7, GRCh38.p13 Primary Assembly;
Serpine1 serine (or cysteine) peptidase inhibitor, clade E, member 1 [Mus musculus (house mouse)], Mus musculus strain C57BL/6J chromosome 5, GRCm38.p6 C57BL/6J, NCBI Reference Sequence: NC_000071.6, >NC_000071.6 (137061504-137072272, complement) Mus musculus strain C57BL/6J chromosome 5, GRCm38.p6 C57BL/6J;
SEQ ID NO: 157, Homo sapiens serpin family E member 1 (SERPINE1), mRNA, NCBI Reference Sequence: NM_000602.4;
SEQ ID NO: 158, Mus musculus serine (or cysteine) peptidase inhibitor, clade E, member 1 (Serpine1), mRNA, NCBI Reference Sequence: NM_008871.2;
SEQ ID NO: 159, plasminogen activator inhibitor 1 precursor [Homo sapiens], NCBI Reference Sequence: NP_000593.1;
SEQ ID NO: 160, plasminogen activator inhibitor 1 precursor [Mus musculus], NCBI Reference Sequence: NP_032897.2.

Exemplary GSN sequences include:
GSN gelsolin [Homo sapiens (human)], Homo sapiens chromosome 9, GRCh38.p13 Primary Assembly, NCBI Reference Sequence: NC_000009.12, >NC_000009.12 (121201483-121332844) Homo sapiens chromosome 9, GRCh38.p13 Primary Assembly;
Gsn gelsolin [Mus musculus (house mouse)], Mus musculus strain C57BL/6J chromosome 2, GRCm38.p6 C57BL/6J, NCBI Reference Sequence: NC_000068.7,>NC_000068.7 (35256359-35307902) Mus musculus strain C57BL/6J chromosome 2, GRCm38.p6 C57BL/6J;
SEQ ID NO: 161, Homo sapiens gelsolin (GSN), transcript variant 1, mRNA, NCBI Reference Sequence: NM_000177.5;
SEQ ID NO: 162, Mus musculus gelsolin (Gsn), transcript variant 2, mRNA, NCBI Reference Sequence: NM_001206367.1;
SEQ ID NO: 163, gelsolin isoform a precursor [Homo sapiens], NCBI Reference Sequence: NP_000168.1;
SEQ ID NO: 164, gelsolin isoform 2 [Mus musculus], NCBI Reference Sequence: NP_001193296.1.

Exemplary OXT sequences include:
OXT oxytocin/neurophysin I prepropeptide [Homo sapiens (human)], Homo sapiens chromosome 20, GRCh38.p13 Primary Assembly, NCBI Reference Sequence: NC_000020.11, >NC_000020.11 (3068871-3072517) Homo sapiens chromosome 20, GRCh38.p13 Primary Assembly;
Oxt oxytocin [Mus musculus (house mouse)], Mus musculus strain C57BL/6J chromosome 2, GRCm38.p6 C57BL/6J, NCBI Reference Sequence: NC_000068.7, >NC_000068.7 (130574519-130577054) Mus musculus strain C57BL/6J chromosome 2, GRCm38.p6 C57BL/6J;
SEQ ID NO: 165, Homo sapiens oxytocin/neurophysin I prepropeptide (OXT), mRNA, NCBI Reference Sequence: NM_000915.4;
SEQ ID NO: 166, Mus musculus oxytocin (Oxt), mRNA, NCBI Reference Sequence: NM_011025.4;
SEQ ID NO: 167, oxytocin-neurophysin 1 preproprotein [Homo sapiens], NCBI Reference Sequence: NP_000906.1;
SEQ ID NO: 168, oxytocin-neurophysin 1 preproprotein [Mus musculus], NCBI Reference Sequence: NP_035155.1.

Exemplary CTF1 sequences include:
CTF1 cardiotrophin 1 [Homo sapiens (human)], Homo sapiens chromosome 16, GRCh38.p13 Primary Assembly, NCBI Reference Sequence: NC_000016.10, >NC_000016.10 (30895824-30903560) Homo sapiens chromosome 16, GRCh38.p13 Primary Assembly;
Ctf1 cardiotrophin 1 [Mus musculus (house mouse)], Mus musculus strain C57BL/6J chromosome 7, GRCm38.p6 C57BL/6J, NCBI Reference Sequence: NC_000073.6, >NC_000073.6 (127712676-127718188) Mus musculus strain C57BL/6J chromosome 7, GRCm38.p6 C57BL/6J;
SEQ ID NO: 169, Homo sapiens cardiotrophin 1 (CTF1), transcript variant 2, mRNA, NCBI Reference Sequence: NM_001142544.2;
SEQ ID NO: 170, Mus musculus cardiotrophin 1 (Ctf1), transcript variant 2, mRNA, NCBI Reference Sequence: NM_001301282.1;
SEQ ID NO: 171, cardiotrophin-1 isoform 2 [Homo sapiens], NCBI Reference Sequence: NP_001136016.1;
SEQ ID NO: 172, cardiotrophin-1 isoform 2 [Mus musculus], NCBI Reference Sequence: NP_001288211.1.

Exemplary MDK sequences include:
MDK midkine [Homo sapiens (human)], Homo sapiens chromosome 11, GRCh38.p13 Primary Assembly
NCBI Reference Sequence: NC_000011.10, >NC_000011.10 (46380784-46383837) Homo sapiens chromosome 11, GRCh38.p13 Primary Assembly;
Mdk midkine [Mus musculus (house mouse)], Mus musculus strain C57BL/6J chromosome 2, GRCm38.p6 C57BL/6J, NCBI Reference Sequence: NC_000068.7, >NC_000068.7 (91929805-91932297, complement) Mus musculus strain C57BL/6J chromosome 2, GRCm38.p6 C57BL/6J;
SEQ ID NO: 173, Homo sapiens midkine (MDK), transcript variant 2, mRNA, NCBI Reference Sequence: NM_001012333.2;
SEQ ID NO: 174, Mus musculus midkine (Mdk), transcript variant 2, mRNA, NCBI Reference Sequence: NM_001012335.2;
SEQ ID NO: 175, midkine isoform a precursor [Homo sapiens], NCBI Reference Sequence: NP_001012333.1;
SEQ ID NO: 176, midkine isoform a precursor [Mus musculus], NCBI Reference Sequence: NP_001012335.1.

Exemplary PLA2G2C sequences include:
PLA2G2C phospholipase A2 group IIC [Homo sapiens (human)], Homo sapiens chromosome 1, GRCh38.p13 Primary Assembly, NCBI Reference Sequence: NC_000001.11, >NC_000001.11 (20163055-20177439, complement) Homo sapiens chromosome 1, GRCh38.p13 Primary Assembly;
Pla2g2c phospholipase A2, group IIC [Mus musculus (house mouse)], Mus musculus strain C57BL/6J chromosome 4, GRCm38.p6 C57BL/6J, NCBI Reference Sequence: NC_000070.6, >NC_000070.6 (138725325-138744575) Mus musculus strain C57BL/6J chromosome 4, GRCm38.p6 C57BL/6J;
SEQ ID NO: 177, Homo sapiens phospholipase A2 group IIC (PLA2G2C), transcript variant 1, mRNA NCBI Reference Sequence: NM_001316722.2;
SEQ ID NO: 178, Mus musculus phospholipase A2, group IIC (Pla2g2c), mRNA, NCBI Reference Sequence: NM_008868.3;
SEQ ID NO: 179, putative inactive group IIC secretory phospholipase A2 isoform 1 precursor [Homo sapiens], NCBI Reference Sequence: NP_001303651.1;
SEQ ID NO: 180, group IIC secretory phospholipase A2 precursor [Mus musculus], NCBI Reference Sequence: NP_032894.2.

Exemplary NHLRC3 sequences include:
NHLRC3 NHL repeat containing 3 [Homo sapiens (human)], Homo sapiens chromosome 13, GRCh38.p13 Primary Assembly, NCBI Reference Sequence: NC_000013.11, >NC_000013.11 (39038311-39050109) Homo sapiens chromosome 13, GRCh38.p13 Primary Assembly;
Nhlrc3 NHL repeat containing 3 [Mus musculus (house mouse)], Mus musculus strain C57BL/6J chromosome 3, GRCm38.p6 C57BL/6J, NCBI Reference Sequence: NC_000069.6, >NC_000069.6 (53451996-53463258, complement) Mus musculus strain C57BL/6J chromosome 3, GRCm38.p6 C57BL/6J;
SEQ ID NO: 181, Homo sapiens NHL repeat containing 3 (NHLRC3), transcript variant 1, mRNA, NCBI Reference Sequence: NM_001012754.4;
SEQ ID NO: 182, Mus musculus NHL repeat containing 3 (Nhlrc3), mRNA, NCBI Reference Sequence: NM_172501.2;
SEQ ID NO: 183, NHL repeat-containing protein 3 isoform a precursor [Homo sapiens], NCBI Reference Sequence: NP_001012772.1;
SEQ ID NO: 184, NHL repeat-containing protein 3 precursor [Mus musculus], NCBI Reference Sequence: NP_766089.1.

Exemplary Glip111 sequences include:
Homo sapiens Glip111, Homo sapiens chromosome 12, GRCh38.p13 Primary Assembly, NCBI Reference Sequence: NC_000012.12, >NC_000012.12:75331834-75370560 Homo sapiens chromosome 12, GRCh38.p13 Primary Assembly;
Mus musculus Glip111, Mus musculus strain C57BL/6J chromosome 10, GRCm38.p6 C57BL/6J NCBI Reference Sequence: NC_000076.6, >NC_000076.6:112060189-112078510 Mus musculus strain, C57BL/6J chromosome 10, GRCm38.p6 C57BL/6J;
SEQ ID NO: 185, Homo sapiens GLIPR1 like 1 (GLIPR1L1), transcript variant 1, mRNA, NCBI Reference Sequence: NM_001304964.2;
SEQ ID NO: 186, Mus musculus GLI pathogenesis-related 1 like 1 (Glipr111), mRNA, NCBI Reference Sequence: NM_027018.1;
SEQ ID NO: 187, GLIPR1-like protein 1 isoform 1 precursor [Homo sapiens], NCBI Reference Sequence: NP_001291893.1;
SEQ ID NO: 188, GLIPR1-like protein 1 precursor [Mus musculus], NCBI Reference Sequence: NP_081294.1.

Exemplary TPSAB1 (Mcpt7) sequences include:
TPSAB1 tryptase alpha/beta 1 [Homo sapiens (human)], Homo sapiens chromosome 16, GRCh38.p13 Primary Assembly, NCBI Reference Sequence: NC_000016.10, >NC_000016.10 (1240705-1242554) Homo sapiens chromosome 16, GRCh38.p13 Primary Assembly;
Tpsab1 tryptase alpha/beta 1 [Mus musculus (house mouse)], Mus musculus strain C57BL/6J chromosome 17, GRCm38.p6 C57BL/6J, NCBI Reference Sequence: NC_000083.6, >NC_000083.6 (25343245-25345562, complement) Mus musculus strain C57BL/6J chromosome 17, GRCm38.p6 C57BL/6J;
SEQ ID NO: 189, Homo sapiens tryptase alpha/beta 1 (TPSAB1), mRNA, NCBI Reference Sequence: NM_003294.4;
SEQ ID NO: 190, Mus musculus tryptase alpha/beta 1 (Tpsab1), transcript variant 1, mRNA, NCBI Reference Sequence: NM_031187.4;
SEQ ID NO: 191, tryptase alpha/beta-1 precursor [Homo sapiens], NCBI Reference Sequence: NP_003285.2;
SEQ ID NO: 192, tryptase precursor [Mus musculus], NCBI Reference Sequence: NP_112464.4.

Exemplary IL12A sequences include:
IL12A interleukin 12A [Homo sapiens (human)], Homo sapiens chromosome 3, GRCh38.p13 Primary Assembly, NCBI Reference Sequence: NC_000003.12, >NC_000003.12 (159988835-159996019) Homo sapiens chromosome 3, GRCh38.p13 Primary Assembly;
Il12a interleukin 12a [Mus musculus (house mouse)], Mus musculus strain C57BL/6J chromosome 3, GRCm38.p6 C57BL/6J, NCBI Reference Sequence: NC_000069.6, >NC_000069.6 (68690644-68698550) Mus musculus strain C57BL/6J chromosome 3, GRCm38.p6 C57BL/6J;
SEQ ID NO: 193, Homo sapiens interleukin 12A (IL12A), transcript variant 1, mRNA, NCBI Reference Sequence: NM_000882.4;
SEQ ID NO: 194, Mus musculus interleukin 12a (Il12a), transcript variant 1, mRNA, NCBI Reference Sequence: NM_001159424.2;
SEQ ID NO: 195, interleukin-12 subunit alpha isoform 1 precursor [Homo sapiens], NCBI Reference Sequence: NP_000873.2;
SEQ ID NO: 196, interleukin-12 subunit alpha isoform 1 [Mus musculus], NCBI Reference Sequence: NP_001152896.1.

Exemplary RTF2 (2410001C21Rik) sequences include:
RTF2 replication termination factor 2 [Homo sapiens (human)], Homo sapiens chromosome 20, GRCh38.p13 Primary Assembly, NCBI Reference Sequence: NC_000020.11,>NC_000020.11 (56468585-56518886) Homo sapiens chromosome 20, GRCh38.p13 Primary Assembly;
Rtf2 replication termination factor 2 [Mus musculus (house mouse)] also known as 2410001C21Rik, Mus musculus strain C57BL/6J chromosome 2, GRCm38.p6 C57BL/6J, NCBI Reference Sequence: NC_000068.7, >NC_000068.7 (172440578-172469899) Mus musculus strain C57BL/6J chromosome 2, GRCm38.p6 C57BL/6J;
SEQ ID NO: 197, Homo sapiens replication termination factor 2 (RTF2), transcript variant 1, mRNA, NCBI Reference Sequence: NM_001283035.1;
SEQ ID NO: 198, Mus musculus replication termination factor 2 (Rtf2), mRNA, NCBI Reference Sequence: NM_025542.2;
SEQ ID NO: 199, replication termination factor 2 isoform a [Homo sapiens], NCBI Reference Sequence: NP_001269964.1;
SEQ ID NO: 200, replication termination factor 2 [Mus musculus], NCBI Reference Sequence: NP_079818.1.

Exemplary FJX1 sequences include:
FJX1 four-jointed box kinase 1 [Homo sapiens (human)], Homo sapiens chromosome 11, GRCh38.p13 Primary Assembly, NCBI Reference Sequence: NC_000011.10, >NC_000011.10 (35618460-35620865) Homo sapiens chromosome 11, GRCh38.p13 Primary Assembly;
Fjx1 four jointed box 1 [Mus musculus (house mouse)], Mus musculus strain C57BL/6J chromosome 2, GRCm38.p6 C57BL/6J, NCBI Reference Sequence: NC_000068.7, >NC_000068.7 (102449366-102451792, complement) Mus musculus strain C57BL/6J chromosome 2, GRCm38.p6 C57BL/6J;
SEQ ID NO: 201, Homo sapiens four-jointed box kinase 1 (FJX1), mRNA, NCBI Reference Sequence: NM_014344.4;
SEQ ID NO: 202, Mus musculus four jointed box 1 (Fjx1), mRNA, NCBI Reference Sequence: NM_010218.2;
SEQ ID NO: 203, four-jointed box protein 1 precursor [Homo sapiens], NCBI Reference Sequence: NP_055159.2;
SEQ ID NO: 204, four-jointed box protein 1 precursor [Mus musculus], NCBI Reference Sequence: NP_034348.2.

Exemplary CNTN4 sequences include:
CNTN4 contactin 4 [Homo sapiens (human)], Homo sapiens chromosome 3, GRCh38.p13 Primary Assembly, NCBI Reference Sequence: NC_000003.12, >NC_000003.12 (2098803-3059080) Homo sapiens chromosome 3, GRCh38.p13 Primary Assembly;
Cntn4 contactin 4 [Mus musculus (house mouse)], Mus musculus strain C57BL/6J chromosome 6, GRCm38.p6 C57BL/6J, NCBI Reference Sequence: NC_000072.6, >NC_000072.6 (105677632-106700141) Mus musculus strain C57BL/6J chromosome 6, GRCm38.p6 C57BL/6J;
SEQ ID NO: 205, Homo sapiens contactin 4 (CNTN4), transcript variant 4, mRNA, NCBI Reference Sequence: NM_001206955.1;
SEQ ID NO: 206, Mus musculus contactin 4 (Cntn4), transcript variant 1, mRNA, NCBI Reference Sequence: NM_001109749.1;
SEQ ID NO: 207, contactin-4 isoform a precursor [Homo sapiens], NCBI Reference Sequence: NP_001193884.1;
SEQ ID NO: 208, contactin-4 isoform 1 precursor [Mus musculus], NCBI Reference Sequence: NP_001103219.1.

Exemplary FETUB sequences include:
FETUB fetuin B [Homo sapiens (human)], Homo sapiens chromosome 3, GRCh38.p13 Primary Assembly, NCBI Reference Sequence: NC_000003.12, >NC_000003.12 (186635828-186653141) Homo sapiens chromosome 3, GRCh38.p13 Primary Assembly;
Fetub fetuin beta [Mus musculus (house mouse)], Mus musculus strain C57BL/6J chromosome 16, GRCm38.p6 C57BL/6J, NCBI Reference Sequence: NC_000082.6, >NC_000082.6 (22918382-22939768) Mus musculus strain C57BL/6J chromosome 16, GRCm38.p6 C57BL/6J;
SEQ ID NO: 209, Homo sapiens fetuin B (FETUB), transcript variant 2, mRNA, NCBI Reference Sequence: NM_001308077.2;
SEQ ID NO: 210, Mus musculus fetuin beta (Fetub), transcript variant 2, mRNA, NCBI Reference Sequence: NM_001083904.1;
SEQ ID NO: 211, fetuin-B isoform 2 precursor [Homo sapiens], NCBI Reference Sequence: NP_001295006.1;
SEQ ID NO: 212, fetuin-B isoform 2 [Mus musculus], NCBI Reference Sequence: NP_001077373.1.

Exemplary CCL6 sequences include:
Ccl6 chemokine (C-C motif) ligand 6 [Mus musculus (house mouse)], Mus musculus strain C57BL/6J chromosome 11, GRCm38.p6 C57BL/6J, NCBI Reference Sequence: NC_000077.6, >NC_000077.6 (83587886-83593087, complement) Mus musculus strain C57BL/6J chromosome 11, GRCm38.p6 C57BL/6J;
SEQ ID NO: 213, see murine homolog, SEQ ID NO: 214;
SEQ ID NO: 214, Mus musculus chemokine (C-C motif) ligand 6 (Ccl6), mRNA, NCBI Reference Sequence: NM_009139.3;
SEQ ID NO: 215, see murine homolog, SEQ ID NO: 216;
SEQ ID NO: 216, C-C motif chemokine 6 precursor [Mus musculus], NCBI Reference Sequence: NP_033165.1.

Exemplary THBS2 sequences include:
THBS2 thrombospondin 2 [Homo sapiens (human)], Homo sapiens chromosome 6, GRCh38.p13 Primary Assembly, NCBI Reference Sequence: NC_000006.12, >NC_000006.12 (169215780-169254114, complement) Homo sapiens chromosome 6, GRCh38.p13 Primary Assembly;
Thbs2 thrombospondin 2 [Mus musculus (house mouse)], Mus musculus strain C57BL/6J chromosome 17, GRCm38.p6 C57BL/6J, NCBI Reference Sequence: NC_000083.6, >NC_000083.6 (14665500-14694262, complement) Mus musculus strain C57BL/6J chromosome 17, GRCm38.p6 C57BL/6J;
SEQ ID NO: 217, Homo sapiens thrombospondin 2 (THBS2), mRNA, NCBI Reference Sequence: NM_003247.3;
SEQ ID NO: 218, Mus musculus thrombospondin 2 (Thbs2), mRNA, NCBI Reference Sequence: NM_011581.3;
SEQ ID NO: 219, thrombospondin-2 precursor [Homo sapiens], NCBI Reference Sequence: NP_003238.2;
SEQ ID NO: 220, thrombospondin-2 precursor [Mus musculus], NCBI Reference Sequence: NP_035711.2.

Exemplary FBLN2 sequences include:
FBLN2 fibulin 2 [Homo sapiens (human)], Homo sapiens chromosome 3, GRCh38.p13 Primary Assembly
NCBI Reference Sequence: NC_000003.12, >NC_000003.12 (13549125-13638408) Homo sapiens chromosome 3, GRCh38.p13 Primary Assembly;
Fbln2 fibulin 2 [Mus musculus (house mouse)], Mus musculus strain C57BL/6J chromosome 6, GRCm38.p6 C57BL/6J, NCBI Reference Sequence: NC_000072.6, >NC_000072.6 (91212460-91272540) Mus musculus strain C57BL/6J chromosome 6, GRCm38.p6 C57BL/6J;
SEQ ID NO: 221, Homo sapiens fibulin 2 (FBLN2), transcript variant 1, mRNA, NCBI Reference Sequence: NM_001004019.2;
SEQ ID NO: 222, Mus musculus fibulin 2 (Fbln2), transcript variant 2, mRNA, NCBI Reference Sequence: NM_001081437.1;
SEQ ID NO: 223, fibulin-2 isoform a precursor [Homo sapiens], NCBI Reference Sequence: NP_001004019.1;
SEQ ID NO: 224, fibulin-2 isoform b precursor [Mus musculus], NCBI Reference Sequence: NP_001074906.1.

Exemplary HTRA1 sequences include:
HTRA1 HtrA serine peptidase 1 [Homo sapiens (human)], Homo sapiens chromosome 10, GRCh38.p13 Primary Assembly, NCBI Reference Sequence: NC_000010.11, >NC_000010.11 (122461553-122514907) Homo sapiens chromosome 10, GRCh38.p13 Primary Assembly;
Htra1 HtrA serine peptidase 1 [Mus musculus (house mouse)], Mus musculus strain C57BL/6J chromosome 7, GRCm38.p6 C57BL/6J, NCBI Reference Sequence: NC_000073.6, >NC_000073.6 (130936203-130985658) Mus musculus strain C57BL/6J chromosome 7, GRCm38.p6 C57BL/6J;
SEQ ID NO: 225, Homo sapiens HtrA serine peptidase 1 (HTRA1), mRNA, NCBI Reference Sequence: NM_002775.5;
SEQ ID NO: 226, Mus musculus HtrA serine peptidase 1 (Htra1), mRNA, NCBI Reference Sequence: NM_019564.3;
SEQ ID NO: 227, serine protease HTRA1 precursor [Homo sapiens], NCBI Reference Sequence: NP_002766.1;
SEQ ID NO: 228, serine protease HTRA1 precursor [Mus musculus], NCBI Reference Sequence: NP_062510.2.

Exemplary SFRP1 sequences include:
SFRP1 secreted frizzled related protein 1 [Homo sapiens (human)], Homo sapiens chromosome 8, GRCh38.p13 Primary Assembly, NCBI Reference Sequence: NC_000008.11, >NC_000008.11 (41261962-41309473, complement) Homo sapiens chromosome 8, GRCh38.p13 Primary Assembly;
Sfrp1 secreted frizzled-related protein 1 [Mus musculus (house mouse)], Mus musculus strain C57BL/6J chromosome 8, GRCm38.p6 C57BL/6J, NCBI Reference Sequence: NC_000074.6, >NC_000074.6 (23411383-23449632) Mus musculus strain C57BL/6J chromosome 8, GRCm38.p6 C57BL/6J;
SEQ ID NO: 229, Homo sapiens secreted frizzled related protein 1 (SFRP1), mRNA, NCBI Reference Sequence: NM_003012.5;
SEQ ID NO: 230, Mus musculus secreted frizzled-related protein 1 (Sfrp1), mRNA, NCBI Reference Sequence: NM_013834.3;
SEQ ID NO: 231, secreted frizzled-related protein 1 precursor [Homo sapiens], NCBI Reference Sequence: NP_003003.3;
SEQ ID NO: 232, secreted frizzled-related protein 1 precursor [Mus musculus], NCBI Reference Sequence: NP_038862.2.

Exemplary WNT6 sequences include:
WNT6 Wnt family member 6 [Homo sapiens (human)], Homo sapiens chromosome 2, GRCh38.p13 Primary Assembly, NCBI Reference Sequence: NC_000002.12, >NC_000002.12 (218859805-218874233) Homo sapiens chromosome 2, GRCh38.p13 Primary Assembly;
Wnt6 wingless-type MMTV integration site family, member 6 [Mus musculus (house mouse)], Mus musculus strain C57BL/6J chromosome 1, GRCm38.p6 C57BL/6J, NCBI Reference Sequence: NC_000067.6
>NC_000067.6 (74754342-74785322) Mus musculus strain C57BL/6J chromosome 1, GRCm38.p6 C57BL/6J;
SEQ ID NO: 233, Homo sapiens Wnt family member 6 (WNT6), mRNA, NCBI Reference Sequence: NM_006522.4;
SEQ ID NO: 234, Mus musculus wingless-type MMTV integration site family, member 6 (Wnt6), mRNA, NCBI Reference Sequence: NM_009526.3;
SEQ ID NO: 235, protein Wnt-6 precursor [Homo sapiens], NCBI Reference Sequence: NP_006513.1;
SEQ ID NO: 236, protein Wnt-6 precursor [Mus musculus], NCBI Reference Sequence: NP_033552.2.

Exemplary IMPG2 sequences include:
IMPG2 interphotoreceptor matrix proteoglycan 2 [Homo sapiens (human)], Homo sapiens chromosome 3, GRCh38.p13 Primary Assembly, NCBI Reference Sequence: NC_000003.12, >NC_000003.12 (101222546-101320575, complement) Homo sapiens chromosome 3, GRCh38.p13 Primary Assembly;
Impg2 interphotoreceptor matrix proteoglycan 2 [Mus musculus (house mouse)], Mus musculus strain C57BL/6J chromosome 16, GRCm38.p6 C57BL/6J, NCBI Reference Sequence: NC_000082.6, >NC_000082.6 (56203839-56273753) Mus musculus strain C57BL/6J chromosome 16, GRCm38.p6 C57BL/6J;
SEQ ID NO: 237, Homo sapiens interphotoreceptor matrix proteoglycan 2 (IMPG2), mRNA, NCBI Reference Sequence: NM_016247.4;
SEQ ID NO: 238, Mus musculus interphotoreceptor matrix proteoglycan 2 (Impg2), mRNA, NCBI Reference Sequence: NM_174876.3;
SEQ ID NO: 239, interphotoreceptor matrix proteoglycan 2 precursor [Homo sapiens], NCBI Reference Sequence: NP_057331.2;
SEQ ID NO: 240, interphotoreceptor matrix proteoglycan 2 precursor [Mus musculus], NCBI Reference Sequence: NP_777365.2.

1. A composition comprising cardiomyocytes in admixture with an isolated polypeptide selected from the group consisting of: the polypeptides listed in Table 1.

2. A composition comprising cardiomyocytes in admixture with two or more isolated polypeptides selected from the group consisting of: the polypeptides listed in Table 1.

3. The composition of claim 1 or claim 2, wherein the cardiomyocytes are human cardiomyocytes.

4. The composition of any one of claims 1-3, wherein the cardiomyocytes are differentiated in vitro from embryonic stem cells or from induced pluripotent stem cells.

5. A transplant composition comprising the composition of any one of claims 1-4.

6. A composition comprising cardiomyocytes that have been contacted with an isolated polypeptide selected from the group consisting of: the polypeptides listed in Table 1.

7. A composition comprising cardiomyocytes that have been contacted with two or more isolated polypeptides selected from the group consisting of: the polypeptides listed in Table 1.

8. The composition of claim 6 or claim 1 or claim 2, wherein the cardiomyocytes are human cardiomyocytes.

9. The composition of any one of claims 1-3, wherein the cardiomyocytes are differentiated in vitro from embryonic stem cells or from induced pluripotent stem cells.

10. A transplant composition comprising the composition of any one of claims 6-9.

11. A composition comprising cardiomyocytes and a nucleic acid construct encoding a polypeptide selected from the group consisting of: the polypeptides listed in Table 1.

12. A composition comprising cardiomyocytes and one or more nucleic acid constructs encoding two or more polypeptides selected from the group consisting of: the polypeptides listed in Table 1.

13. The composition of claim 11 or 12, wherein the construct is in a vector.

14. The composition of claim 11 or 12, wherein the construct or constructs is/are in admixture with a transfection reagent.

15. The composition of any one of claims 11-14, wherein the cardiomyocytes are human cardiomyocytes.

16. The composition of any one of claims 11-15, wherein the cardiomyocytes are differentiated in vitro from embryonic stem cells or induced pluripotent stem cells.

17. A transplant composition comprising the composition of any one of claims 11-16.

18. A composition comprising cardiomyocytes in admixture with an isolated metabolic factor polypeptide that promotes transplant engraftment of the cardiomyocytes, or with a nucleic acid construct that encodes such a factor.

19. The composition of claim 18, wherein the isolated metabolic factor is selected from the group consisting of a polypeptide that promotes lipid hydrolysis and a polypeptide that modulates insulin or IGF signaling.

20. The composition of claim 18, wherein the polypeptide that promotes lipid hydrolysis is selected from LIPM, PSAP and PLA2G2C, and the polypeptide that modulates insulin or IGF signaling is selected from SERPINA12, HTRA1 and FETUB.

21. A composition comprising cardiomyocytes in admixture with an isolated vascular remodeling, extracellular matrix, proteoglycan or cell adhesion polypeptide that promotes transplant engraftment of the cardiomyocytes, or with a nucleic acid construct that encodes such a factor.

22. The composition of claim 21, which comprises two or more polypeptide factors selected from the groups consisting of vascular remodeling, extracellular matrix, proteoglycan and cell adhesion polypeptides.

23. The composition of claim 21, wherein the vascular remodeling polypeptide is selected from the group consisting of the polypeptides listed in Table 6, the extracellular matrix polypeptide is selected from the group consisting of the polypeptides listed in Table 7, the proteoglycan polypeptide is selected from the polypeptides listed in Table 8, and the cell adhesion polypeptide is selected from the polypeptides listed in Table 9.

24. A composition comprising cardiomyocytes in admixture with an isolated canonical Wnt pathway polypeptide.

25. A composition comprising cardiomyocytes that have been contacted with an isolated canonical Wnt pathway polypeptide.

26. The composition of claim 24 or 25, wherein the canonical Wnt pathway polypeptide is selected from the group consisting of the polypeptides listed in Table 2.

27. A composition comprising cardiomyocytes in admixture with an isolated serine protease polypeptide.

28. A composition comprising cardiomyocytes that have been contacted with an isolated serine protease polypeptide.

29. The composition of claim 27 or 28, wherein the serine protease polypeptide is selected from the group consisting of the polypeptides listed in Table 10.

30. A composition comprising cardiomyocytes in admixture with an isolated serine protease inhibitor polypeptide.

31. A composition comprising cardiomyocytes that have been contacted with an isolated serine protease inhibitor polypeptide.

32. The composition of claim 30 or 31, wherein the isolated serine protease inhibitor polypeptide is selected from the group consisting of the polypeptides listed in Table 11.

33. A composition comprising cardiomyocytes in admixture with an isolated signaling polypeptide of the interleukin family, interferon signaling family, or chemokine family.

34. A composition comprising cardiomyocytes that have been contacted with an isolated signaling polypeptide of the interleukin family, interferon signaling family, or chemokine family.

35. The composition of claim 33 or 34, wherein the signaling polypeptide of the interleukin family is selected from the group consisting of the polypeptides listed in Table 12, the signaling polypeptide of the interferon signaling family is selected from the polypeptides listed in Table 13, and the signaling polypeptide of the chemokine family is selected from the group consisting of the polypeptides listed in Table 14.

36. A composition comprising cardiomyocytes in admixture with an isolated TLR binding polypeptide, a lipocalin polypeptide or a secretaglobin polypeptide.

37. A composition comprising cardiomyocytes that have been contacted with an isolated TLR binding polypeptide, a lipocalin polypeptide or a secretaglobin polypeptide.

38. The composition of claim 36 or 37, wherein the TLR binding polypeptide is selected from the polypeptides listed in Table 15, the lipocalin polypeptide is selected from the polypeptides listed in Table 16, and the secretaglobin polypeptide is selected from the polypeptides listed in Table 17.

39. A cardiac delivery device comprising a composition of any one of claims 1-38.

40. A method of transplanting cardiomyocytes, the method comprising administering a composition of any one of claims 1-38 to cardiac tissue, optionally using the cardiac delivery device of claim 39.

41. The method of claim 40, wherein the engraftment of the administered cardiomyocytes is increased relative to engraftment of cardiomyocytes that were not in admixture with or had not been contacted with the polypeptide or polypeptides.

42. A method of enhancing cardiomyocyte transplant engraftment, the method comprising contacting a cardiomyocyte population with a polypeptide selected from the group consisting of the polypeptides listed in Table 1, and transplanting the cardiomyocyte population to cardiac tissue.

43. A method of enhancing cardiomyocyte transplant engraftment, the method comprising contacting a cardiomyocyte population with a nucleic acid that encodes a polypeptide selected from the group consisting of the polypeptides listed in Table 1, and transplanting the cardiomyocyte population to cardiac tissue.

44. A method of enhancing cardiomyocyte transplant engraftment, the method comprising contacting a cardiomyocyte population with a vector that encodes a polypeptide selected from the group consisting of the polypeptides listed in Table 1, and transplanting the cardiomyocyte population to cardiac tissue.

45. The method of claim 44, wherein the vector comprises an AAV vector.

46. The method of any one of claims 42-45, wherein the cardiomyocyte population is a human cardiomyocyte population.

47. The method of any one of claims 42-46, wherein the cardiomyocyte population is differentiated in vitro from embryonic stem cells or induced pluripotent stem cells.

48. The method of claim 47, wherein the induced pluripotent stem cells are differentiated from induced pluripotent stem cells derived from the transplant recipient.
